(19) United States
(12) Patent Application Publication
(10) Pub. No.: US 2004/0097492 A1
(43) Pub. Date: May 20, 2004

(54) Anti-infective agents

(76) Inventors: John K. Pratt, Kenosha, WI (US); David A. Betebenner, Libertyville, IL (US); Pamela L. Donner, Mundelein, IL (US); Brian E. Green, Wonder Lake, IL (US); Dale J. Kempf, Libertyville, IL (US); Keith F. McDaniel, Wauconda, IL (US); Clarence J. Maring, Palatine, IL (US); Vincent S. Stoll, Libertyville, IL (US); Rong Zhang, Skokie, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/285,714

(22) Filed: Nov. 1, 2002

(51) Int. Cl. A61K 31/549; C07D 498/02; C07D 285/16; C07D 285/22; C07D 285/34

(52) U.S. Cl. 514/222.8; 514/223.2; 544/10; 544/12

(57) ABSTRACT

Compounds having the formula

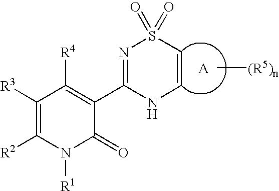

are hepatitis C(HCV) polymerase inhibitors. Also disclosed are a composition and method for inhibiting hepatitis C(HCV) polymerase, processes for making the compounds, and synthetic intermediates employed in the processes.

### TECHNICAL FIELD

The present invention relates to novel anti-infective agents. Specifically, the present invention relates to compounds, a composition, a method for inhibiting hepatitis C virus (HCV) polymerase, a method for inhibiting HCV viral replication, and a method for treating or preventing HCV infection, and processes for making the compounds, and synthetic intermediates employed in the processes.

### BACKGROUND OF THE INVENTION

Infection with hepatitis C virus (HCV) is a major cause of human liver disease throughout the world. More than 85% of all infected individuals become chronically infected. Chronic HCV infection accounts for 30% of all cirrhosis, end-stage liver disease, and liver cancer in the United States. The CDC estimates that the number of deaths due to HCV will increase to 38,000/year by the year 2010.

While initially therapy consisted of interferon alone, the combination of interferon alpha-2b with ribavirin for either 24 or 48 weeks is currently the most efficacious approved therapy for the treatment of chronic HCV infection. However, there are many adverse side effects associated with this therapy (flu-like symptoms, leukopenia, thrombocytopenia, and depression from interferon, as well as anemia induced by ribavirin). Furthermore, this therapy is less effective against infections caused by HCV genotype 1 which constitutes about 75% of all HCV infections.

Based on the foregoing, there exists a significant need to identify compounds with the ability to inhibit HCV. The present invention provides novel anti-infective agents which are HCV polymerase inhibitors.

### SUMMARY OF THE INVENTION

In its principle embodiment, the present invention provides a compound of formula (I)

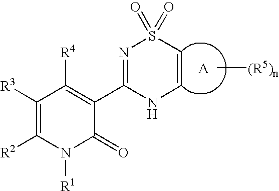

(I),

or a pharmaceutically acceptable salt thereof, wherein

n is 0, 1, 2, 3, or 4;

A is a five- or six-membered ring selected from the group consisting of aryl, cycloalkyl, cycloalkenyl, heteroaryl and heterocycle; wherein the five- or six-membered ring is optionally fused to a second five, or six-membered ring selected from the group consisting of aryl, cycloalkyl, heteroaryl and heterocycle;

R¹ is selected from the group consisting of hydrogen, alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, aryl, arylalkenyl, arylalkyl, arylsulfanylalkyl, arylsulfonylalkyl, carboxyalkyl, cyanoalkyl, cycloalkenyl, cycloalkenylalkyl, cycloalkyl, cycloalkylalkenyl, cycloalkylalkyl, formylalkyl, haloalkoxyalkyl, haloalkyl, heteroaryl, heteroarylalkenyl, heteroarylalkyl, heteroarylsulfonylalkyl, heterocycle, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— and RₖO—;

R² and R³ are independently selected from the group consisting of hydrogen, alkenyl, alkoxyalkyl, alkoxycarbonyl, alkyl, alkylcarbonyl, aryl, arylalkyl, arylcarbonyl, heteroaryl, heteroarylalkyl, heteroarylcarbonyl, heterocyclecarbonyl, cyano, halo and RₐR_{b}NC(O)—; or

R² and R³, together with the carbon atoms to which they are attached form a ring selected from the group consisting of aryl, cycloalkyl, heteroaryl and heterocycle;

R⁴ is selected from the group consisting of alkoxy, arylalkoxy, aryloxy, halo, hydroxy, RₐR_{b}N—, N₃—, R_{c}S—;

each R⁵ is independently selected from the group consisting of alkenyl, alkoxy, alkoxyalkyl, alkoxycarbonyl, alkyl, alkylcarbonyl, alkylsulfonyl, alkynyl, aryl, arylalkyl, arylcarbonyl, aryloxy, aryloxyalkyl, arylalkoxy, arylsulfonyl, halo, haloalkyl, heteroaryl, heteroarylalkyl, heteroarylcarbonyl, heterocycle, heterocyclealkyl, heterocyclecarbonyl, hydroxy, hydoxyalkyl, cycloalkyl, cyano, nitro, RₐR_{b}N—, RₐR_{b}Nalkyl and RₐR_{b}NC(O)—;

Rₐ and R_{b} are independently selected from the group consisting of hydrogen, alkenyl, alkoxyalkyl, alkoxycarbonyl, alkoxycarbonylalkyl, alkyl, alkylcarbonyl, alkylcarbonylalkyl, alkylsulfonyl, alkylsulfonylalkyl, aryl, arylalkenyl, arylalkyl, arylcarbonyl, arylsulfonyl, arylsulfonylalkyl, cyanoalkyl, cycloalkenyl, cycloalkenylalkyl, cycloalkyl, cycloalkylalkyl, cycloalkylalkenyl, formylalkyl, haloalkyl, heteroaryl, heteroarylalkenyl, heteroarylalkyl, heteroarylcarbonyl, heteroarylsulfonylalkyl, heterocycle, heterocyclealkenyl, heterocyclealkyl, heterocyclecarbonyl, heteroarylsulfonyl, hydroxyalkyl, nitroalkyl, R_{c}R_{d}N—, R_{c}R_{d}Nalkyl-, R_{c}R_{d}NalkylC(O)—, R_{c}R_{d}NC(O)Oalkyl-, R_{c}R_{d}NC(O)N(Rₑ)alkyl-;

R_{c}, R_{d}, and Rₑ are independently selected from the group consisting of hydrogen, alkenyl, alkyl and cycloalkyl;

R_{f} and R_{g} are independently selected from the group consisting of hydrogen, alkyl, alkenyl, aryl, arylalkyl, cycloalkyl, cycloalkylalkyl, cycloalkenyl, heterocycle, heterocyclealkyl, heteroaryl and heteroarylalkyl; or

R_{f} and R_{g} together with the carbon atom to which they are attached form a ring selected from the group consisting of cycloalkyl, cycloalkenyl and heterocycle;

Rₖ is selected from the group consisting of alkenyl, alkoxyalkyl, alkoxycarbonyl, alkoxycarbonylalkyl, alkyl, alkylcarbonylalkyl, alkylsulfanylalkyl, alkylsulfonylalkyl, aryl, arylalkyl, arylsulfanylalkyl, carboxyalkyl, cyanoalkyl, cycloalkenyl, cycloalkenylalkyl, cycloalkyl, cycloalkylalkyl, formylalkyl, haloalkoxyalkyl, haloalkyl, heteroaryl, heteroarylalkyl, heterocycle, heterocyclealkyl, hydroxyalkyl, nitroalkyl, R_{c}R_{d}Nalkyl-, R_{c}R_{d}NC(O)— and R_{c}R_{d}NC(O)alkyl;

provided that when R² and R³, together with the carbon atoms to which they are attached, form a phenyl ring, and R¹ is alkyl, alkenyl, arylalkyl, aryl or heteroaryl, and R⁴ is alkoxy, arylalkoxy, aryloxy, hydroxy or R_{c}S—, then A is other than phenyl.

In another embodiment of the present invention there is disclosed a pharmaceutical composition comprising a compound of claim 1 or a pharmaceutically acceptable salt thereof, in combination with a pharmaceutically acceptable carrier.

In a further embodiment of the present invention there is disclosed a method of treating or preventing infection which comprises administering to a patient in need of such treatment a therapeuctially effective amount of a compound of claim 1 or a pharmaceutically acceptable salt thereof.

In a still further embodiment of the present invention there is disclosed a method of treating or preventing infection wherein the infection is hepatitis C virus comprising administering to a patient in need of such treatment a therapeuctially effective amount of a compound of claim 1 or a pharmaceutically acceptable salt thereof.

In a still further embodiment of the present invention there is disclosed a method of inhibiting HCV polymerase comprises administering to a patient in need of such treatment a therapeuctially effective amount of a compound of claim 1 or a pharmaceutically acceptable salt thereof.

### DETAILED DESCRIPTION OF THE INVENTION

As used in the present specification the following terms have the meanings indicated:

As used herein, the singular forms “a”, to “an”, and “the” include plural reference unless the context clearly dictates otherwise.

The term “alkenyl,” as used herein, refers to a straight or branched chain group of two to six carbon atoms containing at least one carbon-carbon double bond. Examples of alkenyl groups include allyl, propenyl, 3-methyl-2-butenyl, and the like.

The term “alkoxy,” as used herein, refers to an alkyl group attached to the parent molecular moiety through an oxygen atom. Examples of alkoxy groups include tert-butoxy, methoxy, isopropoxy, and the like.

The term “alkoxyalkyl,” as used herein, refers to an alkyl group substituted by at least one alkoxy group.

The term “alkoxycarbonyl,” as used herein, refers to an alkoxy group attached to the parent molecular moiety through a carbonyl group. Examples of alkoxycarbonyl groups include tert-butoxycarbonyl, ethoxycarbonyl, methoxycarbonyl, and the like.

The term “alkoxycarbonylalkyl,” as used herein, refers to an alkoxycarbonyl group attached to the parent molecular moiety through an alkyl group.

The term “alkyl,” as used herein, refers to a group derived from a straight or branched chain saturated hydrocarbon containing from one to ten carbon atoms. Examples of alkyl groups include butyl, methyl, 2-methylbutyl, and the like.

The term “alkylcarbonyl,” as used herein, refers to an alkyl group attached to the parent molecular moiety through a carbonyl group. Examples of alkylcarbonyl groups include acyl, butanoyl, 2,2-dimethylpropanoyl, and the like.

The term “alkylcarbonylalkyl,” as used herein, refers to an alkoxycarbonyl group attached to the parent molecular moiety through an alkyl group.

The term “alkynyl,” as used herein, refers to a straight or branched chain hydrocarbon of two to six carbon atoms containing at least one carbon-carbon triple bond. Examples of alkynyl groups include ethynyl, 2-methyl-3-butynyl, 3-pentynyl, and the like.

The term “alkylsulfanyl,” as used herein, refers to an alkyl group attached to the parent molecular moiety through a sulfur atom. Examples of alkylsulfanyl groups include methylsulfanyl, (1-methylethyl)sulfanyl, (2-methylpropyl)sulfanyl, and the like.

The term “alkylsulfanylalkyl,” as used herein, refers to an alkylsulfanyl group attached to the parent molecular moiety through an alkyl group.

The term “alkylsulfinyl,” as used herein, refers to an alkyl group attached to the parent molecular moiety through a —S(O)— group.

The term “alkylsulfinylalkyl,” as used herein, refers to an alkylsulfinyl group attached to the parent molecular moiety through an alkyl group.

The term “alkylsulfonyl,” as used herein, refers to an alkyl group attached to the parent molecular moiety through a —S(O)₂— group.

The term “alkylsulfonylalkyl,” as used herein, refers to an alkylsulfonyl group attached to the parent molecular moiety through an alkyl group.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a monocyclic cycloalkenyl group, as defined herein, a monocyclic cycloalkyl group, as defined herein, or another phenyl group. Tricyclic fused ring systems are exemplified by a bicyclic fused ring system fused to a monocyclic cycloalkenyl group, as defined herein, a monocyclic cycloalkyl group, as defined herein, or another phenyl group. Examples of aryl groups include anthracenyl, azulenyl, fluorenyl, indanyl, indenyl, naphthyl, phenyl, tetrahydronaphthyl, and the like. The aryl groups of the present invention can be substituted with 0, 1, 2, 3, 4 or 5 substituents independently selected from the group consisting of alkoxy, alkoxyalkyl, alkoxycarbonyl, alkyl, alkylcarbonyl, alkylsulfanyl, alkylsulfonyl, a second aryl group, arylalkyl, arylcarbonyl, aryloxy, arylsulfanyl, arylsulfonyl, carboxy, cyano, halo, haloalkoxy, haloalkyl, heteroaryl, heteroarylalkyl, heterocycle, heterocyclealkyl, heterocyclecarbonyl, hydroxy, hydroxyalkyl, nitro, R_{c}R_{d}N—, R_{c}R_{d}Nalkyl and R_{c}R_{d}NC(O)—, wherein R_{c} and R_{d} are defined herein, and wherein the second aryl group, the aryl part of the arylalkyl, the aryl part of the aryloxy, the aryl part of the arylsulfanyl, the aryl part of the arylsulfonyl, the heteroaryl, and the heterocycle can be substituted with 1, 2 or 3 substituents independently selected from the group consisting of alkoxy, alkyl, alkylcarbonyl, cyano, halo, haloalkoxy, haloalkyl, nitro, and oxo.

The term “arylalkenyl,” as used herein, refers to an aryl group attached to the parent molecular moiety through an alkenyl group.

The term “arylalkoxy,” as used herein, refers to an arylalkyl group attached to the parent molecular moiety through an oxygen atom.

The term “arylalkyl,” as used herein, refers to an aryl group attached to the parent molecular moiety through an alkyl group.

The term “arylcarbonyl,” as used herein, refers to an aryl group attached to the parent molecular moiety through a carbonyl group.

The term “aryloxy,” as used herein, refers to an aryl group attached to the parent molecular moiety through an oxygen atom.

The term “aryloxyalkyl,” as used herein, refers to an aryloxy group attached to the parent molecular moiety through an alkyl atom.

The term “arylsulfanyl,” as used herein, refers to an aryl group attached to the parent molecular moiety through a sulfur atom.

The term “arylsulfanylalkyl,” as used herein, refers to an arylsulfanyl group attached to the parent molecular moiety through an alkyl group.

The term “arylsulfonyl,” as used herein, refers to an aryl group attached to the parent molecular moiety through a sulfonyl group.

The term “arylsulfonylalkyl,” as used herein, refers to an arylsulfonyl group attached to the parent molecular moiety through an alkyl group.

The term “carboxy,” as used herein, refers to —CO₂H.

The term “carboxyalkyl,” as used herein, refers to a carboxy group attached to the parent molecular moiety through an alkyl group.

The term “cyano,” as used herein, refers to —CN.

The term “cyanoalkyl,” as used herein, refers to a cyano group attached to the parent molecular moiety through an alkyl group.

The term “cycloalkenyl,” as used herein, refers to a non-aromatic cyclic or bicyclic ring system having three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Examples of cycloalkenyl groups include cyclohexenyl, octahydronaphthalenyl, norbornylenyl, and the like. The cycloalkenyl groups of the present invention can be optionally substituted with one, two, three, four, or five substituents independently selected from the group consisting of alkoxy, alkyl, alkylsulfanyl, alkylsulfonyl, cyano, cyanoalkyl, halo, haloalkoxy, haloalkyl, hydroxy, nitro, and oxo.

The term “cycloalkenylalkyl,” as used herein, refers to a cycloalkenyl group attached to the parent molecular moiety through an alkyl group.

The term “cycloalkyl,” as used herein, refers to a saturated monocyclic, bicyclic, or tricyclic hydrocarbon ring system having three to twelve carbon atoms. Examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkoxycarbonyl, alkyl, alkylcarbonyl, alkylsulfanyl, alkylsulfonyl, cyano, cyanoalkyl, halo, haloalkoxy, haloalkyl, hydroxy, nitro, oxo and R_{c}R_{d}N—, RCRdNalkyl and R_{c}R_{d}NC(O)—, wherein R_{c} and R_{d} are described herein.

The term “cycloalkylalkenyl,” as used herein, refers to a cycloalkyl group attached to the parent molecular moiety through an alkenyl group.

The term “cycloalkylalkyl,” as used herein, refers to a cycloalkyl group attached to the parent molecular moiety through an alkyl group.

The term “formyl,” as used herein, refers to —CHO.

The term “formylalkyl,” as used herein, refers to a formyl group attached to the parent molecular moiety through an alkyl group.

The terms “halo,” and “halogen,” as used herein, refer to F, Cl, Br, and I.

The term “haloalkoxy,” as used herein, refers to a haloalkyl group attached to the parent molecular moiety through an oxygen atom.

The term “haloalkoxyalkyl,” as used herein, refers to a haloalkoxy group attached to the parent molecular moiety through an alkyl group.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes bicyclic systems where a heteroaryl ring is fused to a phenyl group, a monocyclic cycloalkyl group, as defined herein, a heterocycle group, as defined herein, or an additional heteroaryl group. The term “heteroaryl” also includes tricyclic systems where a bicyclic system is fused to a phenyl group, a monocyclic cycloalkyl group, as defined herein, a heterocycle group, as defined herein, or an additional heteroaryl group. Examples of heteroaryl groups include benzothienyl, benzoxadiazolyl, dibenzofuranyl, dihydrobenzothiazolyl, furanyl, imidazolyl, indazolyl, indolyl, isoxazolyl, isoquinolinyl, isothiazolyl, oxadiazolyl, oxadiazolyl, oxazolyl, thiazolyl, thienopyridinyl, thienyl, triazolyl, thiadiazolyl, pyridinyl, pyridazinyl, pyrimidinyl, pyrazinyl, pyrazolyl, pyrrolyl, quinolinyl, tetrahydroquinolinyl, triazinyl, and the like. The heteroaryl groups of the present invention can be substituted with 0, 1, 2, 3, 4 or 5 substituents independently selected from the group consisting of alkoxy, alkoxyalkyl, alkoxycarbonyl, alkyl, alkylcarbonyl, alkylsulfanyl, alkylsulfonyl, a second aryl group, arylalkyl, arylcarbonyl, aryloxy, arylsulfanyl, arylsulfonyl, carboxy, cyano, halo, haloalkoxy, haloalkyl, heteroaryl, heteroarylalkyl, heterocycle, heterocyclealkyl, heterocyclecarbonyl, hydroxy, hydroxyalkyl, nitro, R_{c}R_{d}N—, R_{c}R_{d}Nalkyl and R_{c}R_{d}NC(O)—, wherein R_{c} and R_{d} are defined herein, and wherein the second aryl group, the aryl part of the arylalkyl, the aryl part of the aryloxy, the aryl part of the arylsulfanyl, the aryl part of the arylsulfonyl, the heteroaryl, and the heterocycle can be substituted with 1, 2 or 3 substituents independently selected from the group consisting of alkoxy, alkyl, alkylcarbonyl, cyano, halo, haloalkoxy, haloalkyl, nitro, and oxo.

The term “heteroarylalkenyl,” as used herein, refers to a heteroaryl group attached to the parent molecular moiety through an alkenyl group.

The term “heteroarylalkyl,” as used herein, refers to a heteroaryl group attached to the parent molecular moiety through an alkyl group.

The term “heteroarylsulfonyl,” as used herein, refers to a heteroaryl group attached to the parent molecular moiety through a sulfonyl group.

The term “heteroarylsulfonylalkyl,” as used herein, refers to a heteroarylsulfonyl group attached to the parent molecular moiety through a alkyl group.

The term “heterocycle,” as used herein, refers to cyclic, non-aromatic, five-, six-, or seven-membered rings containing at least one atom selected from the group consisting of oxygen, nitrogen, and sulfur. The five-membered rings have zero or one double bonds and the six- and seven-membered rings have zero, one, or two double bonds. The heterocycle groups of the invention are connected to the parent molecular group through a substitutable carbon or nitrogen atom in the ring. The term “heterocycle” also includes bicyclic systems where a heterocycle ring is fused to a phenyl group, a monocyclic cycloalkenyl group, as defined herein, a monocyclic cycloalkyl group, as defined herein, or an additional monocyclic heterocycle group. The term “heterocycle” also includes tricyclic systems where a bicyclic system is fused to a phenyl group, a monocyclic cycloalkenyl group, as defined herein, a monocyclic cycloalkyl group, as defined herein, or an additional monocyclic heterocycle group. Examples of heterocycle groups include dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups of the present invention can be substituted with 0, 1, 2, 3, 4 or 5 substituents independently selected from the group consisting of alkoxy, alkoxycarbonyl, alkyl, alkylcarbonyl, alkylsulfanyl, alkylsulfonyl, cyano, cyanoalkyl, halo, haloalkoxy, haloalkyl, nitro, oxo and R_{c}R_{d}N—, R_{c}R_{d}Nalkyl and R_{c}R_{d}NC(O)—, wherein R_{c} and R_{d} are described herein.

The term “heterocyclealkenyl,” as used herein, refers to a heterocycle group attached to the parent molecular moiety through an alkenyl group.

The term “heterocyclealkyl,” as used herein, refers to a heterocycle group attached to the parent molecular moiety through an alkyl group.

The term “hydroxy,” as used herein, refers to —OH.

The termn “hydroxyalkyl,” as used herein, refers to an alkyl group substituted by at least one hydroxy group.

The termn “nitro,” as used herein, refers to —NO₂.

The term “nitroalkyl,” as used herein, refers to an alkyl group substituted by at least one nitro group.

The term “oxo,” as used herein, refers to ═O.

The term “sulfanyl,” as used herein, refers to —S—.

The term “sulfinyl,” as used herein, refers to —SO—.

The term “sulfonyl,” as used herein, refers to —SO₂—.

The present invention is directed to compounds of formula (I), wherein A, R¹, R², R³ R⁴, R⁵, and n are defined herein.

The present invention is also directed to a method of treating or preventing disorders mediated by hepatitis C viral infection through the inhibition of hepatitis C RNA dependent RNA polymerase.

According to one embodiment of the present invention there is provided a compound of formula (I), wherein A is phenyl, R and R³, together with the carbon atoms to which they are attached form a heteroaryl ring, R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, arylalkenyl, arylalkyl, arylsulfanylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to an another embodiment of the present invention there is provided a compound of formula (I), wherein A is a five- or six-membered heteroaryl ring, R₂ and R₃, together with the carbon atoms to which they are attached form an aryl ring, R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, arylalkenyl, arylalkyl, arylsulfanylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—; and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is a five- or six-membered heteroaryl ring, R² and R³, together with the carbon atoms to which they are attached form a heteroaryl ring, R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, arylalkenyl, arylalkyl, arylsulfanylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form a heteroaryl ring, R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—, and wherein n, R⁵, Rₐ, R_{b}, Rₒ, R_{d}, Rₑ, R_{f}, R_{g}, Rₖ are as defined therein.

According to a further embodiment of the present invention there is provided a compound of formula (I), wherein A is a five- or six-membered heteroaryl ring, R₂ and R₃, together with the carbon atoms to which they are attached form an aryl ring, R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is a five- or six-membered heteroaryl ring, R² and R³, together with the carbon atoms to which they are attached form a heteroaryl ring, R₁ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroaryalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form a heteroaryl ring, R is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen, alkyl, alkylcarbonyl, or alkylsulfonyl, or HS—, and wherein n, R⁵, Rₐ, R_{b}, Rₒ, R_{d}, Rₑ, R_{f}, R_{g}, Rₖ are as defined therein.

According to a further embodiment of the present invention there is provided a compound of formula (I), wherein A is a five- or six-membered heteroaryl ring, R² and R³, together with the carbon atoms to which they are attached form an aryl ring, R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen, alkyl, alkylcarbonyl, or alkylsulfonyl, or HS—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is a five- or six-membered heteroaryl ring, R₂ and R³, together with the carbon atoms to which they are attached form a heteroaryl ring, R₁ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen, alkyl, alkylcarbonyl, or alkylsulfonyl, or HS—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form a heteroaryl ring, R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen or alkyl, and wherein n, R⁵, R⁵, Rₐ, Rₑ, R_{d}, Rₑ, R_{f}, R_{g}, Rₖ are as defined therein.

According to a further embodiment of the present invention there is provided a compound of formula (I), wherein A is a five- or six-membered heteroaryl ring, R² and R³, together with the carbon atoms to which they are attached form an aryl ring, R₁ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen or alkyl, and wherein n, R⁵, Rₐ, R_{c}, R_{d}, Rₑ, R_{f}, R_{g}, Rₖ are as defined therein.

According to a further embodiment of the present invention there is provided a compound of formula (I), wherein A is a five- or six-membered heteroaryl ring, R² and R³, together with the carbon atoms to which they are attached form a heteroaryl ring, R₁ is alkenyl, alkoxyalkyl, alkoxycarbonylalkyl, alkyl alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—, R⁴ is hydroxy or R_{b}NH—, wherein R_{b} is hydrogen or alky, and wherein n, R⁵, Rₐ, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form an aryl ring, R¹ is RₐR_{b}N—, R_{f}R_{g}C═N— or RₖO—, R⁴ is halo, alkoxy, aryloxy, hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form an aryl ring, R¹ is RₐR_{b}N—, R_{f}R_{g}C═N— or RₖO—, R⁴ is hydroxy, RₐR_{b}N— or R_{c}S—, and wherein n, R⁵, Rₐ, R_{b}, R_{c}, R_{d}, Rₑ, R_{f}, R_{g}, Rₖ are as defined therein.

According to a further embodiment of the present invention there is provided a compound of formula (I), wherein A is phenyl, R² and R³, together with the carbon atoms to which they are attached form an aryl ring, R¹ is RₐR_{b}N—, R_{f}R_{g}C═N— or RₖO—, R⁴ is hydroxy or R_{b}NH—, wherein R_{b} is hydrogen, alky, alkylcarbonyl, or alkylsulfonyl, or HS—, and wherein n, R⁵, Rₐ, R_{c}, R_{d}, Rₑ, R_{f}, R_{g}, Rₖ are as defined therein.

According to a further embodiment of the present invention there is provided a compound of formula (T), wherein A is phenyl, R² and R³, together with the carbon atoms to which they are attached form an aryl ring, R¹ is RₐR_{b}N—, R_{f}R_{g}C═N— or RₖO—, R⁴ is hydroxy or R_{b}NH—, wherein R_{b} is hydrogen or alkyl and wherein n, R⁵, Rₐ, R_{c}, R_{d}, Rₑ, R_{f}, R_{g}, Rₖ are as defined therein.

Specific compounds of the present invention include, but are not limited to:

1-butyl-3-(1,1-dioxido-4H-1,2,4-benzothiadiazin-3-yl)-4-hydroxy-1,8-naphthyridin-2(1H)-one;

1-[(5-chloro-2-thienyl)methyl]-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-1-(2-ethylbutyl)-4-hydroxy-1,8-naphthyridin-2(1H)-one;

1-[(5-bromo-2-thienyl)methyl]-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-(3-methylbenzyl)-1,8-naphthyridin-2(1H)-one;

3-(1,1-dioxido-4H-1,2,4-benzothiadiazin-3-yl)-4-hydroxy-1-(3-nitrobenzyl)-1,8-naphthyridin-2(1H)-one;

3-(1,1-dioxido-4H-1,2,4-benzothiadiazin-3-yl)-4-hydroxy-1-(3-thienylmethyl)-1,8-naphthyridin-2(1H)-one;

1-(3-chlorobenzyl)-3-(1,1-dioxido-4H-1,2,4-benzothiadiazin-3-yl)-4-hydroxy-1,8-naphthyridin-2(1H)-one;

1-(3-bromobenzyl)-3-(1,1-dioxido-4H-1,2,4-benzothiadiazin-3-yl)-4-hydroxy-1,8-naphthyridin-2(1H)-one;

1-[(2-chloro-1,3-thiazol-5-yl)methyl]-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-1-(3-fluorobenzyl)-4-hydroxy-1,8-naphthyridin-2(1H)-one;

3-(1,1-dioxido-4H-1,2,4-benzothiadiazin-3-yl)-4-hydroxy-1-(3-methylbutyl)-1,8-naphthyridin-2(1H)-one;

1-(cyclobutylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[(5-methyl-2-thienyl)methyl]-1,8-naphthyridin-2(1H)-one;

1-benzyl-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[(5-methyl-3-pyridinyl)methyl]-1,8-naphthyridin-2(1H)-one;

1-[(2-chloro-4-pyridinyl)methyl]-3-(1,1-dioxido-4H-1,2,4-benzothiadiazin-3-yl)-4-hydroxy-1,8-naphthyridin-2(1H)-one;

1-[(5-bromo-3-pyridinyl)methyl]-3-(1,1-dioxido-4H-1,2,4-benzothiadiazin-3-yl)-4-hydroxy-1,8-naphthyridin-2(1H)-one;

1-(cyclohexylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[(2S)-2-methylbutyl]-1,8-naphthyridin-2(1H)-one;

3-(1,1-dioxido-4H-1,2,4-benzothiadiazin-3-yl)-4-hydroxy-1-(4-methylbenzyl)-1,8-naphthyridin-2(1H)-one;

3-(1,1-dioxido-4H-1,2,4-benzothiadiazin-3-yl)-4-hydroxy-1-[(5-nitro-2-furyl)methyl]-1,8-naphthyridin-2(1H)-one;

1-(1-benzothien-2-ylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-(3-methoxybenzyl)-1,8-naphthyridin-2(1H)-one;

3-(1,1-dioxido-4H-1,2,4-benzothiadiazin-3-yl)-4-hydroxy-1-(3-iodobenzyl)-1,8-naphthyridin-2(1H)-one;

1-[(3,5-dimethyl-4-isoxazolyl)methyl]-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[2-(3-thienyl)ethyl]-1,8-naphthyridin-2(1H)-one;

3-(1,1-dioxido-4H-1,2,4-benzothiadiazin-3-yl)-4-hydroxy-1-(4-pyridinylmethyl)-1,8-naphthyridin-2(1H)-one;

1-(4-bromobenzyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-neopentyl-1,8-naphthyridin-2(1H)-one;

1-{[(1S,2R,5S)-6,6-dimethylbicyclo[3.1.1]hept-2-yl]methyl}-3-(1,1-dioxido-4H-1,2,4-benzothiadiazin-3-yl)-4-hydroxy-1,8-naphthyridin-2(1H)-one;

3-{[3-(1,1-dioxido-4H-1,2,4-benzothiadiazin-3-yl)-4-hydroxy-2-oxo-1,8-naphthyridin-1 (2H)-yl]methyl}benzonitrile;

3-(1,1-dioxido-4H-1,2,4-benzothiadiazin-3-yl)-4-hydroxy-1-(3-pyridinylmethyl)-1,8-naphthyridin-2(1H)-one;

1-(1-adamantylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[3-(trifluoromethyl)benzyl]-1,8-naphthyridin-2(1H)-one;

3-(1,1-dioxido-4H-1,2,4-benzothiadiazin-3-yl)-4-hydroxy-1-[(2-methyl-1,3-thiazol-5-yl)methyl]-1,8-naphthyridin-2(1H)-one;

1-(2-cyclohexylethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-(4-methoxybenzyl)-1,8-naphthyridin-2(1H)-one;

3-(1,1-dioxido-4H-1,2,4-benzothiadiazin-3-yl)-4-hydroxy-1-(2-methylbenzyl)-1,8-naphthyridin-2(1H)-one;

1-(cyclopropylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-(1,3-thiazol-4-ylmethyl)-1,8-naphthyridin-2(1H)-one;

3-(11,1-dioxido-4H-1,2,4-benzothiadiazin-3-yl)-4-hydroxy-1-[(5-phenyl-2-thienyl)methyl]-1,8-naphthyridin-2(1H)-one;

3-(1,1-dioxido-4H-1,2,4-benzothiadiazin-3-yl)-4-hydroxy-1-(4-methyl-3-pentenyl)-1,8-naphthyridin-2(1H)-one;

4-{[3-(1,1-dioxido-4H-1,2,4-benzothiadiazin-3-yl)-4-hydroxy-2-oxo-1,8-naphthyridin-1 (2H)-yl]methyl}benzonitrile;

1-[2-(1-cyclohexen-1-yl)ethyl]-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[(2-methyl-1,3-thiazol-4-yl)methyl]-1,8-naphthyridin-2(1H)-one;

2-{[3-(1,1-dioxido-4H-1,2,4-benzothiadiazin-3-yl)-4-hydroxy-2-oxo-1,8-naphthyridin-1 (2H)-yl]methyl}benzonitrile;

3-(1,1-dioxido-4H-1,2,4-benzothiadiazin-3-yl)-4-hydroxy-1-[(5-methyl-3-isoxazolyl)methyl]-1,8-naphthyridin-2(1H)-one;

3-(11,1-dioxido-4H-1,2,4-benzothiadiazin-3-yl)-4-hydroxy-1-(1-naphthylmethyl)-1,8-naphthyridin-2(1H)-one;

3-(1,1-dioxido-4H-1,2,4-benzothiadiazin-3-yl)-4-hydroxy-1-(2-pyridinylmethyl)-1,8-naphthyridin-2(1H)-one;

1-(4-tert-butylbenzyl)-3-(1,1-dioxido-4H-1,2,4-benzothiadiazin-3-yl)-4-hydroxy-1,8-naphthyridin-2(1H)-one;

ethyl [3-(1,1-dioxido-4H-1,2,4-benzothiadiazin-3-yl)-4-hydroxy-2-oxo-1,8-naphthyridin-1 (2H)-yl]acetate;

[3-(1,1-dioxido-4H-1,2,4-benzothiadiazin-3-yl)-4-hydroxy-2-oxo-1,8-naphthyridin-1(2H)-yl]acetic acid;

3-(1,1-dioxido-4H-1,2,4-benzothiadiazin-3-yl)-4-hydroxy-1-(3-phenoxybenzyl)-1,8-naphthyridin-2(1H)-one;

1-allyl-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-(2-naphthylmethyl)-1,8-naphthyridin-2(1H)-one;

3-(1,1-dioxido-4H-1,2,4-benzothiadiazin-3-yl)-4-hydroxy-1-[(1R)-1-phenylethyl]1-1,8-naphthyridin-2(1H)-one;

1-[(5-tert-butyl-2-thienyl)methyl]-3-(1,1-dioxido-4H-1,2,4-benzothiadiazin-3-yl)-4-hydroxy-1,8-naphthyridin-2(1H)-one;

1-(1,1 ′-biphenyl-4-ylmethyl)-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[2-(1H-indol-3-yl)ethyl]-1,8-naphthyridin-2(1H)-one;

3-(1,1-dioxido-4H-1,2,4-benzothiadiazin-3-yl)-1-[(6-ethoxy-2-pyridinyl)methyl]-4-hydroxy-1,8-naphthyridin-2(1H)-one;

1-benzyl-3-(1,1-dioxido-4H-1,2,4-benzothiadiazin-3-yl)-4-hydroxy-7-methyl-1,8-naphthyridin-2(1H)-one;

3-(11,1-dioxido-4H-1,2,4-benzothiadiazin-3-yl)-4-hydroxy-1-[(6-methyl-2-pyridinyl)methyl]-1,8-naphthyridin-2(1H)-one;

3-(1,1-dioxido-4H-1,2,4-benzothiadiazin-3-yl)-1-(1-ethylpropyl)-4-hydroxy-1,8-naphthyridin-2(1H)-one;

3-(1,1-dioxido-4H-1,2,4-benzothiadiazin-3-yl)-4-hydroxy-1-[(1S)-1-phenylethyl]-1,8-naphthyridin-2(1H)-one;

2-{2-[3-(1,1-dioxido-4H-1,2,4-benzothiadiazin-3-yl)-4-hydroxy-2-oxo-1,8-naphthyridin-1(2H)-yl]ethyl}-1H-isoindole-1,3(2H)-dione;

3-(1,1-dioxido-4H-1,2,4-benzothiadiazin-3-yl)-4-hydroxy-1-(3-hydroxypropyl)-1,8-naphthyridin-2(1H)-one;

1-cyclopentyl-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-1-[2-(1,3-dioxolan-2-yl)ethyl]-4-hydroxy-1,8-naphthyridin-2(11H)-one;

1-(2,3-dihydroxypropyl)-3-(1,1-dioxido-4H-1,2,4-benzothiadiazin-3-yl)-4-hydroxy-1,8-naphthyridin-2(1H)-one;

1-cycloheptyl-3-(1,1-dioxido-4H-1,2,4-benzothiadiazin-3-yl)-4-hydroxy-1,8-naphthyridin-2(1H)-one;

1-(3-anilinopropyl)-3-(1,1-dioxido-4H-1,2,4-benzothiadiazin-3-yl)-4-hydroxy-1,8-naphthyridin-2(1H)-one;

3-[3-(1,1-dioxido-4H-1,2,4-benzothiadiazin-3-yl)-4-hydroxy-2-oxo-1,8-naphthyridin-1(2H)-yl]propanal;

methyl 4-{[3-(1,1-dioxido-4H-1,2,4-benzothiadiazin-3-yl)-4-hydroxy-2-oxo-1,8-naphthyridin-1 (2H)-yl]methyl}benzoate;

ethyl 5-{[3-(1,1-dioxido-4H-1,2,4-benzothiadiazin-3-yl)-4-hydroxy-2-oxo-1,8-naphthyridin-1 (2H)-yl]methyl}-2-furoate;

1-[3-(dimethylamino)propyl]-3-(1,1-dioxido-4H-1,2,4-benzothiadiazin-3-yl)-4-hydroxy-1,8-naphthyridin-2(1H)-one;

1-{3-[[2-(dimethylamino)ethyl](methyl)amino]propyl}-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[3-(4-methyl-1-piperazinyl)propyl]-1,8-naphthyridin-2(1H)-one;

1-(2-aminoethyl)-3-(1,1-dioxido-4H-1,2,4-benzothiadiazin-3-yl)-4-hydroxy-1,8-naphthyridin-2(1H)-one;

1-[3-(diethylamino)propyl]-3-(1,1-dioxido-4H-1,2,4-benzothiadiazin-3-yl)-4-hydroxy-1,8-naphthyridin-2(1H)-one;

1-cyclohexyl-3-(1,1-dioxido-4H-1,2,4-benzothiadiazin-3-yl)-4-hydroxy-1,8-naphthyridin-2(1H)-one;

3-(1,1-dioxido-4H-1,2,4-benzothiadiazin-3-yl)-4-hydroxy-1-[3-(4-morpholinyl)propyl]-1,8-naphthyridin-2(1H)-one;

5-{[3-(1,1-dioxido-4H-1,2,4-benzothiadiazin-3-yl)-4-hydroxy-2-oxo-1,8-naphthyridin-1 (2H)-yl]methyl}-2-furoic acid;

1-benzyl-3-(7-bromo-1,1-dioxido-4H-1,2,4-benzothiadiazin-3-yl)-4-hydroxy-1,8-naphthyridin-2(1H)-one;

1-benzyl-3-(1,1-dioxido-7-phenyl-4H— 1,2,4-benzothiadiazin-3-yl)-4-hydroxy-1,8-naphthyridin-2(1H)-one;

1-benzyl-3-(7-cyclohexyl-1,1-dioxido-4H-1,2,4-benzothiadiazin-3-yl)-4-hydroxy-1,8-naphthyridin-2(1H)-one;

1-benzyl-3-(7-tert-butyl-1,1-dioxido-4H-1,2,4-benzothiadiazin-3-yl)-4-hydroxy-1,8-naphthyridin-2(1H)-one;

1-benzyl-4-hydroxy-3-(7-methyl-1,1-dioxido-4H-1,2,4-benzothiadiazin-3-yl)-1,8-naphthyridin-2(1H)-one;

1-butyl-3-(6-chloro-1,1-dioxido-4H-1,2,4-benzothiadiazin-3-yl)-4-hydroxy-1,8-naphthyridin-2(1H)-one;

1-benzyl-3-(8-bromo-5-methyl-1,1-dioxido-4H-1,2,4-benzothiadiazin-3-yl)-4-hydroxy-1,8-naphthyridin-2(1H)-one;

1-benzyl-3-(8-fluoro-5-methyl-1,1-dioxido-4H-1,2,4-benzothiadiazin-3-yl)-4-hydroxy-1,8-naphthyridin-2(1H)-one;

1-benzyl-4-hydroxy-3-(5-isopropyl-1,1-dioxido-4H-1,2,4-benzothiadiazin-3-yl)-1,8-naphthyridin-2(1H)-one;

1-benzyl-4-hydroxy-3-(5-methyl-1,1-dioxido-4H-1,2,4-benzothiadiazin-3-yl)-1,8-naphthyridin-2(1H)-one;

1-benzyl-3-(5-bromo-1,1-dioxido-4H-1,2,4-benzothiadiazin-3-yl)-4-hydroxy-1,8-naphthyridin-2(1H)-one;

1-benzyl-3-(1,1-dioxido-5-propyl-4H-1,2,4-benzothiadiazin-3-yl)-4-hydroxy-1,8-naphthyridin-2(1H)-one;

1-benzyl-3-(5-ethyl-1,1-dioxido-4H-1,2,4-benzothiadiazin-3-yl)-4-hydroxy-1,8-naphthyridin-2(1H)-one;

3-(1-benzyl-4-hydroxy-2-oxo-1,2-dihydro-1,8-naphthyridin-3-yl)-4H-1,2,4-benzothiadiazine-5-carbonitrile 1,1-dioxide;

1-butyl-3-(1,1-dioxido-4H-pyrido[3,2-e][1,2,4]thiadiazin-3-yl)-4-hydroxy-2(1H)-quinolinone;

1-benzyl-3-(1,1-dioxido-4H-pyrido[3,2-e][1,2,4]thiadiazin-3-yl)-4-hydroxy-2(1H)-quinolinone;

1-benzyl-3-(1,1-dioxido-4H-pyrido[3,2-e][1,2,4]thiadiazin-3-yl)-4-hydroxy-1,8-naphthyridin-2(1H)-one;

5-chloro-3-(11-dioxido-4H-pyrido[3,2-e][1,2,4]thiadiazin-3-yl)-4-hydroxy-1-(3-methylbutyl)-2(1H)-quinolinone;

1-benzyl-3-(1,1-dioxido-4H-pyrido[3,2-e][1,2,4]thiadiazin-3-yl)-4-hydroxy-5-methyl-2(1H)-quinolinone;

3-(1,1-dioxido-4H-pyrido[3,2-e][1,2,4]thiadiazin-3-yl)-4-hydroxy-1-[(2-methyl-1,3-thiazol-5-yl)methyl]-2(1H)-quinolinone;

1-benzyl-4-hydroxy-3-(7-methyl-1,1-dioxido-4H-pyrido[2,3-e][1,2,4]thiadiazin-3-yl)-2(1H)-quinolinone;

1-butyl-4-hydroxy-3-(7-methyl-1,1-dioxido-4H-pyrido[2,3-e][1,2,4]thiadiazin-3-yl)-1,8-naphthyridin-2(1H)-one;

3-(1,1-dioxido-4H-1,2,4-benzothiadiazin-3-yl)-4-hydroxy-1-(2-thienylmethyl)-1,8-naphthyridin-2(1H)-one;

1-(benzyloxy)-3-(1,1-dioxido-4H-1,2,4-benzothiadiazin-3-yl)-4-hydroxy-1,8-naphthyridin-2(1H)-one;

6-(1,1-dioxido-4H-1,2,4-benzothiadiazin-3-yl)-8-(2-ethylbutyl)-5-hydroxy-2-(methylsulfanyl)pyrido[2,3-d]pyrimidin-7(8H)-one;

6-(1,1-dioxido-4H-1,2,4-benzothiadiazin-3-yl)-8-(2-ethylbutyl)-5-hydroxypyrido[2,3-d]pyrimidin-7(8H)-one;

4-benzyl-6-(1,1-dioxido-4H-1,2,4-benzothiadiazin-3-yl)-7-hydroxythieno[3,2-b]pyridin-5(4H)-one;

4-butyl-6-(1,1-dioxido-4H-1,2,4-benzothiadiazin-3-yl)-7-hydroxythieno[3,2-b]pyridin-5(4H)-one;

6-(1,1-dioxido-4H-1,2,4-benzothiadiazin-3-yl)-7-hydroxy-4-(4-pyridinylmethyl)thieno[3,2-b]pyridin-5(4H)-one;

1-(3-bromobenzyl)-3-(1,1-dioxido-4H-1,2,4-benzothiadiazin-3-yl)-4-hydroxy-5,6,7,8-tetrahydro-2(1H)-quinolinone;

5-(1,1-dioxido-4H-1,2,4-benzothiadiazin-3-yl)-4-hydroxy-7-(4-pyridinylmethyl)thieno[2,3-b]pyridin-6(7H)-one;

6-(1,1-dioxido-4H-1,2,4-benzothiadiazin-3-yl)-7-hydroxy-4-(3-pyridinylmethyl)thieno[3,2-b]pyridin-5(4H)-one;

7-benzyl-5-(1,1-dioxido-4H-1,2,4-benzothiadiazin-3-yl)-4-hydroxythieno[2,3-b]pyridin-6(7H)-one;

4-(cyclopropylmethyl)-6-(1,1-dioxido-4H-1,2,4-benzothiadiazin-3-yl)-7-hydroxythieno[3,2-b]pyridin-5(4H)-one;

5-(1,1-dioxido-4H-1,2,4-benzothiadiazin-3-yl)-4-hydroxy-7-(3-methylbutyl)thieno[2,3-b]pyridin-6(7H)-one;

4-benzyl-6-(1,1-dioxido-4H-1,2,4-benzothiadiazin-3-yl)-7-hydroxy-2-phenylthieno[3,2-b]pyridin-5(4H)-one;

4-benzyl-6-(1,1-dioxido-4H-1,2,4-benzothiadiazin-3-yl)-7-hydroxy-3-methylthieno[3,2-b]pyridin-5(4H)-one;

1-benzyl-3-(1,1-dioxido-4H-1,2,4-benzothiadiazin-3-yl)-4-hydroxy-5,6,7,8-tetrahydro-2(1H)-quinolinone;

1-benzyl-3-(1,1-dioxido-4H-1,2,4-benzothiadiazin-3-yl)-4-hydroxy-2(1H)-pyridinone;

1-benzyl-3-(1,1-dioxido-4H-1,2,4-benzothiadiazin-3-yl)-4-hydroxy-5,6-dimethyl-2(1H)-pyridinone;

7-benzyl-5-(1,1-dioxido-4H-1,2,4-benzothiadiazin-3-yl)-4-hydroxy-3-methylthieno[2,3-b]pyridin-6(7H)-one;

6-(1,1-dioxido-4H-1,2,4-benzothiadiazin-3-yl)-7-hydroxy-4-(3-methylbutyl)thieno[3,2-b]pyridin-5(4H)-one;

6-(1,1-dioxido-4H-1,2,4-benzothiadiazin-3-yl)-4-(2-ethylbutyl)-7-hydroxythieno[3,2-b]pyridin-5(4H)-one;

1-benzyl-3-(1,1-dioxido-4H-1,2,4-benzothiadiazin-3-yl)-4-hydroxy-6-methyl-5-phenyl-2(1H)-pyridinone;

3-(1,1-dioxido-4H-1,2,4-benzothiadiazin-3-yl)-4-hydroxy-5,6-dimethyl-1-(3-methylbutyl)-2(1H)-pyridinone;

3-(1,1-dioxido-4H-1,2,4-benzothiadiazin-3-yl)-1-(2-ethylbutyl)-4-hydroxy-5,6-dimethyl-2(1H)-pyridinone;

1-benzyl-3-(1,1-dioxido-4H-1,2,4-benzothiadiazin-3-yl)-4-hydroxy-6-phenyl-2(1H)-pyridinone;

5-(1,1-dioxido-4H-1,2,4-benzothiadiazin-3-yl)-4-hydroxy-7-(3-methyl-2-butenyl)thieno[2,3-b]pyridin-6(7H)-one;

1,5-dibenzyl-3-(1,1-dioxido-4H-1,2,4-benzothiadiazin-3-yl)-4-hydroxy-6-methyl-2(1H)-pyridinone;

3-(1,1-dioxido-4H-1,2,4-benzothiadiazin-3-yl)-1-(2-ethylbutyl)-4-hydroxy-6-methyl-5-phenyl-2(1H)-pyridinone;

6-(1,1-dioxido-4H-1,2,4-benzothiadiazin-3-yl)-7-hydroxy-4-pentylthieno[3,2-b]pyridin-5(4H)-one;

5-(1,1-dioxido-4H-1,2,4-benzothiadiazin-3-yl)-4-hydroxy-3-methyl-7-(3-methylbutyl)thieno[2,3-b]pyridin-6(7H)-one;

6-(1,1-dioxido-4H-1,2,4-benzothiadiazin-3-yl)-7-hydroxy-4-(4-methylpentyl)thieno[3,2-b]pyridin-5(4H)-one;

4-(3-butenyl)-6-(1,1-dioxido-4H-1,2,4-benzothiadiazin-3-yl)-7-hydroxythieno[3,2-b]pyridin-5(4H)-one;

7-benzyl-5-(1,1-dioxido-4H-1,2,4-benzothiadiazin-3-yl)-4-hydroxy-1-phenyl-1,7-dihydro-6H-pyrazolo[3,4-b]pyridin-6-one;

4-benzyl-6-(1,1-dioxido-4H-1,2,4-benzothiadiazin-3-yl)-2,7-dihydroxy[1,3]thiazolo[4,5-b]pyridin-5(4H)-one;

4-[(2-chloro-1,3-thiazol-5-yl)methyl]-6-(1,1-dioxido-4H-1,2,4-benzothiadiazin-3-yl)-7-hydroxythieno[3,2-b]pyridin-5(4H)-one;

6-(1,1-dioxido-4H-1,2,4-benzothiadiazin-3-yl)-7-hydroxy-4-[(5-methyl-3-pyridinyl)methyl]thieno[3,2-b]pyridin-5(4H)-one;

6-(1,1-dioxido-4H-1,2,4-benzothiadiazin-3-yl)-7-hydroxy-4-[(2-methyl-1,3-thiazol-5-yl)methyl]thieno[3,2-b]pyridin-5(4H)-one;

4-[(5-chloro-2-thienyl)methyl]-6-(1,1-dioxido-4H-1,2,4-benzothiadiazin-3-yl)-7-hydroxythieno[3,2-b]pyridin-5(4H)-one;

6-(1,1-dioxido-4H-1,2,4-benzothiadiazin-3-yl)-7-hydroxy-4-[(2-methyl-1,3-thiazol-4-yl)methyl]thieno[3,2-b]pyridin-5(4H)-one;

4-benzyl-6-(1,1-dioxido-4H-1,2,4-benzothiadiazin-3-yl)-7-hydroxy-2-(methylsulfanyl)[1,3]thiazolo[4,5-b]pyridin-5(4H)-one;

4-benzyl-6-(1,1-dioxido-4H-1,2,4-benzothiadiazin-3-yl)-7-hydroxy-2-(methylsulfonyl)[1,3]thiazolo[4,5-b]pyridin-5(4H)-one;

2-amino-4-benzyl-6-(1,1-dioxido-4H-1,2,4-benzothiadiazin-3-yl)-7-hydroxy[1,3]thiazolo [4,5-b]pyridin-5 (4H)-one;

4-benzyl-6-(1,1-dioxido-4H-1,2,4-benzothiadiazin-3-yl)-7-hydroxy[1,3]thiazolo[4,5-b]pyridin-5(4H)-one;

3-(1,1-dioxido-4H-1,2,4-benzothiadiazin-3-yl)-4-hydroxy-1-(2-phenylpropyl)-1,8-naphthyridin-2(1H)-one;

8-benzyl-6-(1,1-dioxido-4H-1,2,4-benzothiadiazin-3-yl)-5-hydroxy-2-(methylsulfanyl)pyrido[2,3-d]pyrimidin-7(8H)-one;

8-benzyl-6-(1,1-dioxido-4H-1,2,4-benzothiadiazin-3-yl)-5-hydroxypyrido[2,3-d]pyrimidin-7(8H)-one;

3-(1,1-dioxido-4H-1,2,4-benzothiadiazin-3-yl)-4-hydroxy-1-(3-hydroxybutyl)-2(1H)-quinolinone;

1-benzyl-3-(1,1-dioxido-4H-1,2,4-benzothiadiazin-3-yl)-4-hydroxythieno[3,4-b]pyridin-2(1H)-one;

4-[(5-bromo-2-thienyl)methyl]-6-(1,1-dioxido-4H-1,2,4-benzothiadiazin-3-yl)-7-hydroxythieno[3,2-b]pyridin-5(4H)-one;

1-butyl-3-(1,1-dioxido-4H-1,2,4-benzothiadiazin-3-yl)-2(1H)-pyridinone;

3-(1,1-dioxido-4H-1,2,4-benzothiadiazin-3-yl)-1,8-naphthyridine-2,4-diol;

1-(benzyloxy)-3-(1,1-dioxido-4H-1,2,4-benzothiadiazin-3-yl)-4-hydroxy-1,8-naphthynidin-2(1H)-one;

3-(1,1-dioxido-4H-1,2,4-benzothiadiazin-3-yl)-4-hydroxy-1-isobutoxy-1,8-naphthyridin-2(1H)-one;

1-butyl-3-(1,1-dioxido-4H-1,2,4-benzothiadiazin-3-yl)-4-hydroxy-1,5-naphthyridin-2(1H)-one;

1-benzyl-3-(1,1-dioxido-4H-1,2,4-benzothiadiazin-3-yl)-4-hydroxy-1,5-naphthyridin-2(1H)-one;

1-benzyl-4-chloro-3-(1,1-dioxido-4H-1,2,4-benzothiadiazin-3-yl)-1,8-naphthyridin-2(1H)-one;

3-(1,1-dioxido-4H-1,2,4-benzothiadiazin-3-yl)-4-hydroxy-1-{[(1E)-phenylmethylene]amino}-2(1H)-quinolinone;

1-amino-3-(1,1-dioxido-4H-1,2,4-benzothiadiazin-3-yl)-4-hydroxy-2(1H)-quinolinone;

3-(1,1-dioxido-4H-1,2,4-benzothiadiazin-3-yl)-4-hydroxy-1-(2-phenylethyl)-1,8-naphthyridin-2(1H)-one;

1-butyl-4-chloro-3-(1,1-dioxido-4H— 1,2,4-benzothiadiazin-3-yl)-1,8-naphthyridin-2(1H)-one;

4-amino-1-butyl-3-(1,1-dioxido-4H-1,2,4-benzothiadiazin-3-yl)-1,8-naphthyridin-2(1H)-one;

1-butyl-3-(1,1-dioxido-4H-1,2,4-benzothiadiazin-3-yl)-4-(methylamino)-1,8-naphthyridin-2(11H)-one;

1-butyl-4-(dimethylamino)-3-(1,1-dioxido-4H-1,2,4-benzothiadiazin-3-yl)-1,8-naphthyridin-2(1H)-one;

1-butyl-3-(1,1-dioxido-4H-1,2,4-benzothiadiazin-3-yl)-4-hydrazino-1,8-naphthyridin-2(1H)-one;

4-azido-1-butyl-3-(1,1-dioxido-4H-1,2,4-benzothiadiazin-3-yl)-1,8-naphthyridin-2(1H)-one;

1-butyl-3-(1,1-dioxido-4H-1,2,4-benzothiadiazin-3-yl)-4-[(2-hydroxyethyl)amino]-1,8-naphthyridin-2(1H)-one;

3-(1,1-dioxido-4H-1,2,4-benzothiadiazin-3-yl)-4-hydroxy-1-propoxyquinolin-2(1H)-one;

7-benzyl-5-(1,1-dioxido-4H-1,2,4-benzothiadiazin-3-yl)-4-hydroxy-3-(hydroxymethyl)-7,7a-dihydrothieno[2,3-b]pyridin-6(3aH)-one;

1-benzyl-3-(6-chloro-1,1-dioxido-4H-thieno[3,2-e][1,2,4]thiadiazin-3-yl)-4-hydroxy-1,8-naphthyridin-2(1H)-one;

1-benzyl-3-(6-chloro-1,1-dioxido-4H-thieno[3,2-e][1,2,4]thiadiazin-3-yl)-4-hydroxyquinolin-2(1H)-one;

3-[5-(aminomethyl)-1,1-dioxido-4H-1,2,4-benzothiadiazin-3-yl]-1-benzyl-4-hydroxy-1,8-naphthyridin-2(1H)-one;

8-benzyl-3-chloro-6-(1,1-dioxido-4H-1,2,4-benzothiadiazin-3-yl)-5-hydroxypyrido[2,3-c]pyridazin-7(8H)-one;

8-benzyl-6-(1,1-dioxido-4H-1,2,4-benzothiadiazin-3-yl)-5-hydroxy-3-(methylthio)pyrido[2,3-c]pyridazin-7(8H)-one;

8-benzyl-6-(1,1-dioxido-4H-1,2,4-benzothiadiazin-3-yl)-5-hydroxypyrido[2,3-c]pyridazin-7(8H)-one;

1-benzyl-3-(1,1-dioxido-4H-1,2,4-benzothiadiazin-3-yl)-4-hydroxy-1,6-naphthyridin-2(1H)-one;

1-benzyl-3-(1,1-dioxido-4H-1,2,4-benzothiadiazin-3-yl)-4-hydroxy-1,7-naphthyridin-2(1H)-one; and

1-(benzylamino)-3-(1,1-dioxido-4H-1,2,4-benzothiadiazin-3-yl)-4-hydroxyquinolin-2(1H)-one,

or pharmaceutically acceptable salts thereof.

The compounds of the invention can comprise asymmetrically substituted carbon atoms. As a result, all stereoisomers of the compounds of the invention are meant to be included in the invention, including racemic mixtures, mixtures of diastereomers, as well as individual optical isomers, including, enantiomers and single diastereomers of the compounds of the invention substantially free from their enantiomers or other diastereomers. By “substantially free” is meant greater than about 80% free of other enantiomers or diastereomers of the compound, more preferably greater than about 90% free of other enantiomers or diastereomers of the compound, even more preferably greater than about 95% free of other enantiomers or diastereomers of the compound, even more highly preferably greater than about 98% free of other enantiomers or diastereomers of the compound and most preferably greater than about 99% free of other enantiomers or diastereomers of the compound.

In addition, compounds comprising the possible geometric isomers of carbon-carbon double bonds and carbon-nitrogen double are also meant to be included in this invention.

Individual stereoisomers of the compounds of this invention can be prepared by any one of a number of methods which are within the knowledge of one of ordinary skill in the art. These methods include stereospecific synthesis, chromatographic separation of diastereomers, chromatographic resolution of enantiomers, conversion of enantiomers in an enantiomeric mixture to diastereomers and then chromatographically separating the diastereomers and regeneration of the individual enantiomers, enzymatic resolution and the like.

Stereospecific synthesis involves the use of appropriate chiral starting materials and synthetic reactions which do not cause racemization or inversion of stereochemistry at the chiral centers.

Diastereomeric mixtures of compounds resulting from a synthetic reaction can often be separated by chromatographic techniques which are well-known to those of ordinary skill in the art.

Chromatographic resolution of enantiomers can be accomplished on chiral chromatography resins. Chromatography columns containing chiral resins are commercially available. In practice, the racemate is placed in solution and loaded onto the column containing the chiral stationary phase. The enantiomers are then separated by HPLC.

Resolution of enantiomers can also be accomplished by converting the enantiomers in the mixture to diastereomers by reaction with chiral auxiliaries. The resulting diastereomers can then be separated by column chromatography. This technique is especially useful when the compounds to be separated contain a carboxyl, amino or hydroxyl group that will form a salt or covalent bond with the chiral auxiliary. Chirally pure amino acids, organic carboxylic acids or organosulfonic acids are especially useful as chiral auxiliaries. Once the diastereomers have been separated by chromatography, the individual enantiomers can be regenerated. Frequently, the chiral auxiliary can be recovered and used again.

Enzymes, such as esterases, phosphatases and lipases, can be useful for resolution of derivatives of the enantiomers in an enantiomeric mixture. For example, an ester derivative of a carboxyl group in the compounds to be separated can be prepared. Certain enzymes will selectively hydrolyze only one of the enantiomers in the mixture. Then the resulting enantiomerically pure acid can be separated from the unhydrolyzed ester.

The present compounds may exhibit the phenomena of tautomerism or structural isomerism. As the drawings within this specification can only represent one possible tautomeric or structural isomeric form, it should be understood that the invention encompasses any tautomeric or structural isomeric form, or mixtures thereof, which possess the ability to inhibit hepatitis C, and is not limited to any one tautomeric or structural isomeric form utilized within the drawings.

In addition, solvates and hydrates of the compounds of the invention are meant to be included in this invention.

When any variable (for example R¹, R², R³, m, n, etc.) occurs more than one time in any substituent or in the compound of the invention or any other formula herein, its definition on each occurrence is independent of its definition at every other occurrence. In addition, combinations of substituents are permissible only if such combinations result in stable compounds. Stable compounds are compounds which can be isolated in a useful degree of purity from a reaction mixture.

The compounds of the present invention can exist as pharmaceutically acceptable salts. The term “pharmaceutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting a basic group (for example, a nitrogen containing group) with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting an acidic group (for example, a carboxy group or an enol) with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of basic addition salts include ethylenediamine, ethanolamine, diethanolamine, piperidine, and piperazine.

Preferred salts of the compounds of the present invention include sodium and hydrochloride.

The present compounds can also exist as pharmaceutically acceptable prodrugs. The term “pharmaceutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

In accordance with methods of treatment and pharmaceutical compositions of the invention, the compounds can be administered alone or in combination with other antiviral agents. When using the compounds, the specific pharmaceutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The antiviral effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The compounds of the invention inhibit HCV RNA dependent RNA polymerase an enzyme essential for HCV viral replication. They can be administered as the sole active pharmaceutical agent, or they can also be used in combination with one or more agents to treat hepatitis C infections or the symptoms associated with HCV infection. Other agents to be administered in combination with a compound of the present invention include therapies for disease caused by HCV infection that suppresses HCV viral replication by direct or indirect mechanisms. These include agents such as host immune modulators, for example, interferon-α, pegylated interferon-ax and the like, or antiviral compounds that inhibit host cellular functions such as inosine monophosphate dehydrogenase, for example, ribavirin and the like. Also included are agents that interact with host cellular components to block viral protein synthesis by inhibiting the internal ribosome entry site (IRES) initiated translation step of HCV viral replication or to block viral particle maturation and release with agents targeted toward the viroporin family of membrane proteins such as, for example, HCV P7 and the like. Other agents to be administered in combination with a compound of the present invention include any agent or combination of agents that inhibit the replication of HCV by targeting proteins of the viral genome involved in the viral replication. These agents include but are not limited to other inhibitors of HCV RNA dependent RNA polymerase such as, for example, nucleoside type polymerase inhibitors described in WO0190121(A2), or U.S. Pat. No. 6,348,587B1 or WO0160315 or WO0132153 or non-nucleoside inhibitors such as, for example, benzimidazole polymerase inhibitors described in EP1162196A1 or WO0204425 or inhibitors of HCV protease such as, for example, peptidomimetic type inhibitors such as BILN2061 and the like or inhibitors of HCV helicase.

Other agents to be administered in combination with a compound of the present invention include any agent or combination of agents that inhibit the replication of other viruses for co-infected individuals. These agent include but are not limited to therapies for disease caused by hepatitis B (HBV) infection such as, for example, adefovir, lamivudine, and tenofovir or therapies for disease caused by human immunodeficiency virus (HIV) infection such as, for example, protease inhibitors: ritonavir, lopinavir, indinavir, nelfinavir, saquinavir, amprenavir, atazanavir, tipranavir, TMC-114, fosamprenavir; reverse transcriptase inhibitors: zidovudine, lamivudine, didanosine, stavudine, tenofovir, zalcitabine, abacavir, efavirenz, nevirapine, delavirdine, TMC-125; integrase inhibitors: L-870812, S-1360, or entry inhibitors: enfuvirtide (T-20), T-1249.

Other agents to be administered in combination with a compound of the present invention include any agent or combination of agents that treat or alleviate symptoms of HCV infection including cirrhosis and inflammation of the liver.

When administered as a combination, the therapeutic agents can be formulated as separate compositions which are given at the same time or different times, or the therapeutic agents can be given as a single composition.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

### HCV Polymerase Inhibition Assay: Biochemical IC₅₀:

Two-fold serial dilutions of the inhibitors were incubated with 20 mM Tris-Cl pH 7.5, 5 mM MgCl₂, 50 mM NaCl, 1 mM dithiothreitol, 1 mM ethylene diamine tetraacetic acid (EDTA), 300 uM GTP and 150 to 300 nM NS5B (HCV Strain 1B (J4), Genbank accession number AF054247) for 15 minutes at room temperature. The reaction was initiated by the addition of 20 uM CTP, 20 uM ATP, 1 uM 3H-UTP (10 mCi/umol), 150 nM template RNA and 0.4 U/ul RNase inhibitor (RNasin, Promega), and allowed to proceed for 2 to 4 hours at room temperature. Reaction volume was 50 ul. The reaction was terminated by the addition of 1 volume of 4 mM spermine in 10 mM Tris-Cl pH 8.0, 1 mM EDTA. After incubation for at least 15 minutes at room temperature, the precipitated RNA was captured by filtering through a GF/B filter (Millipore) in a 96 well format. The filter plate was washed three times with 200 ul each of 2 mM spermine, 10 mM Tris-Cl pH 8.0, 1 mM EDTA, and 2 times with ethanol. After air drying, 30 ul of Microscint 20 scintillation cocktail (Packard) was added to each well, and the retained cpm were determined by scintillation counting. IC50 values were calculated by a two-variable nonlinear regression equation using an uninhibited control and a fully inhibited control sample to determine the minimum and maximum for the curve.

The sequence of the template RNA used was: 5′ GGGCGAAUUGGGCCCUCUAGAUGCAUGCUCGAGCGGCCGCCAGUGUGAUGG AUAUCUGCAGAAUUCGCCCUUGGUGGCUCCAUCUUAGCCCUAGUCACGGCUAG CUGUGAAAGGUCCGUGAGCCGCUUGACUGCAGAGAGUGCUGAUACUGGCCUCU CUGCAGAUCAAGUC-3′

When tested by the above method, the compounds of the present invention inhibit HCV polymerase 1b with IC₅₀'s in the range of 0.030 μM to 500 μM.

Evaluation of the HCV Inhibitors in HCV Replicon: Cell Culture IC₅₀

The cell lines and assays were conducted according to the methods described according to Ikeda M, Yi M, Li K, Lemon S M., J Virol 2002 March;76(6):2997-3006, and Blight K. J, Kolykhalov A., Rice C. M., Science 2000 Dec, 290:1972-1974) with the following modifications:

### RNA Assay

Replicon cells were plated at 3×10³ cells per well in 96-well plate in DMEM medium containing 5% fetal calf serum. At day 1, culture medium was removed and replaced with fresh medium containing eight serial 2-fold dilutions of compound. The final concentration of DMSO in medium was 0.5%. The untreated control culture was treated in an identical manner except no inhibitor was added to the medium. Plates were incubated in a CO₂ incubator at 37° C. On Day 3, 100 μl lysis buffer (RTL) (Qiagen) was added to each well after removal of culture medium. RNA was purified according to manufacturer's recommendations (Qiagen RNAeasy) and eluted in 200 μl of water. 5 μl of the purified RNA was added into a PCR tube containing TaqMan one-step RT-PCR Master Mix along with primers and probe. The HCV RNA level was quantified from a portion of the purified RNA by real-time RT-PCR method. The primers and probe are derived from specific sequence in the 5′UTR region. RT-PCR reaction was performed at 48° C. 30 min, followed by 40 cycles set to 95° C., 15 s; 54° C., 30 s; and 72° C., 40 s. The percentage reduction of HCV RNA in the presence of compound was calculated and the 50% inhibitory concentration (IC₅₀) was calculated by non-linear regression analysis using the Prism program.

When tested by the above method, the compounds of the present invention inhibit replicon production with EC₅'s in the range of 0.200 μM to >100 μM.

### Cytotoxity Assays

Cytotoxicity assays were performed in replicon cells. Briefly, HCV replicon cells were plated at 3×10³ cells per well in 96-well plate in DMEM medium containing 5% FCS. At day 1, culture medium was removed and replaced with fresh medium containing eight serial 2-fold dilutions of compound. The final concentration of DMSO in medium was 0.5%. All experiments were performed in duplicate. The untreated control culture was treated in an identical manner except no inhibitor was added to the medium. Plates were incubated in a CO₂ incubator at 37° C. On day 5, stock solution of the tetrazolium salt, MTT (4 mg/ml in PBS, Sigma cat.# M 2128)) was added to each well at 25 μl per well. Plates were further incubated for 4 h, treated with 20% SDS plus 0.02 N HCl at 50 μl per well to lyse the cells. After an overnight incubation, optical density was measured by reading the plates at 570/650 nm wavelengths. The percent reduction of formazan blue color formed relative to control was calculated and the cytopathic effect was described as a 50% toxicity concentration (TC₅₀) was calculated by non-linear regression analysis using the Prism program.

When tested by the above method, the compounds of the present invention exhibited CPE reduction with TC₅₀'s in the range of 16 μM to >100 μM.

Cell culture assays for agents targeted toward hepatitis C are not yet available because of the inability to produce infectious virus in a sustained cell line. The hepatitis C virus genome encodes a large polyprotein, which after processing produces the necessary functional components to synthesize progeny RNA. Selectable cell lines that produce high and sustained levels of subgenomic HCV RNA (replicons) have been derived from human hepatoma cells (Huh7) as described in the references above. The mechanism of RNA replication in these cell lines is considered to be identical to the replication of full length HCV RNA in infected hepatocytes. The compounds and methods of this invention are inhibitors of HCV RNA replication in the replicon assay systems described above. This forms the basis of the claim for their potential as therapies in treating disease resulting from hepatitis C viral infection.

Synthetic Methods

Abbreviations which have been used in the descriptions of the scheme and the examples that follow are: THF is tetrahydrofuran.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups A, R¹, R², R³, R⁴, R⁵, and n are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

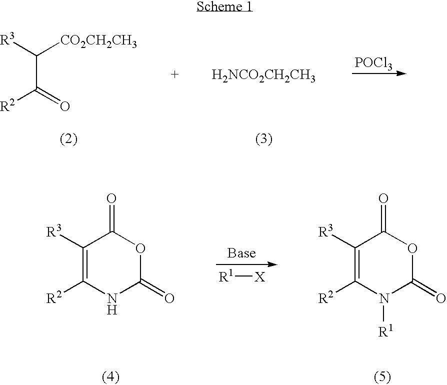

As shown in Scheme 1, compounds of formula (2) can be reacted with compounds of formula (3) in the presence of phosphorous oxychloride under heating conditions to provide compounds of formula (4). Compounds of formula (4) can be reacted with a base such as sodium hydride, potassium hydride, lithium hexamethyldisilazide, and the like in solvent such as but not limited to dimethylacetamide, dimethylformamide, THF, and the like, followed by the addition of R¹-X, (wherein R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, arylalkenyl, arylalkyl, arylsulfanylalkyl, arylsulfonylalkyl, carboxyalkyl, cyanoalkyl, cycloalkenyl, cycloalkenylalkyl, cycloalkyl, cycloalkylalkenyl, cycloalkylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}Nalkyl-, RₐR_{b}NC(O)alkyl-, RₐR_{b}NC(O)Oalkyl- or RₐR_{b}NC(O)NR_{c}alkyl-, and wherein X is Br, Cl, I, CF₃S(O)₂—, CH₃S(O)₂—, or tosyl) to provide compounds of formula (5).

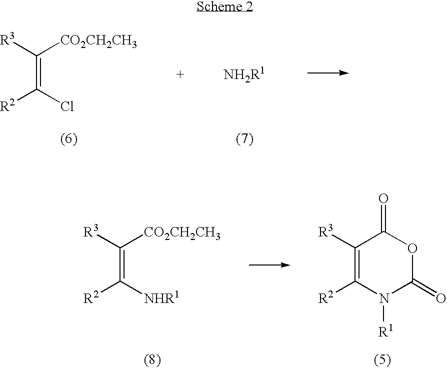

Alternatively, compounds of formula (6) can be treated with compounds of formula (7) (wherein R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, aryl, arylalkenyl, arylalkyl, arylsulfanylalkyl, arylsulfonylalkyl, carboxyalkyl, cyanoalkyl, cycloalkenyl, cycloalkenylalkyl, cycloalkyl, cycloalkylalkenyl, cycloalkylalkyl, formylalkyl, haloalkoxyalkyl, haloalkyl, heteroaryl, heteroarylalkenyl, heteroarylalkyl, heteroarylsulfonylalkyl, heterocycle, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—), under heating conditions to provide compounds of formula (8). Compounds of formula (8) can be treated with reagents including but not limited to phosgene, diphosgene, triphosgene in solvents such as but not limited to 1,2-dichloroethane, carbon tetrachloride, 1,4-dioxane or mixtures thereof, under heating conditions to provide compounds of formula (5).

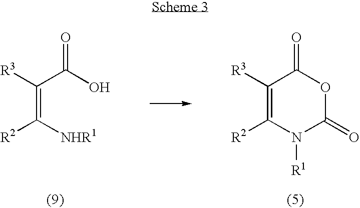

In addition, compounds of formula (9) can also be reacted with reagents including but not limited to phosgene diphosgene, triphosgene, carbonyldiimidazole, ethyl chloroformate and the like in the presence of a base such as potassium hydroxide, pyridine, lithium hydroxide, and the like in solvents such as but notlimited to water, toluene, benzene, and the like under heating conditions to provide compounds of formula (5).

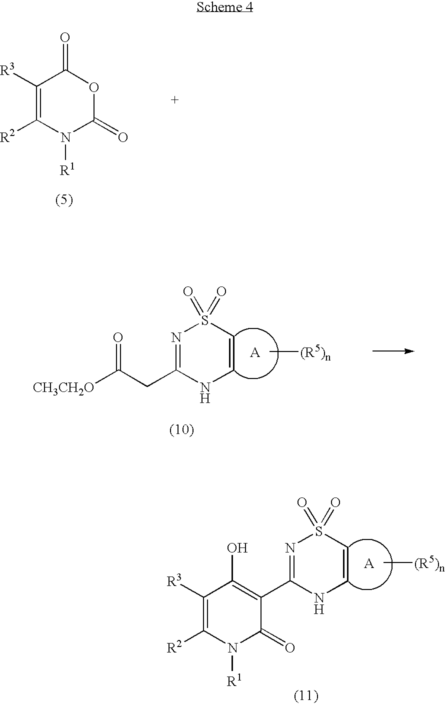

Compounds of formula (5) can be treated with compounds of formula (10) in the presence of a base such as sodium hydride, potassium hydride, lithium hexamethyldisilazide, and the like in a solvent such as but not limited to TBF, diethyl ether, methyl tert-butyl ether followed by the treatment with an acid such as acetic acid, dichloroacetic acid or sulfuric acid to provide compounds of formula (11) which are representative of a compound of formula (I), where R⁴ is hydroxy.

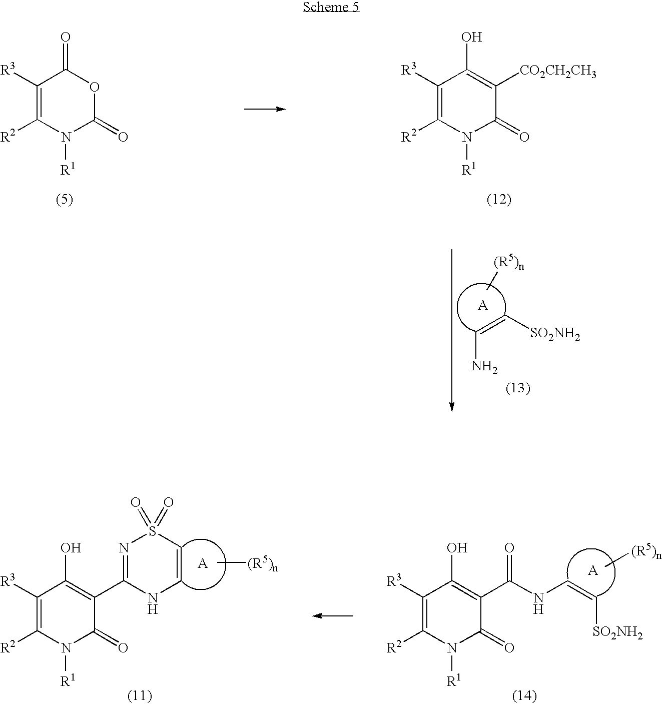

Compounds of formula (5) can be reacted with diethyl malonate that has been pretreated with a base such as sodium hydride, potassium hydride, and the like in solvents such as dimethylacetamide, dimethylformamide, THF, and the like under heated conditions to provide compounds of formula (12). Compounds of formula (12) can be treated with compounds of formula (13) in solvents such as toluene, mesitylene, benzene, and the like under heated conditions to provides compounds of formula (14). Compounds of formula (14) can be treated with a base such as sodium hydroxide, potassium hydroxide, lithium hydroxide, and the like in water under heated conditions to provide compounds of formula (11).

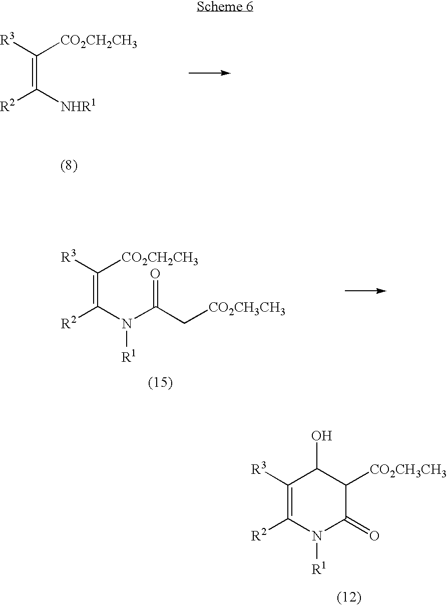

Alternatively, compounds of formula (8) can be treated with ethyl chloromalonate in the presence of a base such as triethylamine, diisopropylethylamine, pyridine, and the like in solvents such as dichloromethane, chloroform, carbon tetrachloride to provide compounds of formula (15). Compounds of formula (15) can be treated with sodium ethoxide in ethanol to provide compounds of formula (12).

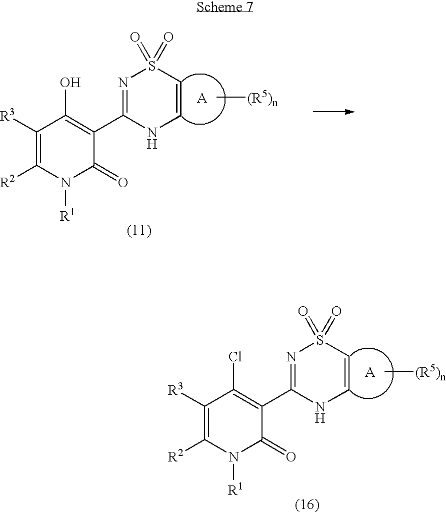

Scheme 7 shows the preparation of compounds of formula (16) (compounds of formula (I) where R₄ is halo). Compounds of formula (11) can be treated with reagents known to those skilled in the art which are commonly used to convert alcohols to chlorides. For example, compounds of formula (11) can be treated with reagents including but not limited to PCl₅, PCl₃, POCl₃, thionyl chloride with or without solvents that may include dichloromethane, chloroform and benzene to provide compounds of formula (16) which are representative of compounds of formula (I) where R⁴ is chlorine. Similar transformations are possible using PBr₃ or DAST to convert the said alcohol to the corresponding compound of formula (I) where R⁴ is bromide and fluoride, respectively. Alternatively, by converting the alcohol to a mesylate (CH₃S(O)₂—), followed by the treatment with N-iodosuccinimide may be used to prepare the compounds of formula (I) where R⁴ is iodo.

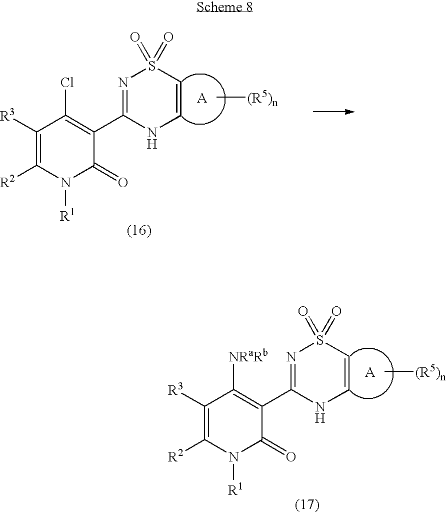

As shown in Scheme 8, compounds of formula (16) can be converted to compounds of formula (17) which are representative of compounds of formula (I) where R⁴ is amino, by treatment with an appropriately substituted amine RaRbNH, (where Rₐ and R_{b} are as defined herein) in a polar solvent such as methanol, ethanol, and the like, under heating conditions to provide compounds of formula (I) where R⁴ is a substituted amine.

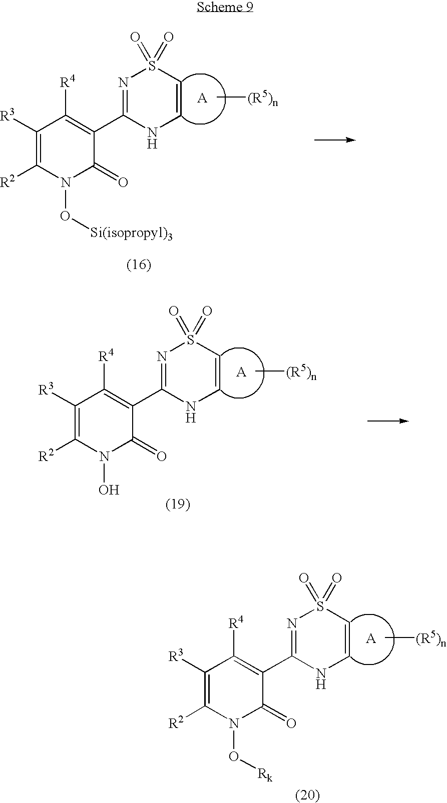

Compounds of formula (18) (where R¹ is O—Si(isopropyl)₃ or some other easily removed ether protecting group) can be treated with a fluoride containing reagent to provide compounds of formula (19). The hydroxylamine portion of compounds of formula (19) can be treated with a base such as sodium hydride in solvents such as dimethylformamide, or lithium hexamethyldisilazide in solvents such as but not limited to THF, dioxane and the like, followed by the addition of Rₖ-X (wherein Rₖ is alkoxyalkyl, alkoxycarbonyl, alkoxycarbonylalkyl, alkyl, alkylcarbonylalkyl, alkylsulfanylalkyl, alkylsulfonylalkyl, aryl, arylalkyl, arylsulfanylalkyl, carboxyalkyl, cyanoalkyl, cycloalkenyl, cycloalkenylalkyl, cycloalkyl, cycloalkylalkyl, formylalkyl, haloalkoxyalkyl, haloalkyl, heteroaryl, heteroarylalkyl, heterocycle, heterocyclealkyl, hydroxyalkyl, nitroalkyl, R_{c}R_{d}Nalkyl- or R_{c}R_{d}NC(O)alkyl, and wherein X is Br, Cl, I, CF₃S(O)₂—, CH₃S(O)₂—, or tosyl) to provide compounds of formula (20) which are representative of compounds of formula (I) where R¹ is defined as RₖO—.

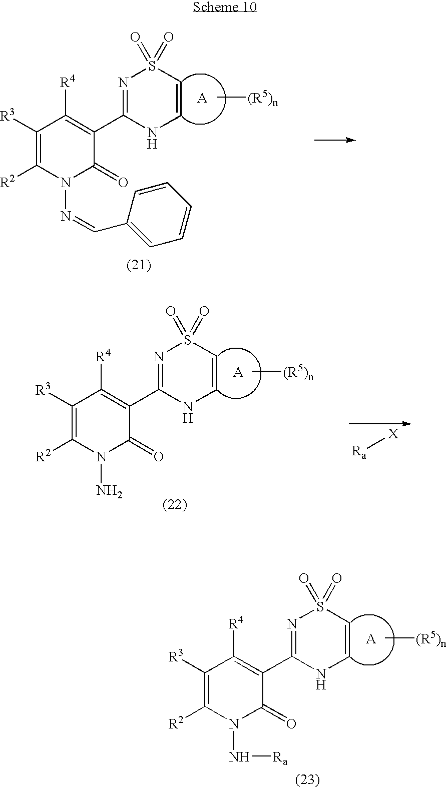

Compounds of formula (21) can be treated with aqueous base to provide compounds of formula (22). Compounds of formula (22) can be treated with a base such as sodium hydride in solvents such as dimethylformamide, or lithium hexamethyldisilazide in solvents such as but limited to THF, dioxane and the like, followed by the addition of RₐX and wherein X is Br, Cl, I, CF₃S(O)₂—, CH₃S(O)₂—, or tosyl) to provide compounds of formula (23) which are representative of compounds of formula (I).

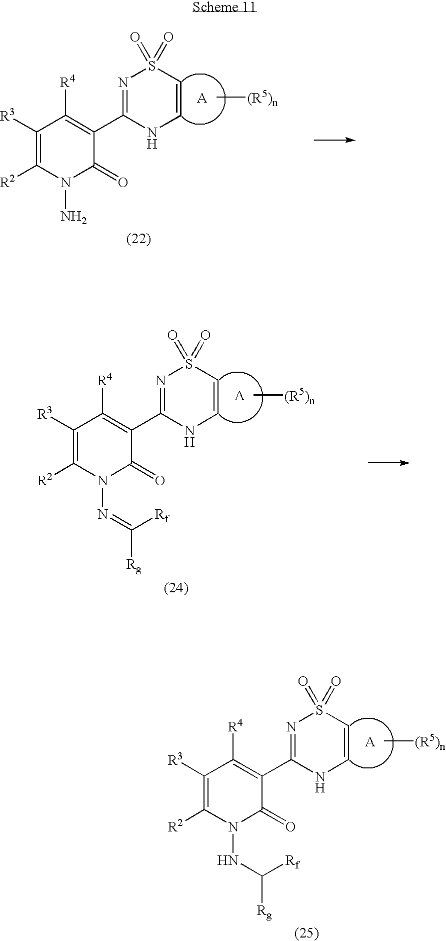

Alternatively, compounds of formula (22) can be treated with aldehydes or ketones of structure R_{f}R_{g}C(O) without solvents or with solvents such as but not limited to THF, dioxane and the like under heated conditions to provide compounds of formula (24). Reduction of compounds of the formula (24) with hydrogen and a catalyst such as palladium and the like or metal hydrides such as sodium borohydride, sodium cyanoborohydride and the like provide compounds of the formula (25).

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purpose of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names consistent with ACD nomenclature.

### EXAMPLE 1

### 1-butyl-3-(1,1-dioxido-4H-1,2,4-benzothiadiazin-3-yl)-4-hydroxy-1,8-naphthyridin-2(1H)-one

### EXAMPLE 1A

### 2H-pyrido[2,3-d][1,3]oxazine-2,4(1H)-dione

The title compound was prepared from 2,3-pyridinecarboxylic anhydride (11.4 g, 76 mmol) and trimethylsilyl azide (11.0 mL, 80 mmol) according to the procedure described in Synthesis, 1982, 972-973 as a white solid (7.27 g, 58%). ¹H NMR (300 MHz, DMSO-d₆) δ 7.31 (dd, J=7.72, 4.78 Hz, 1H), 8.31 (dd, J=7.72, 1.84 Hz, 1H), 8.66 (dd, J=4.78, 1.84 Hz, 1H), 12.27 (s, 1H).

### EXAMPLE 1B

### 1-butyl-2H-pyrido[2,3-d][1,3]oxazine-2,4(1H)-dione

A suspension of sodium hydride (95%, 0.96 g, 40 mmol) in dimethylacetamide (60 mL) at 10° C. under nitrogen was reacted with the product of Example 1A (5.7 g, 34.7 mmol) with stirring for 1 hour then treated with n-butylbromide (5.2 g, 38 mmol) and stirred for an additional 16 hours. The reaction was partitioned between ethyl acetate and water. The organic layer was washed with water and brine, dried over sodium sulfate, filtered and concentrated under vacuum. The crude product was purified by flash column chromatography with silica gel eluting with hexane and ethyl acetate (3:1) to give the title compound as a white solid, (2.5 g, 33% yield). ¹H NMR (300 MHz, DMSO-d₆) δ 0.92 (t, J=7.35 Hz, 3H), 1.36 (m, 2H), 1.65 (m, 2H), 4.13 (m, 2H), 7.38 (dd, J=7.72, 4.78 Hz, 1H), 8.39 (dd, J=7.72, 1.84 Hz, 1H), 8.77 (dd, J=5.15, 1.84 Hz, 1H).

### EXAMPLE 1C

### ethyl(1,1-dioxido-4H-1,2,4-benzothiadiazin-3-yl)acetate

The title compound was prepared as a white solid in two steps (46% yield) from 2-aminobenzenesulfonamide according to the procedure described in Chemistry of Heterocyclic Compounds (English Translation), 1998, 34(7), 791-795. ¹H NMR (300 MHz, DMSO-d₆) δ 1.21 (t, J=7.17 Hz, 3H), 4.16 (q, J=7.23 Hz, 2H), 7.32 (d, J=7.35 Hz, 1H), 7.47 (m, 1H), 7.69 (m, 1H), 7.82 (dd, J=7.91, 1.29 Hz, 1H), 12.27 (s, 1H).

### EXAMPLE 1D

### 1-butyl-3-(1,1-dioxido-4H-1,2,4-benzothiadiazin-3-yl)-4-hydroxy-1,8-naphthyridin-2(1H)-one

To a solution of the product of Example 1B (0.220 g, 1.0 mmol) and the product of Example 1C (0.268 g, 1.0 mmol) in anhydrous THF (10 mL) under nitrogen at 0° C. was added sodium hydride (95%, 0.10 g, 4.0 mmol). The reaction was heated to reflux for 3 hours, cooled to 0° C., and to it was added dropwise glacial acetic acid (2 mL). The resulting mixture was heated to reflux for 2 hours, cooled to ambient temperature, and diluted with aqueous hydrochloric acid (0.1 M, 10 mL). The resulting precipitate was collected by filtration, washed with water and diethyl ether and dried to give the title compound (0.130 g, 33%). MS (ESI−) m/z 397 (M−H)⁻.

A stirred suspension of the title compound (0.130 g, 0.326 mmol) in acetonitrile and water (1:1, 4 mL) was reacted with aqueous sodium hydroxide (1 M, 0.326 mL, 0.326 mmol), for approximately 30 minutes when a clear solution was observed. The solution was lyophilized to give the sodium salt. ¹H NMR (300 MHz, DMSO-d₆) δ 0.92 (t, J=7.35 Hz, 3H), 1.35 (m, 2H), 1.58 (m, 2H), 4.28 (t, J=7.35 Hz, 2H), 7.13 (dd, J=7.72, 4.78 Hz, 1H), 7.28 (m, 2H), 7.55 (m, 1H), 7.66 (dd, J=7.72, 1.47 Hz, 1H), 8.37 (dd, J=7.72, 1.84 Hz, 1H), 8.53 (dd, J=4.60, 2.02 Hz, 1H), 15.92 (s, 1H).

### EXAMPLE 2

### 1-[(5-chloro-2-thienyl)methyl]-3-(1,1-dioxido-4H-1,2,4-benzothiadiazin-3-yl)-4-hydroxy-1,8-naphthyridin-2(1H)-one

### EXAMPLE 2A

### 1-[(5-chloro-2-thienyl)methyl]-2H-pyrido[2,3-d][1,3]oxazine-2,4(1H)-dione

The title compound was prepared according to the procedure of Example 1B substituting 2-chloro-5-chloromethylthiophene for n-butyl bromide (0.195 g, 52%). ¹H NMR (300 MHz, DMSO-d₆) δ 5.38 (s, 2H), 6.98 (d, J=4.04 Hz, 1H), 7.08 (d, J=3.68 Hz, 1H), 7.43 (dd, J=7.72, 4.78 Hz, 1H), 8.41 (dd, J=7.72, 1.84 Hz, 1H), 8.83 (dd, J=4.78, 1.84 Hz, 1H).

### EXAMPLE 2B

### 1-[(5-chloro-2-thienyl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 2A for the product of Example 1B (0.167 g, 58%). MS (ESI−) m/z 471/473 (M−H)⁻.

The sodium salt of the title compound was prepared according to the procedure of Example 1D. ¹H NMR (300 MHz, DMSO-d₆) δ 5.53 (s, 2H), 6.89 (d, J=3.68 Hz, 1H), 7.00 (d, J=3.68 Hz, 1H), 7.20 (dd, J=7.72, 4.78 Hz, 1H), 7.29 (m, 2H), 7.56 (t, J=7.72 Hz, 1H), 7.67 (d, J=7.72 Hz, 1H), 8.40 (dd, J=7.72, 1.84 Hz, 1H), 8.58 (dd, J=4.78, 1.84 Hz, 1H), 15.73 (s, 1H).

### EXAMPLE 3

### 3-(1,1-dioxido-4H-1,2,4-benzothiadiazin-3-yl)-1-(2-ethylbutyl)-4-hydroxy-1,8-naphthyridin-2(1H)-one

### EXAMPLE 3A

### ethyl 2-[(2-ethylbutyl)amino]nicotinate

Ethyl 2-chloronicotinate (0.646 g, 3.48 mmol) and 2-ethylbutylamine (0.74 g, 7.31 mmol) were reacted in a sealed tube at 130° C. for 2 hours. The reaction mixture was partitioned between dichloromethane and water. The aqueous layer was extracted with dichloromethane (2×50 mL). The organic layers were combined and dried over magnesium sulfate, filtered, and concentrated. The residue was purified by column chromatography on silica gel eluting with hexane/ethyl acetate (19:1) to provide the title compound (0.665 g, 76%). MS (ESI+) m/z 251.1 (M+H)⁺; ¹H NMR (300 MHz, CDCl₃) δ 0.93 (t, J=7.54 Hz, 6H), 1.41 (m, 7H), 1.55 (m, 1H), 3.46 (m, 2H), 4.32 (q, J=6.99 Hz, 2H), 6.48 (dd, J=7.72, 4.78 Hz, 1H), 7.99 (s, 1H), 8.11 (dd, J=7.72, 2.21 Hz, 1H), 8.27 (dd, J=4.78, 1.84 Hz, 1H).

### EXAMPLE 3B

### 1-(2-ethylbutyl)-2H-pyrido[2,3-d][1,3]oxazine-2,4(1H)-dione

The product of Example 3A (0.664 g, 2.65 mmol) and diphosgene (1.57 g, 7.96 mmol) in 13 mL of 1,2-dichloroethane and 1.3 mL of 1,4 dioxane were reacted at 80° C. for 16 hours. The reaction was concentrated under vacuum and the residue was purified by flash column chromatography on silica gel eluting with hexane/ethyl acetate (9:1) to provide the title compound (0.235 g, 36%). ¹H NMR (300 MHz, CDCl₃) δ 0.95 (m, 6H), 1.40 (m, 4H), 1.52 (m, 2H), 4.21 (m, 1H), 7.25 (m, 1H), 8.41 (dd, J=7.72, 1.84 Hz, 1H), 8.70 (dd, J=4.78, 1.84 Hz, 1H).

### EXAMPLE 3C

### 3-(1,1-dioxido-4H-1,2,4-benzothiadiazin-3-yl)-1-(2-ethylbutyl)-4-hydroxy-1,8-naphthyridin-2(1H)-one

The title compound was prepared according to the procedure of Example 1D substituting the product of Example 3B for the product of Example 1B (0.041 g, 38%). MS (ESI+) m/z 427.1 (M+H)⁺, (ESI−) m/z 425.1 (M−H)⁻; ¹H NMR (300 MHz, DMSO-d₆) δ 0.87 (t, J=7.54 Hz, 6H), 1.30 (m, 4H), 1.99 (m, 1H), 4.44 (d, J=7.35 Hz, 2H), 7.49 (dd, J=7.72, 4.78 Hz, 1H), 7.55 (t, J=7.35 Hz, 1H), 7.68 (d, J=8.09 Hz, 1H), 7.77 (t, J=7.17 Hz, 1H), 7.92 (d, J=8.09 Hz, 1H), 8.57 (dd, J=7.72, 1.84 Hz, 1H), 8.86 (d, J=4.78 Hz, 1H). The sodium salt of the title compound was prepared according to the procedure of Example 1D. MS (ESI+) m/z 427.1 (M+H)⁺, (ESI−) m/z 425.1 (M−H)⁻; ¹H NMR (300 MHz, DMSO-d₆) δ 0.86 (t, J=7.35 Hz, 6H), 1.28 (m, 4H), 1.91 (m, 1H), 4.25 (d, J=7.35 Hz, 2H), 7.12 (dd, J=7.72, 4.78 Hz, 1H), 7.28 (m, 2H), 7.55 (m, 1H), 7.67 (dd, J=8.09, 1.47 Hz, 1H), 8.37 (dd, J=7.72, 1.84 Hz, 1H), 8.50 (dd, J=4.60, 2.02 Hz, 1H), 15.97 (s, 1H).

### EXAMPLE 4

### 1-[(5-bromo-2-thienyl)methyl]-3-(1,1-dioxido-4H-1,2,4-benzothiadiazin-3-yl)-4-hydroxy-1,8-naphthyridin-2(1H)-one

### EXAMPLE 4A

### 1-[(5-bromo-2-thienyl)methyl]-2H-pyrido[2,3-d][1,3]oxazine-2,4(1H)-dione

The title compound was prepared according to the procedure of Example 1B substituting 2-bromo-5-chloromethylthiophene for n-butyl bromide (0.229 g, 55%). ¹H NMR (300 MHz, DMSO-d₆) δ 5.40 (s, 2H), 7.06 (m, 2H), 7.43 (dd, J=7.72, 4.78 Hz, 1H), 8.41 (dd, J=7.72, 1.84 Hz, 1H), 8.83 (dd, J=4.78, 1.84 Hz, 1H).

### EXAMPLE 4B

### 1-[(5-bromo-2-thienyl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 4A for the product of Example 1B (0.208 g, 60%). MS (ESI−) m/z 515/517 (M−H)⁻. The sodium salt of the title compound was prepared according to the procedure of Example 1D. ¹H NMR (300 MHz, DMSO-d₆) δ 5.55 (s, 2H), 6.97 (d, J=3.68 Hz, 1H), 7.00 (d, J=3.68 Hz, 1H), 7.20 (dd, J=7.73, 4.78 Hz, 1H), 7.29 (m, 2H), 7.56 (m, 1H), 7.68 (d, J=7.72 Hz, 1H), 8.40 (dd, J=7.72, 2.21 Hz, 1H), 8.58 (dd, J=4.78, 2.20 Hz, 1H), 15.73 (s, 1H).

### EXAMPLE 5

### 3-(1,1-dioxido-4H-1,2,4-benzothiadiazin-3-yl)-4-hydroxy-1-(3-methylbenzyl)-1,8-naphthyridin-2(1H)-one

### EXAMPLE 5A

### 1-(3-methylbenzyl)-2H-pyrido[2,3-d][1,3]oxazine-2,4(1H)-dione

The title compound was prepared according to the procedure of Example 1B substituting 3-methylbenzyl bromide for n-butyl bromide (0.305 g, 62%). MS (DCI) m/z 269 (M+H)⁺.

### EXAMPLE 5B

### 3-(1,1-dioxido-4H-1,2,4-benzothiadiazin-3-yl)-4-hydroxy-1-(3-methylbenzyl)-1,8-naphthyridin-2(1H)-one

The title compound was prepared according to the procedure of Example 1D substituting the product of Example 5A for the product of Example 1B (0.112 g, 72%). MS (ESI−) m/z 445 (M−H)^{—}. The sodium salt of the title compound was prepared according to the procedure of Example 1D. ¹H NMR (300 Mz, DMSO-d₆) δ 2.23 (s, 3H), 5.48 (s, 2H), 7.01 (m, 3H), 7.14 (t, J=7.35 Hz, 2H), 7.28 (m, 2H), 7.56 (td, J=7.72, 1.47 Hz, 1H), 7.67 (dd, J=7.72, 1.47 Hz, 1H), 8.41 (dd, J=7.72, 1.84 Hz, 1H), 8.48 (dd, J=4.78, 1.84 Hz, 1H), 15.86 (s, 11H).

### EXAMPLE 6

### 3-(1,1-dioxido-4H-1,2,4-benzothiadiazin-3-yl)-4-hydroxy-1-(3-nitrobenzyl)-1,8-naphthyridin-2(1H)-one

### EXAMPLE 6A

### 1-(3-nitrobenzyl)-2H-pyrido[2,3-d][1,3]oxazine-2,4(1H)-dione

The title compound was prepared according to the procedure of Example 1B substituting 3-nitrobenzyl bromide for n-butyl bromide (0.147 g, 28%). MS (DCI) m/z 300 (M+H)⁺.

### EXAMPLE 6B

### 3-(1,1-dioxido-4H-1,2,4-benzothiadiazin-3-yl)-4-hydroxy-1-(3-nitrobenzyl)-1,8-naphthyridin-2(1H)-one

The title compound was prepared according to the procedure of Example 1D substituting the product of Example 6A for the product of Example 1B (0.032 g, 42%). MS (ESI−) m/z 476 (M−H)^{—}. The sodium salt of the title compound was prepared according to the procedure of Example 1D. ¹H NMR (300 MHz, DMSO-d₆) δ 5.62 (s, 2H), 7.19 (dd, J=7.72, 4.78 Hz, 1H), 7.29 (td, J=8.36, 1.29 Hz, 2H), 7.56 (t, J=8.46 Hz, 1H), 7.58 (t, J=7.72 Hz, 1H), 7.67 (d, J=8.09 Hz, 1H), 7.74 (d, J=8.09 Hz, 1H), 8.08 (m, 2H), 8.43 (dd, J=7.54, 2.02 Hz, 1H), 8.50 (dd, J=4.60, 2.02 Hz, 1H), 15.76 (s, 1H).

### EXAMPLE 7

### 3-(1,1-dioxido-4H-1,2,4-benzothiadiazin-3-yl)-4-hydroxy-1-(3-thienylmethyl)-1,8-naphthyridin-2(1H)-one

### EXAMPLE 7A

### 1-(3-thienylmethyl)-2H-pyrido [2,3-d][2,3]oxazine-2.4(1H)-dione

The title compound was prepared according to the procedure of Example 1B substituting 3-(bromomethyl)thiophene for n-butyl bromide (0.170 g, 52%). ¹H NMR (300 MHz, DMSO-d₆) δ 5.32 (s, 2H), 7.15 (m, 1H), 7.40 (dd, J=7.72, 4.78 Hz, 1H), 7.48 (m, 2H), 8.41 (dd, J=7.72, 1.84 Hz, 1H), 8.76 (dd, J=4.78, 1.84 Hz, 1H).

### EXAMPLE 7B

### 3-(1,1-dioxido-4H-1,2,4-benzothiadiazin-3-yl)-4-hydroxy-1-(3-thienylmethyl)-1,8-naphthyridin-2(1H)-one

The title compound was prepared according to the procedure of Example 1D substituting the product of Example 7A for the product of Example 1B (0.135 g, 48%). MS (ESI−) m/z 437 (M−H)⁻. The sodium salt of the title compound was prepared according to the procedure of Example 1D. ¹H NMR (300 MHz, DMSO-d₆) δ 5.48 (s, 2H), 7.09 (d, J=4.04 Hz, 1H), 7.16 (dd, J=7.72, 4.78 Hz, 1H), 7.27 (m, 3H), 7.38 (dd, J=4.96, 3.13 Hz, 1H), 7.56 (t, J=7.72 Hz, 1H), 7.67 (d, J=8.09 Hz, 1H), 8.39 (dd, J=7.72, 1.66 Hz, 1H), 8.53 (dd, J=4.78, 1.66 Hz, 1H), 15.85 (s, 1H).

### EXAMPLE 8

### 1-(3-chlorobenzyl)-3-(1,1-dioxido-4H-1,2,4-benzothiadiazin-3-yl)-4-hydroxy-1,8-naphthyridin-2(1H)-one

### EXAMPLE 8A

### 1-(3-chlorobenzyl)-2H-pyrido[2,3-d][1,3]oxazine-2,4(1H)-dione

The title compound was prepared according to the procedure of Example 1B substituting 3-chlorobenzyl bromide for n-butyl bromide (0.405 g, 77%). MS (DCI) m/z 289 (M+H)⁺.

### EXAMPLE 8B

### 1-(3-chlorobenzyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 8A for the product of Example 1B (0.050 g, 45%). MS (ESI−) m/z 465 (M−H)⁻. The sodium salt of the title compound was prepared according to the procedure of Example 1D. ¹H NMR (300 MHz, DMSO-d₆) δ 5.50 (s, 2H), 7.18 (dd, J=7.72, 4.78 Hz, 1H), 7.27 (m, 6H), 7.56 (td, J=7.91, 1.47 Hz, 1H), 7.67 (d, J=7.72 Hz, 1H), 8.42 (dd, J=7.72, 1.84 Hz, 1H), 8.49 (dd, J=4.78, 2.21 Hz, 1H), 15.78 (s, 1H).

### EXAMPLE 9

### 1-(3-bromobenzyl)-3-(1,1-dioxido-4H-1,2,4-benzothiadiazin-3-yl)-4-hydroxy-1,8-naphthyridin-2(1H)-one

### EXAMPLE 9A

### 1-(3-bromobenzyl)-2H-pyrido [2,3-d][1,3]oxazine-2,4(1H)-dione

The title compound was prepared according to the procedure of Example 1B substituting 3-bromobenzyl bromide for n-butyl bromide (0.500 g, 82%). MS (DCI) m/z 333 (M+H)⁺.

### EXAMPLE 9B

### 1-(3-bromobenzyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 9A for the product of Example 1B (0.050 g, 45%). MS (ESI−) m/z 465 (M−H)⁻. The sodium salt of the title compound was prepared according to the procedure of Example 1D. ¹H NMR (300 MHz, DMSO-d₆) δ 5.50 (s, 2H), 7.18 (dd, J=7.72, 4.78 Hz, 1H), 7.27 (m, 6H), 7.56 (td, J=7.91, 1.47 Hz, 1H), 7.67 (d, J=7.72 Hz, 1H), 8.42 (dd, J=7.72, 1.84 Hz, 1H), 8.49 (dd, J=4.78, 2.21 Hz, 1H), 15.78 (s, 1H).

### EXAMPLE 10

### 1-[(2-chloro-1,3-thiazol-5-yl)methyl]-3-(1,1-dioxido-4H-1,2,4-benzothiadiazin-3-yl)-4-hydroxy-1,8-naphthyridin-2(1H)-one

### EXAMPLE 10A

### 1-[(2-chloro-1,3-thiazol-5-yl)methyl]-2H-pyrido[2,3-d][1,3]oxazine-2,4(1H)-dione

The title compound was prepared according to the procedure of Example 1B substituting 2-chloro-5-bromomethylthiazole for n-butyl bromide (0.360 g, 60%). MS (APCI) m/z 296 (M+H)⁺; ¹H NMR (300 MHz, DMSO-d₆) δ 5.45 (s, 2H), 7.44 (dd, J=7.72, 4.78 Hz, 1H), 7.76 (s, 1H), 8.42 (dd, J=7.91, 1.65 Hz, 1H), 8.82 (dd, J=4.78, 1.84 Hz, 1H).

### EXAMPLE 10B

### 1-[(2-chloro-1,3-thiazol-5-yl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 10A for the product of Example 1B (0.136 g, 60%). MS (ESI−) m/z 477 (M−H)⁻; ¹H NMR (300 MHz, DMSO-d₆) δ 5.76 (s, 2H), 7.56 (m, 2H), 7.65 (d, J=7.35 Hz, 1H), 7.77 (s, 1H), 7.78 (m, 1H), 7.92 (d, J=8.09 Hz, 1H), 8.59 (dd, J=8.09, 1.84 Hz, 1H), 8.92 (dd, J=4.78, 1.84 Hz, 1H), 13.72 (s, 1H).

### EXAMPLE 11

### 3-(1,1-dioxido-4H-1,2,4-benzothiadiazin-3-yl)-1-(3-fluorobenzyl)-4-hydroxy-1,8-naphthyridin-2(1H)-one

### EXAMPLE 11A

### 1-(3-fluorobenzyl)-2H-pyrido[2,3-d][1,3]oxazine-2,4(1H)-dione

The title compound was prepared according to the procedure of Example 1B substituting 3-fluorobenzyl bromide for n-butyl bromide (0.382 g, 76%). MS (DCI) m/z 273 (M+H)⁺.

### EXAMPLE 11B

### 3-(1,1-dioxido-4H-1,2,4-benzothiadiazin-3-yl)-1-(3-fluorobenzyl)-4-hydroxy-1,8-naphthyridin-2(1H)-one

The title compound was prepared according to the procedure of Example 1D substituting the product of Example 11A for the product of Example 1B (0.040 g, 37%). MS (ESI−) m/z 449 (M−H)⁻. The sodium salt of the title compound was prepared according to the procedure of Example 1D. ¹H NMR (300 MHz, DMSO-d₆) δ 5.52 (s, 2H), 7.02 (m, 2H), 7.08 (d, J=7.72 Hz, 1H), 7.17 (dd, J=7.72, 4.41 Hz, 1H), 7.29 (m, 3H), 7.56 (td, J=7.91, 1.47 Hz, 1H), 7.67 (d, J=8.09 Hz, 1H), 8.41 (dd, J=7.72, 1.84 Hz, 1H), 8.49 (dd, J=4.78, 1.84 Hz, 1H), 15.79 (s, 1H).

### EXAMPLE 12

### 3-(1,1-dioxido-4H-1,2,4-benzothiadiazin-3-yl)-4-hydroxy-1-(3-methylbutyl)-1,8-naphthyridin-2(1H)-one

### EXAMPLE 12A

### 1-(3-methylbutyl)-2H-pyrido[2,3-d][1,3]oxazine-2,4(1H)-dione

The title compound was prepared according to the procedure of Example 1B substituting 1-bromo-3-methylbutane for n-butyl bromide. (0.218 g, 51%). MS (ESI−) m/z 233 (M−H)⁻; ¹H NMR (300 MHz, DMSO-d₆) δ 0.93 (s, 3H), 0.96 (s, 3H), 1.55 (m, 2H), 1.66 (m, 1H), 4.14 (t, J=7.72 Hz, 2H), 7.37 (dd, J=7.91, 4.96 Hz, 1H), 8.38 (dd, J=7.72, 1.84 Hz, 1H), 8.78 (dd, J=4.78, 1.84 Hz, 1H).

### EXAMPLE 12B

### 3-(1,1-dioxido-4H-1,2,4-benzothiadiazin-3-yl)-4-hydroxy-1-(3-methylbutyl)-1,8-naphthyrdin-2(1H)-one

The title compound was prepared according to the procedure of Example 1D substituting the product of Example 12A for the product of Example 1B (0.031 g, 18%). MS (ESI−) m/z 411 (M−H)⁻. The sodium salt of the title compound was prepared according to the procedure of Example 1D. MS (ESI−) m/z 411 (M−H)⁻; ¹H NMR (300 MHz, DMSO-d₆) δ 0.95 (s, 3H), 0.98 (s, 3H), 1.47 (m, 2H), 1.64 (m, 1H), 4.30 (t, J=7.72 Hz, 2H), 7.13 (dd, J=7.72, 4.78 Hz, 1H), 7.26 (d, J=8.09 Hz, 1H), 7.30 (d, J=7.72 Hz, 1H), 7.55 (t, J=7.72 Hz, 1H), 7.66 (d, J=8.09 Hz, 1H), 8.37 (dd, J=7.72, 1.84 Hz, 1H), 8.53 (dd, J=4.78, 2.21 Hz, 1H), 15.94 (s, 1H).

### EXAMPLE 13

### 1-(cyclobutylmethyl)-3-(1,1-dioxido-4H-1,2,4-benzothiadiazin-3-yl)-4-hydroxy-1,8-naphthyridin-2(1H)-one

### EXAMPLE 13A

### 1-(cyclobutylmethyl)-2H-pyrido[2,3-d][1,3]oxazine-2,4(1H)-dione

The title compound was prepared according to the procedure of Example 1B substituting bromomethyl-cyclobutane for n-butyl bromide (0.255 g, 60%). MS (DCI) m/z 233 (M+H)⁺.

### EXAMPLE 13B

### 1-(cyclobutylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 13A for the product of Example 1B (0.120 g, 52%). MS (ESI−) m/z 409 (M−H)⁻. The sodium salt of the title compound was prepared according to the procedure of Example 1D. ¹H NMR (300 MHz, DMSO-d₆) δ 1.83 (m, 6H), 2.79 (m, 1H), 4.38 (d, J=6.99 Hz, 2H), 7.13 (dd, J=7.72, 4.78 Hz, 1H), 7.29 (t, J=7.54 Hz, 2H), 7.55 (t, J=7.72 Hz, 1H), 7.67 (d, J=7.72 Hz, 1H), 8.36 (dd, J=7.72, 2.21 Hz, 1H), 8.51 (dd, J=4.78, 1.84 Hz, 1H), 15.92 (s, 1H).

### EXAMPLE 14

### 3-(1,1-dioxido-4H-1,2,4-benzothiadiazin-3-yl)-4-hydroxy-1-[(5-methyl-2-thienyl)methyl]-1,8-naphthyridin-2(1H)-one

### EXAMPLE 14A

### 1-[(5-methyl-2-thienyl)methyl]-2H-pyrido[2,3-d][1,3]oxazine-2,4(1H)-dione

The title compound was prepared according to the procedure of Example 1B substituting 2-bromomethyl-5-methylthiophene for n-butyl bromide (0.181 g, 54%). ¹H NMR (300 MHz, DMSO-d₆) δ 2.36 (s, 3H), 5.38 (s, 2H), 6.63 (m, 1H), 6.98 (d, J=3.68 Hz, 1H), 7.42 (dd, J=7.72, 4.78 Hz, 1H), 8.41 (dd, J=7.72, 1.84 Hz, 1H), 8.82 (dd, J=4.78, 1.84 Hz, 1H).

### EXAMPLE 14B

### 3-(1,1-dioxido-4H-1,2,4-benzothiadiazin-3-yl)-4-hydroxy-1-[(5-methyl-2-thienyl)methyl]-1,8-naphthyridin-2(1H)-one

The title compound was prepared according to the procedure of Example 1D substituting the product of Example 14A for the product of Example 1B (0.172 g, 58%). MS (ESI−) m/z 451 (M−H)⁻. The sodium salt of the title compound was prepared according to the procedure of Example 1D. ¹H NMR (300 MHz, DMSO-d₆) δ 2.32 (s, 3H), 5.54 (s, 2H), 6.56 (d, 1H), 6.88 (d, J=3.31 Hz, 1H), 7.17 (dd, J=7.72, 4.78 Hz, 1H), 7.28 (m, 2H), 7.56 (t, J=7.72 Hz, 1H), 7.67 (d, J=7.72 Hz, 1H), 8.38 (dd, J=7.72, 1.84 Hz, 1H), 8.56 (dd, J=4.78, 1.84 Hz, 1H), 15.81 (s, 1H).

### EXAMPLE 15

### 1-benzyl-3-(1,1-dioxido-4H-1,2,4-benzothiadiazin-3-yl)-4-hydroxy-1,8-naphthyridin-2(1H)-one

### EXAMPLE 15A

### 1-benzyl-2H-pyrido[2,3-d][1,3]oxazine-2,4(1H)-dione

The title compound was prepared according to the procedure of Example 1B substituting benzyl bromide for n-butyl bromide (0.393 g, 51%). MS (DCI) m/z 255 (M+H)⁺.

### EXAMPLE 15B

### 1-benzyl-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 15A for the product of Example 1B (0.217 g, 62%). MS (ESI−) m/z 431 (M−H)⁻. The sodium salt of the title compound was prepared according to the procedure of Example 1D. ¹H NMR (300 MHz, DMSO-d₆) δ 5.52 (s, 2H), 7.16 (m, 2H), 7.25 (d, J=4.41 Hz, 4H), 7.29 (m, 2H), 7.56 (td, J=7.91, 1.47 Hz, 1H), 7.67 (dd, J=7.91, 1.65 Hz, 1H), 8.41 (dd, J=7.54, 2.02 Hz, 1H), 8.48 (dd, J=4.78, 2.21 Hz, 1H), 15.84 (s, 1H).

### EXAMPLE 16

### 3-(1,1-dioxido-4H-1,2,4-benzothiadiazin-3-yl)-4-hydroxy-1-[(5-methyl-3-pyridinyl)methyl]-1,8-naphthyridin-2(1H)-one

### EXAMPLE 16A 1-[(5-methyl-3-pyridinyl)methyl]-2H-pyrido[2,3-d][1,3]oxazine-2,4(1H)-dione

The title compound was prepared according to the procedure of Example 1B substituting 3-chloromethyl-5-methylpyridine for n-butyl bromide (0.080 g, 24%). ¹H NMR (300 MHz, DMSO-d₆) δ 2.25 (s, 3H), 5.34 (s, 2H), 7.40 (dd, J=7.72, 4.78 Hz, 1H), 7.63 (br s, 1H), 8.30 (br s, 1H), 8.43 (dd, J=7.72, 1.84 Hz, 1H), 8.46 (br s, 1H), 8.73 (dd, J=4.78, 1.84 Hz, 1H).

### EXAMPLE 16B

### 3-(1,1-dioxido-4H-1,2,4-benzothiadiazin-3-yl)-4-hydroxy-1-[(5-methyl-3-pyridinyl)methyl]-1,8-naphthyridin-2(1H)-one

The title compound was prepared according to the procedure of Example 1D substituting the product of Example 16A for the product of Example 1B (0.013 g, 13%). MS (ESI−) m/z 446 (M−H)⁻. The sodium salt of the title compound was prepared according to the procedure of Example 1D. ¹H NMR (300 MHz, DMSO-d₆) δ 2.22 (s, 3H), 5.49 (s, 2H), 7.18 (dd, J=7.72, 4.78 Hz, 1H), 7.29 (m, 2H), 7.44 (s, 1H), 7.56 (m, 1H), 7.67 (d, J=8.09 Hz, 1H), 8.23 (d, J=1.47 Hz, 11H), 8.36 (d, J=1.47 Hz, 1H), 8.41 (dd, J=7.72, 1.84 Hz, 1H), 8.51 (dd, J=4.78, 1.84 Hz, 1H), 15.80 (s, 1H).

### EXAMPLE 17

### 1-[(2-chloro-4-pyridinyl)methyl]-3-(1,1-dioxido-4H-1,2,4-benzothiadiazin-3-yl)-4-hydroxy-1,8-naphthyridin-2(1H)-one

### EXAMPLE 17A

### 1-[(2-chloro-4-pyridinyl)methyl]-2H-pyrido[2,3-d][1,3]oxazine-2,4(1H)-dione

The title compound was prepared according to the procedure of Example 1B substituting 4-bromomethyl-2-chloropyridine for n-butyl bromide (0.219 g, 62%). ¹H NMR (300 MHz, DMSO-d₆) δ 5.37 (s, 2H), 7.40 (m, 1H), 7.48 (s, 1H), 7.60 (s, 1H), 8.34 (dd, J=4.60, 2.39 Hz, 1H), 8.45 (m, 1H), 8.68 (m, 1H).

### EXAMPLE 17B

### 1-[(2-chloro-4-pyridinyl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 17A for the product of Example 1B (0.255 g, 73%). MS (ESI−) m/z 466/468 (M−H)⁻. The sodium salt of the title compound was prepared according to the procedure of Example 1D. ¹H NMR (300 MHz, DMSO-d₆) δ 5.52 (s, 2H), 7.19 (m, 2H), 7.30 (m, 3H), 7.56 (t, J=7.54 Hz, 1H), 7.67 (d, J=7.72 Hz, 1H), 8.27 (d, J=5.15 Hz, 1H), 8.46 (m, 2H), 15.72 (s, 1H).

### EXAMPLE 18

### 1-[(5-bromo-3-pyridinyl)methyl]-3-(1,1-dioxido-4H-1,2,4-benzothiadiazin-3-yl)-4-hydroxy-1,8-naphthyridin-2(1H)-one

### EXAMPLE 18A

### di-tert-butyl(5-bromo-3-pyridinyl)methylimidodicarbonate

A solution of 5-bromo-3-chloromethylpyridinium hydrochloride (716 mg, 4.189 mmol) in anhydrous DMF (15 mL) under nitrogen at 0° C. was treated with triethylamine (0.65 mL, 4.61 mmol), tetrabutylammonium bromide (273 mg, 0.838 mmol), and potassium di-tert-butyl imidodicarbonate (1.284 g, 5.027 mmol). The reaction was heated to 50° C.-55° C. for 3.5 hours, then cooled to room temperature, diluted with ethyl acetate (150 mL), and washed with water (2×50 mL) and saturated aqueous sodium chloride. The combined extracts were dried over anhydrous Na₂SO₄, filtered, and concentrated by rotary evaporation. The residue was purified by flash column chromatography on silica gel with 6% ethyl acetate/dichloromethane to give the title compound as a colorless oil (0.980 g, 60%). MS (ESI+) m/z 387/389 (M+H)⁺; ¹H NMR (300 MHz, CDCl₃) δ 1.49 (s, 18H), 4.75 (s, 2H), 7.83 (t, J=2.02 Hz, 1H), 8.50 (d, J=1.84 Hz, 1H), 8.58 (d, J=2.21 Hz, 1H).

### EXAMPLE 18B

### (5-bromo-3-pyridinyl)methylamine

The product of Example 18A (0.98 g, 2.53 mmol) was treated with trifluoroacetic acid and dichloromethane (1:1 v/v, 20 mL) for 2 hours at room temperature. The solvent was removed by rotary evaporation and the resulting oil was chased with benzene/dichloromethane (3 times) to give a waxy solid. The salt was dissolved in anhydrous methanol (20 mL) and stirred with Amberlite IRA-400(OH), resin (10 g) for 2 hrs. The resin was removed by vacuum filtration and thoroughly washed with dry methanol. The filtrate was concentrated by rotary evaporation to give the title compound (0.415 g, 88%). MS (DCI/NH₃) m/z 187/189 (M+H)⁺; ¹H NMR (300 MHz, DMSO-d₆) δ 3.73 (s, 2H), 8.02 (t, J=2.02 Hz, 1H), 8.50 (d, J=1.47 Hz, 1H), 8.53 (d, J=2.21 Hz, 1H).

### EXAMPLE 18C

### ethyl 2-{[(5-bromo-3-pyridinyl)methyl]amino}nicotinate

The title compound was prepared according to the procedure of Example 3A substituting the product of Example 18B for 2-ethylbutylamine (0.116 g, 68%). MS (DCI/NH₃) m/z 336/338 (M+H)⁺; ¹H NMR (300 MHz, DMSO-d₆) δ 1.32 (t, J=6.99 Hz, 3H), 4.31 (q, J=7.23 Hz, 2H), 4.71 (d, J=5.88 Hz, 2H), 6.67 (dd, J=7.72, 4.78 Hz, 1H), 7.97 (t, J=2.02 Hz, 1H), 8.12 (dd, J=7.72, 2.21 Hz, 1H), 8.26 (dd, J=4.78, 1.84 Hz, 1H), 8.45 (t, J=6.07 Hz, 1H), 8.55 (m, 2H).

### EXAMPLE 18D

### 1-[(5-bromo-3-pyridinyl)methyl]-2H-pyrido[2,3-d][1,3]oxazine-2,4(1H)-dione

The title compound was prepared according to the procedure of Example 3B substituting the product of Example 18C for the product of Example 3A and purifying by flash column chromatography on silica gel eluting with 10% ethyl acetate/dichloromethane (0.057 g, 51%). ¹H NMR (300 MHz, DMSO-d₆) δ 5.38 (s, 2H), 7.41 (dd, J=7.72, 5.15 Hz, 1H), 8.10 (t, J=2.02 Hz, 1H), 8.43 (dd, J=7.72, 1.84 Hz, 1H), 8.60 (d, J=2.21 Hz, 1H), 8.66 (d, J=1.84 Hz, 1H), 8.72 (dd, J=5.15, 1.84 Hz, 1H).

### EXAMPLE 18E

### 1-[(5-bromo-3-pyridinyl)methyl]-3-(1,1-dioxido-4H-1,2,4-benzothiadiazin-3-yl)-4-hydroxy-1,8-naphthyridin-2(11H)-one

The title compound was prepared according to the procedure of Example 1D substituting the product of Example 18D for the product of Example 1B (0.037 g, 43%). MS (ESI−) m/z 510/512 (M−H)⁻. The sodium salt of the title compound was prepared according to the procedure of Example 1D. ¹H NMR (300 MHz, DMSO-d₆) δ 5.52 (s, 2H), 7.20 (dd, J=7.72, 4.78 Hz, 1H), 7.29 (m, 2H), 7.56 (t, J=7.54 Hz, 1H), 7.68 (d, J=7.35 Hz, 1H), 7.89 (br s, 1H), 8.42 (dd, J=7.72, 1.84 Hz, 1H), 8.52 (dd, J=4.78, 1.84 Hz, 1H), 8.55 (br s, 2H), 15.73 (s, 1H).

### EXAMPLE 19

### 1-(cyclohexylmethyl)-3-(1,1-dioxido-4H-1,2,4-benzothiadiazin-3-yl)-4-hydroxy-1,8-naphthyridin-2(1H)-one

### EXAMPLE 19A

### 1-(cyclohexylmethyl)-2H-pyrido[2,3-d][1,3]oxazine-2,4(1H)-dione

The title compound was prepared according to the procedure of Example 1B substituting (bromomethyl)cyclohexane for n-butyl bromide (0.05 g, 11%).

### EXAMPLE 19B

### 1-(cyclohexylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 19A for the product of Example 1B (0.025 g, 30%). MS (ESI−) m/z 437 (M−H)^{—}. The sodium salt of the title compound was prepared according to the procedure of Example 1D. MS (ESI−) m/z 437 (M−H)⁻; ¹H NMR (300 MHz, DMSO-d₆/TFA) δ 0.99 (m, 5H), 1.50 (m, 5H), 1.87 (m, 1H), 4.32 (d, J=7.35 Hz, 2H), 7.23 (dd, J=8.09, 4.78 Hz, 1H), 7.38 (m, 2H), 7.57 (m, 1H), 7.78 (d, J=8.09 Hz, 1H), 8.40 (dd, J=8.09, 1.84 Hz, 1H), 8.66 (dd, J=4.78, 1.84 Hz, 1H).

### EXAMPLE 20

### 3-(1,1-dioxido-4H-1,2,4-benzothiadiazin-3-yl)-4-hydroxy-1-[(2S)-2-methylbutyl]-1,8-naphthyridin-2(1H)-one

### EXAMPLE 20A

### ethyl 2-[{1(2S)-2-methylbutyl]amino}nicotinate

The title compound was prepared according to the procedure of Example 3A substituting (S)-(−)-2-methylbutylamine for 2-ethylbutylamine (1.6 g, 77%). ¹H NMR (300 MHz, DMSO-d₆) δ 0.89 (t, J=7.23, 3H), 0.91 (d, J=6.62 Hz, 3H), 1.18 (m, 1H), 1.31 (t, J=6.99 Hz, 3H), 1.42 (m, 1H), 1.66 (m, 1H), 3.35 (m, 2H), 4.29 (q, J=7.23 Hz, 2H), 6.59 (dd, J=7.72, 4.78 Hz, 1H), 8.01 (t, J=5.52 Hz, 1H), 8.08 (dd, J=7.72, 1.84 Hz, 1H), 8.27 (dd, J=4.60, 2.02 Hz, 1H).

### EXAMPLE 20B

### 1-[(2S)-2-methylbutyl]-2H-pyrido[2,3-d][1,3]oxazine-2,4(1H)-dione

The title compound was prepared according to the procedure of Example 3B substituting the product of Example 20A for the product of Example 3A (0.400 g, 68%). MS (DCI) m/z 252 (M+NH₄)⁺.

### EXAMPLE 20C

### 3-(1,1-dioxido-4H-1,2,4-benzothiadiazin-3-yl)-4-hydroxy-1-[(2S)-2-methylbutyl]-1,8-naphthyridin-2(1H)-one

The title compound was prepared according to the procedure of Example 1D substituting the product of Example 20B for the product of Example 1B (0.116 g, 43%). MS (ESI−) m/z 411 (M−H)⁻. The sodium salt of the title compound was prepared according to the procedure of Example 1D. ¹H NMR (300 MHz, DMSO-d₆) δ 0.80 (d, J=6.99 Hz, 3H), 0.87 (t, J=7.54 Hz, 3H), 1.15 (m, 1H), 1.37 (m, 1H), 2.02 (m, 1H), 4.20 (d, J=7.35 Hz, 2H), 7.12 (dd, J=7.72, 4.78 Hz, 1H), 7.27 (m, 2H), 7.55 (m, 1H), 7.66 (d, J=7.72 Hz, 1H), 8.37 (dd, J=7.72, 2.21 Hz, 1H), 8.51 (dd, J=4.60, 2.02 Hz, 1H), 15.95 (s, 1H).

### EXAMPLE 21

### 3-(1,1-dioxido-4H-1,2,4-benzothiadiazin-3-yl)-4-hydroxy-1-(4-methylbenzyl)-1,8-naphthyridin-2(1H)-one

### EXAMPLE 21A

### 1-(4-methylbenzyl)-2H-pyrido[2,3-d][1,3]oxazine-2.4(1H)-dione

The title compound was prepared according to the procedure of Example 1B substituting 4-methylbenzyl bromide for n-butyl bromide (0.402 g, 82%). MS (DCI) m/z 269 (M+H)⁺.

Example 21B 3-(1,1-dioxido-4H-1,2,4-benzothiadiazin-3-yl)-4-hydroxy-1-(4-methylbenzyl)-1,8-naphthyridin-2(1H)-one

The title compound was prepared according to the procedure of Example 1D substituting the product of Example 21A for the product of Example 1B (0.099 g, 60%). MS (ESI−) m/z 445 (M−H)⁻. The sodium salt of the title compound was prepared according to the procedure of Example 1D. ¹H NMR (300 MHz, DMSO-d₆) δ 2.22 (s, 3H), 5.47 (s, 2H), 7.04 (d, J=7.72 Hz, 2H), 7.14 (m, 3H), 7.29 (t, J=7.35 Hz, 2H), 7.55 (t, J=7.72 Hz, 1H), 7.67 (d, J=7.72 Hz, 1H), 8.39 (dd, J=7.72, 1.84 Hz, 1H), 8.48 (dd, J=4.78, 1.84 Hz, 1H), 15.85 (s, 1H).

### EXAMPLE 22

### 3-(1,1-dioxido-4H-1,2,4-benzothiadiazin-3-yl)-4-hydroxy-1-[(5-nitro-2-furyl)methyl]-1,8-naphthyridin-2(1H)-one

### EXAMPLE 22A

### 1-[(5-nitro-2-furyl)methyl]-2H-pyrido[2,3-d][1,3]oxazine-2,4(1H)-dione

The title compound was prepared according to the procedure of Example 1B substituting 2-bromomethyl-5-nitrofuran for n-butyl bromide (0.120 g, 34%). ¹H NMR (300 MHz, DMSO-d₆) δ 5.45 (s, 2H), 6.90 (d, J=3.68 Hz, 1H), 7.44 (dd, J=7.72, 5.15 Hz, 1H), 7.65 (d, J=3.68 Hz, 1H), 8.45 (m, 1H), 8.77 (m, 1H).

### EXAMPLE 22B

### 3-(1,1-dioxido-4H-1,2,4-benzothiadiazin-3-yl)-4-hydroxy-1-[(5-nitro-2-furyl)methyl]-1,8-naphthyridin-2(1H)-one

The title compound was prepared according to the procedure of Example 1D substituting the product of Example 21A for the product of Example 1B (0.040 g, 21%). MS (DCI/NH₃) m/z 468 (M+H)⁺. The sodium salt of the title compound was prepared according to the procedure of Example 1D. ¹H NMR (300 MHz, DMSO-d₆) δ 5.60 (s, 2H), 6.54 (d, J=3.68 Hz, 1H), 7.22 (dd, J=7.72, 4.78 Hz, 1H), 7.29 (m, 2H), 7.56 (m, 1H), 7.58 (d, J=3.68 Hz, 1H), 7.67 (d, J=8.09 Hz, 1H), 8.43 (dd, J=7.72, 1.84 Hz, 1H), 8.53 (dd, J=4.78, 1.84 Hz, 1H), 15.68 (s, 1H).

### EXAMPLE 23

### 1-(1-benzothien-2-ylmethyl)-3-(1,1-dioxido-4H-1,2,4-benzothiadiazin-3-yl)-4-hydroxy-1,8-naphthyridin-2(1H)-one

### EXAMPLE 23A

### 1-(1-benzothien-2-ylmethyl)-2H-pyrido[2,3-d][1,3]oxazine-2,4(1H)-dione

The title compound was prepared according to the procedure of Example 1B substituting 2-chloromethyl-benzo[b]thiophene for n-butyl bromide (0.160 g, 42%). ¹H NMR (300 MHz, DMSO-d₆) δ 5.58 (s, 2H), 7.33 (m, 2H), 7.44 (dd, J=7.72, 4.78 Hz, 1H), 7.51 (s, 1H), 7.77 (m, 1H), 7.90 (m, 1H), 8.44 (dd, J=7.72, 1.84 Hz, 1H), 8.83 (dd, J=4.78, 1.84 Hz, 1H).

### EXAMPLE 23B

### 1-(1-benzothien-2-ylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 23A for the product of Example 1B (0.148 g, 60%). MS (ESI−) m/z 487 (M−H)⁻. The sodium salt of the title compound was prepared according to the procedure of Example 1D. ¹H NMR (300 MHz, DMSO-d₆) δ 5.74 (s, 2H), 7.20 (dd, J=7.72, 4.78 Hz, 1H), 7.28 (m, 4H), 7.36 (s, 1H), 7.56 (m, 1H), 7.68 (dd, J=7.72, 1.47 Hz, 1H), 7.75 (m, 1H), 7.82 (dd, J=7.72, 1.47 Hz, 1H), 8.41 (dd, J=7.72, 1.84 Hz, 1H), 8.58 (dd, J=4.78, 1.84 Hz, 1H), 15.77 (s, 1H).

### EXAMPLE 24

### 3-(1,1-dioxido-4H-1,2,4-benzothiadiazin-3-yl)-4-hydroxy-1-(3-methoxybenzyl)-1,8-naphthyridin-2(1H)-one

### EXAMPLE 24A

### 1-(3-methoxybenzyl)-2H-pyrido[2,3-d][1,3]oxazine-2,4(1H)-dione

The title compound was prepared according to the procedure of Example 1B substituting 3-methoxybenzyl bromide for n-butyl bromide (0.446 g, 86%). MS (DCI) m/z 285 (M+H)⁺.

### EXAMPLE 24B

### 3-(1,1-dioxido-4H-1,2,4-benzothiadiazin-3-yl)-4-hydroxy-1-(3-methoxybenzyl)-1,8-naphthyridin-2(1H)-one

The title compound was prepared according to the procedure of Example 1D substituting the product of Example 24A for the product of Example 1B (0.086 g, 53%). MS (ESI−) m/z 461 (M−H)⁻. The sodium salt of the title compound was prepared according to the procedure of Example 1D. ¹H NMR (300 MHz, DMSO-d₆) δ 3.69 (s, 3H), 5.49 (s, 2H), 6.75 (m, 3H), 7.15 (m, 2H), 7.29 (td, J=8.46, 1.84 Hz, 2H), 7.56 (td, J=7.72, 1.47 Hz, 1H), 7.66 (d, J=7.72 Hz, 1H), 8.41 (dd, J=7.72, 1.84 Hz, 1H), 8.48 (dd, J=4.78, 1.84 Hz, 1H), 15.82 (s, 1H).

### EXAMPLE 25

### 3-(1,1-dioxido-4H-1,2,4-benzothiadiazin-3-yl)-4-hydroxy-1-(3-iodobenzyl)-1,8-naphthyridin-2(H)-one

### EXAMPLE 25A

### 1-(3-iodobenzyl)-2H-pyrido[2,3-d][1,3]oxazine-2,4(1H)-dione

The title compound was prepared according to the procedure of Example 1B substituting 3-iodobenzyl bromide for n-butyl bromide (0.614 g, 88%). MS (DCI) m/z 381 (M+H)⁺.

### EXAMPLE 25B

### 3-(1,1-dioxido-4H-1,2,4-benzothiadiazin-3-yl)-4-hydroxy-1-(3-iodobenzyl)-1,8-naphthyridin-2(1H)-one

The title compound was prepared according to the procedure of Example 1D substituting the product of Example 25A for the product of Example 1B (0.176 g, 60%). MS (ESI−) m/z 557 (M−H)⁻. The sodium salt of the title compound was prepared according to the procedure of Example 1D. ¹H NMR (300 MHz, DMSO-d₆) δ 5.47 (s, 2H), 7.07 (t, J=7.72 Hz, 1H), 7.17 (dd, J=7.72, 4.78 Hz, 1H), 7.28 (m, 3H), 7.55 (m, 2H), 7.66 (m, 2H), 8.41 (dd, J=7.72, 1.84 Hz, 1H), 8.49 (dd, J=4.78, 1.84 Hz, 1H), 15.79 (s, 1H).

### EXAMPLE 26

### 1-[(3,5-dimethyl-4-isoxazolyl)methyl]-3-(1,1-dioxido-4H-1,2,4-benzothiadiazin-3-yl)-4-hydroxy-1,8-naphthyridin-2(1H)-one

### EXAMPLE 26A

### 1-[(3,5-dimethyl-4-isoxazolyl)methyl]-2H-pyrido[2,3-d][1,3]oxazine-2,4(1H)-dione

The title compound was prepared according to the procedure of Example 1B substituting 4-chloromethyl-3,5-dimethylisoxazole for n-butyl bromide (0.199 g, 60%). ¹H NMR (300 MHz, DMSO-d₆) δ 2.20 (s, 3H), 2.45 (s, 3H), 5.10 (s, 2H), 7.40 (dd, J=7.72, 4.78 Hz, 1H), 8.40 (dd, J=7.72, 1.84 Hz, 1H), 8.80 (dd, J=4.78, 1.84 Hz, 1H).

### EXAMPLE 26B

### 1-[(3,5-dimethyl-4-isoxazolyl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 26A for the product of Example 1B (0.187 g, 63%). MS (DCI/NH₃) m/z 452 (M+H)⁺; ¹H NMR (300 MHz, DMSO-d₆) δ 2.20 (s, 3H), 2.38 (s, 3H), 5.44 (s, 2H), 7.51 (dd, J=7.90, 4.60 Hz, 1H), 7.55 (t, J=7.17 Hz, 1H), 7.64 (d, J=7.72 Hz, 1H), 7.77 (t, J=7.17 Hz, 1H), 7.92 (d, J=7.72 Hz, 1H), 8.58 (dd, J=7.90, 1.66 Hz, 1H), 8.88 (dd, J=4.60, 1.66 Hz, 1H), 13.95 (s, 1H). The sodium salt of the title compound was prepared according to the procedure of Example 1D. ¹H NMR (300 MHz, DMSO-d₆) δ 2.17 (s, 3H), 2.29 (s, 3H), 5.26 (s, 2H), 7.17 (dd, J=7.73, 4.78 Hz, 1H), 7.29 (m, 2H), 7.56 (t, J=7.72 Hz, 1H), 7.67 (d, J=7.72 Hz, 1H), 8.39 (dd, J=7.72, 1.84 Hz, 1H), 8.53 (dd, J=4.78, 1.84 Hz, 1H), 15.78 (s, 1H).

### EXAMPLE 27

### 3-(1,1-dioxido-4H-1,2,4-benzothiadiazin-3-yl)-4-hydroxy-1-[2-(3-thienyl)ethyl]-1,8-naphthyridin-2(1H)-one

### EXAMPLE 27A

### 1-[2-(3-thienyl)ethyl]-2H-pyrido[2,3-d][1,3]oxazine-2,4(1H)-dione

The title compound was prepared according to the procedure of Example 1B substituting 3-(2-bromoethyl)thiophene for n-butyl bromide (0.156 g, 46%). ¹H NMR (300 MHz, DMSO-d₆) δ 2.98 (t, 2H), 4.36 (t, 2H), 7.07 (d, J=5.15 Hz, 1H), 7.31 (m, 1H), 7.39 (dd, J=7.72, 5.15 Hz, 1H), 7.49 (m, 1H), 8.41 (dd, J=7.72, 1.84 Hz, 1H), 8.78 (dd, J=4.78, 1.84 Hz, 1H).

### EXAMPLE 27B

### 3-(1,1-dioxido-4H-1,2,4-benzothiadiazin-3-yl)-4-hydroxy-1-[2-(3-thienyl)ethyl]-1,8-naphthyridin-2(1H)-one

The title compound was prepared according to the procedure of Example 1D substituting the product of Example 27A for the product of Example 1B (0.123 g, 48%). MS (ESI−) m/z 451 (M−H)⁻. The sodium salt of the title compound was prepared according to the procedure of Example 1D. ¹H NMR (300 MHz, DMSO-d₆) δ 2.90 (t, J=7.90 Hz, 2H), 4.51 (t, J=7.90 Hz, 2H), 7.10 (d, J=4.78 Hz, 1H), 7.16 (dd, J=7.72, 4.78 Hz, 1H), 7.29 (m, 3H), 7.49 (dd, J=4.78, 2.94 Hz, 1H), 7.56 (m, 1H), 7.68 (d, J=7.72 Hz, 1H), 8.39 (dd, J=7.72, 1.84 Hz, 1H), 8.55 (dd, J=4.78, 1.84 Hz, 1H), 15.89 (s, 1H).

### EXAMPLE 28

### 3-(1,1-dioxido-4H-1,2,4-benzothiadiazin-3-yl)-4-hydroxy-1-(4-pyridinylmethyl)-1,8-naphthyridin-2(1H)-one

### EXAMPLE 28A

### 1-(4-pyridinylmethyl)-2H-pyrido[2,3-d][1,3]oxazine-2.4(1H)-dione

The title compound was prepared according to the procedure of Example 1B substituting 4-(chloromethyl)pyridine for n-butyl bromide (0.089 g, 29%). ¹H NMR (300 MHz, DMSO-d₆) δ 5.37 (s, 2H), 7.41 (m, 3H), 8.45 (dd, J=7.72, 1.84 Hz, 1H), 8.49 (m, 2H), 8.69 (dd, J=4.78, 1.84 Hz, 1H).

### EXAMPLE 28B

### 3-(1,1-dioxido-4H-1,2,4-benzothiadiazin-3-yl)-4-hydroxy-1-(4-pyridinylmethyl)-1,8-naphthyridin-2(1H)-one

The title compound was prepared according to the procedure of Example 1D substituting the product of Example 28A for the product of Example 1B (0.034 g, 19%). MS (DCI/NH₃) m/z 434 (M+H)⁺; ¹H NMR (300 MHz, DMSO-d₆) δ 5.83 (s, 2H), 7.52 (m, 2H), 7.60 (d, J=7.72 Hz, 1H), 7.69 (d, J=6.25 Hz, 2H), 7.73 (m, 1H), 7.91 (d, J=6.99 Hz, 1H), 8.62 (dd, J=7.72, 1.84 Hz, 1H), 8.68 (d, J=6.25 Hz, 2H), 8.75 (dd, J=4.78, 1.84 Hz, 1H), 13.98 (s, 1H). The sodium salt of the title compound was prepared according to the procedure of Example 1D. ¹H NMR (300 MHz, DMSO-d₆) δ 5.56 (s, 2H), 7.22 (m, 3H), 7.33 (m, 2H), 7.59 (m, 1H), 7.71 (m, 1H), 8.45 (m, 3H), 8.50 (dd, J=4.78, 1.83 Hz, 1H), 15.54 (s, 1H).

### EXAMPLE 29

### 1-(4-bromobenzyl)-3-(1,1-dioxido-4H-1,2,4-benzothiadiazin-3-yl)-4-hydroxy-1,8-naphthyridin-2(1H)-one

### EXAMPLE 29A

### 1-(4-bromobenzyl)-2H-pyrido[2,3-d][1,3]oxazine-2,4(1H)-dione

The title compound was prepared according to the procedure of Example 1B substituting 4-bromobenzyl bromide for n-butyl bromide (1.460 g, 72%). MS (DCI) m/z 333 (M+H)⁺.

### EXAMPLE 29B

### 1-(4-bromobenzyl)-3-(111-dioxido-4H-1,2,4-benzothiadiazin-3-yl)-4-hydroxy-1,8-naphthyridin-2(1H)-one

The title compound was prepared according to the procedure of Example 1D substituting the product of Example 29A for the product of Example 1B (0.060 g, 59%). MS (ESI−) m/z 509 (M−H)⁻. The sodium salt of the title compound was prepared according to the procedure of Example 1D. ¹H NMR (300 MHz, DMSO-d₆) δ 5.47 (s, 2H), 7.16 (dd, J=7.72, 4.78 Hz, 1H), 7.22 (d, J=8.46 Hz, 2H), 7.27 (t, J=7.72 Hz, 2H), 7.44 (d, J=8.46 Hz, 2H), 7.56 (td, J=7.72, 1.47 Hz, 1H), 7.67 (d, J=7.72 Hz, 1H), 8.41 (dd, J=7.72, 1.84 Hz, 1H), 8.48 (dd, J=4.78, 1.84 Hz, 1H), 15.80 (s, 1H).

### EXAMPLE 30

### 3-(1,1-dioxido-4H— 1,2,4-benzothiadiazin-3-yl)-4-hydroxy-1-neopentyl-1,8-naphthyridin-2(1H)-one

### EXAMPLE 30A

### ethyl 2-(neopentylamino)nicotinate

The title compound was prepared according to the procedure of Example 3A substituting 2,2-dimethylpropylamine for 2-ethylbutylamine (0.407 g, 57%). MS (ESI+) 237 (M+H)⁺; ¹H NMR (300 MHz, CDCl₃), 1.02 (s, 9H), 1.38 (t, J=7.17 Hz, 3H), 3.36 (d, J=5.52 Hz, 2H), 4.33 (q, J=7.35 Hz, 2H), 6.48 (dd, J=7.91, 4.60 Hz, 1H), 8.12 (dd, J=7.72, 2.21 Hz, 1H), 8.16 (s, 1H), 8.26 (dd, J=4.78, 2.21 Hz, 1H).

### EXAMPLE 30B

### 1-neopentyl-2H-pyrido[2,3-d][1,3]oxazine-2,4(1H)-dione

The title compound was prepared according to the procedure of Example 3B substituting the product of Example 30A for the product of Example 3A (0.182 g, 89%). ¹H NMR (300 MHz, CDCl₃) δ 1.12 (s, 9H), 4.28 (s, 2H), 7.25 (dd, J=6.99, 4.04 Hz, 1H), 8.41 (dd, J=7.91, 2.02 Hz, 1H), 8.69 (dd, J=4.78, 1.84 Hz, 1H).

### EXAMPLE 30C

### 3-(1,1-dioxido-4H-1,2,4-benzothiadiazin-3-yl)-4-hydroxy-1-neopentyl-1,8-naphthyridin-2(1H)-one

The title compound was prepared according to the procedure of Example 1D substituting the product of Example 30B for the product of Example 1B (0.070 g, 22%). MS (ESI+) m/z 413 (M+H)⁺; ¹H NMR (300 MHz, DMSO-d₆) δ 0.96 (s, 9H), 4.52 (s, 2H), 7.49 (dd, J=8.09, 4.41 Hz, 1H), 7.56 (t, J=7.54 Hz, 1H), 7.68 (d, J=8.09 Hz, 1H), 7.78 (m, 1H), 7.94 (d, J=6.99 Hz, 1H), 8.57 (dd, J=8.09, 1.84 Hz, 1H), 8.85 (dd, J=4.41, 1.84 Hz, 1H), 14.11 (s, 1H). The sodium salt of the title compound was prepared according to the procedure of Example 1D. MS (ESI+) m/z 413 (M+H)⁺; ¹H NMR (300 MHz, DMSO-d₆) δ 0.91 (s, 9H), 4.34 (s, 2H), 7.11 (dd, J=7.72, 4.78 Hz, 1H), 7.27 (m, 2H), 7.55 (m, 1H), 7.66 (m, 1H), 8.36 (dd, J=7.54, 2.02 Hz, 1H), 8.48 (dd, J=4.60, 2.02 Hz, 1H), 15.95 (s, 1H).

### EXAMPLE 31

### 1-{[(1S,2R,5S)-6,6-dimethylbicyclo[3.1.1]hept-2-yl]methyl}-3-(1,1-dioxido-4H-1,2,4-benzothiadiazin-3-yl)-4-hydroxy-1,8-naphthyridin-2(1H)-one

### EXAMPLE 31A

### ethyl 2-({[(1S,2R,5S)-6,6-dimethylbicyclo[3.1.1]hept-2-yl]methyl}amino)nicotinate

The title compound was prepared according to the procedure of Example 3A substituting (−)-cis-myrtanylamine for 2-ethylbutylamine (0.604 g, 40%). MS (ESI+) m/z 303 (M+H)⁺.

### EXAMPLE 31B

### 1-{[(1S,2R,5S)-6,6-dimethylbicyclo[3.1.1]hept-2-yl]methyl}-2H-pyrido[2,3-d][1,3]oxazine-2,4(1H)-dione

The title compound was prepared according to the procedure of Example 3B substituting the product of Example 31A for the product of Example 3A (0.570 g, 95%). ¹H NMR (300 MHz, DMSO-d₆) δ 0.79 (d, J=9.56 Hz, 1H), 1.14 (s, 3H), 1.22 (s, 3H), 1.62 (m, 1H), 1.87 (m, 5H), 2.26 (m, 1H), 2.53 (m, 1H), 4.04 (dd, J=13.05, 6.07 Hz, 1H), 4.28 (dd, J=13.24, 9.19 Hz, 1H), 7.37 (dd, J=7.72, 4.78 Hz, 1H), 8.38 (dd, J=7.72, 1.84 Hz, 1H), 8.76 (dd, J=4.78, 1.84 Hz, 1H).

### EXAMPLE 31C

### 1-{[(S,2R,5S)-6,6-dimethylbicyclo[3.1.1]hept-2-yl]methyl}-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 31B for the product of Example 1B (0.050 g, 21%). MS (ESI−) m/z 477 (M−H)⁻. The sodium salt of the title compound was prepared according to the procedure of Example 1D. ¹H NMR (300 MHz, DMSO-d₆) δ 0.78 (d, J=9.56 Hz, 1H), 1.15 (m, 3H), 1.30 (s, 3H), 1.80 (m, 6H), 2.24 (m, 1H), 2.54 (m, 1H), 4.37 (m, 2H), 7.12 (dd, J=7.54, 4.60 Hz, 1H), 7.27 (m, 2H), 7.55 (m, 1H), 7.67 (dd, J=7.72, 1.47 Hz, 1H), 8.36 (dd, J=7.54, 2.02 Hz, 1H), 8.50 (dd, J=4.60, 2.02 Hz, 1H), 15.95 (s, 1H).

### EXAMPLE 32

### 3-{[3-(1,1-dioxido-4H-1,2,4-benzothiadiazin-3-yl)-4-hydroxy-2-oxo-1,8-naphthyridin-1 (2H)-yl]methyl}benzonitrile

### EXAMPLE 32A

### 3-[(2,4-dioxo-2H-pyrido[2,3-d][1,3]oxazin-1 (4H)-yl)methyl]benzonitrile

The title compound was prepared according to the procedure of Example 1B substituting 3-cyanobenzyl bromide for n-butyl bromide (0.363 g, 71%). MS (DCI) m/z 280 (M+H)⁺.

### EXAMPLE 32B

### 3-{[3-(1,1-dioxido-4H-1,2,4-benzothiadiazin-3-yl)-4-hydroxy-2-oxo-1,8-naphthyridin-1(2H)-yl]methyl}benzonitrile

The title compound was prepared according to the procedure of Example 1D substituting the product of Example 32A for the product of Example 1B (0.024 g, 22%). MS (ESI−) m/z 456 (M−H)⁻. The sodium salt of the title compound was prepared according to the procedure of Example 1D. ¹H NMR (300 MHz, DMSO-d₆) δ 5.54 (s, 2H), 7.18 (dd, J=7.72, 4.78 Hz, 1H), 7.29 (m, 2H), 7.48 (t, J=7.72 Hz, 1H), 7.56 (td, J=7.91, 1.47 Hz, 2H), 7.68 (m, 3H), 8.42 (dd, J=7.72, 1.84 Hz, 1H), 8.49 (dd, J=4.60, 2.02 Hz, 1H), 15.77 (s, 1H).

### EXAMPLE 33

### 3-(1,1-dioxido-4H-1,2,4-benzothiadiazin-3-yl)-4-hydroxy-1-(3-pyridinylmethyl)-1,8-naphthyridin-2(1H)-one

### EXAMPLE 33A

### 1-(3-pyridinylmethyl)-2H-pyrido[2,3-d][1,3]oxazine-2,4(1H)-dione

The title compound was prepared according to the procedure of Example 1B substituting 3-(bromomethyl)pyridine for n-butyl bromide (0.153 g, 49%). ¹H NMR (300 MHz, DMSO-d₆) δ 5.38 (s, 2H), 7.34 (dd, J=7.72, 4.78 Hz, 1H), 7.40 (dd, J=7.72, 4.78 Hz, 1H), 7.82 (m, 1H), 8.42 (dd, J=7.72, 1.84 Hz, 1H), 8.47 (dd, J=4.78, 1.10 Hz, 1H), 8.66 (d, J=1.84 Hz, 1H), 8.74 (dd, J=5.15, 1.84 Hz, 1H).

### EXAMPLE 33B

### 3-(1,1-dioxido-4H-1,2,4-benzothiadiazin-3-yl)-4-hydroxy-1-(3-pyridinylmethyl)-1,8-naphthyridin-2(1H)-one

The title compound was prepared according to the procedure of Example 1D substituting the product of Example 33A for the product of Example 1B (0.098 g, 41%). MS (DCI/NH₃) m/z 434 (M+H)⁺; ¹H NMR (300 MHz, DMSO-d₆) δ 5.72 (s, 2H), 7.41 (dd, J=7.72, 4.78 Hz, 1H), 7.50 (m, 2H), 7.61 (d, J=8.09 Hz, 1H), 7.74 (m, 1H), 7.84 (d, J=7.72 Hz, 1H), 7.89 (d, J=8.09 Hz, 1H), 8.50 (d, J=4.04 Hz, 1H), 8.58 (dd, J=7.73, 1.84 Hz, 1H), 8.67 (s, 1H), 8.80 (dd, J=4.78, 1.84 Hz, 1H), 14.15 (s, 1H). The sodium salt of the title compound was prepared according to the procedure of Example 1D. ¹H NMR (300 MHz, DMSO-d₆) δ 5.53 (s, 2H), 7.18 (dd, J=7.72, 4.41 Hz, 1H), 7.29 (m, 3H), 7.56 (m, 1H), 7.65 (m, 2H), 8.40 (m, 2H), 8.51 (dd, J=4.60, 2.02 Hz, 1H), 8.57 (d, J=1.47 Hz, 1H), 15.78 (s, 1H).

### EXAMPLE 34

### 1-(1-adamantylmethyl)-3-(1,1-dioxido-4H-1,2,4-benzothiadiazin-3-yl)-4-hydroxy-1,8-naphthyridin-2(1H)-one

### EXAMPLE 34A

### 2-[(1-adamantylmethyl)amino]nicotinic Acid

The title compound was prepared according to the procedure of Example 3A substituting 2-chloronicotinic acid for ethyl 2-chloronicotinate and 1-adamantanemethylamine for 2-ethylbutylamine (0.185 g, 79%). MS (ESI+) m/z 287.1 (M+H)⁺; ¹H NMR (300 MHz, DMSO-d₆) δ 1.74 (m, 12H), 2.00 (s, 3H), 3.31 (m, 2H), 6.60 (dd, J=7.35, 5.52 Hz, 1H), 7.96 (dd, J=5.33, 2.02 Hz, 1H), 8.26 (dd, J=7.35, 1.84 Hz, 1H).

### EXAMPLE 34B

### 1-(1-adamantylmethyl)-2H-pyrido[2,3-d][1,3]oxazine-2,4(1H)-dione

The title compound was prepared according to the procedure of Example 3B substituting the product of Example 34A for the product of Example 3A (0.025 g, 20%). ¹H NMR (300 MHz, CDCl₃) δ 1.74 (m, 12H), 2.04 (s, 3H), 3.65 (d, J=5.88 Hz, 2H), 6.91 (dd, J=7.72, 5.52 Hz, 1H), 8.51 (d, J=4.78 Hz, 1H), 8.77 (d, J=7.72 Hz, 1H).

### EXAMPLE 34C

### 1-(1-adamantylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 34B for the product of Example 1B (0.018 g, 47%). MS (ESI+) m/z 491.1 (M+H)⁺; ¹H NMR (300 MHz, DMSO-d₆) δ 1.59 (s, 12H), 1.90 (m, 3H), 4.41 (br s, 2H), 7.48 (m, 1H), 7.56 (t, J=7.54 Hz, 1H), 7.69 (m, 1H), 7.77 (m, 1H), 7.94 (d, J=7.72 Hz, 1H), 8.56 (dd, J=8.09, 1.84 Hz, 1H), 8.85 (dd, J=4.60, 1.65 Hz, 1H). The sodium salt of the title compound was prepared according to the procedure of Example 1D. MS (ESI+) m/z 491.1 (M+H)⁺; ¹H NMR (300 MHz, DMSO-d₆) δ 1.56 (m, 12H), 1.87 (s, 3H), 4.21 (br s, 2H), 7.10 (dd, J=7.72, 4.78 Hz, 1H), 7.28 (m, 2H), 7.55 (m, 1H), 7.66 (dd, J=7.72, 1.47 Hz, 1H), 8.35 (dd, J=7.72, 1.84 Hz, 1H), 8.48 (dd, J=4.60, 2.02 Hz, 1H), 15.97 (br s, 1H).

### EXAMPLE 35

### 3-(1,1-dioxido-4H-1,2,4-benzothiadiazin-3-yl)-4-hydroxy-1-[3-(trifluoromethyl)benzyl]-1,8-naphthyridin-2(1H)-one

### EXAMPLE 35A

### 1-[3-(trifluoromethyl)benzyl]-2H-pyrido[2,3-d][1,3]oxazine-2,4(1H)-dione

The title compound was prepared according to the procedure of Example 1B substituting 3-(trifluoromethyl)benzyl bromide for n-butyl bromide (0.250 g, 42%). ¹H NMR (300 MHz, DMSO-d₆) δ 5.43 (s, 2H), 7.40 (dd, J=7.72, 4.78 Hz, 1H), 7.55 (m, 1H), 7.64 (m, 1H), 7.73 (d, J=7.72 Hz, 1H), 7.81 (s, 1H), 8.43 (dd, J=7.72, 1.84 Hz, 1H), 8.72 (dd, J=4.78, 1.84 Hz, 1H).

### EXAMPLE 35B

### 3-(1,1-dioxido-4H-1,2,4-benzothiadiazin-3-yl)-4-hydroxy-1-[3-(trifluoromethyl)benzyl]-1,8-naphthyridin-2(1H)-one

The title compound was prepared according to the procedure of Example 1D substituting the product of Example 35A for the product of Example 1B (0.22 g, 57%). MS (ESI−) m/z 499 (M−H)⁻; ¹H NMR (300 MHz, DMSO-d₆) δ 5.77 (s, 2H), 7.54 (m, 6H), 7.66 (d, J=7.72 Hz, 1H), 7.76 (m, 2H), 7.92 (d, J=8.09 Hz, 1H), 8.61 (dd, J=8.09, 1.84 Hz, 1H), 8.83 (dd, J=4.41, 1.84 Hz, 1H), 13.91 (br s, 1H). The sodium salt of the title compound was prepared according to the procedure of Example 1D. MS (ESI−) m/z 499 (M−H)⁻; ¹H NMR (300 MHz, DMSO-d₆) δ 5.58 (s, 2H), 7.18 (dd, J=7.72, 4.78 Hz, 1H), 7.29 (m, 2H), 7.54 (m, 4H), 7.66 (m, 2H), 8.42 (dd, J=7.72, 1.84 Hz, 1H), 8.49 (dd, J=4.60, 2.02 Hz, 1H), 15.78 (m, 1H).

### EXAMPLE 36

### 3-(1,1-dioxido-4H-1,2,4-benzothiadiazin-3-yl)-4-hydroxy-1-[(2-methyl-1,3-thiazol-5-yl)methyl]-1,8-naphthyridin-2(1H)-one

### EXAMPLE 36A

### 1-[(2-methyl-1,3-thiazol-5-yl)methyl]-2H-pyrido[2,3-d][1,3]oxazine-2,4(1H)-dione

The title compound was prepared according to the procedure of Example 1B substituting 2-methyl-5-chloromethylthiazole for n-butyl bromide (0.300 g, 54%).

### EXAMPLE 36B

### 3-(1,1-dioxido-4H-1,2,4-benzothiadiazin-3-yl)-4-hydroxy-1-[(2-methyl-1,3-thiazol-5-yl)methyl]-1,8-naphthyridin-2(1H)-one

The title compound was prepared according to the procedure of Example 1D substituting the product of Example 36A for the product of Example 1B (0.123 g, 25%). MS (ESI−) m/z 452 (M−H)⁻; ¹H NMR (300 MHz, DMSO-d₆) δ 2.54 (s, 3H), 5.76 (s, 1H), 7.53 (m, 1H), 7.52 (d, J=7.72 Hz, 1H), 7.65 (m, 2H), 7.76 (t, J=7.72 Hz, 1H), 7.91 (d, J=7.72 Hz, 1H), 8.57 (d, J=7.72 Hz, 1H), 8.90 (d, J=4.04 Hz, 1H), 13.92 (s, 1H). The sodium salt of the title compound was prepared according to the procedure of Example 1D.

### EXAMPLE 37

### 1-(2-cyclohexylethyl)-3-(1,1-dioxido-4H-1,2,4-benzothiadiazin-3-yl)-4-hydroxy-1,8-naphthyridin-2(1H)-one

### EXAMPLE 37A

### 1-(2-cyclohexylethyl)-2H-pyrido[2,3-d][1,3]oxazine-2,4(1H)-dione

The title compound was prepared according to the procedure of Example 1B substituting 1-bromo-2-cyclohexylethane for n-butyl bromide (0.196 g, 39%).

### EXAMPLE 37B

### 1-(2-cyclohexylethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 37A for the product of Example 1B (0.030 g, 18% after column purification). MS (ESI−) m/z 451 (M−H)⁻. The sodium salt of the title compound was prepared according to the procedure of Example 1D. MS (ESI−) m/z 451 (M−H)⁻; ¹H NMR (300 MHz, DMSO-d₆/TFA) δ 0.78 (m, 2H), 0.98 (m, 3H), 1.18 (m, 1H), 1.40 (m, 5H), 1.59 (d, J=12.50 Hz, 2H), 4.33 (m, 2H), 7.23 (m, 3H), 7.47 (t, J=7.54 Hz, 1H), 7.67 (d, J=7.72 Hz, 1H), 8.43 (m, 1H), 8.57 (dd, J=4.78, 1.47 Hz, 1H).

### EXAMPLE 38

### 3-(1,1-dioxido-4H-1,2,4-benzothiadiazin-3-yl)-4-hydroxy-1-(4-methoxybenzyl)-1,8-naphthyridin-2(1H)-one

### EXAMPLE 38A

### 1-(4-methoxybenzyl)-2H-pyrido[2,3-d][1,3]oxazine-2,4(1H)-dione

The title compound was prepared according to the procedure of Example 1B substituting 4-methoxybenzyl chloride for n-butyl bromide (0.364 g, 70%). MS (DCI) m/z 285 (M+H)⁺.

### EXAMPLE 38B

### 3-(1,1-dioxido-4H-1,2,4-benzothiadiazin-3-yl)-4-hydroxy-1-(4-methoxybenzyl)-1,8-naphthyridin-2(1H)-one

The title compound was prepared according to the procedure of Example 1D substituting the product of Example 38A for the product of Example 1B (0.098 g, 51%). MS (ESI−) m/z 461 (M−H)⁻. The sodium salt of the title compound was prepared according to the procedure of Example 1D. ¹H NMR (300 MHz, DMSO-d₆) δ 3.68 (s, 3H), 5.45 (s, 2H), 6.80 (dt, J=8.82, 2.21 Hz, 2H), 7.15 (dd, J=7.72, 4.78 Hz, 1H), 7.26 (m, 4H), 7.55 (td, J=7.72, 1.47 Hz, 1H), 7.67 (dd, J=7.91, 1.65 Hz, 1H), 8.39 (dd, J=7.72, 2.21 Hz, 1H), 8.50 (dd, J=4.78, 1.84 Hz, 1H), 15.86 (s, 1H).

### EXAMPLE 39

### 3-(1.1-dioxido-4H-1,2,4-benzothiadiazin-3-yl)-4-hydroxy-1-(2-methylbenzyl)-1,8-naphthyridin-2(1H)-one

### EXAMPLE 39A

### 1-(2-methylbenzyl)-2H-pyrido[2,3-d][1,3]oxazine-2,4(1H)-dione

The title compound was prepared according to the procedure of Example 1B substituting 2-methylbenzyl bromide for n-butyl bromide (0.353 g, 72%). MS (DCI) m/z 269 (M+H)⁺.

### EXAMPLE 39B

### 3-(1,1-dioxido-4H-1,2,4-benzothiadiazin-3-yl)-4-hydroxy-1-(2-methylbenzyl)-1,8-naphthyridin-2(1H)-one

The title compound was prepared according to the procedure of Example 1D substituting the product of Example 39A for the product of Example 1B (0.165 g, 62%). MS (ESI−) m/z 445 (M−H)⁻. The sodium salt of the title compound was prepared according to the procedure of Example 1D. ¹H NMR (300 MHz, DMSO-d₆) δ 2.44 (s, 3H), 5.45 (s, 2H), 6.59 (d, J=7.35 Hz, 1H), 6.96 (t, J=7.17 Hz, 1H), 7.06 (t, J=6.80 Hz, 1H), 7.16 (m, 2H), 7.29 (t, J=7.54 Hz, 2H), 7.56 (td, J=7.72, 1.47 Hz, 1H), 7.66 (d, J=7.72 Hz, 1H), 8.43 (d, J=6.25 Hz, 2H), 15.84 (s, 1H).

### EXAMPLE 40

### 1-(cyclopropylmethyl)-3-(1,1-dioxido-4H-1,2,4-benzothiadiazin-3-yl)-4-hydroxy-1,8-naphthyridin-2(1H)-one

### EXAMPLE 40A

### 1-(cyclopropylmethyl)-2H-pyrido[2,3-d][1,3]oxazine-2,4(1H)-dione

The title compound was prepared according to the procedure of Example 1B substituting (bromomethyl)cyclopropane for n-butyl bromide (0.278 g, 70%). MS (APCI+) m/z 219 (M+H); ¹H NMR (300 MHz, DMSO-d₆) δ 0.46 (m, 4H), 1.27 (m, 1H), 4.04 (d, J=6.99 Hz, 2H), 7.39 (dd, J=7.91, 4.96 Hz, 1H), 8.40 (dd, J=7.72, 1.84 Hz, 1H), 8.78 (dd, J=4.78, 1.84 Hz, 1H).

### EXAMPLE 40B

### 1-(cyclopropylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 40A for the product of Example 1B (0.06 g, 20% after column purification). MS (ESI−) m/z 395 (M−H)⁻. The sodium salt of the title compound was prepared according to the procedure of Example 1D. MS (ESI−) m/z 395 (M−H)⁻; ¹H NMR (300 MHz, DMSO-d₆) δ 0.40 (m, 4H), 1.32 (m, 1H), 4.19 (d, J=6.99 Hz, 2H), 7.14 (dd, J=7.54, 4.60 Hz, 1H), 7.28 (m, 2H), 7.55 (t, J=7.35 Hz, 1H), 7.67 (dd, J=7.72, 1.10 Hz, 1H), 8.38 (dd, J=7.72, 1.84 Hz, 1H), 8.52 (dd, J=4.60, 2.02 Hz, 1H), 15.93 (s, 1H).

### EXAMPLE 41

### 3-(1,1-dioxido-4H-1,2,4-benzothiadiazin-3-yl)-4-hydroxy-1-(1,3-thiazol-4-ylmethyl)-1,8-naphthyridin-2(1H)-one

### EXAMPLE 41A

### 1-(1,3-thiazol-4-ylmethyl)-2H-pyrido[2,3-d][1,3]oxazine-2,4(1H)-dione

The title compound was prepared according to the procedure of Example 1B substituting 4-(chloromethyl)thiazole for n-butyl bromide (0.049 g, 15%). ¹H NMR (300 MHz, DMSO-d₆) δ 5.48 (s, 2H), 7.40 (dd, J=7.72, 4.78 Hz, 1H), 7.66 (s, 1H), 8.45 (dd, J=7.72, 1.84 Hz, 1H), 8.72 (dd, J=4.78, 1.84 Hz, 1H), 9.06 (d, J=2.21 Hz, 1H).

### EXAMPLE 41B 3-(1,1-dioxido-4H-1,2,4-benzothiadiazin-3-yl)-4-hydroxy-1-(1,3-thiazol-4-ylmethyl)-1,8-naphthyridin-2(1H)-one

The title compound was prepared according to the procedure of Example 1D substituting the product of Example 41A for the product of Example 1B (0.046 g, 59%). MS (ESI−) m/z 438 (M−H)⁻. The sodium salt of the title compound was prepared according to the procedure of Example 1D. ¹H NMR (300 MHz, DMSO-d₆) δ 5.65 (s, 2H), 7.04 (d, J=2.21 Hz, 1H), 7.16 (dd, J=7.72, 4.78 Hz, 1H), 7.29 (m, 2H), 7.56 (m, 1H), 7.66 (d, J=7.35 Hz, 1H), 8.42 (dd, J=7.72, 2.21 Hz, 1H), 8.46 (dd, J=4.78, 2.20 Hz, 1H), 8.98 (d, J=1.84 Hz, 1H), 15.85 (s, 1H).

### EXAMPLE 42

### 3-(1,1-dioxido-4H-1,2,4-benzothiadiazin-3-yl)-4-hydroxy 1-[(5-phenyl-2-thienyl)methyl]-1,8-naphthyridin-2(1H)-one

The product of Example 4B (100 mg, 0.193 mmol), phenylboronic acid (49 mg, 0.387 mmol), 2M aqueous Na₂CO₃ (0.45 mL), absolute ethanol (0.5 mL), and tetrakis(triphenylphosphine)palladium (14 mg, 0.012 mmol) in N₂-sparged DMF (2 mL) was heated to reflux for 2.5 hours, cooled to 0° C., diluted with H₂O (15 mL), adjusted to pH 3 with 1N HCl, and extracted with ethyl acetate (3×25 mL). The combined extracts were washed with saturated NaCl, dried over anhydrous Na₂SO₄, filtered, and concentrated. The residue was purified by flash column chromatography on silica gel with 3% ethyl acetate/dichloromethane to give the title compound (0.039 g, 40%). MS (ESI−) m/z 513 (M−H)⁻. The sodium salt of the title compound was prepared according to the procedure of Example 1D. ¹H NMR (300 MHz, DMSO-d₆) δ 5.64 (s, 2H), 7.12 (d, J=3.68 Hz, 1H), 7.20 (dd, J=7.72, 4.78 Hz, 1H), 7.31 (m, 6H), 7.57 (m, 3H), 7.68 (d, J=7.72 Hz, 1H), 8.41 (dd, J=7.72, 1.84 Hz, 1H), 8.60 (dd, J=4.78, 1.84 Hz, 1H), 15.80 (s, 1H).

### EXAMPLE 43

### 3-(1,1-dioxido-4H-1,2,4-benzothiadiazin-3-yl)-4-hydroxy-1-(4-methyl-3-pentenyl)-1,8-naphthyridin-2(1H)-one

### EXAMPLE 43A

### 1-(4-methyl-3-pentenyl)-2H-pyrido[2,3-d][1,3]oxazine-2,4(1H)-dione

The title compound was prepared according to the procedure of Example 1B substituting 5-bromo-2-methyl-2-pentene for n-butyl bromide (0.157 g, 35%). MS (DCI+) m/z 247 (M+H)⁺; ¹H NMR (300 MHz, DMSO-d₆) δ 1.59 (s, 3H), 1.66 (s, 3H), 2.35 (m, 2H), 4.09 (m, 2H), 5.18 (t, J=7.54 Hz, 1H), 7.39 (dd, J=7.72, 5.15 Hz, 1H), 8.40 (dd, J=7.72, 1.84 Hz, 1H), 8.79 (dd, J=5.15, 1.84 Hz, 1H).

### EXAMPLE 43B

### 3-(1,1-dioxido-4H-1,2,4-benzothiadiazin-3-yl)-4-hydroxy-1-(4-methyl-3-pentenyl)-1,8-naphthyridin-2(1H)-one

The title compound was prepared according to the procedure of Example 1D substituting the product of Example 43A for the product of Example 1B (0.030 g, 20% after recrystallization). MS (ESI−) m/z 423 (M−H)⁻. The sodium salt of the title compound was prepared according to the procedure of Example 1D. MS (ESI−) m/z 423 (M−H)⁻; ¹H NMR (300 MHz, DMSO-d₆) δ 1.63 (s, 3H), 1.67 (s, 3H), 2.26 (m, 2H), 4.23 (m, 2H), 5.21 (m, 1H), 7.14 (dd, J=7.72, 4.78 Hz, 1H), 7.28 (m, 2H), 7.55 (t, J=7.35 Hz, 1H), 7.67 (d, J=7.72 Hz, 1H), 8.37 (dd, J=7.72, 2.21 Hz, 1H), 8.53 (dd, J=4.60, 2.02 Hz, 1H), 15.92 (s, 1H).

### EXAMPLE 44

### 4-{[3-(1,1-dioxido-4H-1,2,4-benzothiadiazin-3-yl)-4-hydroxy-2-oxo-1,8-naphthyridin-1(21)-yl]methyl}benzonitrile

### EXAMPLE 44A

### 4-[(2,4-dioxo-2H-pyrido[2,3-d][1,3]oxazin-[(4H)-yl)methyl]benzonitrile

The title compound was prepared according to the procedure of Example 1B substituting 4-cyanobenzyl bromide for n-butyl bromide (1.02 g, 60%). MS (DCI) m/z 280 (M+H)⁺.

### EXAMPLE 44B

### 4-{[3-(1,1-dioxido-4H-1,2,4-benzothiadiazin-3-yl)-4-hydroxy-2-oxo-1,8-naphthyridin-1(2H)-yl]methyl}benzonitrile

The title compound was prepared according to the procedure of Example 1D substituting the product of Example 44A for the product of Example 1B (0.197 g, 60%). MS (ESI−) m/z 456 (M−H). The sodium salt of the title compound was prepared according to the procedure of Example 1D. ¹H NMR (300 MHz, DMSO-d₆) δ 5.58 (s, 2H), 7.18 (dd, J=7.54, 4.60 Hz, 1H), 7.29 (td, J=8.46, 1.84 Hz, 2H), 7.41 (d, J=8.46 Hz, 2H), 7.56 (td, J=7.81, 1.65 Hz, 1H), 7.67 (dd, J=7.91, 1.29 Hz, 1H), 7.72 (d, J=8.46 Hz, 2H), 8.42 (dd, J=7.72, 1.84 Hz, 1H), 8.46 (dd, J=4.60, 2.02 Hz, 1H), 15.77 (s, 1H).

### EXAMPLE 45

### 1-[2-(1-cyclohexen-1-yl)ethyl]-3-(1,1-dioxido-4H-1,2,4-benzothiadiazin-3-yl)-4-hydroxy-1,8-naphthyridin-2(1H)-one

### EXAMPLE 45A

### ethyl 2-{[2-(1-cyclohexen-1-yl)ethyl]amino}nicotinate

The title compound was prepared according to the procedure of Example 3A substituting 2-(1-cyclohexenyl)ethylamine for 2-ethylbutylamine (2.2 g, 80%). MS (DCI) m/z 275 (M+H)⁺.

### EXAMPLE 45B

### 1-[2-(1-cyclohexen-1-yl)ethyl]-2H-pyrido[2,3-d][1,3]oxazine-2,4(1H)-dione

The title compound was prepared according to the procedure of Example 3B substituting the product of Example 45A for the product of Example 3A (0.493 g, 91%). MS (DCI) m/z 290 (M+NH₄)⁺.

### EXAMPLE 45C

### 1-[2-(1-cyclohexen-1-yl)ethyl]-3-(111-dioxido-4H-1,2,4-benzothiadiazin-3-yl)-4-hydroxy-1,8-naphthyridin-2(1H)-one

The title compound was prepared according to the procedure of Example 1D substituting the product of Example 45B for the product of Example 1B (0.048 g, 14%). MS (ESI−) m/z 449 (M−H)⁻. The sodium salt of the title compound was prepared according to the procedure of Example 1D. ¹H NMR (300 MHz, DMSO-d₆) δ 1.53 (m, 4H), 1.90 (m, 2H), 2.05 (m, 2H), 2.18 (t, J=7.54 Hz, 2H), 4.36 (m, 2H), 5.38 (s, 1H), 7.13 (dd, J=7.72, 4.78 Hz, 1H), 7.28 (m, 2H), 7.55 (td, J=7.72, 1.47 Hz, 1H), 7.66 (dd, J=7.72, 1.47 Hz, 1H), 8.37 (dd, J=7.54, 2.02 Hz, 1H), 8.52 (dd, J=4.60, 2.02 Hz, 1H), 15.91 (s, 1H).

### EXAMPLE 46

### 3-(1,1-dioxido-4H-1,2,4-benzothiadiazin-3-yl)-4-hydroxy-1-[(2-methyl-1,3-thiazol-4-yl)methyl]-1,8-naphthyridin-2(1H)-one

### EXAMPLE 46A

### 1-[(2-methyl-1,3-thiazol-4-yl)methyl]-2H-pyrido[2,3-d][1,3]oxazine-2,4(1H)-dione

The title compound was prepared according to the procedure of Example 1B substituting 4-chloromethyl-2-methylthiazole for n-butyl bromide (0.087 g, 26%). ¹H NMR (300 MHz, DMSO-d₆) δ 2.63 (s, 3H), 5.37 (d, J=1.47 Hz, 2H), 7.39 (s, 1H), 7.40 (dd, J=7.72, 4.78 Hz, 1H), 8.44 (dd, J=7.72, 1.84 Hz, 1H), 8.72 (dd, J=4.78, 1.84 Hz, 1H).

### EXAMPLE 46B

### 3-(1,1-dioxido-4H-1,2,4-benzothiadiazin-3-yl)-4-hydroxy-1-[(2-methyl-1,3-thiazol-4-yl)methyl]-1,8-naphthyridin-2(1H)-one

The title compound was prepared according to the procedure of Example 1D substituting the product of Example 46A for the product of Example 1B (0.078 g, 56%). MS (DCI/NH₃) m/z 454 (M+H)⁺. The sodium salt of the title compound was prepared according to the procedure of Example 1D. ¹H NMR (300 MHz, DMSO-d₆) δ 2.61 (s, 3H), 5.55 (s, 2H), 6.74 (s, 1H), 7.16 (dd, J=7.73, 4.78 Hz, 1H), 7.29 (m, 2H), 7.55 (m, 1H), 7.67 (d, J=7.72 Hz, 1H), 8.41 (dd, J=7.72, 1.84 Hz, 1H), 8.47 (dd, J=4.78, 2.21 Hz, 1H), 15.85 (s, 1H).

### EXAMPLE 47

### 2-{[3-(1,1-dioxido-4H-1,2,4-benzothiadiazin-3-yl)-4-hydroxy-2-oxo-1,8-naphthyridin-1(2H)-yl]methyl}benzonitrile

### EXAMPLE 47A

### 2-[(2,4-dioxo-2H-pyrido[2,3-d][1,3]oxazin-1 (4H)-yl)methyl]benzonitrile

The title compound was prepared according to the procedure of Example 1B substituting 2-cyanobenzyl bromide for n-butyl bromide (0.332 g, 65%). MS (DCI) m/z 280 (M+H)⁺.

### EXAMPLE 47B

### 2-{[3-(1,1-dioxido-4H-1,2,4-benzothiadiazin-3-yl)-4-hydroxy-2-oxo-1,8-naphthyridin-1 (2H)-yl]methyl}benzonitrile

The title compound was prepared according to the procedure of Example 1D substituting the product of Example 47A for the product of Example 1B (0.183 g, 66%). MS (ESI−) m/z 456 (M−H)⁻. The sodium salt of the title compound was prepared according to the procedure of Example 1D. ¹H NMR (300 MHz, DMSO-d₆) δ 5.68 (s, 2H), 7.00 (d, J=8.09 Hz, 1H), 7.19 (dd, J=7.35, 4.78 Hz, 1H), 7.30 (t, J=8.09 Hz, 2H), 7.39 (t, J=7.54 Hz, 1H), 7.56 (t, J=7.85 Hz, 2H), 7.67 (d, J=7.72 Hz, 1H), 7.84 (d, J=7.72 Hz, 1H), 8.44 (m, 2H), 15.75 (s, 1H).

### EXAMPLE 48

### 3-(1,1-dioxido-4H-1,2,4-benzothiadiazin-3-yl)-4-hydroxy-1-[(5-methyl-3-isoxazolyl)methyl]-1,8-naphthyridin-2(1H)-one

### EXAMPLE 48A

### 1-[(5-methyl-3-isoxazolyl)methyl]-2H-pyrido[2.3-d][1,3]oxazine-2,4(1H)-dione

The title compound was prepared according to the procedure of Example 1B substituting 3-chloromethyl-5-methylisoxazole for n-butyl bromide (0.047 g, 15%). ¹H NMR (300 MHz, DMSO-d₆) δ 2.34 (s, 3H), 5.35 (s, 2H), 6.26 (d, J=1.10 Hz, 1H), 7.42 (dd, J=7.72, 4.78 Hz, 1H), 8.44 (dd, J=7.72, 1.84 Hz, 1H), 8.75 (dd, J=5.15, 1.84 Hz, 1H).

### EXAMPLE 48B

### 3-(1,1-dioxido-4H-1,2,4-benzothiadiazin-3-yl)-4-hydroxy-1-[(5-methyl-3-isoxazolyl)methyl]-1,8-naphthyridin-2(1H)-one

The title compound was prepared according to the procedure of Example 1D substituting the product of Example 48A for the product of Example 1B (0.051 g, 67%). MS (ESI−) m/z 436 (M−H)⁻. The sodium salt of the title compound was prepared according to the procedure of Example 1D. ¹H NMR (300 MHz, DMSO-d₆) δ 2.29 (s, 3H), 5.50 (s, 2H), 5.94 (s, 1H), 7.18 (dd, J=7.72, 4.78 Hz, 1H), 7.29 (m, 2H), 7.56 (t, J=8.09 Hz, 1H), 7.67 (d, J=8.09 Hz, 1H), 8.41 (dd, J=7.72, 1.84 Hz, 1H), 8.49 (dd, J=4.78, 2.21 Hz, 1H), 15.76 (s, 1H).

### EXAMPLE 49

### 3-(1,1-dioxido-4H-1,2,4-benzothiadiazin-3-yl)-4-hydroxy-1-(1-naphthylmethyl)-1,8-naphthyridin-2(1H)-one

### EXAMPLE 49A

### 1-(2-naphthylmethyl)-2H-pyrido[2,3-d][1,3]oxazine-2,4(1H)-dione

The title compound was prepared according to the procedure of Example 1B substituting 1-(bromomethyl)naphthalene for n-butyl bromide (0.391 g, 71%). MS (DCI) m/z 305 (M+H)⁺.

### EXAMPLE 49B

### 3-(1,1-dioxido-4H-1,2,4-benzothiadiazin-3-yl)-4-hydroxy-1-(1-naphthylmethyl)-1,8-naphthyridin-2(1H)-one

The title compound was prepared according to the procedure of Example 1D substituting the product of Example 49A for the product of Example 1B (0.087 g, 60%). MS (ESI−) m/z 481 (M−H)⁻; ¹H NMR (300 MHz, DMSO-d₆) δ 5.88 (s, 2H), 7.45 (m, 2H), 7.54 (t, J=7.72 Hz, 3H), 7.65 (d, J=7.72 Hz, 1H), 7.75 (m, 2H), 7.81 (dd, J=6.07, 3.49 Hz, 1H), 7.86 (d, J=8.46 Hz, 2H), 7.93 (d, J=7.35 Hz, 1H), 8.63 (dd, J=7.72, 1.84 Hz, 1H), 8.83 (dd, J=4.78, 1.84 Hz, 1H), 14.04 (s, 1H). The sodium salt of the title compound was prepared according to the procedure of Example 1D. ¹H NMR (300 MHz, DMSO-d₆) δ 5.69 (s, 2H), 7.16 (dd, J=7.54, 4.60 Hz, 1H), 7.29 (t, J=7.72 Hz, 2H), 7.43 (m, 2H), 7.49 (dd, J=8.64, 1.65 Hz, 1H), 7.56 (td, J=7.72, 1.47 Hz, 1H), 7.67 (d, J=7.35 Hz, 2H), 7.83 (m, 3H), 8.42 (dd, J=7.54, 2.02 Hz, 1H), 8.48 (dd, J=4.60, 2.02 Hz, 1H), 15.86 (s, 1H).

### EXAMPLE 50

### 3-(1,1-dioxido-4H-1,2,4-benzothiadiazin-3-yl)-4-hydroxy-1-(2-pyridinylmethyl)-1,8-naphthyridin-2(1H)-one

### EXAMPLE 50A

### 1-(2-pyridinylmethyl)-2H-pyrido[2,3-d][1,3]oxazine-2,4(1H)-dione

The title compound was prepared according to the procedure of Example 1B substituting 2-(bromomethyl)pyridine for n-butyl bromide (0.060 g, 19%). ¹H NMR (300 MHz, DMSO-d₆) δ 5.45 (s, 2H), 7.26 (m, 1H), 7.39 (dd, J=7.72, 4.78 Hz, 1H), 7.45 (d, J=8.09 Hz, 1H), 7.73 (m, 1H), 8.46 (dd, J=7.72, 1.84 Hz, 1H), 8.47 (m, 1H), 8.68 (dd, J=4.78, 1.47 Hz, 1H).

### EXAMPLE 50B

### 3-(1,1-dioxido-4H-1,2,4-benzothiadiazin-3-yl)-4-hydroxy-1-(2-pyridinylmethyl)-1,8-naphthyridin-2(1H)-one

The title compound was prepared according to the procedure of Example 1D substituting the product of Example 50A for the product of Example 1B (0.072 g, 72%). MS (ESI−) m/z 432 (M−H)⁻. The sodium salt of the title compound was prepared according to the procedure of Example 1D. ¹H NMR (300 MHz, DMSO-d₆) δ 5.62 (s, 2H), 6.97 (d, J=8.09 Hz, 1H), 7.18 (m, 2H), 7.31 (m, 2H), 7.62 (m, 3H), 8.44 (d, J=6.62 Hz, 3H), 15.71 (s, 1H).

### EXAMPLE 51

### 1-(4-tert-butylbenzyl)-3-(1,1-dioxido-4H-1,2,4-benzothiadiazin-3-yl)-4-hydroxy-1,8-naphthyridin-2(1H)-one

### EXAMPLE 51A

### 1-(4-tert-butylbenzyl)-2H-pyrido[2,3-d][1,3]oxazine-2,4(1H)-dione

The title compound was prepared according to the procedure of Example 1B substituting 4-(tert-butyl)benzyl bromide for n-butyl bromide (0.410 g, 72%). MS (DCI) m/z 311 (M+H)⁺.

### EXAMPLE 51B

### 1-(4-tert-butylbenzyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 51A for the product of Example 1B (0.109 g, 70%). MS (ESI−) m/z 487 (M−H)⁻. The sodium salt of the title compound was prepared according to the procedure of Example 1D. ¹H NMR (300 MHz, DMSO-d₆) δ 1.23 (s, 9H), 5.49 (s, 2H), 7.16 (m, 3H), 7.28 (m, 4H), 7.55 (td, J=7.91, 1.47 Hz, 1H), 7.66 (d, J=6.25 Hz, 1H), 8.40 (dd, J=7.72, 2.21 Hz, 1H), 8.49 (dd, J=4.60, 2.02 Hz, 1H), 15.84 (s, 1H).

### EXAMPLE 52

### ethyl [3-(1,1-dioxido-4H-1,2,4-benzothiadiazin-3-yl)-4-hydroxy-2-oxo-1,8-naphthyridin-1(2H)-yl]acetate

### EXAMPLE 52A

### ethyl(2,4-dioxo-2H-pyrido[2,3-d][1,3]oxazin-1 (4H)-yl)acetate

The title compound was prepared according to the procedure of Example 1B substituting ethyl bromoacetate for n-butyl bromide (0.174 g, 43%). ¹H NMR (300 MHz, DMSO-d₆) δ 1.21 (t, J=7.17 Hz, 3H), 4.18 (q, J=7.11 Hz, 2H), 4.92 (s, 2H), 7.45 (dd, J=7.72, 4.78 Hz, 1H), 8.47 (dd, J=7.91, 1.65 Hz, 1H), 8.77 (dd, J=4.78, 1.84 Hz, 1H).

### EXAMPLE 52B

### ethyl [3-(1,1-dioxido-4H-1,2,4-benzothiadiazin-3-yl)-4-hydroxy-2-oxo-1,8-naphthyridin-1(2H)-yl]acetate

The title compound was prepared according to the procedure of Example 1D substituting the product of Example 52A for the product of Example 1B (0.200 g, 52%). MS (ESI−) m/z 427 (M−H)⁻; ¹H NMR (300 MHz, DMSO-d₆/TFA) δ 1.26 (t, J=6.99 Hz, 3H), 4.22 (q, J=7.11 Hz, 2H), 5.34 (s, 2H), 7.44 (dd, J=7.91, 4.60 Hz, 1H), 7.54 (m, 2H), 7.74 (m, 1H), 7.96 (m, 1H), 8.63 (dd, J=8.09, 1.84 Hz, 1H), 8.79 (dd, J=4.78, 1.84 Hz, 1H).

### EXAMPLE 53

### [3-(1,1-dioxido-4H-1,2,4-benzothiadiazin-3-yl)-4-hydroxy-2-oxo-1,8-naphthyndin-1 (2H)-yl]acetic Acid

To a suspension of the product of Example 52B in 1:1 THF:methanol (6 mL) was added 0.5 N aqueous lithium hydroxide (6 mL). The mixture was stirred at room temperature for 2 hours, adjusted to pH 3 with 1.0 N HCl, and filtered. The filter cake was washed with water and dried to give the title compound (0.133 g, 86%). MS (ESI−) m/z 399 (M−H)⁻; ¹H NMR (300 MHz, DMSO-d₆) δ 5.16 (s, 2H), 7.54 (m, 2H), 7.67 (d, J=7.72 Hz, 1H), 7.77 (t, J=7.72 Hz, 1H), 7.92 (d, J=7.72 Hz, 1H), 8.60 (dd, J=8.09, 1.84 Hz, 1H), 8.84 (dd, J=4.60, 1.65 Hz, 1H), 13.11 (br s, 1H), 13.79 (br s, 1H).

### EXAMPLE 54

### 3-(1,1-dioxido-4H-1,2,4-benzothiadiazin-3-yl)-4-hydroxy-1-(3-phenoxybenzyl)-1,8-naphthyridin-2(1H)-one

### EXAMPLE 54A

### 1-(3-phenoxybenzyl)-2H-pyrido[2,3-d][1,3]oxazine-24(1H)-dione

The title compound was prepared according to the procedure of Example 1B substituting 3-phenoxybenzyl chloride for n-butyl bromide (0.190 g, 31%). MS (DCI) m/z 347 (M+H)⁺.

### EXAMPLE 54B

### 3-(1,1-dioxido-4H-1,2,4-benzothiadiazin-3-yl)-4-hydroxy-1-(3-phenoxybenzyl)-1,8-naphthyridin-2(1H)-one

The title compound was prepared according to the procedure of Example 1D substituting the product of Example 54A for the product of Example 1B (0.063 g, 52%). MS (ESI−) m/z 523 (M−H)⁻. The sodium salt of the title compound was prepared according to the procedure of Example 1D. ¹H NMR (300 MHz, DMSO-d₆) δ 5.51 (s, 2H), 6.77 (dd, J=8.09, 1.47 Hz, 1H), 6.91 (s, 1H), 6.99 (t, J=8.46 Hz, 2H), 7.10 (t, J=7.35 Hz, 1H), 7.19 (m, 1H), 7.31 (m, 6H), 7.57 (t, J=7.72 Hz, 1H), 7.68 (d, J=7.72 Hz, 1H), 8.41 (dd, J=7.72, 1.84 Hz, 1H), 8.48 (d, J=2.94 Hz, 1H), 15.74 (s, 1H).

### EXAMPLE 55

### 1-allyl-3-(1,1-dioxido-4H-1,2,4-benzothiadiazin-3-yl)-4-hydroxy-1,8-naphthyridin-2(1H)-one

### EXAMPLE 55A

### 1-allyl-2H-pyrido[2,3-d][1,3]oxazine-2,4(1H)-dione

The title compound was prepared according to the procedure of Example 1B substituting allyl bromide for n-butyl bromide (5.12 g, 82%). MS (DCI/NH₃) m/z 205 (M+H)⁺. ¹H NMR (300 MHz, DMSO-d₆) δ 4.75 (m, 2H), 5.14 (dd, J=10.66, 1.47 Hz, 1H), 5.27 (dd, J=17.28, 1.47 Hz, 1H), 5.92 (m, 1H), 7.39 (dd, J=7.72, 4.78 Hz, 1H), 8.40 (dd, J=7.91, 2.02 Hz, 1H), 8.75 (dd, J=4.78, 1.84 Hz, 1H).

### EXAMPLE 55B

### 1-allyl-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 55A for the product of Example 1B (1.4g, 34.5%). MS (DCI/NH₃) m/z 383 (M+H)⁺. ¹H NMR (300 MHz, DMSO-d₆) δ 5.03 (m, 1H), 5.11-5.15 (m, 3H), 5.93-6.07 (m, 1H), 7.45-7.60 (m, 2H), 7.65-7.72 (m, J=8.46 Hz, 1H), 7.73-7.80 (t, J=7.72 Hz, 1H), 7.92 (d, J=7.35 Hz, 1H), 8.58 (dd, J=8.09, 1.84 Hz, 1H), 8.85 (dd, J=4.60, 1.65 Hz, 1H).

### EXAMPLE 56

### 3-(1,1-dioxido-4H-1,2,4-benzothiadiazin-3-yl)-4-hydroxy-1-(2-naphthylmethyl)-1,8-naphthyridin-2(1H)-one

### EXAMPLE 56A

### 1-(2-naphthylmethyl)-2H-pyrido[2,3-d][1,3]oxazine-2,4(1H)-dione

The title compound was prepared according to the procedure of Example 1B substituting 2-(bromomethyl)naphthalene for n-butyl bromide (0.417 g, 75%). MS (DCI) m/z 305 (M+H)⁺.

### EXAMPLE 56B

### 3-(1,1-dioxido-4H-1,2,4-benzothiadiazin-3-yl)-4-hydroxy-1-(2-naphthylmethyl)-1,8-naphthyridin-2(1H)-one

The title compound was prepared according to the procedure of Example 1D substituting the product of Example 56A for the product of Example 1B (0.022 g, 42%). MS (ESI−) m/z 481 (M−H); ¹H NMR (300 Mz, DMSO-d₆) δ 6.18 (s, 2H), 6.83 (d, J=6.62 Hz, 1H), 7.28 (m, 1H), 7.53 (t, J=7.54 Hz, 2H), 7.68 (m, 4H), 7.81 (d, J=8.09 Hz, 1H), 7.92 (d, J=7.35 Hz, 1H), 8.00 (d, J=8.09 Hz, 1H), 8.32 (d, J=8.46 Hz, 1H), 8.66 (dd, J=8.09, 1.84 Hz, 1H), 8.74 (dd, J=4.78, 1.84 Hz, 1H), 14.04 (s, 1H). The sodium salt of the title compound was prepared according to the procedure of Example 1D. ¹H NMR (300 MHz, DMSO-d₆) δ 6.00 (s, 2H), 6.76 (d, J=6.25 Hz, 1H), 7.18 (dd, J=7.54, 4.96 Hz, 1H), 7.30 (m, 3H), 7.63 (m, 4H), 7.75 (d, J=8.09 Hz, 1H), 7.97 (d, J=6.99 Hz, 1H), 8.31 (d, J=8.46 Hz, 1H), 8.40 (d, J=3.68 Hz, 1H), 8.47 (dd, J=7.72, 1.84 Hz, 1H), 15.78 (s, 1H).

### EXAMPLE 57

### 3-(1,1-dioxido-4H-1,2,4-benzothiadiazin-3-yl)-4-hydroxy-1-[(1R)-1-phenylethyl]-1,8-naphthyridin-2(1H)-one

### EXAMPLE 57A

### ethyl 2-{[(1R)-1-phenylethyl]amino}nicotinate

The title compound was prepared according to the procedure of Example 3A substituting (R)-(+)-α-methylbenzylamine for 2-ethylbutylamine (2.23 g, 82%). MS (DCI) m/z 271 (M+H)⁺.

### EXAMPLE 57B

### 1-[(1R)-1-phenylethyl]-2H-pyrido[2,3-d][1,3]oxazine-2,4(1H)-dione

The title compound was prepared according to the procedure of Example 3B substituting the product of Example 57A for the product of Example 3A (0.250 g, 62%). ¹H NMR (300 MHz, DMSO-d₆) δ 1.86 (d, J=6.99 Hz, 3H), 6.65 (q, J=6.99 Hz, 1H), 7.27 (m, 3H), 7.40 (m, 3H), 8.43 (dd, J=7.72, 1.84 Hz, 11H), 8.73 (dd, J=4.96, 2.02 Hz, 11H).

### EXAMPLE 57C

### 3-(1,1-dioxido-4H-1,2,4-benzothiadiazin-3-yl)-4-hydroxy-1-[(1R)-1-phenylethyl]-1,8-naphthyridin-2(1H)-one

The title compound was prepared according to the procedure of Example 1D substituting the product of Example 57B for the product of Example 1B (0.080 g, 36%). MS (ESI−) m/z 445 (M−H)⁻. The sodium salt of the title compound was prepared according to the procedure of Example 1D. ¹H NMR (300 MHz, DMSO-d₆) δ 1.90 (d, J=7.35 Hz, 3H), 6.87 (m, 1H), 7.12 (m, 2H), 7.25 (m, 6H), 7.55 (m, 1H), 7.65 (d, J=7.72 Hz, 1H), 8.40 (d, J=6.25 Hz, 2H), 15.92 (s, 1H).

### EXAMPLE 58

### 1-[(5-tert-butyl-2-thienyl)methyl]-3-(1,1-dioxido-4H-1,2,4-benzothiadiazin-3-yl)-4-hydroxy-1,8-naphthyridin-2(1H)-one

### EXAMPLE 58A

### 1-[(5-tert-butyl-2-thienyl)methyl]-2H-pyrido[2,3-d][1,3]oxazine-2,4(1H)-dione

The title compound was prepared according to the procedure of Example 1B substituting 2-bromomethyl-5-tert-butylthiophene for n-butyl bromide (0.098 g, 25%). ¹H NMR (300 MHz, DMSO-d₆) δ 1.28 (s, 9H), 5.39 (s, 2H), 6.71 (d, J=3.68 Hz, 1H), 7.00 (d, J=3.68 Hz, 1H), 7.42 (dd, J=7.72, 4.78 Hz, 1H), 8.40 (dd, J=7.72, 1.47 Hz, 1H), 8.83 (dd, J=4.78, 1.84 Hz, 1H).

### EXAMPLE 58B

### 1-[(5-tert-butyl-2-thienyl)methyl]-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 58A for the product of Example 1B (0.082 g, 54%). MS (ESI−) m/z 493 (M−H)⁻. The sodium salt of the title compound was prepared according to the procedure of Example 1D. ¹H NMR (300 MHz, DMSO-d₆) δ 1.25 (s, 9H), 5.55 (s, 2H), 6.63 (d, J=3.31 Hz, 1H), 6.89 (d, J=3.31 Hz, 1H), 7.18 (dd, J=7.72, 4.78 Hz, 1H), 7.28 (m, 2H), 7.56 (m, 1H), 7.67 (d, J=7.72 Hz, 1H), 8.39 (dd, J=7.72, 1.84 Hz, 1H), 8.57 (dd, J=4.78, 1.84 Hz, 1H), 15.83 (s, 1H).

### EXAMPLE 59

### 1-(1,1 ′-biphenyl-4-ylmethyl)-3-(1,1-dioxido-4H-1,2,4-benzothiadiazin-3-yl)-4-hydroxy-1,8-naphthyridin-2(1H)-one

### EXAMPLE 59A

### 1-(1,1 ′-biphenyl-4-ylmethyl)-2H-pyrido[2,3-d][1,3]oxazine-2,4(1H)-dione

The title compound was prepared according to the procedure of Example 1B substituting 4-phenylbenzyl chloride for n-butyl bromide (0.119 g, 20%). MS (DCI) m/z 331 (M+H)⁺.

### EXAMPLE 59B

### 1-(1,1 ′-biphenyl-4-ylmethyl)-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 59A for the product of Example 1B (0.061 g, 50%). MS (ESI−) m/z 507 (M−H)⁻. The sodium salt of the title compound was prepared according to the procedure of Example 1D. ¹H NMR (300 MHz, DMSO-d₆) δ 5.57 (s, 2H), 7.17 (dd, J=7.72, 4.78 Hz, 1H), 7.31 (m, 5H), 7.42 (t, J=7.54 Hz, 2H), 7.57 (m, 5H), 7.67 (d, J=8.09 Hz, 1H), 8.42 (dd, J=7.72, 1.84 Hz, 1H), 8.50 (dd, J=4.60, 2.02 Hz, 1H), 15.84 (s, 1H).

### EXAMPLE 60

### 3-(1,1-dioxido-4H-1,2,4-benzothiadiazin-3-yl)-4-hydroxy-1-[2-(1H-indol-3-yl)ethyl]-1,8-naphthyridin-2(1H)-one

### EXAMPLE 60A

### ethyl 2-{[2-(1H-indol-3-yl)ethyl]amino}nicotinate

The title compound was prepared according to the procedure of Example 3A substituting tryptamine for 2-ethylbutylamine (1.24 g, 80%). MS (DCI) m/z 310 (M+H)⁺.

### EXAMPLE 60B

### 1-[2-(1H-indol-3-yl)ethyl]-2H-pyrido[2,3-d][1,3]oxazine-2,4(1H)-dione

The title compound was prepared according to the procedure of Example 3B substituting the product of Example 60A for the product of Example 3A (0.164 g, 53%). MS (DCI) m/z 325 (M+NH₄)⁺.

### EXAMPLE 60C

### 3-(1,1-dioxido-4H-1,2,4-benzothiadiazin-3-yl)-4-hydroxy-1-[2-(1H-indol-3-yl)ethyl]-1,8-naphthyridin-2(1H)-one

The title compound was prepared according to the procedure of Example 1D substituting the product of Example 60B for the product of Example 1B (0.140 g, 54%). MS (ESI−) m/z 484 (M−H)⁻. The sodium salt of the title compound was prepared according to the procedure of Example 1D. ¹H NMR (300 MHz, DMSO-d₆) δ 3.09 (m, 2H), 4.75 (m, 2H), 7.08 (m, 2H), 7.27 (d, J=2.57 Hz, 1H), 7.36 (d, J=6.99 Hz, 1H), 7.54 (m, 2H), 7.77 (m, 3H), 7.94 (d, J=7.72 Hz, 1H), 8.60 (dd, J=8.09, 1.84 Hz, 1H), 8.95 (dd, J=4.78, 1.84 Hz, 1H), 10.88 (s, 1H).

### EXAMPLE 61

### 3-(1,1-dioxido-4H-1,2,4-benzothiadiazin-3-yl)-1-[(6-ethoxy-2-pyridinyl)methyl]-4-hydroxy-1,8-naphthyridin-2(1H)-one

### EXAMPLE 61A

### 1-[(6-chloro-2-pyridinyl)methyl]-2H-pyrido[2,3-d][1,3]oxazine-2,4(1H)-dione

The title compound was prepared according to the procedure of Example 1B substituting 2-bromomethyl-6-chloropyridine for n-butyl bromide (0.159 g, 45%). ¹H NMR (300 MHz, DMSO-d₆) δ 5.40 (s, 2H), 7.41 (m, 2H), 7.49 (d, J=7.72 Hz, 1H), 7.80 (t, J=7.72 Hz, 1H), 8.46 (dd, J=7.72, 1.84 Hz, 1H), 8.68 (dd, J=5.15, 1.84 Hz, 1H).

### EXAMPLE 61B

### 3-(1,1-dioxido-4H-1,2,4-benzothiadiazin-3-yl)-1-[(6-ethoxy-2-pyridinyl)methyl]-4-hydroxy-1,8-naphthyridin-2(1H)-one

The title compound was prepared according to the procedure of Example 1D substituting the product of Example 61A for the product of Example 1B (0.109 g, 42%). MS (ESI−) m/z 476 (M−H)⁻. The sodium salt of the title compound was prepared according to the procedure of Example 1D. ¹H NMR (300 MHz, DMSO-d₆) δ 1.19 (t, J=6.99 Hz, 3H), 4.17 (q, J=6.99 Hz, 2H), 5.52 (s, 2H), 6.45 (d, J=7.35 Hz, 1H), 6.54 (d, J=7.72 Hz, 1H), 7.15 (m, 1H), 7.29 (t, J=7.72 Hz, 2H), 7.54 (m, 2H), 7.66 (d, J=8.09 Hz, 1H), 8.42 (m, 2H), 15.83 (s, 1H).

### EXAMPLE 62

### 1-benzyl-3-(1,1-dioxido-4H-1,2,4-benzothiadiazin-3-yl)-4-hydroxy-7-methyl-1,8-naphthyridin-2(1H)-one

### EXAMPLE 62A

### phenylmethanaminium 2-(benzylamino)-6-methylnicotinate

The title compound was prepared as a benzylamine salt according to the procedure of Example 3A substituting 2-chloro-6-methyl-nicotinic acid for 2-chloro-nicotinic acid ethyl ester and benzyl amine for 2-ethylbutylamine (0.480 g, 46%). MS (ESI+) m/z 243.03 (M+H)⁺; ¹H NMR (300 MHz, CDCl₃) δ 2.35 (s, 3H), 3.67 (s, 2H), 4.65 (s, 2H), 5.73 (br s, 3H), 6.16 (d, J=7.72 Hz, 1H), 7.17 (m, 10H), 7.76 (d, J=7.35 Hz, 1H), 8.66 (br s, 1H).

### EXAMPLE 62B

### 1-benzyl-7-methyl-2H-pyrido[2,3-d][1,3]oxazine-2,4(1H)-dione

The title compound was prepared according to the procedure of Example 3B substituting the product of Example 62A for the product of Example 3A (0.150 g, 50%). ¹H NMR (300 MHz, CDCl₃) δ 2.66 (s, 3H), 5.47 (s, 2H), 7.10 (d, J=8.09 Hz, 1H), 7.31 (m, 3H), 7.55 (dd, J=7.54, 1.65 Hz, 2H), 8.26 (d, J=8.09 Hz, 1H).

### EXAMPLE 62C

### 1-benzyl-3-(1,1-dioxido-4H-1,2,4-benzothiadiazin-3-yl)-4-hydroxy-7-methyl-1,8-naphthyridin-2(1H)-one

The title compound was prepared according to the procedure of Example 1D substituting the product of Example 62B for the product of Example 1B (0.53 g, 51%). ¹H NMR (300 MHz, DMSO-d₆) δ 2.61 (s, 3H), 5.69 (s, 2H), 7.30 (m, 6H), 7.53 (m, 1H), 7.64 (m, J=7.35 Hz, 1H), 7.74 (t, J=7.54 Hz, 1H), 7.90 (d, J=8.82 Hz, 1H), 8.46 (d, J=8.46 Hz, 1H). The sodium salt of the title compound was prepared according to the procedure of Example 1D. MS (ESI+) m/z 447.0 (M+H)⁺; ¹H NMR (300 MHz, DMSO-d₆) δ 2.48 (s, 3H), 5.50 (s, 2H), 7.25 (m, 7H), 7.54 (m, 1H), 7.66 (d, J=6.25 Hz, 1H), 8.28 (d, J=7.72 Hz, 1H), 15.95 (s, 1H).

### EXAMPLE 63

### 3-(1,1-dioxido-4H-1,2,4-benzothiadiazin-3-yl)-4-hydroxy-1-[(6-methyl-2-pyridinyl)methyl]-1,8-naphthyridin-2(1H)-one

### EXAMPLE 63A

### 1-[(6-methyl-2-pyridinyl)methyl]-2H-pyrido[2,3-d][1,3]oxazine-2,4(1H)-dione

The title compound was prepared according to the procedure of Example 1B substituting 2-bromomethyl-6-methylpyridine for n-butyl bromide (0.088 g, 27%). ¹H NMR (300 MHz, DMSO-d₆) δ 2.42 (s, 3H), 5.38 (s, 2H), 7.19 (d, J=7.72 Hz, 1H), 7.37 (m, 2H), 7.58 (t, J=7.72 Hz, 1H), 8.45 (dd, J=7.72, 1.84 Hz, 1H), 8.67 (dd, J=4.78, 1.84 Hz, 1H).

### EXAMPLE 63B

### 3-(1,1-dioxido-4H-1,2,4-benzothiadiazin-3-yl)-4-hydroxy-1-[(6-methyl-2-pyridinyl)methyl]-1,8-naphthyridin-2(1H)-one

The title compound was prepared according to the procedure of Example 1D substituting the product of Example 63A for the product of Example 1B (0.081 g, 40%). MS (ESI−) m/z 446 (M−H)⁻. The sodium salt of the title compound was prepared according to the procedure of Example 1D. ¹H NMR (300 MHz, DMSO-d₆) δ 2.45 (s, 3H), 5.54 (s, 2H), 6.57 (d, J=7.72 Hz, 1H), 7.04 (d, J=7.35 Hz, 1H), 7.16 (dd, J=7.17, 4.96 Hz, 1H), 7.29 (t, J=7.72 Hz, 2H), 7.47 (t, J=7.72 Hz, 1H), 7.56 (m, 1H), 7.66 (d, J=7.72 Hz, 1H), 8.43 (m, 2H), 15.82 (s, 1H).

### EXAMPLE 64

### 3-(1,1-dioxido-4H-1,2,4-benzothiadiazin-3-yl)-1-(1-ethylpropyl)-4-hydroxy-1,8-naphthyridin-2(1H)-one

### EXAMPLE 64A

### ethyl 2-[(1-ethylpropyl)amino]nicotinate

The title compound was prepared according to the procedure of Example 3A substituting 2-ethyl-propyllamine for 2-ethylbutylamine (1.45 g, 88%). MS (ESI+) 237.1 (M+H)⁺. ¹H NMR (300 MHz, CDCl₃) δ 0.93 (t, J=7.35 Hz, 6H), 1.38 (t, J=7.17 Hz, 3H), 1.60 (m, 4H), 4.17 (m, 1H), 4.32 (q, J=7.11 Hz, 2H), 6.45 (dd, J=7.72, 4.78 Hz, 1H), 7.89 (br d, J=8.09 Hz, 1H), 8.10 (dd, J=7.72, 1.84 Hz, 1H), 8.24 (dd, J=4.78, 2.21 Hz, 1H).

### EXAMPLE 64B

### 1-(1-ethylpropyl)-2H-pyrido[2,3-d][1,3]oxazine-2,4(1H)-dione

The title compound was prepared according to the procedure of Example 3B substituting the product of Example 64A for the product of Example 3A (0.120 g, 57%). MS (ESI+) m/z 223.1 (M+H)⁺; ¹H NMR (300 MHz, CDCl₃) δ 0.87 (t, J=7.54 Hz, 6H), 1.88 (m, 2H), 2.21 (s, 2H), 5.43 (s, 1H), 7.24 (dd, J=6.99, 4.04 Hz, 1H), 8.42 (dd, J=7.72, 1.84 Hz, 1H), 8.68 (dd, J=4.78, 1.84 Hz, 1H).

### EXAMPLE 64C

### 3-(1,1-dioxido-4H-1,2,4-benzothiadiazin-3-yl)-1-(1-ethylpropyl)-4-hydroxy-1.8-naphthyridin-2(1H)-one

The title compound was prepared according to the procedure of Example 1D substituting the product of Example 64B for the product of Example 1B (0.030 g, 15%). MS (ESI+) m/z 413.04 (M+H)⁺; ¹H NMR (300 MHz, DMSO-d₆) δ 0.76 (t, J=7.54 Hz, 6H), 1.90 (m, 2H), 2.29 (m, 2H), 5.37 (m, 0.5H), 5.92 (m, 0.5H), 7.50 (m, 1H), 7.56 (t, J=7.54 Hz, 1H), 7.69 (m, 1H), 7.78 (t, J=7.17 Hz, 1H), 7.93 (d, J=7.35 Hz, 1H), 8.58 (d, J=8.09 Hz, 1H), 8.84 (m, 1H), 14.11 (s, 1H). The sodium salt of the title compound was prepared according to the procedure of Example 1D. MS (ESI+) m/z 413.07 (M+H—Na)⁺; ¹H NMR (300 MHz, DMSO-d₆) δ 0.74 (t, J=7.35 Hz, 6H), 1.88 (br s, 2H), 2.30 (br s, 2H), 5.35 (br s, 0.5H), 5.78 (br s, 0.5H), 7.28 (br s, 1H), 7.42 (m, J=7.35 Hz, 2H), 7.66 (m, 1H), 7.79 (br d, J=7.35 Hz, 1H), 8.47 (br d, J=7.35 Hz, 1H), 8.64 (br s, 1H).

### EXAMPLE 65

### 3-(1,1-dioxido-4H-1,2,4-benzothiadiazin-3-yl)-4-hydroxy-1-[(1S)-1-phenylethyl]-1,8-naphthyridin-2(1H)-one

### EXAMPLE 65A

### ethyl 2-{[(1S)-1-phenylethyl]amino}nicotinate

The title compound was prepared according to the procedure of Example 3A substituting (S)-(−)-α-methylbenzylamine for 2-ethylbutylamine (2.2 g, 81%). MS (DCI) m/z 271 (M+H)⁺.

### EXAMPLE 65B

### 1-[(1S)-1-phenylethyl]-2H-pyrido[2,3-d][1,3]oxazine-2,4(1H)-dione

The title compound was prepared according to the procedure of Example 3B substituting the product of Example 65A for the product of Example 3A (0.320 g, 80%). ¹H NMR (300 MHz, DMSO-d₆) δ 1.86 (d, J=6.99 Hz, 3H), 6.65 (q, J=6.99 Hz, 1H), 7.27 (m, 3H), 7.40 (m, 3H), 8.43 (dd, J=7.72, 1.84 Hz, 1H), 8.73 (dd, J=4.96, 2.02 Hz, 1H).

### EXAMPLE 65C

### 3-(1,1-dioxido-4H-1,2,4-benzothiadiazin-3-yl)-4-hydroxy-1-[(1S)-1-phenylethyl]-1,8-naphthyridin-2(1H)-one

The title compound was prepared according to the procedure of Example 1D substituting the product of Example 65B for the product of Example 1B (0.122 g, 36%). MS (ESI−) m/z 445 (M−H)⁻. The sodium salt of the title compound was prepared according to the procedure of Example 1D. ¹H NMR (300 MHz, DMSO-d₆) δ 1.90 (d, J=7.35 Hz, 3H), 6.87 (m, 1H), 7.12 (m, 2H), 7.25 (m, 6H), 7.55 (m, 1H), 7.65 (d, J=7.72 Hz, 1H), 8.40 (d, J=6.25 Hz, 2H), 15.92 (s, 1H).

### EXAMPLE 66

### 2-{2-[3-(1,1-dioxido-4H-1,2,4-benzothiadiazin-3-yl)-4-hydroxy-2-oxo-1,8-naphthyridin-1(2H)-yl]ethyl}-1H-isoindole-1,3(2H)-dione

### EXAMPLE 66A

### 1-[2-(1,3-dioxo-1,3-dihydro-2H-isoindol-2-yl)ethyl]-2H-pyrido[2,3-d][1,3]oxazine-2,4(1H)-dione

The title compound was prepared according to the procedure of Example 1B substituting N-(2-bromoethyl)phthalimide for n-butyl bromide (0.121 g, 20%). ¹H NMR (300 MHz, DMSO-d₆) δ 4.00 (t, J=5.52 Hz, 2H), 4.46 (t, J=5.52 Hz, 2H), 7.28 (dd, J=7.72, 4.78 Hz, 1H), 7.80 (s, 4H), 8.38 (dd, J=7.72, 1.84 Hz, 1H), 8.53 (dd, J=4.78, 1.84 Hz, 1H).

### EXAMPLE 66B

### 2-{2-[3-(1,1-dioxido-4H-1,2,4-benzothiadiazin-3-yl)-4-hydroxy-2-oxo-1,8-naphthyridin-1(2H)-yl]ethyl}-1H-isoindole-1,3(2H)-dione

The title compound was prepared according to the procedure of Example 1D substituting the product of Example 65B for the product of Example 1B (0.085 g, 46%). MS (ESI−) m/z 514 (M−H)⁻; ¹H NMR (300 MHz, DMSO-d₆/TFA) δ 4.09 (t, J=5.15 Hz, 2H), 4.87 (m, 2H), 7.11 (dd, J=7.91, 4.60 Hz, 1H), 7.19 (d, J=8.09 Hz, 1H), 7.44 (t, J=7.72 Hz, 1H), 7.58 (m, 5H), 7.84 (d, J=8.09 Hz, 1H), 8.34 (dd, J=4.41, 1.84 Hz, 1H), 8.42 (dd, J=7.91, 1.65 Hz, 1H).

### EXAMPLE 67

### 3-(1,1-dioxido-4H-1,2,4-benzothiadiazin-3-yl)-4-hydroxy-1-(3-hydroxypropyl)-1,8-naphthyridin-2(1H)-one

A solution of the product of Example 73 in THF (5 mL) was reacted with sodium borohydride (0.022 g, 0.58 mmol) at 0° C. for 30 minutes. The solution was poured into water and extracted with ethyl acetate. The extract was dried over sodium sulfate, filtered, concentrated and purified by preparative HPLC on a Waters Symmetry C8 column (40 mm×100 mm, 7 μm particle size) using a gradient of 10% to 100% acetonitrile/0.1% aqueous TFA over 12 minutes (15 minute run time) at a flow rate of 70 mL/min to produce the title compound. MS (DCI/NH₃) m/z 401 (M+H)⁺; ¹H NMR (300 MHz, DMSO-d₆) δ 1.87 (m, 2H), 3.54 (t, J=6.43 Hz, 2H), 4.55 (m, 2H), 7.52 (dd, J=8.09, 4.78 Hz, 1H), 7.56 (m, 1H), 7.71 (d, J=8.09 Hz, 1H), 7.79 (m, 1H), 7.94 (d, J=8.09 Hz, 1H), 8.58 (dd, J=8.09, 1.84 Hz, 1H), 8.90 (dd, J=4.60, 1.65 Hz, 1H), 14.13 (s, 1H).

### EXAMPLE 68

### 1-cyclopentyl-3-(1,1-dioxido-4H-1,2,4-benzothiadiazin-3-yl)-4-hydroxy-1,8-naphthyridin-2(1H)-one

### EXAMPLE 68A

### ethyl 2-(cyclopentylamino)nicotinate

The title compound was prepared according to the procedure of Example 3A substituting cyclopentylamine for 2-ethylbutylamine (0.231 g, 67%). MS (ESI+) m/z 235.1 (M+H)⁺; ¹H NMR (300 MHz, CDCl₃) δ 1.37 (t, J=7.17 Hz, 3H), 1.64 (m, 6H), 2.08 (m, 2H), 4.31 (q, J=7.23 Hz, 2H), 4.45 (m, 1H), 6.48 (dd, J=7.72, 4.78 Hz, 1H), 8.02 (d, J=5.88 Hz, 1H), 8.10 (dd, J=7.91, 2.02 Hz, 1H), 8.28 (dd, J=4.78, 1.84 Hz, 1H).

### EXAMPLE 68B

### 1-cyclopentyl-2H-pyrido[2,3-d][1,3]oxazine-2,4(1H)-dione

The title compound was prepared according to the procedure of Example 3B substituting the product of Example 68A for the product of Example 3A (0.130 g, 56%). MS (ESI+) m/z 221.08 (M+H)⁺; ¹H NMR (300 MHz, CDCl₃) δ 1.66 (m, 1H), 1.99 (m, 4H), 2.21 (m, 2H), 5.79 (m, 1H), 7.25 (dd, J=8.09, 4.78 Hz, 1H), 8.42 (dd, J=7.72, 2.21 Hz, 1H), 8.70 (dd, J=4.78, 1.84 Hz, 1H).

### EXAMPLE 68C

### 1-cyclopentyl-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 68B for the product of Example 1B (0.133 g, 60%). MS (ESI+) m/z 433.06 (M+H)⁺; ¹H NMR (300 MHz, DMSO-d₆) δ 1.70 (m, 2H), 1.85 (m, 2H), 2.07 (s, 2H), 2.28 (m, 2H), 6.17 (m, J=8.64, 8.64 Hz, 1H), 7.52 (m, 2H), 7.65 (d, J=7.72 Hz, 1H), 7.77 (m, 1H), 7.93 (d, J=6.99 Hz, 1H), 8.57 (dd, J=7.9L, 2.02 Hz, 1H), 8.86 (dd, J=4.78, 1.84 Hz, 1H), 14.05 (br s, 1H). The sodium salt of the title compound was prepared according to the procedure of Example 1D. ¹H NMR (300 MHz, DMSO-d₆) δ 1.63 (m, 2H), 1.79 (br s, 2H), 2.04 (m, 2H), 2.23 (m, 2H), 6.08 (m, 1H), 7.31 (br s, 1H), 7.43 (br s, 2H), 7.65 (d, J=6.25 Hz, 1H), 7.80 (br s, 1H), 8.48 (d, J=7.72 Hz, 1H), 8.69 (br s, 1H).

### EXAMPLE 69

### 3-(1,1-dioxido-4H-1,2,4-benzothiadiazin-3-yl)-1-[2-(1,3-dioxolan-2-yl)ethyl]-4-hydroxy-1,8-naphthyridin-2(1H)-one

### EXAMPLE 69A

### 1-[2-(1,3-dioxolan-2-yl)ethyl]-2H-pyrido[2,3-d][1,3]oxazine-2.4(1H)-dione

The title compound was prepared according to the procedure of Example 1B substituting 2-(2-bromomethyl)-1,3-dioxolane for n-butyl bromide (0.86 g, 53%). MS (DCI/NH₃) m/z 265 (M+H)⁺; ¹H NMR (300 MHz, DMSO-d₆) δ 1.98 (m, 2H), 3.83 (m, 4H), 4.25 (m, 2H), 4.92 (m, 1H), 7.38 (m, 1H), 8.39 (dd, J=7.72, 1.84 Hz, 1H), 8.78 (dd, J=4.96, 2.02 Hz, 1H).

### EXAMPLE 69B

### 3-(1,1-dioxido-4H-1,2,4-benzothiadiazin-3-yl)-1-[2-(1,3-dioxolan-2-yl)ethyl]-4-hydroxy-1,8-naphthyridin-2(1H)-one

The title compound was prepared according to the procedure of Example 1D substituting the product of Example 69A for the product of Example 1B (0.89 g, 62%). MS (DCI/NH₃) m/z 443 (M+H)⁺; ¹H NMR (300 MHz, DMSO-d₆) δ 2.03 (m, 2H), 2.50 (m, 2H), 3.84 (m, 2H), 4.59 (m, 2H), 4.97 (t, J=4.60 Hz, 1H), 7.51 (dd, J=7.91, 4.60 Hz, 1H), 7.56 (m, 1H), 7.77 (m, 2H), 7.94 (m, 1H), 8.56 (dd, J=8.09, 1.84 Hz, 1H), 8.89 (dd, J=4.78, 1.84 Hz, 1H), 14.09 (s, 1H).

### EXAMPLE 70

### 1-(2,3-dihydroxypropyl)-3-(1,1-dioxido-4H-1,2,4-benzothiadiazin-3-yl)-4-hydroxy-1,8-naphthyridin-2(1H)-one

The product of Example 55B (1.08 g, 0.028 mol) was reacted with osmium tetroxide (0.0007 mol) and N-methylmorpholine N-oxide (4.96 g, 0.043 mol) in a 1:1 mixture of water and TBF (50 mL) at room temperature for 18 hours. The reaction mixture was treated with sodium bisulfite and diluted with water. The product precipitated from the aqueous mixture and was collected by vacuum filtration to give the title compound as a a white solid (1.09 g, 93%). MS (DCI/NH₃) m/z 417 (M+H)⁺; ¹H NMR (300 MHz, DMSO-d₆) δ 3.33 (m, 2H), 3.87 (m, 1H), 4.37 (m, 2H), 4.52 (t, J=6.07 Hz, 1H), 4.78 (d, J=5.52 Hz, 1H), 7.17 (dd, J=7.72, 4.78 Hz, 1H), 7.29 (m, 2H), 7.56 (m, 1H), 7.67 (d, J=6.99 Hz, 1H), 8.40 (dd, J=7.72, 1.84 Hz, 1H), 8.53 (dd, J=4.78, 1.84 Hz, 1H), 15.80 (m, 1H).

### EXAMPLE 71

### 1-cycloheptyl-3-(1,1-dioxido-4H-1,2,4-benzothiadiazin-3-yl)-4-hydroxy-1,8-naphthyridin-2(1H)-one

### EXAMPLE 71A

### ethyl 2-(cycloheptylamino)nicotinate

The title compound was prepared according to the procedure of Example 3A substituting cycloheptylamine for 2-ethylbutylamine (1.01 g, 83%). MS (ESI+) m/z 263.1 (M+H)⁺. ¹H NMR (300 MHz, CDCl₃) δ 1.37 (t, J=7.17 Hz, 3H), 1.62 (m, 10H), 2.02 (m, 2H), 4.29 (m, 1H), 4.31 (q, J=7.35 Hz, 2H), 6.45 (dd, J=7.72, 4.78 Hz, 1H), 8.05 (d, J=6.99 Hz, 1H), 8.10 (dd, J=7.91, 2.02 Hz, 1H), 8.26 (dd, J=4.78, 1.84 Hz, 1H).

### EXAMPLE 71B

### 1-cycloheptyl-2H-pyrido[2,3-d][1,3]oxazine-2,4(1H)-dione

The title compound was prepared according to the procedure of Example 3B substituting the product of Example 71A for the product of Example 3A (0.205 g, 55%). MS (ESI+) m/z 249.1 (M+H)⁺; ¹H NMR (300 MHz, CDCl₃) δ 1.63 (m, 6H), 1.84 (m, 4H), 2.43 (m, 2H), 5.39 (s, 1H), 7.24 (dd, J=7.72, 4.78 Hz, 1H), 8.40 (m, 1H), 8.71 (dd, J=4.78, 1.84 Hz, 1H).

### EXAMPLE 71C

### 1-cycloheptyl-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 71B for the product of Example 1B (0.041 g, 15%). MS (ESI+) m/z 439.07 (M+H)⁺. ¹H NMR (300 MHz, DMSO-d₆) δ 1.23 (m, 6H), 1.58 (m, 4H), 1.79 (m, 2H), 5.90 (m, 1H), 7.45 (m, 1H), 7.53 (m, 1H), 7.66 (m, J=9.56 Hz, 1H), 7.74 (d, J=7.72 Hz, 1H), 7.90 (d, J=6.25 Hz, 1H), 8.54 (d, J=7.35 Hz, 1H), 8.85 (s, 1H). The sodium salt of the title compound was prepared according to the procedure of Example 1D. ¹H NMR (300 MHz, DMSO-d₆) δ 1.61 (m, 8H), 1.77 (m, 4H), 1.94 (m, 2H), 5.60 (m, 1H), 7.10 (dd, J=7.54, 4.60 Hz, 1H), 7.54 (m, 1H), 7.66 (dd, J=7.72, 1.47 Hz, 1H), 8.36 (dd, J=7.72, 2.21 Hz, 1H), 8.51 (dd, J=4.78, 2.21 Hz, 1H), 15.99 (s, 1H).

### EXAMPLE 72

### 1-(3-anilinopropyl)-3-(1,1-dioxido-4H-1,2,4-benzothiadiazin-3-yl)-4-hydroxy-1,8-naphthyridin-2(1H)-one

A solution of the product of Example 73 (0.090 g, 0.23 mmol) and aniline (0.15 mL, 0.23 mmol) in THF (6 mL) was treated with sodium triacetoxyborohydride (0.08 g, 0.38 mmol) and glacial acetic acid (0.025 mL, 0.43 mmol) at ambient temperature for 24 hours. The solvent was removed under vacuum and the resulting solid was purified by preparative HPLC on a Waters Symmetry C8 column (40 mm×100 mm, 7 μm particle size) using a gradient of 10% to 100% acetonitrile/0.1% aqueous TFA over 12 minutes (15 minutes run time) at a flow rate of 70 mL/min to give the title compound. MS (DCI/NH₃) m/z 476 (M+H)⁺. The title compound was dissolved in 1,4-dioxane (6 mL) and 4M HCl in dioxane (2 mL). After stirring at room temperature for 3 hours, the mixture was filtered and the filter cake was dried to yield the hydrochloride salt. ¹H NMR (300 MHz, DMSO-d₆) δ 2.11 (m, 2H), 3.32 (m, 2H), 4.59 (t, J=6.80 Hz, 2H), 7.18 (s, 3H), 7.34 (d, J=7.35 Hz, 2H), 7.52 (m, 1H), 7.57 (m, 1H), 7.67 (d, J=7.35 Hz, 1H), 7.80 (m, 1H), 7.94 (d, J=7.72 Hz, 1H), 8.59 (dd, J=7.72, 1.84 Hz, 1H), 8.89 (dd, J=4.78, 1.84 Hz, 1H), 13.96 (s, 1H).

### EXAMPLE 73

### 3-[3-(1,1-dioxido-4H-1,2,4-benzothiadiazin-3-yl)-4-hydroxy-2-oxo-1,8-naphthyridin-1 (2H)-yl]propanal

A stirred suspension of the product of Example 69B (0.65 g, 0.15 mmol) in water (3 mL) and glacial acetic acid (12 mL) at ambient temperature was treated dropwise with sulfuric acid (1 mL). The mixture was heated to 60° C. for 1 hour, and then diluted with water. The mixture was filtered and the filter cake was washed with water and dried to produce the title compound (0.455 g, 78%). MS (DCI/NH₃) m/z 399 (M+H)⁺; ¹H NMR (300 MHz, DMSO-d₆) δ 2.72 (m, 2H), 4.59 (t, J=6.62 Hz, 2H), 7.17 (dd, J=7.72, 4.78 Hz, 1H), 7.28 (m, 1H), 7.55 (m, 1H), 7.67 (d, J=7.72 Hz, 1H), 8.39 (dd, J=7.72, 1.84 Hz, 1H), 8.52 (dd, J=4.78, 1.84 Hz, 1H), 9.76 (t, J=2.21 Hz, 1H), 9.76 (t, J=2.21 Hz, 1H).

### EXAMPLE 74

### methyl 4-{[3-(1,1-dioxido-4H-1,2,4-benzothiadiazin-3-yl)-4-hydroxy-2-oxo-1,8-naphthyridin-1(2H)-yl]methyl}benzoate

### EXAMPLE 74A

### methyl 4-[(2,4-dioxo-2H-pyrido[2,3-d][1,3]oxazin-1 (4H)-yl)methyl]benzoate

The title compound was prepared according to the procedure of Example 1B substituting methyl 4-(bromomethyl)benzoate for n-butyl bromide (1.5 g, 75%). MS (DCI) m/z 313 (M+H)⁺.

### EXAMPLE 74B

### methyl 4-{F3-(1,1-dioxido-4H-1,2,4-benzothiadiazin-3-yl)-4-hydroxy-2-oxo-1,8-naphthyridin-1(2H)-yl]methyl}benzoate

The title compound was prepared according to the procedure of Example 1D substituting the product of Example 74A for the product of Example 1B (0.130 g, 37%). MS (ESI−) m/z 489 (M−H)⁻; ¹H NMR (300 MHz, DMSO-d₆) δ 3.82 (s, 3H), 5.76 (s, 2H), 7.42 (d, J=8.09 Hz, 2H), 7.50 (m, 2H), 7.63 (d, J=7.72 Hz, 1H), 7.74 (t, J=7.72 Hz, 1H), 7.88 (d, J=8.46 Hz, 2H), 7.93 (m, 1H), 8.60 (dd, J=8.09, 1.84 Hz, 1H), 8.78 (d, J=3.31 Hz, 1H), 14.12 (br s, 1H).

### EXAMPLE 75

### ethyl 5-{[3-(1,1-dioxido-4H-1,2,4-benzothiadiazin-3-yl)-4-hydroxy-2-oxo-1,8-naphthyridin-1(2H)-yl]methyl}-2-furoate

### EXAMPLE 75A

### ethyl 5-[(2,4-dioxo-2H-pyrido[2,3-d][1,3]oxazin-1 (4H)-yl)methyl]-2-furoate

The title compound was prepared according to the procedure of Example 1B substituting ethyl 5-chloromethyl-2-furancarboxylate for n-butyl bromide (0.073 g, 19%). ¹H NMR (300 MHz, DMSO-d₆) δ 1.34 (t, J=7.17 Hz, 3H), 4.32 (q, J=7.35 Hz, 2H), 5.56 (s, 2H), 6.49 (d, J=3.68 Hz, 1H), 7.09 (d, J=3.31 Hz, 1H), 7.31 (dd, J=7.72, 4.78 Hz, 1H), 8.44 (dd, J=7.72, 1.84 Hz, 1H), 8.76 (dd, J=4.78, 1.84 Hz, 1H).

### EXAMPLE 75B

### ethyl 5-{[3-(1,1-dioxido-4H-1,2,4-benzothiadiazin-3-yl)-4-hydroxy-2-oxo-1,8-naphthyridin-1 (2H)-yl]methyl}-2-furoate

The title compound was prepared according to the procedure of Example 1D substituting the product of Example 75A for the product of Example 1B (0.074 g, 69%). MS (DCI/NH₃) m/z 495 (M+H)⁺. ¹H NMR (300 MHz, DMSO-d₆) δ 1.26 (t, J=7.17 Hz, 3H), 4.26 (q, J=7.23 Hz, 2H), 5.73 (s, 2H), 6.45 (d, J=3.68 Hz, 1H), 7.19 (d, J=3.68 Hz, 1H), 7.55 (m, 2H), 7.75 (m, 2H), 7.93 (d, J=7.72 Hz, 1H), 8.60 (dd, J=7.91, 1.83 Hz, 1H), 8.86 (dd, J=4.78, 1.84 Hz, 1H), 13.80 (s, 1H). The sodium salt of the title compound was prepared according to the procedure of Example 1D. ¹H NMR (300 MHz, DMSO-d₆) δ 1.27 (t, J=7.17 Hz, 3H), 4.25 (q, J=7.23 Hz, 2H), 5.55 (s, 2H), 6.22 (d, J=3.31 Hz, 1H), 7.14 (d, J=3.31 Hz, 1H), 7.20 (dd, J=7.72, 4.60 Hz, 1H), 7.29 (m, 2H), 7.56 (m, 1H), 7.67 (d, J=8.09 Hz, 1H), 8.42 (dd, J=7.72, 2.20 Hz, 1H), 8.52 (dd, J=4.60, 2.21 Hz, 1H), 15.73 (s, 1H).

### EXAMPLE 76

### 1-[3-(dimethylamino)propyl]-3-(1,1-dioxido-4H-1,2,4-benzothiadiazin-3-yl)-4-hydroxy-1,8-naphthyridin-2(1H)-one

A solution of the product of Example 73 (0.085 g, 0.21 mmol) and dimethylamine (2.0 M in THF, 0.110 mL, 0.22 mmol) in tetrahyrofuran (4 mL) was reacted with sodium triacetoxyborohydride (0.06 g, 0.28 mmol) at room temperature for 1 hour. The solvent was removed under vacuum and the resulting solid was triturated with methanol and dimethylsulfoxide (1:1), filtered, and dried to product the title compound (0.56 g, 61%). (DCI/NH₃) m/z 428 (M+H)⁺.

The sodium salt of the title compound was prepared according to the procedure of Example 1D. ¹H NMR (300 MHz, DMSO-d₆) δ 1.72 (m, 2H), 2.15 (s, 6H), 2.29 (t, J=7.17 Hz, 2H), 4.28 (m, 2H), 7.14 (dd, J=7.72, 4.78 Hz, 1H), 7.27 (m, 2H), 7.55 (ddd, J=8.27, 7.17, 1.47 Hz, 1H), 7.67 (dd, J=8.09, 1.47 Hz, 1H), 8.37 (dd, J=7.54, 2.02 Hz, 1H), 8.53 (dd, J=4.78, 1.84 Hz, 1H), 15.93 (s, 1H).

### EXAMPLE 77

### 1-{3-[[2-(dimethylamino)ethyl](methyl)amino]propyl}-3-(1,1-dioxido-4H-1,2,4-benzothiadiazin-3-yl)-4-hydroxy-1,8-naphthyridin-2(1H)-one

The title compound was prepared according to the procedure of Example 72 substituting N,N,N-trimethylethylenediamine for aniline. MS (DCI/NH₃) m/z 485 (M+H)⁺. The dihydrochloride salt of the title compound was prepared according to the procedure of Example 72. ¹H NMR (300 MHz, DMSO-d₆) δ 2.20 (s, 2H), 2.84 (m, J=4.41 Hz, 6H), 3.50 (m, 9H), 4.54 (m, 2H), 7.54 (m, 3H), 7.77 (m, 1H), 7.91 (d, J=8.09 Hz, 1H), 8.59 (dd, J=8.09, 1.84 Hz, 1H), 8.85 (dd, J=4.60, 1.65 Hz, 1H), 10.43 (s, 1H), 14.25 (s, 1H).

### EXAMPLE 78

### 3-(1,1-dioxido-4H-1,2,4-benzothiadiazin-3-yl)-4-hydroxy-1-[3-(4-methyl-1-piperazinyl)propyl]-1,8-naphthyridin-2(1H)-one

The title compound was prepared according to the procedure of Example 72 substituting 4-methylpiperazine for aniline. MS (ESI−) m/z 450 (M−H)⁻. The dihydrochloride salt of the title compound was prepared according to the procedure of Example 72. ¹H NMR (300 MHz, DMSO-d₆) δ 2.03 (m, 2H), 3.10 (m, 4H), 3.69 (m, 4H), 3.90 (m, 2H), 4.39 (s, 2H), 7.20 (dd, J=7.72, 4.41 Hz, 1H), 7.30 (m, 2H), 7.58 (m, 1H), 7.68 (d, J=7.72 Hz, 1H), 8.42 (dd, J=7.72, 1.84 Hz, 1H), 8.55 (dd, J=4.41, 1.84 Hz, 1H), 15.71 (s, 1H).

### EXAMPLE 79

### 1-(2-aminoethyl)-3-(1,1-dioxido-4H-1,2,4-benzothiadiazin-3-yl)-4-hydroxy-1,8-naphthyridin-2(1H)-one

A solution of Example 66 (45.0 mg, 0.087 mmol) in a mixture of absolute ethanol (1.5 mL), N,N-dimethylformamide (0.8 mL) and dimethyl sulfoxide (1.0 mL) was treated with hydrazine monohydrate (13.42 mg, 0.261 mmol) at room temperature. The mixture was then heated to reflux at 80° C. for 5 hours, cooled to room temperature, and concentrated. The concentrate was purified by a C8 HPLC column eluting with 20% to 80% acetonitrile in water with 1% trifluoroacetic acid to give the TFA salt of the title compound (0.010 g, 23%). MS (APCI+) m/z 386 (M+H)⁻; ¹H NMR (300 MHz, DMSO-d₆) δ 3.20 (dd, J=11.95, 6.80 Hz, 2H), 4.62 (t, J=5.52 Hz, 2H), 7.27 (m, 1H), 7.39 (m, 2H), 7.65 (t, J=7.35 Hz, 1H), 7.75 (d, J=7.72 Hz, 1H), 7.82 (br s, 3H), 8.42 (d, J=9.56 Hz, 1H), 8.61 (d, J=3.31 Hz, 1H), 15.18 (br s, 1H).

### EXAMPLE 80

### 1-[3-(diethylamino)propyl]-3-(1,1-dioxido-4H-1,2,4-benzothiadiazin-3-yl)-4-hydroxy-1,8-naphthyridin-2(1H)-one

The title compound was prepared according to the procedure of Example 72 substituting diethylamine for aniline. MS (DCI/NH₃) m/z 456 (M+H)⁺. The hydrochloride salt of the title compound was prepared according to the procedure of Example 72. ¹H NMR (300 MHz, DMSO-d₆) δ 1.19 (t, J=7.17 Hz, 6H), 2.15 (m, 2H), 3.12 (m, 6H), 4.55 (t, J=6.62 Hz, 2H), 7.57 (m, 2H), 7.66 (m, 1H), 7.80 (m, 1H), 7.95 (d, J=8.09 Hz, 1H), 8.61 (dd, J=7.72, 1.84 Hz, 1H), 8.90 (dd, J=4.60, 1.65 Hz, 1H), 10.05 (s, 1H), 13.92 (s, 1H).

### EXAMPLE 81

### 1-cyclohexyl-3-(1,1-dioxido-4H-1,2,4-benzothiadiazin-3-yl)-4-hydroxy-1,8-naphthyridin-2(1H)-one

### EXAMPLE 81A

### ethyl 2-(cyclohexylamino)nicotinate

The title compound was prepared according to the procedure of Example 3A substituting cyclohexylamine for 2-ethylbutylamine (1.92 g, 61%). MS (ESI+) m/z 249.1 (M+H)⁺; ¹H NMR (300 MHz, CDCT₃) δ 1.38 (m, 7H), 1.61 (m, 2H), 1.75 (m, 2H), 2.02 (m, 2H), 4.08 (m, 1H), 4.31 (q, J=7.11 Hz, 2H), 6.46 (dd, J=7.72, 4.78 Hz, 1H), 7.99 (d, J=7.72 Hz, 1H), 8.10 (dd, J=7.72, 2.21 Hz, 1H), 8.25 (dd, J=4.78, 1.84 Hz, 1H).

### EXAMPLE 81B

### 1-cyclohexyl-2H-pyrido[2,3-d][1,3]oxazine-2,4(1H)-dione

The title compound was prepared according to the procedure of Example 3B substituting the product of Example 81A for the product of Example 3A (0.171 g, 35%). ¹H NMR (300 MHz, CDCl₃) δ 1.37 (m, 4H), 1.73 (m, 2H), 1.91 (m, 2H), 2.47 (ddd, J=24.82, 12.32, 3.31 Hz, 2H), 5.28 (tt, J=12.27, 3.72 Hz, 1H), 7.24 (dd, J=6.99, 4.04 Hz, 1H), 8.41 (dd, J=7.72, 2.21 Hz, 1H), 8.70 (dd, J=4.78, 2.21 Hz, 1H).

### EXAMPLE 81C

### 1-cyclohexyl-3-(1,1-dioxido-4H-1,2,4-benzothiadiazin-3-yl)-4-hydroxy-1,8-naphthyridin-2(1H)-one

The title compound was prepared according to the procedure of Example 1D substituting the product of Example 81B for the product of Example 1B (0.073 g, 26%). MS (ESI+) m/z 425.04 (M+H)⁺; ¹H NMR (300 MHz, DMSO-d₆) δ 1.25 (m, 4H), 1.76 (m, 4H), 1.91 (s, 2H), 5.64 (s, 1H), 7.48 (dd, J=8.09, 4.78 Hz, 1H), 7.55 (t, J=7.54 Hz, 1H), 7.69 (m, J=8.09 Hz, 1H), 7.77 (m, 1H), 7.92 (d, J=8.09 Hz, 1H), 8.56 (dd, J=8.09, 1.84 Hz, 1H), 8.86 (d, J=2.21 Hz, 1H), 14.12 (s, 1H). The sodium salt of the title compound was prepared according to the procedure of Example 1D. MS (ESI+) m/z 425.04 (M+H)⁺, 447.1 (M+Na)⁺; ¹H NMR (300 MHz, DMSO-d₆) δ 1.31 (m, 4H), 1.52 (d, J=10.66 Hz, 2H), 1.63 (m, 2H), 1.83 (m, J=12.50 Hz, 2H), 5.41 (t, J=11.03 Hz, 1H), 7.11 (dd, J=7.72, 4.78 Hz, 1H), 7.27 (m, 2H), 7.55 (m, 1H), 7.66 (d, J=6.62 Hz, 1H), 8.37 (dd, J=7.72, 2.21 Hz, 1H), 8.50 (dd, J=4.60, 2.02 Hz, 1H), 15.94 (s, 1H).

### EXAMPLE 82

### 3-(1,1-dioxido-4H-1,2,4-benzothiadiazin-3-yl)-4-hydroxy-1-[3-(4-morpholinyl)propyl]-1,8-naphthyridin-2(1H)-one

The title compound was prepared according to the procedure of Example 72 substituting morpholine for aniline (0.053 g 60%). MS (ESI−) m/z 450 (M−H)⁻. The sodium salt of the title compound was prepared according to the procedure of Example 1D. ¹H NMR (300 MHz, DMSO-d₆) δ 2.03 (m, 2H), 3.10 (m, 4H), 3.69 (m, 4H), 3.90 (m, 2H), 4.39 (s, 2H), 7.20 (dd, J=7.72, 4.41 Hz, 1H), 7.30 (m, 2H), 7.58 (m, 1H), 7.68 (d, J=7.72 Hz, 1H), 8.42 (dd, J=7.72, 1.84 Hz, 1H), 8.55 (dd, J=4.41, 1.84 Hz, 1H), 15.71 (s, 1H).

### EXAMPLE 83

### 5-{1[3-(1,1-dioxido-4H-1,2,4-benzothiadiazin-3-yl)-4-hydroxy-2-oxo-1,8-naphthyridin-1 (2H)-yl]methyl}-2-furoic Acid

A solution of the product of Example 75B (23 mg, 0.046 mmol) in THF (1 mL) was treated with 1N NaOH (0.2 mL) at room temperature. After 3 hours, the mixture was treated with H₂O (5 mL), adjusted to pH 4 with 1N HCl, and extracted with ethyl acetate (2×25 mL). The extracts were washed with saturated NaCl, dried over anhydrous Na₂SO₄, filtered, and concentrated. The resulting solid was purified by preparative HPLC on a Waters Symmetry C8 column (25 mm×100 mm, 7 μm particle size) using a gradient of 10% to 100% acetonitrile/0.1% aqueous TFA over 8 minutes (10 minute run time) at a flow rate of 40 mL/min to give the title compound (0.039 g, 83%). MS (ESI−) m/z 465 (M−H)⁻; ¹H NMR (300 MHz, DMSO-d₆) δ 5.72 (s, 2H), 6.42 (d, J=3.68 Hz, 1H), 7.11 (d, J=3.31 Hz, 1H), 7.53 (m, 2H), 7.68 (d, J=7.72 Hz, 1H), 7.76 (m, 1H), 7.92 (d, J=7.72 Hz, 1H), 8.60 (dd, J=8.09, 1.84 Hz, 1H), 8.86 (dd, J=4.78, 1.84 Hz, 1H), 13.90 (s, 1H).

### EXAMPLE 84

### 1-benzyl-3-(7-bromo-1,1-dioxido-4H-1,2,4-benzothiadiazin-3-yl)-4-hydroxy-1,8-naphthyridin-2(1H)-one

### EXAMPLE 84A

### 2-amino-5-bromobenzenesulfonamide

The title compound was prepared from 4-bromoaniline using the procedure described in JCS Perkin 1, 1979, 1043.

### EXAMPLE 84B

### ethyl 1-benzyl-4-hydroxy-2-oxo-1,2-dihydro-1,8-naphthyridine-3-carboxylate

To a slurry of sodium hydride (60%, 0.118 g, 2.95 mmol) in anhydrous dimethylacetamide (6 mL) at 0° C. under N₂ was added diethyl malonate (0.472 g, 2.95 mmol) dropwise over 5 minutes. The mixture was stirred at ambient temperature for 1 hour, reacted with the product of Example 15A (0.50 g, 1.97 mmol), and heated at 120° C. for 3 hours. The mixture was cooled to ambient temperature and partitioned between ethyl acetate and cold water, and adjusted to pH to 5 with 1 M HCl. The aqueous layer was extracted with ethyl acetate (2×100 mL) and the combined extracts were washed with brine, dried over magnesium sulfate, filtered, and concentrated under vacuum. The residue was recrystallized from methanol to give the title compound as a white solid (0.439 g, 68%). MS (ESI+) m/z 325.0 (M+H)⁺, 347.0 (M+Na)⁺; ¹H NMR (300 MHz, DMSO-d₆) δ 1.30 (t, J=7.17 Hz, 3H), 4.32 (q, J=7.23 Hz, 2H), 5.55 (s, 2H), 7.23 (m, 5H), 7.37 (dd, J=7.91, 4.60 Hz, 1H), 8.45 (dd, J=7.91, 2.02 Hz, 1H), 8.71 (dd, J=4.78, 1.84 Hz, 1H), 13.00 (s, 1H).

### EXAMPLE 84C

### N-[2-(aminosulfonyl)-4-bromophenyl]-1-benzyl-4-hydroxy-2-oxo-1,2-dihydro-1,8-naphthyridine-3-carboxamide

The product of Example 84B (0.065 g, 0.20 mmol) was reacted with the product of Example 84A (0.050 g, 0.20 mmol) in toluene (4 mL) at reflux for 3 hours. The reaction was cooled and the resulting precipitate was collected by filtration and dried to give to give the title compound as an off-white solid (0.074 g, 70%). MS (ESI+) m/z 528.9 (M+H)⁺, 530.9 (M+H)⁺, 551.1 (M+Na)⁺, 552.9 (M+Na)⁺; ¹H NMR (300 MHz, DMSO-d₆) δ 5.67 (s, 2H), 7.23 (m, 2H), 7.29 (m, 3H), 7.48 (dd, J=8.09, 4.78 Hz, 1H), 7.69 (s, 2H), 7.87 (dd, J=8.82, 2.21 Hz, 1H), 7.97 (m, 1H), 8.01 (d, J=2.21 Hz, 1H), 8.55 (dd, J=7.91, 1.65 Hz, 1H), 8.82 (dd, J=4.60, 1.65 Hz, 1H), 12.44 (s, 1H), 16.45 (s, 1H).

### EXAMPLE 84D

### 1-benzyl-3-(7-bromo-1,1-dioxido-4H-1,2,4-benzothiadiazin-3-yl)-4-hydroxy-1,8-naphthyridin-2(1H)-one

A mixture of the product of Example 84C (0.074 g, 0.14 mmol) in aqueous potassium hydroxide (10%, 5 mL) was heated to reflux for 16 hours, cooled to room temperature and adjusted to pH 3 with 6 M HCl. The mixture was filtered and the filter cake was washed with water, triturated with tetrahydrofuran/water, filtered, and dried under vacuum to give the title compound (0.060 g, 84%). MS (ESI+) m/z 511.0 (M+H)⁺, 512.9 (M+H)⁺; ¹H NMR (300 MHz, DMSO-d₆) δ 5.62 (s, 2H), 7.21 (m, 1H), 7.27 (m, J=4.41 Hz, 5H), 7.36 (m, 1H), 7.50 (d, J=8.82 Hz, 1H), 7.84 (dd, J=8.82, 1.84 Hz, 1H), 7.95 (s, 1H), 8.51 (dd, J=7.91, 1.65 Hz, 1H), 8.68 (m, 1H). The sodium salt of the title compound was prepared according to the procedure of Example 1D. MS (ESI+) m/z 511.0 (M+H—Na)⁺, 512.9 (M+H—Na)⁺; ¹H NMR (300 MHz, DMSO-d₆) δ 5.52 (s, 2H), 7.17 (m, 2H), 7.24 (m, 5H), 7.71 (m, 1H), 7.76 (d, J=2.21 Hz, 1H), 8.40 (dd, J=7.72, 1.84 Hz, 1H), 8.49 (dd, J=4.60, 2.02 Hz, 1H), 16.09 (s, 1H).

### EXAMPLE 85

### 1-benzyl-3-(1,1-dioxido-7-phenyl-2H-1,2,4-benzothiadiazin-3-yl)-4-hydroxy-1,8-naphthyridin-2(1H)-one

### EXAMPLE 85A

### N-[3-(aminosulfonyl)-1,1 ′-biphenyl-4-yl]-1-benzyl-4-hydroxy-2-oxo-1,2-dihydro-1,8-naphthyridine-3-carboxamide

The title compound was prepared according to the procedure of Example 84C substituting 2-amino-5-phenylbenzenesulfonamide for 2-amino-5-bromobenzenesulfonamide (0.084 g, 79%). MS (ESI+) m/z 527.1 (M+H)⁺, 549.1 (M+Na)⁺; ¹H NMR (300 MHz, DMSO-d₆) δ 5.68 (s, 2H), 7.2-7.8 (m, 13H), 7.98 (s, 1H), 8.09 (s, 1H), 8.17 (s, 1H), 8.54 (s, 1H), 8.81 (s, 1H), 12.49 (s, 1H), 16.67 (s, 1H).

### EXAMPLE 85B

### 1-benzyl-3-(1,1-dioxido-7-phenyl-2H-1,2,4-benzothiadiazin-3-yl)-4-hydroxy-1,8-naphthyridin-2(1H)-one

The title compound was prepared according to the procedure of Example 84D substituting the product of Example 85A for the product of Example 84C (0.055 g, 69%). MS (ESI+) m/z 509.1 (M+H)⁺; ¹H NMR (300 MHz, DMSO-d₆) δ 5.71 (s, 2H), 7.24 (m, 1H), 7.30 (m, 3H), 7.49 (m, 4H), 7.79 (m, J=7.35 Hz, 3H), 8.07 (m, J=11.03, 2.21 Hz, 2H), 8.60 (dd, J=7.91, 1.65 Hz, 1H), 8.81 (m, J=3.68 Hz, 1H). The sodium salt of the title compound was prepared according to the procedure of Example 1D. MS (ESI+) m/z 531.0 (M+), 509.1 (M−Na⁺ H)⁺; ¹H NMR (300 MHz, DMSO-d₆) δ 5.53 (s, 2H), 7.17 (m, 2H), 7.25 (m, J=4.41 Hz, 4H), 7.39 (m, 2H), 7.49 (t, J=7.54 Hz, 2H), 7.71 (d, J=6.99 Hz, 2H), 7.89 (m, 2H), 8.42 (dd, J=7.72, 1.84 Hz, 1H), 8.49 (dd, J=4.60, 2.02 Hz, 1H), 15.99 (s, 1H).

### EXAMPLE 86

### 1-benzyl-3-(7-cyclohexyl-1,1-dioxido-4H-1,2,4-benzothiadiazin-3-yl)-4-hydroxy-1,8-naphthyridin-2(1H)-one

### EXAMPLE 86A

### 2-amino-5-cyclohexylbenzenesulfonamide

A solution of 4-cyclohexylaniline (0.877 g, 5.0 mmol, 1.0 eq) in nitroethane (5 mL) was cooled to −40° C., treated dropwise with chlorosulfonyl isocyanate (0.87 g, (0.523 mL, 6.15 mmol, 1.23 eq), warmed to 0° C., treated with aluminum trichloride (0.85 g, 6.35 mmol, 1.27 eq), heated in a 110° C. oil bath for 30 minutes, cooled to ambient temperature, and poured into 200 mL of ice water. The mixture was filtered and the filter cake was rinsed with cold water, dissolved in 50% H₂SO₄ (25 mL), heated to reflux for 4 hours, cooled to ambient temperature, poured into 200 mL of ice water, and carefully neutralized to pH 7 with 40% NaOH. The reaction mixture was extracted with ethyl acetate (3×100 mL) and the combined extracts were washed with brine, dried (MgSO₄), filtered, and concentrated to give 0.40 g of the desired product (31% yield). MS (ESI+) m/z 255.0 (M+H)⁺, 272.1 (M+H₂O)⁺, 277.0 (M+Na)⁺; ¹H NMR (300 MHz, DMSO-d₆) δ 1.32 (m 4H), 1.71 (m, 6H), 2.36 (m, 1H), 5.64 (s, 2H), 6.72 (d, J=8.09 Hz, 1H), 7.11 (dd, J=8.46, 2.21 Hz, 1H), 7.16 (s, 2H), 7.38 (d, J=2.21 Hz, 1H).

### EXAMPLE 86B

### N-[2-(aminosulfonyl)-4-cyclohexylphenyl]-1-benzyl-4-hydroxy-2-oxo-1.2-dihydro-1,8-naphthyridine-3-carboxamide

The title compound was prepared according to the procedure of Example 84C substituting the product of Example 86A for 2-amino-5-bromobenzenesulfonamide (0.081 g, 76%). MS (ESI+) m/z 533.1 (M+H)⁺, 555.2 (M+Na)⁺; ¹H NMR (300 MHz, DMSO-d₆) δ 1.27 (m, 1H), 1.45 (m, 4H), 1.72 (m, 1H), 1.85 (m, 4H), 2.61 (m, 1H), 5.68 (s, 2H), 7.26 (m, 4H), 7.50 (m, 4H), 7.76 (d, J=1.84 Hz, 1H), 7.86 (d, J=8.46 Hz, 2H), 8.54 (dd, J=8.09, 1.47 Hz, 1H), 8.80 (s, 1H), 12.31 (s, 1H), 16.78 (s, 1H).

### EXAMPLE 86C

### 1-benzyl-3-(7-cyclohexyl-1,1-dioxido-4H-1,2,4-benzothiadiazin-3-yl)-4-hydroxy-1,8-naphthyridin-2(1H)-one

The title compound was prepared according to the procedure of Example 84D substituting the product of Example 86B for the product of Example 84C (0.040 g, 53%). MS (ESI+) m/z 533.1 (M+H+H₂O)⁺, 555.1 (M+H₂O+Na)⁺, 515.1 (M+H)⁺; ¹H NMR (300 MHz, DMSO-d₆) δ 1.34 (m, 5H), 1.77 (m, 5H), 2.60 (m, 1H), 5.66 (s, 2H), 7.25 (m, 4H), 7.51 (m, J=9.56 Hz, 4H), 7.88 (s, 1H), 8.54 (s, 1H), 8.80 (s, 1H), 12.31 (s, 1H), 16.78 (s, 1H). The sodium salt of the title compound was prepared according to the procedure of Example 1D. ¹H NMR (300 MHz, DMSO-d₆) δ 1.41 (m, 5H), 1.70 (m, 5H), 3.79 (m, 1H), 5.52 (s, 2H), 7.12 (dd, J=7.54, 4.60 Hz, 1H), 7.17 (m, 1H), 7.23 (m, 4H), 7.41 (dd, J=8.64, 2.02 Hz, 1H), 7.67 (d, J=2.21 Hz, 1H), 8.33 (d, J=8.46 Hz, 1H), 8.38 (dd, J=7.72, 1.84 Hz, 1H), 8.43 (dd, J=4.60, 2.02 Hz, 1H), 11.15 (s, 1H).

### EXAMPLE 87

### 1-benzyl-3-(7-tert-butyl-1,1-dioxido-4H-1,2,4-benzothiadiazin-3-yl)-4-hydroxy-1,8-naphthyridin-2(1H)-one

### EXAMPLE 87A

### N-[2-(aminosulfonyl)-4-tert-butylphenyl]-1-benzyl-4-hydroxy-2-oxo-1,2-dihydro-1,8-naphthyridine-3-carboxamide

The title compound was prepared according to the procedure of Example 84C substituting 2-amino-5-tert-butylbenzenesulfonamide for 2-amino-5-bromobenzenesulfonamide (0.072 g, 79%). MS (ESI+) m/z 507.12 (M+H)⁺, 524.2 (M+H₂O)⁺, 529.1 (M+Na)⁺; ¹H NMR (300 MHz, DMSO-d₆) δ 1.33 (s, 9H), 5.68 (s, 2H), 7.22 (m, 1H), 7.29 (m, J=3.68 Hz, 4H), 7.47 (m, 3H), 7.70 (dd, J=8.64, 2.39 Hz, 1H), 7.88 (d, J=8.82 Hz, 1H), 7.91 (d, J=2.21 Hz, 1H), 8.54 (dd, J=8.09, 1.84 Hz, 1H), 8.81 (dd, J=4.60, 1.65 Hz, 1H), 12.33 (s, 1H), 16.79 (s, 1H).

### EXAMPLE 87B

### 1-benzyl-3-(7-tert-butyl-1,1-dioxido-4H-1,2,4-benzothiadiazin-3-yl)-4-hydroxy-1,8-naphthyridin-2(1H)-one

The title compound was prepared according to the procedure of Example 84D substituting the product of Example 87A for the product of Example 84C (0.040 g, 100%). MS (ESI+) m/z 489.1 (M+H)⁺, 511.1 (M+Na)⁺; ¹H NMR (300 MHz, DMSO-d₆) δ 1.34 (s, 9H), 5.70 (s, 2H), 7.22 (m, 1H), 7.29 (m, J=4.41 Hz, 4H), 7.48 (m, 1H), 7.59 (d, J=8.82 Hz, 1H), 7.75 (s, 1H), 7.81 (d, J=10.66 Hz, 1H), 8.58 (d, J=6.62 Hz, 1H), 8.79 (s, 1H). The sodium salt of the title compound was prepared according to the procedure of Example 1D. ¹H NMR (300 MHz, DMSO-d₆) δ 1.32 (s, 9H), 5.53 (s, 2H), 7.18 (m, 2H), 7.25 (m, J=4.41 Hz, 5H), 7.59 (s, 1H), 7.65 (m, 1H), 8.42 (d, J=7.35 Hz, 1H), 8.50 (m, J=3.86, 2.02 Hz, 1H).

### EXAMPLE 88

### 1-benzyl-4-hydroxy-3-(7-methyl-1,1-dioxido-4H-1,2,4-benzothiadiazin-3-yl)-1,8-naphthyridin-2(1H)-one

### EXAMPLE 88A

### N-[2-(aminosulfonyl)-4-methylphenyl]-1-benzyl-4-hydroxy-2-oxo-1,2-dihydro-1,8-naphthyridine-3-carboxamide

The title compound was prepared according to the procedure of Example 84C substituting 2-amino-5-methylbenzenesulfonamide for 2-amino-5-bromobenzenesulfonamide (0.075 g, 90%). MS (ESI+) m/z 465.1 (M+H)⁺, 482.0 (M+H₂O)⁺, 487.1 (M+Na)⁺. ¹H NMR (300 MHz, DMSO-d₆) δ 2.39 (s, 3H), 5.68 (s, 2H), 7.23 (m, 1H), 7.29 (m, 4H), 7.47 (m, 4H), 7.73 (d, J=1.47 Hz, 1H), 7.84 (d, J=8.09 Hz, 1H), 8.54 (dd, J=7.72, 1.84 Hz, 1H), 8.81 (dd, J=4.60, 1.65 Hz, 1H), 12.30 (s, 1H), 16.78 (s, 1H).

### EXAMPLE 88B

### 1-benzyl-4-hydroxy-3-(7-methyl-1,1-dioxido-4H-1,2,4-benzothiadiazin-3-yl)-1,8-naphthyridin-2(1H)-one

The title compound was prepared according to the procedure of Example 84D substituting the product of Example 88A for the product of Example 84C (0.031 g, 42%). MS (ESI+) m/z 447.0 (M+H)⁺, 469.1 (M+Na)⁺. ¹H NMR (300 MHz, DMSO-d₆) δ 2.41 (s, 3H), 5.65 (s, 2H), 7.24 (m, 5H), 7.45 (m, 3H), 7.66 (s, 1H), 8.54 (d, J=7.72 Hz, 1H), 8.72 (s, 1H). The sodium salt of the title compound was prepared according to the procedure of Example 1D. ¹H NMR (300 MHz, DMSO-d₆) δ 2.37 (s, 3H), 5.55 (s, 2H), 7.21 (m, 7H), 7.41 (d, J=8.46 Hz, 1H), 7.51 (s, 1H), 8.43 (d, J=8.09 Hz, 1H), 8.53 (s, 1H).

### EXAMPLE 89

### 1-butyl-3-(6-chloro-1,1-dioxido-4H-1,2,4-benzothiadiazin-3-yl)-4-hydroxy-1,8-naphthyridin-2(1H)-one

### EXAMPLE 89A

### ethyl 1-butyl-4-hydroxy-2-oxo-1,2-dihydro-1,8-naphthyridine-3-carboxylate

To a slurry of NaH (95%, 0.44 g, 18.2 mmol) in 15 mL anhydrous DMA at 10° C. under N₂ was added diethyl malonate (2.9 g, 18.2 mmol) dropwise over 10 minutes. The mixture was stirred at ambient temperature for 30 minutes, treated with the product of Example 1B (2.0 g, 9.1 mmol) and heated at 120° C. for 3 hours. The mixture was cooled to ambient temperature and partitioned between ethyl acetate and cold water adjusting the pH to 5 with 1 M HCl. The organic layer was washed 2×100 mL with water, 2×100 mL with saturated brine, dried (Na₂SO₄), filtered and the filtrate was concentrated under vacuum. The residue was recrystallized from hexane/ethyl acetate to give the desired compound as a white solid (1.84 g, 70% yield). MS (APCI+) m/z 291 (M+H)⁺.

### EXAMPLE 89B

### N-[2-(aminosulfonyl)-4-chlorophenyl]-1-butyl-4-hydroxy-2-oxo-1,2-dihydro-1,8-naphthyridine-3-carboxamide

A mixture of the product of Example 89A (87 mg, 0.3 mmol) and 2-amino-4-chlorobenzenesulfonamide (62 mg, 0.3 mmol) in toluene (5 mL) was refluxed for 16 hours, cooled, and the resulting precipitate was collected by filtration and dried to give the desired amide as an off-white solid (80 mg, 59% yield). MS (APCI+) m/z 451 (M+H)⁺.

### EXAMPLE 89C

### ethyl 1-butyl-4-hydroxy-2-oxo-1,2-dihydro-1,8-naphthyridine-3-carboxylate

The title compound was prepared according to the procedure of Example 84C substituting the product of Example 89B for the product of Example 84B (0.037 g, 53%). MS (ESI−) m/z 431 (M−H)⁻. The sodium salt of the title compound was prepared according to the procedure of Example 1D. ¹H NMR (300 MHz, DMSO-d₆) δ 0.93 (t, J=7.17 Hz, 3H), 1.34 (m, 2H), 1.58 (m, 2H), 4.27 (m, 2H), 7.14 (dd, J=7.72, 4.78 Hz, 1H), 7.32 (dd, J=8.27, 2.02 Hz, 1H), 7.42 (d, J=1.84 Hz, 1H), 7.68 (d, J=8.46 Hz, 1H), 8.37 (dd, J=7.54, 2.02 Hz, 1H), 8.54 (dd, J=4.78, 1.84 Hz, 1H), 16.09 (s, 1H).

### EXAMPLE 90

### 1-benzyl-3-(8-bromo-5-methyl-1,1-dioxido-4H-1,2,4-benzothiadiazin-3-yl)-4-hydroxy-1,8-naphthyridin-2(1H)-one

### EXAMPLE 90A

### N-[2-(aminosulfonyl)-3-bromo-6-methylphenyl]-1-benzyl-4-hydroxy-2-oxo-1,2-dihydro-1,8-naphthyridine-3-carboxamide

The title compound was prepared according to the procedure of Example 84C substituting 2-amino-6-bromo-3-methylbenzenesulfonamide for 2-amino-5-bromobenzenesulfonamide to give the crude title compound (0.1 g, 98%).

### EXAMPLE 90B

### 1-benzyl-3-(8-bromo-5-methyl-1,1-dioxido-4H— 1,2,4-benzothiadiazin-3-yl)-4-hydroxy-1,8-naphthyridin-2(1H)-one

The title compound was prepared according to the procedure of Example 84D substituting the product of Example 90A for the product of Example 84C. The crude product was purified by column chromatography with silica gel eluting with dichloromethane and methanol (98:2) to give the title compound as a white solid, (0.03 g, 31% yield). MS (ESI−) m/z 525 (M−H)⁻. The sodium salt of the title compound was prepared according to the procedure of Example 1D. ¹H NMR (300 MHz, DMSO-d₆) δ 16.0 (br s, 1H), 8.49 (dd, J=4.8, 1.8 Hz, 1H), 8.44 (dd, J=7.7, 1.8 Hz, 1H), 7.45 (br s, 1H), 7.37 (m, 1H), 7.23 (m, 3H), 7.16 (dd, J=4.8, 3.3 Hz, 1H), 7.01 (m, 1H), 6.85 (d, J=7.7 Hz, 1H), 5.53 (br s, 2H), 2.43 (s, 3H).

### EXAMPLE 91

### 1-benzyl-3-(8-fluoro-5-methyl-1,1-dioxido-4H-1,2,4-benzothiadiazin-3-yl)-4-hydroxy-1,8-naphthyridin-2(1H)-one

### EXAMPLE 91A

### N-[2-(aminosulfonyl)-3-fluoro-6-methylphenyl]-1-benzyl-4-hydroxy-2-oxo-1,2-dihydro-1,8-naphthyridine-3-carboxamide

The title compound was prepared according to the procedure of Example 84C substituting 2-amino-6-fluoro-3-methylbenzenesulfonamide for 2-amino-5-bromobenzenesulfonamide to give the crude title compound (0.120 g, 100%).

### EXAMPLE 91B

### 1-benzyl-3-(8-fluoro-5-methyl-1,1-dioxido-4H-1,2,4-benzothiadiazin-3-yl)-4-hydroxy-1,8-naphthyridin-2(1H)-one

The title compound was prepared according to the procedure of Example 84D substituting the product of Example 91A for the product of Example 84C. The crude product was purified by column chromatography with silica gel eluting with dichloromethane and methanol (98:2) as a white solid, (0.05 g, 44% yield). MS (ESI−) m/z 463 (M−H)⁻. The sodium salt of the title compound was prepared according to the procedure of Example 1D. ¹H NMR (300 MHz, DMSO-d₆) δ 16.1 (br s, 1H), 8.49 (dd, J=4.6, 2.0 Hz, 1H), 8.44 (dd, J=7.7, 1.8 Hz, 1H), 7.59 (m, 1H), 7.47 (dd, J=7.3, 5.8 Hz, 1H), 7.38 (m, 1H), 7.21 (m, 3H), 7.16 (dd, J=7.7, 5.8 Hz, 1H), 6.99 (t, J=8.8 Hz, 1H), 5.53 (s, 2H), 2.42 (s, 3H).

### EXAMPLE 92

### 1-benzyl-4-hydroxy-3-(5-isopropyl-1,1-dioxido-4H-1,2,4-benzothiadiazin-3-yl)-1,8-naphthyridin-2(1H)-one

### EXAMPLE 92A

### N-[2-(aminosulfonyl)-6-isopropylphenyl]-1-benzyl-4-hydroxy-2-oxo-1,2-dihydro-1,8-naphthyridine-3-carboxamide

The title compound was prepared according to the procedure of Example 84C substituting 2-amino-3-isopropylbenzenesulfonamide for 2-amino-5-bromobenzenesulfonamide (0.050 g, 55%) after chromatrography on silica gel (eluting with 4:1 hexane/ethyl acetate). ¹H NMR (300 MHz, DMSO-d₆) 6 ¹H NMR (300 MHz, DMSO-d₆) δ 1.12 (d, J=6.62 Hz, 3H), 1.26 (d, J=6.99 Hz, 3H), 3.06 (m, 1H), 5.69 (m, 2H), 7.27 (m, 5H), 7.39 (s, 2H), 7.48 (dd, J=7.72, 4.78 Hz, 1H), 7.55 (t, J=7.72 Hz, 1H), 7.71 (d, J=8.09 Hz, 1H), 7.80 (dd, J=7.72, 1.10 Hz, 1H), 8.53 (dd, J=7.91, 1.65 Hz, 1H), 8.83 (dd, J=4.78, 1.84 Hz, 1H), 11.75 (s, 1H), 16.83 (s, 1H).

### EXAMPLE 92B

### 1-benzyl-4-hydroxy-3-(5-isopropyl-1,1-dioxido-4H-1,2,4-benzothiadiazin-3-yl)-1,8-naphthyridin-2(1H)-one

The title compound was prepared according to the procedure of Example 84D substituting the product of Example 92a for the product of Example 84C (0.038 g, 75%). MS (ESI+) m/z 475.1 (M+H)⁺, 492.1 (M+H₂O)⁺, 497.1 (M+Na)⁺. ¹H NMR (300 MHz, DMSO-d₆) 1.34 (d, J=6.62 Hz, 6H), 3.30 (m, 1H), 5.73 (s, 2H), 7.27 (m, 5H), 7.54 (m, 2H), 7.78 (m, J=16.18, 7.72 Hz, 2H), 8.62 (dd, J=7.91, 1.65 Hz, 1H), 8.84 (s, 1H), 14.64 (s, 1H). The sodium salt of the title compound was prepared according to the procedure of Example 1D. ¹H NMR (300 MHz, DMSO-d₆) δ 1.32 (d, J=6.62 Hz, 6H), 3.42 (m, 1H), 5.53 (s, 2H), 7.15 (m, 2H), 7.25 (m, J=4.41 Hz, 4H), 7.29 (m, 1H), 7.53 (m, J=7.72, 1.84 Hz, 2H), 8.46 (m, 2H), 16.06 (s, 1H).

### EXAMPLE 93

### 1-benzyl-4-hydroxy-3-(5-methyl-1,1-dioxido-4H— 1,2,4-benzothiadiazin-3-yl)-1,8-naphthyridin-2(1H)-one

### EXAMPLE 93A

### N-[2-(aminosulfonyl)-6-methylphenyl]-1-benzyl-4-hydroxy-2-oxo-1,2-dihydro-1,8-naphthyridine-3-carboxamide

The title compound was prepared according to the procedure of Example 84C substituting 2-amino-3-methylbenzenesulfonamide for 2-amino-5-bromobenzenesulfonamide (0.059 g, 100%). ¹H NMR (300 MHz, DMSO-d₆) δ 2.27 (s, 3H) 5.68 (m, 2H) 7.24 (m, 5H) 7.46 (m, 4H) 7.59 (d, J=6.99 Hz, 1H) 7.79 (d, J=7.72 Hz, 1H) 8.54 (dd, J=8.09, 1.84 Hz, 1H) 8.83 (dd, J=4.78, 1.84 Hz, 1H) 11.90 (s, 1H) 16.79 (s, 1H).

### EXAMPLE 93B

### 1-benzyl-4-hydroxy-3-(5-methyl-1,1-dioxido-4H-1,2,4-benzothiadiazin-3-yl)-1,8-naphthyridin-2(1H)-one

The title compound was prepared according to the procedure of Example 84D substituting the product of Example 93A for the product of Example 84C (0.015 g, 25%) after silica gel chromatography (eluting with 98:2 dichloromethane/methanol). MS (ESI+) m/z 447.0 (M+H)⁺, 469.1 (M+Na)⁺. ¹H NMR (300 MHz, DMSO-d₆) δ 2.52 (m, 3H) 5.75 (m, 2H) 7.23 (m, 1H) 7.30 (m, 4H) 7.47 (t, J=7.72 Hz, 1H) 7.53 (dd, J=8.09, 4.78 Hz, 1H) 7.69 (d, J=7.35 Hz, 1H) 7.79 (d, J=8.09 Hz, 1H) 8.63 (dd, J=7.72, 1.84 Hz, 1H) 8.85 (dd, J=4.78, 1.84 Hz, 1H) 14.41 (s, 1H). The sodium salt of the title compound was prepared according to the procedure of Example 1d. ¹H NMR (300 MHz, DMSO-d₆) δ 2.48 (s, 3H) 5.56 (s, 2H) 7.21 (m, 6H) 7.49 (d, J=7.35 Hz, 1H) 7.56 (d, J=7.35 Hz, 1H) 8.47 (d, J=7.72 Hz, 1H) 8.53 (s, 1H) 11.98 (s, 1H).

### EXAMPLE 94

### 1-benzyl-3-(5-bromo-1,1-dioxido-4H-1,2,4-benzothiadiazin-3-yl)-4-hydroxy-1,8-naphthyridin-2(1H)-one

### EXAMPLE 94A

### N-[2-(aminosulfonyl)-6-bromophenyl]-1-benzyl-4-hydroxy-2-oxo-1,2-dihydro-1,8-naphthyridine-3-carboxamide

The title compound was prepared according to the procedure of Example 84C substituting 2-amino-3-methylbenzenesulfonamide for 2-amino-5-bromobenzenesulfonamide (0.080 g, 25%) after silica gel chromatography (eluting with 2:1 hexane/ethyl acetate). MS (ESI+) m/z 529.0 (M+H)⁺, 530.9 (M+H)⁺. ¹H NMR (300 MHz, DMSO-d₆) δ 5.71 (m, 2H) 7.23 (m, 1H) 7.32 (m, 4H) 7.50 (m, 2H) 7.62 (s, 2H) 7.96 (dd, J=7.91, 1.29 Hz, 1H) 8.02 (dd, J=7.91, 1.29 Hz, 1H) 8.55 (dd, J=7.91, 1.65 Hz, 1H) 8.85 (dd, J=4.78, 1.84 Hz, 1H) 11.95 (s, 1H) 16.51 (s, 1H).

### EXAMPLE 94B

### 1-benzyl-3-(5-bromo-1,1-dioxido-4H-1,2,4-benzothiadiazin-3-yl)-4-hydroxy-1,8-naphthyridin-2(1H)-one

The title compound was prepared according to the procedure of Example 84D substituting the product of Example 94A for the product of Example 84C (0.040 g, 54%). MS (ESI+) m/z 510.9 (M+H)⁺, 512.9 (M+H)⁺. ¹H NMR (300 MHz, DMSO-d₆) δ 5.56 (s, 2H) 7.23 (m, 8H) 7.76 (d, J=8.46 Hz, 1H) 7.94 (d, J=8.09 Hz, 1H) 8.46 (dd, J=7.72, 1.84 Hz, 1H) 8.55 (m, 1H) 16.17 (s, 1H). The sodium salt of the title compound was prepared according to the procedure of Example 1d. ¹H NMR (300 MHz, DMSO-d₆) δ 5.53 (s, 2H) 7.17 (m, 2H) 7.25 (m, 5H) 7.71 (d, J=6.99 Hz, 1H) 7.90 (m, 1H) 8.43 (dd, J=7.72, 1.84 Hz, 1H) 8.49 (dd, J=4.60, 2.02 Hz, 1H) 16.38 (s, 1H).

### EXAMPLE 95

### 1-benzyl-3-(1,1-dioxido-5-propyl-2H-1,2,4-benzothiadiazin-3-yl)-4-hydroxy-1,8-naphthyridin-2(1H)-one

### EXAMPLE 95A

### N-[2-(aminosulfonyl)-6-propylphenyl]-1-benzyl-4-hydroxy-2-oxo-1,2-dihydro-1,8-naphthyridine-3-carboxamide

The title compound was prepared according to the procedure of Example 84C substituting 2-amino-3-propylbenzenesulfonamide for 2-amino-5-bromobenzenesulfonarmide (0.062 g, 59%). MS (DCI/NH₃) m/z 493 (M+H)⁺. ¹H NMR (300 MHz, DMSO-d₆) δ ¹H NMR (300 MHz, DMSO-d₆) δ 0.83 and 0.93 (two t, J=7.35 Hz, 3H), 1.57 (m, 2H), 2.57 (m, 2H), 5.66 (m, 2H), 7.28 (m, 5H), 7.41 (s, 2H), 7.48 (m, 2H), 7.61 (m, 1H), 7.81 (dd, J=7.72, 1.47 Hz, 1H), 8.53 (dd, J=7.91, 1.65 Hz, 1H), 8.83 (dd, J=4.78, 1.84 Hz, 1H), 11.82 (s, 1H), 16.80 (s, 1H).

### EXAMPLE 95B

### 1-benzyl-3-(1,1-dioxido-5-propyl-2H-1,2,4-benzothiadiazin-3-yl)-4-hydroxy-1,8-naphthyridin-2(1H)-one

The title compound was prepared according to the procedure of Example 84D substituting the product of Example 95A for the product of Example 84C (0.029 g, 50%). MS (ESI−) m/z 473 (M−H). The sodium salt of the title compound was prepared according to the procedure of Example 1D. ¹H NMR (300 MHz, DMSO-d₆) δ 1.03 (t, J=7.17 Hz, 3H), 1.68 (m, 2H), 2.83 (t, J=7.72 Hz, 2H), 5.53 (s, 2H), 7.19 (m, 7H), 7.44 (d, J=6.25 Hz, 1H), 7.53 (d, J=7.35 Hz, 1H), 8.43 (dd, J=7.54, 1.84 Hz, 1H), 8.48 (dd, J=4.78, 1.84 Hz, 1H), 16.02 (s, 1H).

### EXAMPLE 96

### 1-benzyl-3-(5-ethyl-1,1-dioxido-4H-1,2,4-benzothiadiazin-3-yl)-4-hydroxy-1,8-naphthyridin-2(1H)-one

### EXAMPLE 96A

### N-[2-(aminosulfonyl)-6-ethylphenyl]-1-benzyl-4-hydroxy-2-oxo-1,2-dihydro-1,8-naphthyridine-3-carboxamide

The title compound was prepared according to the procedure of Example 84C substituting 2-amino-3-ethylbenzenesulfonamide for 2-amino-5-bromobenzenesulfonamide (0.070 g, 74%). MS (ESI−) m/z 479 (M+H)⁺. ¹H NMR (300 MHz, DMSO-d₆) δ 1.17 (t, J=7.54 Hz, 3H), 2.61 (q, J=7.60 Hz, 2H), 5.70 (m, 2H), 7.27 (m, 5H), 7.42 (s, 2H), 7.49 (m, 2H), 7.63 (m, 1H), 7.81 (dd, J=7.91, 1.29 Hz, 1H), 8.53 (dd, J=7.91, 1.65 Hz, 1H), 8.83 (dd, J=4.41, 1.84 Hz, 1H), 11.82 (s, 1H), 16.80 (s, 1H).

### EXAMPLE 96B

### 1-benzyl-3-(5-ethyl-1,1-dioxido-4H-1,2,4-benzothiadiazin-3-yl)-4-hydroxy-1,8-naphthyridin-2(1H)-one

The title compound was prepared according to the procedure of Example 84D substituting the product of Example 96A for the product of Example 84C (0.060 g, 93%). MS (ESI−) m/z 459 (M−H)⁻. The sodium salt of the title compound was prepared according to the procedure of Example 1D. ¹H NMR (300 MHz, DMSO-d₆) δ 1.30 (t, J=7.54 Hz, 3H), 2.86 (q, J=7.35 Hz, 2H), 5.53 (s, 2H), 7.14 (dd, J=7.72, 4.78 Hz, 1H), 7.22 (m, 6H), 7.46 (d, J=7.72 Hz, 1H), 7.53 (d, J=7.72 Hz, 1H), 8.44 (dd, J=7.72, 1.84 Hz, 1H), 8.48 (dd, J=4.78, 1.83 Hz, 1H), 15.98 (s, 1H).

### EXAMPLE 97

### 3-(1-benzyl-4-hydroxy-2-oxo-1,2-dihydro-1,8-naphthyridin-3-yl)-2H-1,2,4-benzothiadiazine-5-carbonitrile1,1-dioxide

The product of Example 94B (0.329 g, 0.643 mmol) and CuCN (0.29 g, 3.21 mmol) in anhydrous DMF (5 mL) were heated under N₂ at 145° for 22 hrs. The reaction was cooled to room temperature, diluted with CH₂Cl₂ (50 mL) and 1N aq HCl (10 mL), and vigorously stirred for 15 minutes. The layers were separated and the aqueous phase extracted with CH₂Cl₂ (2×50 mL). The organic extracts were washed with 1N aqueous HCl (20 mL) and saturated aqueous NaCl, then dried over anhydrous Na₂SO₄. After filtration and concentration by rotary evaporation, the residue was purified by silica gel flash chromatography (2.5×14 cm, 5% EtOAc/CH₂Cl₂) to give the title compound (0.136 g, 46%). MS (ESI−) m/z 456 (M−H)⁻. ¹H NMR (300 MHz, DMSO-d₆) δ 5.69 (s, 2H) 7.26 (m, 5H) 7.47 (dd, J=7.91, 4.60 Hz, 1H) 7.62 (t, J=7.91 Hz, 1H) 8.25 (m, 2H) 8.59 (dd, J=7.91, 2.02 Hz, 1H) 8.80 (dd, J=4.60, 1.65 Hz, 1H) 15.67 (s, 1H). The sodium salt of the title compound was prepared according to the procedure of Example 1D. ¹H NMR (300 MHz, DMSO-d₆) δ 5.53 (s, 2H) 7.20 (m, 6H) 7.41 (t, J=7.91 Hz, 1H) 8.00 (d, J=7.35 Hz, 1H) 8.07 (d, J=7.72 Hz, 1H) 8.43 (dd, J=7.54, 1.65 Hz, 1H) 8.51 (dd, J=4.60, 1.65 Hz, 1H) 17.35 (s, 1H).

### EXAMPLE 98

### 1-butyl-3-(1,1-dioxido-4H-pyrido[3,2-e][1,2,4]thiadiazin-3-yl)-4-hydroxy-2(1H)-quinolinone

### EXAMPLE 98A

### 3-nitropyridine-2-thiol

2-mercapto-3-nitropyridine was prepared by treating 3-nitro-2-chloro-pyridine (50 g, 0.0317 mol) with thiourea (24 g, 0.0317 mol) in 200 mL of ethanol at reflux for several hours. After the reaction mixture was allowed to cool, 7.19 mL solution of KOH (42.8 g in 115 mL of water) was added and the resulting mixture was heated at reflux for 3 hours. The crude reaction mixture was cooled to room temperature and then concentrated to 50% of its volume in vacuo. After diluting with 300 mL of water, the product was isolated by vacuum filtration as an orange solid that was used without further purification. MS (DCI/NH₃) m/z 157 (M+H)⁺. ¹H NMR (300 MHz, DMSO-d₆) δ 5.76 (m, 1H), 7.67 (dd, J=8.46, 4.78 Hz, 1H), 8.63 (dd, J=8.46, 1.47 Hz, 1H), 8.73 (dd, J=4.60, 1.65 Hz, 1H).

### EXAMPLE 98B

### 3-aminopyridine-2-sulfonamide

The title compound, (3-aminopyrid-2-yl)sulfonamide was prepared in 3 steps (80% yield) from 2-mercapto-3-nitropyridine according to the procedure of R. Lejeune and co-workers as described in J.pharm. Belg., 39, 217-224, 1984. MS (DCI/NH₃) m/z 174 (M+H)⁺.

¹H NMR (300 MHz, DMSO-d₆) δ 6.00 (s, 2H), 7.25 (m, 2H), 7.34 (s, 2H), 7.82 (dd, J=4.04, 1.47 Hz, 1H).

### EXAMPLE 98C

### N-[2-(aminosulfonyl)pyridin-3-yl]-1-butyl-4-hydroxy-2-oxo-1,2-dihydroquinoline-3-carboxamide

The title compound was prepared according to the procedure of Example 89B substituting 3-amino-pyridine-2-sulfonamide for 2-amino-4-chlorobenzenesulfonamide.

### EXAMPLE 98D

### 1-butyl-3-(1,1-dioxido-4H-pyrido[3,2-e][1,2,4]thiadiazin-3-yl)-4-hydroxy-2(1H)-quinolinone

The title compound was prepared according to the procedure of Example 84D substituting the product of Example 98C for the product of Example 84C as a white solid (0.065 g, 22%). MS (ESI−) m/z 397 (M−H)⁻; ¹H NMR (300 MHz, DMSO-d₆) δ 0.96 (t, J=7.35 Hz, 3H), 1.46 (m, 2H), 1.66 (m, 2H), 4.34 (m, 2H), 7.46 (t, J=7.54 Hz, 1H), 7.82 (m, 3H), 8.23 (d, J=6.99 Hz, 1H), 8.26 (d, J=7.72 Hz, 1H), 8.70 (d, J=3.68 Hz, 1H), 14.38 (s, 1H), 15.12 (s, 1H).

### EXAMPLE 99

### 1-benzyl-3-(1,1-dioxido-4H-pyrido [32-e][1,2,4]thiadiazin-3-yl)-4-hydroxy-2(1H)-quinolinone

### EXAMPLE 99A

### ethyl 1-benzyl-4-hydroxy-2-oxo-1,2-dihydroquinoline-3-carboxylate

The title compound was prepared according to the procedure of Example 84B substituting 1-benzyl-1H-benzo[d][1,3]oxazine-2,4-dione for the product of Example ISA.

### EXAMPLE 99B

### N-[2-(aminosulfonyl)pyridin-3-yl]-1-benzyl-4-hydroxy-2-oxo-1,2-dihydroquinoline-3-carboxamide

The title compound was prepared according to the procedure of Example 84C substituting the product of Example 99A for the product of Example 84B and substituting (3-amino-pyrid-2-yl)sulfonamide for 2-amino-5-bromobenzenesulfonamide to give the crude product as an off white solid.

### EXAMPLE 99C

### 1-benzyl-3-(1,1-dioxido-4H-pyrido[3,2-e][1,2,4]thiadiazin-3-yl)-4-hydroxy-2(1H)-quinolinone

The title compound was prepared according to the procedure of Example 84D substituting the product of Example 99B for the product of Example 84C (0.076 g, 38%). ¹H NMR (300 MHz, DMSO-d₆) δ 5.64 (s, 2H), 7.29 (m, 5H), 7.43 (m, J=7.72, 7.72 Hz, 1H), 7.54 (d, J=8.46 Hz, 1H), 7.80 (m, 2H), 8.23 (m, 2H), 8.69 (d, J=3.31 Hz, 1H).

### EXAMPLE 100

### 1-benzyl-3-(1,1-dioxido-4H-pyrido[3,2-e][1,2,4]thiadiazin-3-yl)-4-hydroxy-1,8-naphthyridin-2(1H)-one

### EXAMPLE 100A

### N-[2-(aminosulfonyl)pyridin-3-yl]-1-benzyl-4-hydroxy-2-oxo-1,2-dihydro-1,8-naphthyridine-3-carboxamide

The title compound was prepared according to the procedure of Example 84C substituting 3-amino-pyridine-2-sulfonamide for 2-amino-5-bromobenzenesulfonamide. MS (ESI−) m/z 452 (M+H)⁺. ¹H NMR (300 MHz, DMSO-d₆) δ 5.66 (s, 2H), 7.22 (m, 1H), 7.28 (m, 3H), 7.43 (m, 1H), 7.70 (m, 3H), 8.52 (m, 2H), 8.77 (s, 3H), 12.56 (s, 1H), 16.34 (s, 1H).

### EXAMPLE 100B

### 1-benzyl-3-(1,1-dioxido-4H-pyrido[3,2-e][1,2,4]thiadiazin-3-yl)-4-hydroxy-1,8-naphthyridin-2(1H)-one

The title compound was prepared according to the procedure of Example 84D substituting the product of Example 100A for the product of Example 84C to give after purification by reverse phase HPLC (water/acetonitrile/0.1% NH₄OAc gradient) the title compound as a white solid (0.053 g, 10%). MS (ESI−) m/z 432 (M−H)⁻; ¹H NMR (300 MHz, DMSO-d₆) δ 5.70 (m, 2H), 7.25 (m, 7H), 7.50 (dd, J=7.91, 4.60 Hz, 1H), 7.80 (dd, J=8.46, 4.41 Hz, 1H), 8.17 (d, J=8.46 Hz, 1H), 8.60 (m, J=5.79, 1.88, 1.88 Hz, 1H), 8.68 (dd, J=4.41, 1.10 Hz, 1H), 8.82 (dd, J=4.78, 1.84 Hz, 1H).

### EXAMPLE 101

### 5-chloro-3-(1,1-dioxido-4H-pyrido[3,2-e][1,2,4]thiadiazin-3-yl)-4-hydroxy-1-(3-methylbutyl)-2(1H)-quinolinone

### EXAMPLE 101A

### 5-chloro-2H-3,1-benzoxazine-2,4(1H)-dione

A solution of potassium hydroxide (1.68 g, 30 mmol) and 2-amino-6-chlorobenzoic acid (3.43 g, 20 mmol) in water (25 mL) at 0° C. was treated dropwise with 20% phosgene in toluene (16.8 mL, 32 mmol) resulting in a precipitate. The mixture was stirred for 1 hour and the solid was collected by filtration, washed with water and dried to give the title compound (3.6 g, 91%). ¹H NMR (300 MHz, DMSO-d₆) δ 7.11 (d, J=7.35 Hz, 1H), 7.31 (d, J=6.99 Hz, 1H), 7.66 (t, J=8.09 Hz, 1H), 11.83 (s, 1H).

### EXAMPLE 101B

### 5-chloro-1-(3-methylbutyl)-2H-3,1-benzoxazine-2,4(1H)-dione

The title compound was prepared according to the procedure of Example 1B substituting 1-bromo-3-methylbutane for n-butyl bromide and substituting the product of Example 101A for the product of Example 1A (0.610 g, 45%). MS (DCI) m/z 285 (M+NH₄)⁺.

### EXAMPLE 101C

### ethyl 5-chloro-4-hydroxy-1-(3-methylbutyl)-2-oxo-1,2-dihydroquinoline-3-carboxylate

The title compound was prepared according to the procedure of Example 89A substituting the product of Example 101B for the product of Example 1B (0.600 g, 80%). ¹H NMR (300 MHz, DMSO-d₆) δ 0.96 (d, J=6.62 Hz, 6H), 1.32 (t, J=7.17 Hz, 3H), 1.44 (m, 2H), 1.70 (m, J=13.24, 6.62 Hz, 1H), 4.18 (m, 2H), 4.35 (q, J=6.99 Hz, 2H), 7.35 (d, J=6.99 1H), 7.48 (d, J=8.09 Hz, 1H), 7.67 (m, 1H), 13.88 (s, 1H).

### EXAMPLE 101D

### N-[2-(aminosulfonyl)pyridin-3-yl]-5-chloro-4-hydroxy-1-(3-methylbutyl)-2-oxo-1,2-dihydroquinoline-3-carboxamide

The product of Example 101C (0.170 g, 0.50 mmol) was reacted with the product of Example 98A (0.086 g, 0.50 mmol) in toluene (6 mL) at reflux for 16 hours. The reaction was cooled and the resulting precipitate was collected by filtration and dried to give the title compound (0.200 g, 86%). MS (DCI) m/z 465 (M+H)⁺. 1 ¹H NMR (300 MHz, DMSO-d₆) δ 0.99 (d, J=6.62 Hz, 6H), 1.51 (m, 2H), 1.76 (m, 1H), 4.32 (m, 2H), 7.45 (d, J=7.35 Hz, 1H), 7.61 (d, J=8.82 Hz, 1H), 7.71 (s, 2H), 7.77 (m, 2H), 8.45 (dd, J=8.46, 1.47 Hz, 1H), 8.53 (dd, J=4.60, 1.29 Hz, 1H), 12.84 (s, 1H), 17.22 (s, 1H).

### EXAMPLE 101E

### 5-chloro-3-(1,1-dioxido-4H-pyrido[3,2-e][1,2,4]thiadiazin-3-yl)-4-hydroxy-1-(3-methylbutyl)-2(1H)-quinolinone

The title compound was prepared according to the procedure of Example 84D substituting the product of Example 10D for the product of Example 84C (0.200 g, 98%). MS (ESI−) m/z 445 (M−H)⁻. The sodium salt of the title compound was prepared according to the procedure of Example 1D. ¹H NMR (300 MHz, DMSO-d₆) δ 0.98 (d, J=6.62 Hz, 6H), 1.45 (m, 2H), 1.71 (m, 1H), 4.11 (m, 2H), 7.08 (d, J=7.35 Hz, 1H), 7.23 (d, J=8.09 Hz, 1H), 7.43 (t, J=8.27 Hz, 1H), 7.57 (dd, J=8.46, 4.41 Hz, 1H), 7.78 (d, J=8.09 Hz, 1H), 8.45 (d, J=4.41 Hz, 1H), 15.77 (s, 1H).

### EXAMPLE 102

### 1-benzyl-3-(1,1-dioxido-4H-pyrido[3,2-e][1,2,4]thiadiazin-3-yl)-4-hydroxy-5-methyl-2(1H)quinolinone

### EXAMPLE 102A

### 1-Benzyl-(4-methyl)benzo[2,3-d][1,3]oxazine-2,4-dione

The title compound was prepared according to the procedure of Example 1B substituting benzyl bromide for n-butyl bromide and substituting (4-methyl)benzo[2,3-d][1,3]oxazine-2,4-dione for the product of Example 1A (0.67 g, 60%). MS (DCI+) m/z 268 (M+H)⁻; ¹H NMR (300 MHz, DMSO-d₆) δ 2.66 (s, 3H), 5.28 (s, 2H), 7.07 (d, J=8.48 Hz, 1H), 7.14 (d, J=7.80 Hz, 1H), 7.33 (m, 5H), 7.57 (t, J=7.46 Hz, 1H).

### EXAMPLE 102B

### ethyl 1-benzyl-4-hydroxy-5-methyl-2-oxo-1,2-dihydroquinoline-3-carboxylate

The title compound was prepared according to the procedure of Example 84A substituting the product of Example 102A for the product of Example 15A (0.71 g, 89%). MS (DCI+) m/z 338 (M+H)⁻; ¹H NMR (300 MHz, DMSO-d₆) δ 1.33 (t, J=7.17 Hz, 3H), 2.77 (s, 3H), 4.39 (q, J=7.23 Hz, 2H), 5.47 (s, 2H), 7.05 (d, J=7.35 Hz, 1H), 7.20 (m, 4H), 7.31 (m, 2H), 7.47 (t, J=8.09 Hz, 1H), 14.43 (s, 1H).

### EXAMPLE 102C

### N-[2-(aminosulfonyl)pyridin-3-yl]-1-benzyl-4-hydroxy-5-methyl-2-oxo-1,2-dihydroquinoline-3-carboxamide

The title compound was prepared according to the procedure of Example 84C substituting the product of Example 102B for the product of Example 84B and substituting the product of Example 98A for 2-amino-5-bromobenzenesulfonamide (0.163 g, 41%). MS (ESI+) m/z 465 (M+H)⁻; ¹H NMR (300 MHz, DMSO-d₆) δ 2.82 (s, 3H), 5.59 (s, 2H), 7.15 (d, J=7.35 Hz, 1H), 7.24 (m, 3H), 7.33 (m, 3H), 7.56 (t, J=7.91 Hz, 1H), 7.72 (m, 3H), 8.51 (m, 1H), 8.53 (s, 1H), 12.93 (s, 1H), 17.16 (m, 1H).

### EXAMPLE 102D

### 1-benzyl-3-(1,1-dioxido-4H-pyrido[3,2-e][1,2,4]thiadiazin-3-yl)-4-hydroxy-5-methyl-2(1H)-quinolinone

The title compound was prepared according to the procedure of Example 84D substituting the product of Example 102C for the product of Example 84C (0.064 g, 41%). MS (ESI+) m/z 447 (M+H)⁻. The sodium salt of the title compound was prepared according to the procedure of Example 1D. MS (ESI−) m/z 445 (M−H); ¹H NMR (300 MHz, DMSO-d₆) δ 2.81 (s, 3H), 5.37 (dd, J=6.07, 2.02 Hz, 2H), 6.80 (d, J=7.35 Hz, 1H), 6.95 (d, J=8.09 Hz, 1H), 7.20 (m, 4H), 7.29 (m, 2H), 7.57 (dd, J=8.46, 4.41 Hz, 1H), 7.75 (dd, J=8.46, 1.47 Hz, 1H), 8.44 (dd, J=4.41, 1.47 Hz, 1H), 16.29 (s, 1H).

### EXAMPLE 103

### 3-(1,1-dioxido-4H-pyrido[3,2-e][1,2,4]thiadiazin-3-yl)-4-hydroxy-1-[(2-methyl-1,3-thiazol-5-yl)methyl]-2(1H)-quinolinone

### EXAMPLE 103A

### 1-[(2-methyl-1,3-thiazol-5-yl)methyl]-2H-3,1-benzoxazine-2,4(1H)-dione

The title compound was prepared according to the procedure of Example 1B substituting isatoic anhydride for the product of Example 1A and 2-methyl-5-chloromethylthiazole for n-butyl bromide to give (0.410 g, 73%).

### EXAMPLE 103B

### ethyl 1-[(2-methyl-1,3-thiazol-5-yl)methyl]-2,4-dioxo-1,2,3,4-tetrahydroquinoline-3-carboxylate

The title compound was prepared according to the procedure of Example 84A substituting the product of Example 103A for the product of Example 15B (0.132 g, 25%). MS (ESI−) m/z 343 (M−H)⁻; ¹H NMR (300 MHz, CHLOROFORM-D) δ 1.49 (t, J=6.99 Hz, 3H), 2.61 (s, 3H), 4.53 (q, J=7.23 Hz, 2H), 5.54 (s, 2H), 7.27 (t, J=8.09 Hz, 1H), 7.41 (d, J=8.46 Hz, 1H), 7.62 (s, 1H), 7.67 (m, 1H), 8.21 (dd, J=8.09, 1.47 Hz, 1H), 14.32 (s, 1H).

### EXAMPLE 103C

### N-[2-(aminosulfonyl)pyridin-3-yl]-1-[(2-methyl-1,3-thiazol-5-yl)methyl]-2,4-dioxo-1,2,3,4-tetrahydroquinoline-3-carboxamide

The title compound was prepared as described in the procedure of Example 84C substituting the product of Example 99A for the product of Example 84B and substituting 3-amino-pyridine-2-sulfonamide for 2-amino-5-bromobenzenesulfonamide (0.148 g, 79%). MS (APCI) m/z 472 (M+H)⁺. ¹H NMR (300 MHz, DMSO-d₆) δ 2.55 (s, 3H), 5.69 (s, 2H), 7.25 (m, 1H), 7.35 (s, 1H), 7.42 (t, J=6.62 Hz, 1H), 7.81 (m, 4H), 8.17 (d, J=7.72 Hz, 1H), 8.52 (d, J=2.57 Hz, 1H), 8.54 (s, 1H), 12.67 (s, 1H), 16.28 (s, 1H).

### EXAMPLE 103D

### 3-(1,1-dioxido-4H-pyrido[3,2-e][1,2,4]thiadiazin-3-yl)-4-hydroxy-1-[(2-methyl-1,3-thiazol-5-yl)methyl]-2(1H)-quinolinone

The title compound was prepared according to the procedure of Example 84D substituting the product of Example 103C for the product of Example 84C (0.033 g, 68%). MS (APCI) m/z 454 (M+H)⁺. ¹H NMR (300 MHz, DMSO-d₆) δ 2.56 (s, 3H), 5.74 (s, 2H), 7.48 (t, J=6.80 Hz, 1H), 7.82 (s, 1H), 7.88 (m, 4H), 8.24 (m, 2H), 8.71 (dd, J=4.41, 1.47 Hz, 1H), 14.01 (s, 1H).

### EXAMPLE 104

### 1-benzyl-4-hydroxy-3-(7-methyl-1,1-dioxido-4H-pyrido[2,3-e][1,2,4]thiadiazin-3-yl)-2(1H)-quinolinone

### EXAMPLE 104A

### (2-Amino-5-methylpyrid-3-yl)sulfonyl chloride

(2-Amino-5-methylpyrid-3-yl)sulfonyl chloride was prepared from 2-amino-5-picoline by the method described by Weller, H. N. in U.S. Pat. No. 5,378,704. ¹H NMR (300 MHz, DMSO-d₆) δ 2.20 (s, 3H), 7.70 (br. s., 2H), 7.85 (d, J=5.52 Hz, 1H), 8.07 (d, J=2.21 Hz, 1H).

### EXAMPLE 104B

### 2-amino-5-methylpyridine-3-sulfonamide

The product of Example 104A was reacted with concentrated ammonium hydroxide at ambient temperature overnight. The reaction mixture was concentrated to give the title compound as a light yellow solid in quantitative yield. MS (DCI/NH₃) m/z 188 (M+H)⁺. ¹H NMR (300 MHz, DMSO-d₆) δ 2.17 (m, 3H), 6.28 (s, 2H), 7.43 (s, 2H), 7.70 (d, J=1.84 Hz, 1H), 8.00 (d, J=2.21 Hz, 1H).

### EXAMPLE 104C

### N-[F3-(aminosulfonyl)-5-methylpyridin-2-yl]-1-benzyl-4-hydroxy-2-oxo-1,2-dihydroquinoline-3-carboxamide

The title compound was prepared according to the procedure of Example 84C substituting product of Example 104B for 2-amino-5-bromobenzenesulfonamide.

### EXAMPLE 104D

### 1-benzyl-4-hydroxy-3-(7-methyl-1,1-dioxido-4H-pyrido[2,3-el [1,2,4]thiadiazin-3-yl)-2(1H)-quinolinone

The title compound was prepared according to the procedure of Example 84D substituting the product of Example 104C for the product of Example 84C (0.20 g, 35%). ¹H NMR (300 MHz, DMSO-d₆) δ 3.32 (m, 3H), 5.45 (s, 2H), 5.97 (s, 1H), 7.22 (m, 9H), 7.50 (m, 1H), 7.91 (dd, J=7.91, 1.65 Hz, 1H), 11.50 (m, 1H). The sodium salt of the title compound was prepared according to the procedure of Example 1D. ¹H NMR (300 MHz, DMSO-d₆) δ 2.37 (m, 3H), 5.39 (m, 2H), 7.07 (m, 1H), 7.24 (m, 6H), 7.39 (m, 1H), 7.94 (d, J=1.47 Hz, 1H), 8.13 (dd, J=7.91, 1.65 Hz, 1H), 8.43 (d, J=1.84 Hz, 1H), 16.49 (m, 1H)

### EXAMPLE 105

### 1-butyl-4-hydroxy-3-(7-methyl-1.1-dioxido-4H-pyrido[2,3-e][1,2,4]thiadiazin-3-yl)-1,8-naphthyridin-2(1H)-one

### EXAMPLE 105A

### ethyl 3-{[3-(aminosulfonyl)-5-methylpyridin-2-yl]amino}-3-oxopropanoate

The product of Example 104B (1.0 g, 0.0053 mol) in 10 mL of THF containing 5 mL of pyridine was treated with ethyl 3-chloro-3-oxopropionate (0.97 g, 0.0064 mol) at ambient temperature for several hours. The reaction mixture was concentrated to half its original volume and then diluted with water. The resulting precipitate was collected by filtration and washed with water and dried under vacuum to give the title compound as an off white solid (1.19 g, 75% yield). MS (ESI) m/z 300 (M−H)⁻. ¹H NMR (300 MHz, DMSO-d₆) δ 1.19 (t, J=7.17 Hz, 3H), 2.35 (s, 3H), 3.65 (s, 2H), 4.11 (q, J=7.23 Hz, 2H), 7.60 (s, 2H), 8.06 (d, J=1.47 Hz, 1H), 8.41 (d, J=1.84 Hz, 1H), 9.79 (s, 1H).

### EXAMPLE 105C

### ethyl(7-methyl-1,1-dioxido-4H-pyrido[2,3-e][1,2,4]thiadiazin-3-yl)acetate

The product of Example 105A (0.363 g, 0.0012 mol) in 20 mL of ethanol was reacted with sodium carbonate (0.350 g, 0.0033 mol). The reaction mixture was heated at reflux for 2 hours. After cooling, the reaction mixture was diluted with dichloromethane, filtered to remove the excess sodium carbonate, and concentrated. The residue was purified chromatography on silica gel with ethyl acetate in hexanes (1:1) followed by 4% methanol in dichloromethane as the mobile phase to give the title compound as a white solid (0.296 g, 87% yield). MS (ESI) m/z 282 (M−H)⁻; ¹H NMR (300 MHz, DMSO-d₆) δ 1.21 (t, J=6.99 Hz, 3H), 2.40 (s, 3H), 3.73 (s, 2H), 4.15 (q, J=7.11 Hz, 2H), 8.20 (s, 1H), 8.58 (d, J=1.84 Hz, 1H), 12.79 (s, 1H).

### EXAMPLE 105D

### 1-butyl-4-hydroxy-3-(7-methyl-1,1-dioxido-4H-pyrido[2,3-e][1,2,4]thiadiazin-3-yl)-1,8-naphthyridin-2(1H)-one

The title compound was prepared according to the procedure of Example 1D substituting the product of Example 105C for the product of Example 1C (0.065 g, 58% yield). ¹H NMR (300 MHz, DMSO-d₆) δ 0.94 (t, J=7.35 Hz, 3H), 1.40 (m, 2H), 1.67 (m, 2H), 2.44 (m, 3H), 4.46 (dd, J=7.91, 7.17 Hz, 2H), 7.48 (dd, J=8.09, 4.78 Hz, 1H), 8.29 (s, 1H), 8.56 (dd, J=7.91, 1.65 Hz, 1H), 8.64 (d, J=1.47 Hz, 1H), 8.87 (d, J=5.52 Hz, 1H). The sodium salt of the title compound was prepared according to the procedure of Example 1D. ¹H NMR (300 MHz, DMSO-d₆) δ 0.93 (t, J=7.35 Hz, 3H), 1.34 (m, 2H), 1.58 (m, 2H), 2.36 (s, 3H), 4.26 (d, J=7.72 Hz, 1H), 4.29 (d, J=6.99 Hz, 1H), 7.13 (dd, J=7.72, 4.78 Hz, 1H), 7.95 (s, 1H), 8.37 (dd, J=7.72, 2.21 Hz, 1H), 8.42 (d, J=1.84 Hz, 1H), 8.53 (dd, J=4.60, 2.02 Hz, 1H), 16.11 (s, 1H).

### EXAMPLE 106

### 3-(1,1-dioxido-4H-1,2,4-benzothiadiazin-3-yl)-4-hydroxy-1-(2-thienylmethyl)-1,8-naphthyridin-2(1H)-one

### EXAMPLE 106A

### 1-(thien-2-ylmethyl)-2H-pyrido[2,3-d][1,3]oxazine-2,4(1H)-dione

The title compound was prepared according to the procedure of Example 1B substituting 2-(bromomethyl)-thiophene for n-butyl bromide (0.165 g, 51%). ¹H NMR (300 MHz, DMSO-d₆) δ 5.48 (s, 2H), 6.97 (dd, J=5.15, 3.31 Hz, 1H), 7.21 (d, J=3.31 Hz, 1H), 7.43 (m, 2H), 8.41 (dd, J=7.72, 1.84 Hz, 1H), 8.83 (dd, J=5.15, 1.84 Hz, 1H).

### EXAMPLE 106B

### 3-(1,1-dioxido-4H-1,2,4-benzothiadiazin-3-yl)-4-hydroxy-1-(2-thienylmethyl)-1,8-naphthyridin-2(1H)-one

The title compound was prepared according to the procedure of Example 1D substituting the product of Example 106A for the product of Example 1B (0.162 g, 60%). MS (ESI−) m/z 437 (M−H)⁻. The sodium salt of the title compound was prepared according to the procedure of Example 1D. ¹H NMR (300 MHz, DMSO-d₆) δ 5.64 (s, 2H), 6.90 (m, J=5.15, 3.68 Hz, 1H), 7.11 (m, J=3.49, 0.92 Hz, 1H), 7.18 (dd, J=7.72, 4.78 Hz, 1H), 7.29 (m, 3H), 7.56 (m, 1H), 7.67 (dd, J=7.72, 1.10 Hz, 1H), 8.39 (dd, J=7.72, 2.21 Hz, 1H), 8.57 (dd, J=4.78, 1.84 Hz, 1H), 15.80 (s, 1H).

### EXAMPLE 107

### 1-(benzyloxy)-3-(1,1-dioxido-4H-1,2,4-benzothiadiazin-3-yl)-4-hydroxy-1,8-naphthyridin-2(1H)-one

### EXAMPLE 107A

### ethyl 2-[(benzyloxy)amino]nicotinate

2-Chloro-nicotinic acid ethyl ester (4.55 g, 24.6 mmol), O-benzylhydroxyamine hydrochloride (7.85 g, 49.2 mmol) and N,N-diisopropylethylamine (6.36 g, 49.2 mmol) in 10 mL 1,4-dioxane were reacted in a sealed tube at 120° C. for 48 hours. The reaction mixture was partitioned between ethyl acetate and 5% aqueous sodium bicarbonate. The aqueous layer was re-extracted with ethyl acetate (2×50 mL). The organic layers were combined and dried over sodium sulfate, filtered, and concentrated. The residue was purified by column chromatography on silica gel eluting with hexane and ethyl acetate (9:1) to provide the title compound (3.5 g, 53%). MS (DCI) m/z 273 (M+H)⁺.

### EXAMPLE 107B

### ethyl 2-[(benzyloxy)(3-ethoxy-3-oxopropanoyl)amino]nicotinate

A solution of the product of Example 107a (1.2 g, 4.4 mmol) and triethylamine (0.49 g, 4.8 mmol) in dichloromethane (25 mL) was treated dropwise with ethyl chloromalonate (0.73 g, 4.8 mmol), stirred for 2 hr and partitioned between ethyl acetate and water and the layers were separated. The ethyl acetate layer was washed with brine, dried (Na₂SO₄), and concentrated. The residue was purified by column chromatography on silica gel eluting with hexane and ethyl acetate (3:1) to provide the title compound (1.1 g, 65%). MS (DCI) m/z 387 (M+H)⁺.

### EXAMPLE 107C

### ethyl 1-(benzyloxy)-4-hydroxy-2-oxo-1,2-dihydro-1,8-naphthyridine-3-carboxylate

A solution of the product of Example 107b (0.386 g, 1.0 mmol) in ethanol (5 mL) was treated with 21% sodium ethoxide in ethanol (0.324 g, 1.0 mmol), stirred for 30 minutes and partitioned between ethyl acetate and 5% aqueous HCl and the layers were separated. The ethyl acetate layer was washed with brine, dried (Na₂SO₄), and concentrated to provide the title compound (0.28 g, 82%). MS (DCI) m/z 341(M+H)⁺.

### EXAMPLE 107D

### N-[2-(aminosulfonyl)phenyl]-1-(benzyloxy)-4-hydroxy-2-oxo-1,2-dihydro-1,8-naphthyridine-3-carboxamide

A mixture of the product of Example 107c (340 mg, 0.82 mmol) and 2-aminobenzenesulfonamide (141 mg, 0.82 mmol) in toluene (10 mL) was refluxed for 16 hours, cooled, and the resulting precipitate was collected by filtration and dried to give the title compound (340 mg, 89%). MS (DCI) m/z 467 (M+H)⁺.

### EXAMPLE 107E

### 1-(benzyloxy)-3-(1,1-dioxido-4H-1,2,4-benzothiadiazin-3-yl)-4-hydroxy-1,8-naphthyridin-2(1H)-one

The title compound was prepared according to the procedure of Example 84d substituting the product of Example 107d for the product of Example 84c to give the title compound (0.082 g, 87%). MS (ESI−) m/z 447 (M−H)⁻. The sodium salt of the title compound was prepared according to the procedure of Example 1d. ¹H NMR (300 MHz, DMSO-d₆) δ 5.12 (s, 2H) 7.22 (dd, J=7.72, 4.78 Hz, 1H) 7.30 (m, 2H) 7.44 (m, 3H) 7.57 (m, 1H) 7.70 (m, 3H) 8.41 (dd, J=7.72, 1.84 Hz, 1H) 8.61 (dd, J=4.78, 1.84 Hz, 1H) 15.70 (s, 1H).

### EXAMPLE 108

### 6-(1,1-dioxido-4H-1,2,4-benzothiadiazin-3-yl)-8-(2-ethylbutyl)-5-hydroxy-2-(methylsulfanyl)pyrido[2,3-d]pyrimidin-7(8H)-one

### EXAMPLE 108A

### methyl 4-[(2-ethylbutyl)amino]-2-(methylthio)pyrimidine-5-carboxylate

Ethyl 4-chloro-2-methylthio-5-pyrimidinecarboxylate (0.40 g, 1.72 mmol) was reacted with 1-amino-2-ethyl butane (0.175 g, 1.72 mmol) and triethylamine (0.60 mL, 4.32 mmol) at ambient temperature for 18 hours. The reaction was partitioned between water and dichloromethane. The organic layer was dried over sodium sulfate, filtered, and the concentrated to give the title compound (0.50 g, 98%).

### EXAMPLE 108B

### 4-[(2-ethylbutyl)amino]-2-(methylthio)pyrimidine-5-carboxylic Acid

The product of Example 108A (0.50 g, 1.68 mmol)) in water and ethanol (1:2) was reacted with sodium hydroxide (0.22 g, 5.50 mmol) at ambient temperature for 3 hours. The reaction was concentrated under vacuum to remove the ethanol and neutralized with aqueous hydrochloric acid (1 M). The resulting precipitate was collected by filtration and dried to yield the title compound (0.41 g, 91%). MS (DCI/NH₃) m/z 270 (M+H)⁺; ¹H NMR (300 MHz, DMSO-d₆) δ 0.88 (t, J=7.54 Hz, 6H), 1.31 (dt, J=14.25, 7.03 Hz, 4H), 1.53 (m, 1H), 2.47 (s, 3H), 3.46 (t, J=6.07 Hz, 2H), 8.50 (s, 1H), 13.22 (s, 1H).

### EXAMPLE 108C

### 1-(2-ethylbutyl)-7-(methylthio)-2H-pyrimido[4,5-d][1,3]oxazine-2,4(1H)-dione

The product of Example 108B (0.41 g, 1.52 mmol) was reacted with ethyl chloroformate (0.445 mL, 4.65 mmol) and pyridine (0.405 mL, 5.56 mmol) in toluene (8 ML) at 90° C. for 24 hours. The reaction was concentrated under vacuum. The residue was extracted with ethyl acetate and filtered. Concentration of the filtrate gave the title compound (0.394 g, 88%).

### EXAMPLE 108D

### 6-(1,1-dioxido-4H-1,2,4-benzothiadiazin-3-yl)-8-(2-ethylbutyl)-5-hydroxy-2(methylsulfanyl)pyrido[2,3-d]pyrimidin-7(8H)-one

The title compound was prepared according to the procedure of Example 1D substituting the product of Example 108C for the product of Example 1B (0.153 g, 24%). MS (ESI−) m/z 472 (M−H)⁻. The sodium salt of the title compound was prepared according to the procedure of Example 1D. ¹H NMR (300 MHz, DMSO-d₆) δ 0.87 (t, J=7.54 Hz, 6H), 1.28 (m, 4H), 1.87 (ddd, J=13.05, 6.80, 6.62 Hz, 1H), 2.56 (s, 3H), 4.15 (d, J=7.35 Hz, 2H), 7.26 (d, J=8.46 Hz, 1H), 7.31 (m, 1H), 7.56 (ddd, J=8.27, 7.17, 1.47 Hz, 1H), 7.67 (dd, J=7.72, 1.47 Hz, 1H), 8.89 (s, 1H), 15.52 (s, 1H).

### EXAMPLE 109

### 6-(1,1-dioxido-4H-1,2,4-benzothiadiazin-3-yl)-8-(2-ethylbutyl)-5-hydroxypyrido[2,3-d]pyrimidin-7(8H)-one

The product of Example 108D (0.15 g, 0.30 mmol) was reacted with an excess of Raney nickel (slurry in water, 2 mL) in ethanol (5 mL) and heated at 600C for 1 hour. The mixture was filtered through celite, rinsed with ethanol, and the filtrate concentrated under vacuum to yield the title compound. MS (ESI−) m/z 448 (M−H); ¹H NMR (300 MHz, DMSO-d₆) δ 0.86 (t, J=7.35 Hz, 6H), 1.28 (m, 4H), 1.87 (m, 1H), 4.18 (d, J=7.35 Hz, 2H), 7.30 (m, 2H), 7.57 (ddd, J=8.27, 7.17, 1.47 Hz, 1H), 7.68 (dd, J=7.91, 1.29 Hz, 1H), 8.94 (s, 1H), 9.09 (s, 1H), 15.43 (s, 1H).

### EXAMPLE 110

### 4-benzyl-6-(1,1-dioxido-4H-1,2,4-benzothiadiazin-3-yl)-7-hydroxythieno[3,2-b]pyridin-5(4H)-one

### EXAMPLE 110A

### 2H-thieno[3,2-d][1,3]oxazine-2,4(1H)-dione

The title compound was prepared according to the procedure of Fabis, and co-workers as described in Tetrahedron, 1998, 54, 10789-10800. MS (DCI/NH₃) m/z 186.9 (M+NH₄)⁺; ¹H NMR (300 MHz, DMSO-d₆) δ 6.95 (d, J=6 Hz, 1H) 8.25 (d, J=6 Hz, 1H) 12.22 (brs, 1H).

### EXAMPLE 110B

### 1-benzyl-2H-thieno[3,2-d][1,3]oxazine-2,4(1H)-dione

The product of Example 110A (0.137 g, 0.81 mmol) was reacted with benzyl bromide (0.10 mL, 0.85 mmol), and potassium carbonate (0.134 g, 0.97 mmol) in dimethylformamide (5 mL) at ambient temperature for 20 hours. The reaction mixture was diluted with water and the resulting precipitate was collecte by filtration, and dried to give the title compound (0.165 g, 80%). MS (DCI/NH₃) m/z 277 (M+NH₄)⁺; ¹H NMR (300 MHz, DMSO-d₆) δ 5.21 (s, 2H) 7.25 (d, J=5.52 Hz, 1H) 7.35 (m, 5H) 8.28 (d, J=5.52 Hz, 1H).

### EXAMPLE 110C

### 4-benzyl-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 110B for the product of Example 1B (0.137 g, 49%). MS (ESI−) m/z 438 (M−H)⁻. The sodium salt of the title compound was prepared according to the procedure of Example 1D. ¹H NMR (300 MHz, DMSO-d₆) δ 5.26 (s, 2H) 7.03 (d, J=5.52 Hz, 1H) 7.21 (m, 2H) 7.28 (m, 5H) 7.54 (ddd, J=8.27, 7.17, 1.47 Hz, 1H) 7.65 (dd, J=7.91, 1.29 Hz, 1H) 7.73 (d, J=5.52 Hz, 1H) 15.89 (s, 1H).

### EXAMPLE 111

### 4-butyl-6-(1,1-dioxido-4H-1,2,4-benzothiadiazin-3-yl)-7-hydroxythieno[3,2-b]pyridin-5(4H)-one

### EXAMPLE 111A

### 1-butyl-2H-thieno[3,2-d][1,3]oxazine-2,4(1H)-dione

The title compound was prepared according to the procedure of Example 110B substituting n-butyl bromide for benzyl bromide (0.059 g, 22%). MS (DCI) m/z 226 (M+H)⁺; ¹H NMR (300 MHz, DMSO-d₆) δ 0.91 (t, J=7.17 Hz, 3H) 1.36 (dt, J=22.70, 7.22 Hz, 2H) 1.62 (m, 2H) 3.94 (m, 2H) 7.39 (d, J=5.52 Hz, 1H) 8.34 (d, J=5.15 Hz, 1H).

### EXAMPLE 111B

### 4-butyl-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 111A for the product of Example 1B (0.050 g, 47%). MS (DCI/NH₃) m/z 404 (M+H)⁺; ¹H NMR (300 MHz, DMSO-d₆) δ 0.93 (m, 3H) 1.40 (td, J=14.98, 7.17 Hz, 2H) 1.67 (m, 2H) 4.27 (m, 2H) 7.54 (m, 1H) 7.60 (d, J=5.52 Hz, 1H) 7.67 (d, J=7.72 Hz, 1H) 7.77 (ddd, J=8.36, 7.08, 1.47 Hz, 1H) 7.92 (d, J=7.72 Hz, 1H) 8.39 (d, J=5.52 Hz, 1H) 14.46 (s, 1H) 14.90 (s, 1H).

### EXAMPLE 112

### 6-(1,1-dioxido-4H-1,2,4-benzothiadiazin-3-yl)-7-hydroxy-4-(4-pyridinylmethyl)thieno[3,2-b]pyridin-5(4H)-one

### EXAMPLE 112A

### 1-(pyridin-4-ylmethyl)-2H-thieno[3,2-d][1,3]oxazine-2,4(1H)-dione

The title compound was prepared according to the procedure of Example 1B substituting the product of Example 110A for the product of Example 1A and substituting 4-bromomethyl pyridine hydrobromide for n-butyl bromide (0.205 g, 80%).

### EXAMPLE 112B

### 6-(1,1-dioxido-4H-1,2,4-benzothiadiazin-3-yl)-7-hydroxy-4-(4-pyridinylmethyl)thieno[3,2-b]pyridin-5(4H)-one

The title compound was prepared according to the procedure of Example 1D substituting the product of Example 112A for the product of Example 1B (0.155 g, 45%). MS (DCI/NH₃) m/z 439 (M+H)⁺; ¹H NMR (300 MHz, DMSO-d₆) δ 5.78 (s, 2H) 7.49 (d, J=5.52 Hz, 1H) 7.55 (t, J=7.54 Hz, 1H) 7.62 (d, J=7.35 Hz, 1H) 7.76 (m, 4H) 7.93 (d, J=7.72 Hz, 1H) 8.38 (d, J=5.52 Hz, 1H) 8.75 (d, J=6.25 Hz, 1H) 14.06 (s, 1H).

### EXAMPLE 113

### 1-(3-bromobenzyl)-3-(1,1-dioxido-4H-1,2,4-benzothiadiazin-3-yl)-4-hydroxy-5,6,7,8-tetrahydro-2(1H)-quinolinone

### EXAMPLE 113A

### 5,6,7,8-tetrahydro-2H-3,1-benzoxazine-2,4(1H)-dione

The title compound was prepared according to the procedure of Example 3B substituting ethyl 2-aminocyclohex-1-ene-1-carboxylate for the product of Example 3A (0.960 g, 97%). MS (ESI−) m/z 166 (M−H)⁺.

### EXAMPLE 113B

### 1-(3-bromobenzyl)-5,6,7,8-tetrahydro-2H-3,1-benzoxazine-2,4(1H)-dione

The title compound was prepared according to the procedure of Example 1B substituting the product of Example 113A for the product of Example 1A and substituting 3-bromobenzyl bromide for n-butyl bromide (0.049 g, 46%). MS (ESI−) m/z 334 (M−H)⁺.

### EXAMPLE 113C

### 1-(3-bromobenzyl)-3-(1,1-dioxido-4H-1,2,4-benzothiadiazin-3-yl)-4-hydroxy-5,6,7,8-tetrahydro-2(1H)-quinolinone

The title compound was prepared according to the procedure of Example 1D substituting the product of Example 113B for the product of Example 1B (0.021 g, 34%). MS (ESI−) m/z 514 (M−H)⁺; ¹H NMR (300 MHz, DMSO-d₆) δ 1.68 (m, 4H), 2.51 (m, 2H), 2.67 (m, 2H), 5.40 (s, 2H), 7.13 (d, J=7.72 Hz, 1H), 7.30 (t, J=7.91 Hz, 1H), 7.51 (m, 3H), 7.61 (d, J=8.09 Hz, 1H), 7.73 (t, J=7.17 Hz, 1H), 7.90 (d, J=8.46 Hz, 1H), 14.40 (s, 1H), 14.56 (s, 1H). The sodium salt of the title compound was prepared according to the procedure of Example 1D. ¹H NMR (300 MHz, DMSO-d₆) δ 1.59 (m, 4H), 2.33 (t, J=5.70 Hz, 2H), 2.41 (m, 2H), 5.14 (s, 2H), 7.11 (d, J=8.09 Hz, 1H), 7.18 (d, J=8.09 Hz, 1H), 7.25 (m, 3H), 7.41 (d, J=7.72 Hz, 1H), 7.50 (td, J=7.72, 1.47 Hz, 1H), 7.62 (dd, J=7.72, 1.47 Hz, 1H), 17.13 (s, 1H).

### EXAMPLE 114

### 5-(1,1-dioxido-4H-1,2,4-benzothiadiazin-3-yl)-4-hydroxy-7-(4-pyridinylmethyl)thieno[2,3-b]pyridin-6(7H)-one

### EXAMPLE 114A

### 2H-thieno[2,3-d][1,3]oxazine-2,4(1H)-dione

The title compound was prepared by the method of Fabis, and coworkers in Tetrahedron 1998 54 10789-10800. MS (DCI/NH₃) M/Z 187 (M+NH₄)⁺; ¹H NMR (300 MHz, DMSO-d₆) δ 7.17 (d, J=16.8 Hz, 1H) 7.21 (d, J=16.8 Hz 1H) 12.56 (brs, 1H).

### EXAMPLE 114B

### 1-(pyridin-4-ylmethyl)-2H-thieno[2,3-d][1,3]oxazine-2,4(1H)-dione

The title compound was prepared according to the procedure of Example 1B substituting the product of Example 114A for the product of Example 1A and substituting 4-bromomethyl pyridine hydrobromide for n-butyl bromide (0.22 g, 95%).

### EXAMPLE 114C

### 5-(1,1-dioxido-4H-1,2,4-benzothiadiazin-3-yl)-4-hydroxy-7-(4-pyridinylmethyl)thieno[2,3-b]pyridin-6(7H)-one

The title compound was prepared according to the procedure of Example 1D substituting the product of Example 114B for the product of Example 1B (0.047 g, 13%). MS (DCI/NH₃) m/z 439 (M+H)⁺; ¹H NMR (300 MHz, DMSO-d₆) δ 5.62 (s, 2H) 7.48 (m, 2H) 7.55 (t, J=7.54 Hz, 1H) 7.62 (d, J=7.72 Hz, 1H) 7.68 (d, J=6.25 Hz, 1H) 7.76 (ddd, J=8.55, 7.26, 1.47 Hz, 1H) 7.93 (d, J=8.09 Hz, 1H) 8.71 (d, J=6.62 Hz, 1H) 13.87 (s, 1H).

### EXAMPLE 115

### 6-(1,1-dioxido-4H-1,2,4-benzothiadiazin-3-yl)-7-hydroxy-4-(3-pyridinylmethyl)thieno[3,2-b]pyridin-5(4H)-one

### EXAMPLE 115A

### 1-(pyridin-3-ylmethyl)-2H-thieno[3,2-d][1,3]oxazine-2,4(1H)-dione

The title compound was prepared according to the procedure of Example 1B substituting the product of Example 110A for the product of Example 1A and substituting 3-bromomethyl pyridine hydrobromide for n-butyl bromide (0.28 g, 90%). MS (DCI/NH₃) m/z 261 (M+H)⁺; ¹H NMR (300 MHz, DMSO-d₆) δ 5.24 (s, 2H) 7.34 (d, J=5.52 Hz, 1H) 7.38 (m, 1H) 7.81 (dt, J=8.00, 1.88 Hz, 1H) 8.30 (d, J=5.15 Hz, 1H) 8.51 (dd, J=4.78, 1.47 Hz, 1H) 8.67 (d, J=1.84 Hz, 1H).

### EXAMPLE 115B

### 6-(1,1-dioxido-4H-1,2,4-benzothiadiazin-3-yl)-7-hydroxy-4-(3-pyridinylmethyl)thieno[3,2-b]pyridin-5(4H)-one

The title compound was prepared according to the procedure of Example 1D substituting the product of Example 115A for the product of Example 1B (0.237 g, 50%). MS (DCI/NH₃) m/z 439 (M+H)⁺. The sodium salt of the title compound was prepared according to the procedure of Example 1D. ¹H NMR (300 MHz, DMSO-d₆) δ 5.49 (s, 2H) 7.34 (dd, J=7.91, 4.60 Hz, 1H) 7.45 (m, 3H) 7.68 (m, 2H) 7.83 (d, J=8.09 Hz, 1H) 8.16 (s, 1H) 8.47 (d, J=3.68 Hz, 1H) 8.62 (s, 1H) 14.83 (brs, 1H).

### EXAMPLE 116

### 7-benzyl-5-(1,1-dioxido-4H-1,2,4-benzothiadiazin-3-yl)-4-hydroxythieno[2,3-b]pyridin-6(7H)-one

### EXAMPLE 116A

### 1-benzyl-2H-thieno[2,3-d][1,3]oxazine-2,4(1H)-dione

The title compound was prepared according to the procedure of Example 110B substituting the product of Example 114A for the product of Example 110A (0.26g, 100%).

### EXAMPLE 116B

### 7-benzyl-5-(1,1-dioxido-4H-1,2,4-benzothiadiazin-3-yl)-4-hydroxythieno[2,3-b]pyridin-6(7H)-one

The title compound was prepared according to the procedure of Example 1D substituting the product of Example 116A for the product of Example 1B (0.144 g, 38%). MS (ESI−) m/z 436 (M−H)⁻. The sodium salt of the title compound was prepared according to the procedure of Example 1D. MS (ESI−) m/z 436 (M−H⁻); δ ¹H NMR (300 MHz, DMSO-d₆)δ 5.14 (s, 2H) 6.90 (d, J=5.52 Hz, 1H) 7.20 (m, 2H) 7.30 (m, 6H) 7.54 (m, 1H) 7.65 (dd, J=7.72, 1.47 Hz, 1H) 16.25 (s, 1H).

### EXAMPLE 117

### 4-(cyclopropylmethyl)-6-(1,1-dioxido-4H-1,2,4-benzothiadiazin-3-yl)-7-hydroxythieno[3,2-b]pyridin-5(4H)-one

### EXAMPLE 117A

### 1-(cyclopropylmethyl)-2H-thieno[3,2-d][1,3]oxazine-2,4(1H)-dione

The title compound was prepared according to the procedure of Example 1B substituting the product of Example 110A for the product of Example 1A and substituting bromomethyl cyclopropane for n-butyl bromide (0.23 g, 87%). MS (DCI/NH₃) m/z 241 (M+NH₄)⁺; ¹H NMR (300 MHz, DMSO-d₆) δ 0.47 (m, 4H) 1.21 (m, 1H) 3.89 (d, J=6.99 Hz, 2H) 7.45 (d, J=5.15 Hz, 1H) 8.35 (d, J=5.15 Hz, 1H).

### EXAMPLE 117B

### 4-(cyclopropylmethyl)-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 117A for the product of Example 1B (0.252 g, 60%). MS (ESI−) m/z 400 (M−H)⁻. The sodium salt of the title compound was prepared according to the procedure of Example 1D. ¹H NMR (300 MHz, DMSO-d₆) δ 0.41 (m, 4H) 1.20 (m, 1H) 3.92 (d, J=6.99 Hz, 2H) 7.19 (m, 2H) 7.26 (t, J=7.54 Hz, 1H) 7.53 (m, 1H) 7.64 (d, J=6.62 Hz, 1H) 7.79 (d, J=5.52 Hz, 1H) 15.97 (s, 1H).

### EXAMPLE 118

### 5-(1,1-dioxido-4H-1,2,4-benzothiadiazin-3-yl)-4-hydroxy-7-(3-methylbutyl)thieno[2,3-b]pyridin-6(7H)-one

### EXAMPLE 118A

### 1-(3-methylbutyl)-2H-thieno[2,3-d][1,3]oxazine-2,4(1H)-dione

The title compound was prepared according to the procedure of Example 1B substituting the product of Example 114A for the product of Example 1A and substituting isobutyl bromide for n-butyl bromide (0.074 g, 35%).

### EXAMPLE 118B

### 5-(1,1-dioxido-4H-1,2,4-benzothiadiazin-3-yl)-4-hydroxy-7-(3-methylbutyl)thieno[2,3-b]pyridin-6(7H)-one

The title compound was prepared according to the procedure of Example 1D substituting the product of Example 118A for the product of Example 1B (0.063 g, 49%). MS (ESI−) m/z 416 (M−H)⁻. The sodium salt of the title compound was prepared according to the procedure of Example 1D. ¹H NMR (300 MHz, DMSO-d₆) δ 0.96 (s, 3H) 0.98 (s, 3H) 1.53 (m, 2H) 1.66 (m, 1H) 3.87 (m, 2H) 6.95 (d, J=5.52 Hz, 1H) 7.19 (m, 2H) 7.25 (m, 1H) 7.52 (ddd, J=8.27, 7.17, 1.47 Hz, 1H) 7.64 (dd, J=7.72, 1.47 Hz, 1H) 16.30 (s, 1H).

### EXAMPLE 119

### 4-benzyl-6-(1,1-dioxido-4H-1,2,4-benzothiadiazin-3-yl)-7-hydroxy-2-phenylthieno[3,2-b]pyridin-5(4H)-one

### EXAMPLE 119A

### 6-phenyl-2H-thieno[3,2-d[]1,3]oxazine-2,4(1H)-dione

Methyl-3-amino-5-phenylthiophene-2-carboxylate (0.25 g, 1.07 mmol) in water (6 mL) was reacted with potassium hydroxide (0.12 g, 2.14 mmol) at 90° C. for 24 hours. The reaction was cooled to 0° C. and phosgene (1.9M in toluene, 0.70 mL, 1.40 mmol) was added dropwise. After stirring at room temperature for 1 hour, the resulting solid was collected by filtration, washed with excess water and dried to give the title compound as a tan solid (0.175 g, 65%).

### EXAMPLE 119B

### 1-benzyl-6-phenyl-2H-thieno[3,2-d][1,3]oxazine-2,4(1H)-dione

The title compound was prepared according to the procedure of Example 1B substituting the product of Example 119A for the product of Example 1A (0.19g, 80%). MS (DCI/NH₃) m/z 353 (M+NH)⁺; ¹H NMR (300 MHz, DMSO-d₆) δ 5.26 (s, 2H) 7.43 (m, 8H) 7.82 (m, 3H).

### EXAMPLE 119C

### 4-benzyl-6-(1,1-dioxido-4H-1,2,4-benzothiadiazin-3-yl)-7-hydroxy-2-phenylthieno[3,2-b]pyridin-5(4H)-one

The title compound was prepared according to the procedure of Example 1D substituting the product of Example 119B for the product of Example 1B (0.062 g, 22%). MS (ESI−) m/z 512 (M−H)⁻. The sodium salt of the title compound was prepared according to the procedure of Example 1D. ¹H NMR (300 MHz, DMSO-d₆) δ 5.34 (s, 2H) 7.24 (m, 2H) 7.33 (m, 5H) 7.43 (m, 4H) 7.56 (t, J=7.35 Hz, 1H) 7.71 (m, 3H) 15.82 (m, 1H).

### EXAMPLE 120

### 4-benzyl-6-(1,1-dioxido-4H-1,2,4-benzothiadiazin-3-yl)-7-hydroxy-3-methylthieno[3,2-b]pyridin-5(4H)-one

### EXAMPLE 120A

### 7-methyl-2H-thieno3,2-d][1,3]oxazine-2,4(1H)-dione

The title compound was prepared according to the procedure of Example 119A substituting methyl-3-amino-4-methylthiophene-2-carboxylate for methyl-3-amino-5-phenylthiophene-2-carboxylate.

### EXAMPLE 120B

### 1-benzyl-7-methyl-2H-thieno[3,2-d][1,3]oxazine-2.4(1H)-dione

The title compound was prepared according to the procedure of Example 110B substituting the product of Example 120A for the product of Example 110A (0.22 g, 73%). MS (DCI/NH₃) m/z 291 (M+NIL)+

### EXAMPLE 120C

### 4-benzyl-6-(1,1-dioxido-4H-1,2,4-benzothiadiazin-3-yl)-7-hydroxy-3-methylthieno[3,2-b]pyridin-5(4H)-one

The title compound was prepared according to the procedure of Example 1D substituting the product of Example 120B for the product of Example 1B (0.110 g, 30%). MS (ESI−) m/z 450 (M−H)⁻. The sodium salt of the title compound was prepared according to the procedure of Example 1D. ¹H NMR (300 MHz, DMSO-d₆) δ 2.21 (s, 3H) 5.47 (s, 2H) 7.05 (m, 1H) 7.25 (m, 6H) 7.37 (d, J=0.74 Hz, 1H) 7.54 (ddd, J=8.27, 7.17, 1.47 Hz, 1H) 7.64 (dd, J=7.91, 1.29 Hz, 1H) 15.92 (s, 1H).

### EXAMPLE 121

### 1-benzyl-3-(1,1-dioxido-4H-1,2,4-benzothiadiazin-3-yl)-4-hydroxy-5,6,7,8-tetrahydro-2(1H)-quinolinone

### EXAMPLE 121A

### 1-benzyl-5,6,7,8-tetrahydro-2H-3,1-benzoxazine-2,4(1H)-dione

The title compound was prepared according to the procedure of Example 1B substituting the product of Example 113A for the product of Example 1A and substituting benzyl bromide for n-butyl bromide (0.620 g, 67%). MS (ESI−) m/z 256 (M−H)⁻.

### EXAMPLE 121B

### 1-benzyl-3-(1,1-dioxido-4H-1,2,4-benzothiadiazin-3-yl)-4-hydroxy-5,6,7,8-tetrahydro-2(1H)-quinolinone

The title compound was prepared according to the procedure of Example 1D substituting the product of Example 121A for the product of Example 1B (0.039 g, 37%). MS (ESI−) m/z 434 (M−H)⁻. The sodium salt of the title compound was prepared according to the procedure of Example 1D. ¹H NMR (300 MHz, DMSO-d₆) δ 1.54 (m, 4H), 2.33 (t, J=5.88 Hz, 2H), 2.42 (m, 2H), 5.15 (s, 2H), 7.10 (d, J=6.99 Hz, 2H), 7.20 (m, 3H), 7.30 (t, J=7.35 Hz, 2H), 7.50 (td, J=7.72, 1.47 Hz, 1H), 7.61 (dd, J=7.72, 1.10 Hz, 1H), 17.20 (s, 1H).

### EXAMPLE 122

### 1-benzyl-3-(1,1-dioxido-4H-1,2,4-benzothiadiazin-3-yl)-4-hydroxy-2(1H)-pyridinone

### EXAMPLE 122A

### 2H-1,3-oxazine-2,6(3H)-dione

The title compound was prepared by the method described by Warren, and coworkers in Journal of Organic Chemistry 1975 40(6) 743-746. MS (DCI/NH₃) M/z 131 (M+NH₄)⁺; ¹H NMR (300 MHz, DMSO-d₆) δ 5.61 (d, J=7.72 Hz, 1H) 7.65 (d, J=7.35 Hz, 1H) 11.55 (s, 1H).

### EXAMPLE 122B

### 3-benzyl-2H-1,3-oxazine-2,6(3H)-dione

The title compound was prepared according to the procedure of Example 110B substituting the product of Example 122A for the product of Example 110A (0.156 g, 25%). MS (DCI/NH₃) m/z 221 (M+NH₄)⁺; ¹H NMR (300 MHz, DMSO-d₆) δ 4.89 (s, 2H) 5.78 (d, J=7.72 Hz, 1H) 7.37 (m, 5H) 7.97 (d, J=8.09 Hz, 1H).

### EXAMPLE 122C

### 1-benzyl-3-(1,1-dioxido-4H-1,2,4-benzothiadiazin-3-yl)-4-hydroxy-2(1H)-pyridinone

The title compound was prepared according to the procedure of Example 1D substituting the product of Example 122B for the product of Example 1B (0.13 g, 5%). MS (ESI−) m/z 380 (M−H)⁻; ¹H NMR (300 MHz, DMSO-d₆) δ 4.89 (s, 2H) 5.53 (d, J=7.35 Hz, 1H) 7.11 (d, J=7.72 Hz, 1H) 7.28 (m, 6H) 7.39 (d, J=7.72 Hz, 1H) 7.50 (m, 1H) 7.61 (dd, J=7.72, 1.10 Hz, 1H) 16.83 (s, 1H).

### EXAMPLE 123

### 1-benzyl-3-(1,1-dioxido-4H-1,2,4-benzothiadiazin-3-yl)-4-hydroxy-5,6-dimethyl-2(1H)-pyridinone

### EXAMPLE 123A

### 4,5-dimethyl-2H-1,3-oxazine-2,6(3H)-dione

The title compound was prepared by the method described by Washburne, et. al. Tetrahedron Letters 1976 17(4) 243-246. MS (DCI/NH₃) m/z 204 (M+H)⁺

### EXAMPLE 123B

### 3-benzyl-4,5-dimethyl-2H-1,3-oxazine-2,6(3H)-dione

The title compound was prepared according to the procedure of Example 1B substituting the product of Example 123A for the product of Example 1A and substituting benzyl bromide for n-butyl bromide (0.109 g, 27%). MS (DCI/NH₃) m/z 249 (M+NH₄)⁺; ¹H NMR (300 MHz, DMSO-d₆) δ 1.86 (s, 3H) 2.14 (s, 3H) 5.09 (s, 2H) 7.32 (m, 5H).

### EXAMPLE 123C

### 1-benzyl-3-(1,1-dioxido-4H-1,2,4-benzothiadiazin-3-yl)-4-hydroxy-5,6-dimethyl-2(1H)-pyridinone

The title compound was prepared according to the procedure of Example 1D substituting the product of Example 123B for the product of Example 1B (0.070 g, 36%). MS (ESI−) m/z 408 (M−H)⁻. The sodium salt of the title compound was prepared according to the procedure of Example 1D. ¹H NMR (300 MHz, DMSO-d₆) δ 1.88 (s, 3H) 2.10 (s, 3H) 5.20 (s, 2H) 7.13 (m, 2H) 7.21 (m, 2H) 7.31 (m, J=7.17, 7.17 Hz, 3H) 7.50 (m, 1H) 7.62 (dd, J=7.91, 1.29 Hz, 1H)17.28 (s, 1H).

### EXAMPLE 124

### 7-benzyl-5-(1,1-dioxido-4H-1,2,4-benzothiadiazin-3-yl)-4-hydroxy-3-methylthieno[2,3-b]pyridin-6(7H)-one

### EXAMPLE 124A

### 5-methyl-2H-thieno[2,3-d][1,3]oxazine-2,4(1H)-dione

The title compound was prepared according to the procedure of Fabis, and co-workers as described in Tetrahedron, 1998, 54, 10789-10800. MS (ESI−) m/z 182 (M−H)⁻.

### EXAMPLE 124B

### 1-benzyl-5-methyl-2H-thieno[2,3-d][1,3]oxazine-2,4(1H)-dione

The title compound was prepared according to the procedure of Example 1B substituting the product of Example 124A for the product of Example 1A and substituting benzyl bromide for n-butyl bromide (0.075 g, 50%). MS (DCI/NH₃) m/z 291 (M+NH₄)⁺.

### EXAMPLE 124C

### 7-benzyl-5-(1,1-dioxido-4H-1,2,4-benzothiadiazin-3-yl)-4-hydroxy-3-methylthieno[2,3-b]pyridin-6(7H)-one

The title compound was prepared according to the procedure of Example 1D substituting the product of Example 124B for the product of Example 1B (0.025 g, 23%). MS (ESI−) m/z 450 (M−H)⁻. The sodium salt of the title compound was prepared according to the procedure of Example 1D. ¹H NMR (300 MHz, DMSO-d₆) δ 2.46 (s, 3H), 5.12 (s, 2H), 6.47 (d, J=1.10 Hz, 1H), 7.28 (m, 7H), 7.52 (td, J=7.72, 1.47 Hz, 1H), 7.64 (dd, J=7.72, 1.47 Hz, 1H), 16.31 (s, 1H).

### EXAMPLE 125

### 6-(1,1-dioxido-4H-1,2,4-benzothiadiazin-3-yl)-7-hydroxy-4-(3-methylbutyl)thieno[3,2-b]pyridin-5(4H)-one

### EXAMPLE 125A

### 1-(3-methylbutyl)-2H-thieno[3,2-d][1,3]oxazine-2.4(1H)-dione

The title compound was prepared according to the procedure of Example 1B substituting the product of Example 110A for the product of Example 1A and substituting 1-bromo-3-methyl butane for n-butyl bromide (0.246 g, 68%).

### EXAMPLE 125B

### 6-(1,1-dioxido-4H-1,2,4-benzothiadiazin-3-yl)-7-hydroxy-4-(3-methylbutyl)thieno[3,2-b]pyridin-5(4H)-one

The title compound was prepared according to the procedure of Example 1D substituting the product of Example 125A for the product of Example 1B (0.223 g, 52%). MS (ESI−) m/z 416 (M−H)⁻. The sodium salt of the title compound was prepared according to the procedure of Example 1D. ¹H NMR (300 MHz, DMSO-d₆) δ 0.96 (d, J=6.90 Hz, 6H) 1.45 (m, 2H) 1.67 (m, 1H) 3.99 (m, 2H) 7.09 (d, J=5.52 Hz, 1H) 7.19 (d, J=7.72 Hz, 1H) 7.26 (m, 1H) 7.53 (ddd, J=8.55, 7.26, 1.47 Hz, 1H) 7.64 (dd, J=7.72, 1.47 Hz, 1H) 7.80 (d, J=5.52 Hz, 1H) 15.95 (s, 1H).

### EXAMPLE 126

### 6-(1,1-dioxido-4H-1,2,4-benzothiadiazin-3-yl)-4-(2-ethylbutyl)-7-hydroxythieno[3,2-b]pyridin-5(4H)-one

### EXAMPLE 126A

### 1-(2-ethylbutyl)-2H-thieno[3,2-d][1,3]oxazine-2,4(1H)-dione

The title compound was prepared according to the procedure of Example 1B substituting the product of Example 110A for the product of Example 1A and substituting 1-bromo-2-ethyl butane form-butyl bromide (0.116 g, 31%). MS (DCI/NH₃) m/z 271 (M+NH₄)⁺.

### EXAMPLE 126B

### 6-(1,1-dioxido-4H-1,2,4-benzothiadiazin-3-yl)-4-(2-ethylbutyl)-7-hydroxythieno[3,2-b]pyridin-5(4H)-one

The title compound was prepared according to the procedure of Example 1D substituting the product of Example 126A for the product of Example 1B (0.052 g, 26%). MS (ESI−) m/z 430 (M−H)⁻. The sodium salt of the title compound was prepared according to the procedure of Example 1D. ¹H NMR (300 MHz, DMSO-d₆) δ 0.87 (t, J=7.35 Hz, 6H) 1.29 (m, 4H) 1.73 (m, J=13.24, 6.99 Hz, 1H) 3.91 (d, J=7.35 Hz, 2H) 7.08 (d, J=5.52 Hz, 1H) 7.18 (d, J=8.09 Hz, 1H) 7.25 (m, J=7.54, 7.54 Hz, 1H) 7.53 (ddd, J=8.55, 7.26, 1.47 Hz, 1H) 7.64 (dd, J=7.72, 1.47 Hz, 1H) 7.78 (d, J=5.15 Hz, 1H) 15.99 (s, 1H).

### EXAMPLE 127

### 1-benzyl-3-(1,1-dioxido-4H-1,2,4-benzothiadiazin-3-yl)-4-hydroxy-6-methyl-5-phenyl-2(1H)-pyridinone

### EXAMPLE 127A

### 4-methyl-5-phenyl-2H-1,3-oxazine-2,6(3H)-dione

Ethyl 2-phenylacetoacetate (1.0 g, 4.85 mmol) and urethane (0.43 g, 4.85 mmol) were heated, neat, with Phosphorous oxychloride (3 mL) at 90° C. for 3 hours. The excess reagents were removed under vacuum and the resulting residue was triturated with benzene and filtered. This solid was triturated with diethyl ether, filtered, and dried to yield 0.818 g (83%). MS (DC/NH₃) m/z 204 (M+H)⁺; ¹H NMR (300 MHz, DMSO-d₆) δ 1.98 (s, 3H) 7.28 (m, 2H) 7.39 (m, 3H) 11.65 (s, 1H).

### EXAMPLE 127B

### 3-benzyl-4-methyl-5-phenyl-2H-1,3-oxazine-2,6(3H)-dione

The title compound was prepared according to the procedure of Example 1B substituting the product of Example 127A for the product of Example 1A and substituting benzyl bromide for n-butyl bromide (0.257 g, 71%). MS (DCI/NH₃) m/z 311 (M+NH₄)⁺; ¹H NMR (300 MHz, DMSO-d₆) δ 2.03 (s, 3H) 5.16 (s, 2H) 7.34 (m, 10H).

### EXAMPLE 127C

### 1-benzyl-3-(1,1-dioxido-4H-1,2,4-benzothiadiazin-3-yl)-4-hydroxy-6-methyl-5-phenyl-2(1H)-pyridinone

The title compound was prepared according to the procedure of Example 1D substituting the product of Example 127B for the product of Example 1B (0.022 g, 5%). MS (ESI−) m/z 470 (M−H)⁻. The sodium salt of the title compound was prepared according to the procedure of Example 1D. ¹H NMR (300 MHz, DMSO-d₆) δ 1.92 (s, 3H) 5.24 (s, 2H) 7.19 (m, 10H) 7.33 (m, 2H) 7.46 (ddd, J=8.27, 7.17, 1.47 Hz, 1H) 7.61 (dd, J=7.91, 1.29 Hz, 1H) 16.97 (s, 1H).

### EXAMPLE 128

### 3-(1,1-dioxido-4H-1,2,4-benzothiadiazin-3-yl)-4-hydroxy-5,6-dimethyl-1-(3-methylbutyl)-2(1H)-pyridinone

### EXAMPLE 128A

### 4,5-dimethyl-3-(3-methylbutyl)-2H-1,3-oxazine-2,6(3H)-dione

The title compound was prepared according to the procedure of Example 1B substituting the product of Example 123A for the product of Example 1A and substituting 1-bromo-3-methyl butane for n-butyl bromide (0.224 g, 60%). MS (DCI/NH₃) m/z 255 (M+NH₄)⁺; ¹H NMR (300 MHz, DMSO-d₆) δ 0.92 (d, J=6.62 Hz, 6H) 1.46 (m, 2H) 1.59 (dt, J=13.14, 6.48 Hz, 1H) 1.85 (s, 3H) 2.26 (s, 3H) 3.77 (m, 2H).

### EXAMPLE 128B

### 3-(1,1-dioxido-4H-1,2,4-benzothiadiazin-3-yl)-4-hydroxy-5,6-dimethyl-1-(3-methylbutyl)-2(1H)-pyridinone

The title compound was prepared according to the procedure of Example 1D substituting the product of Example 128A for the product of Example 1B (0.132 g, 32%). MS (ESI−) m/z 388 (M−H)⁻. The sodium salt of the title compound was prepared according to the procedure of Example 1D. ¹H NMR (300 MHz, DMSO-d₆) δ 0.94 (d, J=2.5 Hz, 6H) 1.87 (s, 3H) 2.24 (s, 3H) 3.84 (m, 2H) 7.16 (m, 1H) 7.21 (m, 1H) 7.49 (m, 1H) 7.61 (m, 1H) 17.41 (s, 1H).

### EXAMPLE 129

### 3-(1,1-dioxido-4H-1,2,4-benzothiadiazin-3-yl)-1-(2-ethylbutyl)-4-hydroxy-5,6-dimethyl-2(1H)-pyridinone

### EXAMPLE 129A

### 3-(2-ethylbutyl)-4,5-dimethyl-2H-1,3-oxazine-2,6(3H)-dione

The title compound was prepared according to the procedure of Example 1B substituting the product of Example 123A for the product of Example 1A and substituting 1-bromo-2-ethyl-butane for n-butyl bromide (0.181 g, 45%). ¹H NMR (300 MHz, DMSO-d₆) δ 0.85 (t, J=7.35 Hz, 6H) 1.29 (m, 4H) 1.65 (m, 1H) 1.86 (s, 3H) 2.25 (s, 3H) 3.73 (d, J=7.35 Hz, 2H).

### EXAMPLE 129B

### 3-(1,1-dioxido-4H-1,2,4-benzothiadiazin-3-yl)-1-(2-ethylbutyl)-4-hydroxy-5,6-dimethyl-2(1H)-pyridinone

The title compound was prepared according to the procedure of Example 1D substituting the product of Example 129A for the product of Example 1B (0.027 g, 9%). MS (ESI−) m/z 402 (M−H)⁻. The sodium salt of the title compound was prepared according to the procedure of Example 1D. ¹H NMR (300 MHz, DMSO-d₆) δ 0.85 (t, J=7.35 Hz, 6H) 1.25 (m, 4H) 1.62 (m, 1H) 1.88 (s, 3H) 2.22 (s, 3H) 3.82 (m, 2H) 7.14 (d, J=7.72 Hz, 1H) 7.21 (m, 1H) 7.48 (ddd, J=8.46, 7.17, 1.65 Hz, 1H) 7.60 (dd, J=7.91, 1.29 Hz, 1H) 17.42 (s, 1H).

### EXAMPLE 130

### 1-benzyl-3-(1,1-dioxido-4H-12,4-benzothiadiazin-3-yl)-4-hydroxy-6-phenyl-2(1H)-pyridinone

### EXAMPLE 130A

### 4-phenyl-2H-1,3-oxazine-2,6(3H)-dione

The title compound was prepared according to the procedure of Example 127A, substituting ethyl benzoylacetate for ethyl 2-phenylacetoacetate to yield the desired product (0.99 g, 47%). MS (DCI/NH₃) M/Z 188 (M+H)⁺; ¹H NMR (300 MHz, DMSO-d₆) δ 6.03 (s, 1H) 7.56 (m, 3H) 7.79 (m, 2H) 11.80 (s, 1H).

### EXAMPLE 130B

### 3-benzyl-4-phenyl-2H-1,3-oxazine-2,6(3H)-dione

The title compound was prepared according to the procedure of Example 1B substituting the product of Example 130A for the product of Example 1A and benzyl bromide for n-butyl bromide (0.223 g, 78%).

### EXAMPLE 130C

### 1-benzyl-3-(1,1-dioxido-4H-1,2,4-benzothiadiazin-3-yl)-4-hydroxy-6-phenyl-2(1H)-pyridinone

The title compound was prepared according to the procedure of Example 1D substituting the product of Example 130B for the product of Example 1B (0.021 g, 6%). MS (ESI−) m/z 456 (M−H)⁻. The sodium salt of the title compound was prepared according to the procedure of Example 1D. ¹H NMR (300 MHz, DMSO-d₆) δ 4.89 (s, 2H) 5.44 (s, 1H) 6.87 (d, J=6.99 Hz, 1H) 7.20 (m, 9H) 7.35 (m, 2H) 7.52 (ddd, J=8.55, 7.26, 1.47 Hz, 1H) 7.63 (dd, J=7.72, 1.47 Hz, 1H) 16.78 (s, 1H).

### EXAMPLE 131

### 5-(1,1-dioxido-4H-1,2,4-benzothiadiazin-3-yl)-4-hydroxy-7-(3-methyl-2-butenyl)thieno[2,3-b]pyridin-6(7H)-one

### EXAMPLE 131A

### 1-(3-methylbut-2-enyl)-2H-thieno[2,3-d][1,3]oxazine-2,4(1H)-dione

The title compound was prepared according to the procedure of Example 1B substituting the product of Example 114A for the product of Example 1A and substituting 1-bromo-3-methyl-but-2-ene for n-butyl bromide (0.23 g, 82%). MS (DCI/NH₃) m/z 255 (M+NH₄)⁺; ¹H NMR (300 MHz, DMSO-dr₆) δ 1.72 (d, J=1.10 Hz, 3H) 1.79 (d, J=0.74 Hz, 3H) 4.50 (d, J=6.62 Hz, 2H) 5.23 (m, 1H) 7.28 (d, J=1.10 Hz, 2H).

### EXAMPLE 131B

### 5-(1,1-dioxido-4H-1,2,4-benzothiadiazin-3-yl)-4-hydroxy-7-(3-methyl-2-butenyl)thieno[2,3-b]pyridin-6(7H)-one

The title compound was prepared according to the procedure of Example 1D substituting the product of Example 131A for the product of Example 1B (0.178 g, 44%). MS (ESI−) m/z 414 (M−H)⁻. The sodium salt of the title compound was prepared according to the procedure of Example 1D. ¹H NMR (300 MHz, DMSO-d₆) δ 1.69 (s, 3H) 1.82 (s, 3H) 4.51 (d, J=6.62 Hz, 2H) 5.13 (m, 1H) 6.94 (d, J=5.52 Hz, 1H) 7.20 (m, 2H) 7.25 (m, 1H) 7.53 (ddd, J=8.27, 7.17, 1.47 Hz, 1H) 7.64 (dd, J=7.72, 1.47 Hz, 1H) 16.30 (s, 1H).

### EXAMPLE 132

### 1,5-dibenzyl-3-(11-dioxido-4H-1,2,4-benzothiadiazin-3-yl)-4-hydroxy-6-methyl-2(1H)pyridinone

### EXAMPLE 132A

### 5-benzyl-4-methyl-2H-1,3-oxazine-2,6(3H)-dione

The title compound was prepared according to the procedure of in Example 127A, substituting ethyl 2-benzyl-3-oxo-butyric acid ethyl ester for ethyl 2-phenylacetoacetate to yield the desired product. MS (DCI/NH₃) m/z 218 (M+H)+

### EXAMPLE 132B

### 3,5-dibenzyl-4-methyl-2H-1,3-oxazine-2,6(3H)-dione

The title compound was prepared according to the procedure of Example 1B substituting the product of Example 132A for the product of Example 1A and benzyl bromide for n-butyl bromide (0.215 g, 76%).

### EXAMPLE 132C

### 1,5-dibenzyl-3-(1,1-dioxido-4H-1,2,4-benzothiadiazin-3-yl)-4-hydroxy-6-methyl-2(1H)-pyridinone

The title compound was prepared according to the procedure of Example 1D substituting the product of Example 132B for the product of Example 1B (0.051 g, 15%). MS (ESI−) m/z 484 (M−H)⁻. The sodium salt of the title compound was prepared according to the procedure of Example 1D. ¹H NMR (300 MHz, DMSO-d₆) δ 2.05 (s, 3H) 3.84 (s, 2H) 5.21 (s, 2H) 7.21 (m, 12H) 7.49 (m, 1H) 7.62 (dd, J=7.91, 1.29 Hz, 1H) 17.10 (s, 1H).

### EXAMPLE 133

### 3-(1.1-dioxido-4H-1,2,4-benzothiadiazin-3-yl)-1-(2-ethylbutyl)-4-hydroxy-6-meth-5-phenyl-2(1H)-pyridinone

### EXAMPLE 133A

### 3-(2-ethylbutyl)-4-methyl-5-phenyl-2H-1,3-oxazine-2,6(3H)-dione

The title compound was prepared according to the procedure of Example 1B substituting the product of Example 127A for the product of Example 1A and substituting 1-bromo-2-ethyl butane for n-butyl bromide (0.145 g, 41%). MS (DCI/NH₃) m/z 288 (M+H)+

### EXAMPLE 133B

### 3-(1,1-dioxido-4H-1,2,4-benzothiadiazin-3-yl)-1-(2-ethylbutyl)-4-hydroxy-6-methyl-5-phenyl-2(1H)-pyridinone

The title compound was prepared according to the procedure of Example 1D substituting the product of Example 133A for the product of Example 1B (0.019 g, 8%). MS (ESI−) m/z 464 (M−H)⁻. The sodium salt of the title compound was prepared according to the procedure of Example 1D. ¹H NMR (300 MHz, DMSO-dr) δ 0.88 (t, J=7.35 Hz, 6H) 1.30 (m, 4H) 1.71 (m, 1H) 2.03 (s, 3H) 3.83 (m, 2H) 7.10 (m, 3H) 7.22 (m, 2H) 7.34 (t, J=7.17 Hz, 2H) 7.45 (ddd, J=8.27, 7.17, 1.47 Hz, 1H) 7.60 (dd, J=7.91, 1.29 Hz, 1H) 17.10 (s, 1H).

### EXAMPLE 134

### 6-(1,1-dioxido-4H-1,2,4-benzothiadiazin-3-yl)-7-hydroxy-4-pentylthieno[3,2-b]pyridin-5(4H)-one

### EXAMPLE 134A

### 1-pentyl-2H-thieno[3,2-d][1,3]oxazine-2,4(1H)-dione

The title compound was prepared according to the procedure of Example 1B substituting the product of Example 110A for the product of Example 1A and substituting n-pentyl bromide for n-butyl bromide (0.205 g, 72%).

### EXAMPLE 134B

### 6-(1.1-dioxido-4H-1,2,4-benzothiadiazin-3-yl)-7-hydroxy-4-pentylthieno[3,2-b]pyridin-5(4H)-one

The title compound was prepared according to the procedure of Example 1D substituting the product of Example 134A for the product of Example 1B (0.189 g, 53%). MS (ESI−) m/z 416 (M−H)⁻. The sodium salt of the title compound was prepared according to the procedure of Example 1D. ¹H NMR (300 MHz, DMSO-d₆) δ 0.88 (m, 3H) 1.33 (m, 4H) 1.57 (m, 2H) 3.97 (m, 2H) 7.14 (d, J=5.52 Hz, 1H) 7.18 (d, J=8.09 Hz, 1H) 7.25 (m, 1H) 7.53 (m, 1H) 7.64 (dd, J=7.91, 1.29 Hz, 1H) 7.79 (d, J=5.52 Hz, 1H) 15.96 (s, 1H).

### EXAMPLE 135

### 5-(1,1-dioxido-4H-1,2,4-benzothiadiazin-3-yl)-4-hydroxy-3-methyl-7-(3-methylbutyl)thieno[2,3-b]pyridin-6(7H)-one

### EXAMPLE 135A

### 5-methyl-2H-thieno[2.3-d][1,3]oxazine-2,4(1H)-dione

The title compound was prepared from 2-amino-4-methyl-thiophene-3-carboxylic acid ethyl ester according to the procedure of Fabis, and co-workers as described in Tetrahedron, 1998, 54, 10789-10800MS (ESI) m/z 182 (M−H)⁻; ¹H NMR (300 MHz, DMSO-D₆) δ ppm 2.30 (d, J=1.47 Hz, 3H), 6.78 (s, 1H), 12.51 (s, 1H).

### EXAMPLE 135B

### 5-methyl-1-(3-methylbutyl)-2H-thieno[2,3-d][1,3]oxazine-2,4(1H)-dione

The title compound was prepared according to the procedure of Example 1B substituting the product of Example 135A for the product of Example 1A and substituting isopentyl bromide for n-butyl bromide (0.048 g, 30%). MS (DCI/NH₃) m/z 254 (M+H)⁺. ¹H NMR (300 MHz, DMSO-D₆) δ 0.94 (d, J=6.25 Hz, 6H), 1.61 (m, 3H), 2.33 (d, J=1.10 Hz, 3H), 3.83 (m, 2H), 6.92 (d, J=1.10 Hz, 1H).

### EXAMPLE 135C

### 5-(1,1-dioxido-4H-1,2,4-benzothiadiazin-3-yl)-4-hydroxy-3-methyl-7-(3-methylbutyl)thieno[2,3-b]pyridin-6(7H)-one

The title compound was prepared according to the procedure of Example 1D substituting the product of Example 135B for the product of Example 1B (0.043 g, 52%). MS (DCI/NH₃) m/z 430 (M+H)⁺; ¹H NMR (300 MHz, DMSO-d₆) δ 0.98 (d, J=6.25 Hz, 6H), 1.67 (m, 3H), 2.50 (s, 3H), 4.13 (m, 2H), 7.08 (s, 1H), 7.55 (t, J=7.54 Hz, 1H), 7.68 (d, J=8.46 Hz, 1H), 7.77 (t, J=7.17 Hz, 1H), 7.92 (d, J=7.35 Hz, 1H), 14.30 (s, 1H), 15.22 (s, 1H). The sodium salt of the title compound was prepared according to the procedure of Example 1D. ¹H NMR (300 MHz, DMSO-d₆) δ 0.97 (d, J=6.62 Hz, 6H), 1.56 (m, 3H), 3.89 (m, 2H), 7.53 (m, 5H), 16.37 (brs, 1H).

### EXAMPLE 136

### 6-(1,1-dioxido-4H-1,2,4-benzothiadiazin-3-yl)-7-hydroxy-4-(4-methylpentyl)thieno[3,2-b]pyridin-5(4H)-one

### EXAMPLE 136A

### 1-(4-methylpentyl)-2H-thieno[3,2-d][1,3]oxazine-2,4(1H)-dione

The title compound was prepared according to the procedure of Example 1B substituting the product of Example 110A for the product of Example 1A and substituting 1-bromo-4-methyl-pentane for n-butyl bromide (0.110 g, 61%).

### EXAMPLE 136B

### 6-(1,1-dioxido-4H-1,2,4-benzothiadiazin-3-yl)-7-hydroxy-4-(4-methylpentyl)thieno[3,2-b]pyridin-5(4H)-one

The title compound was prepared according to the procedure of Example 1D substituting the product of Example 136A for the product of Example 1B (0.064 g, 34%). MS (ESI−) m/z 430 (M−H)⁻. The sodium salt of the title compound was prepared according to the procedure of Example 1D. ¹H NMR (300 MHz, DMSO-d₆) δ 0.86 (s, 3H) 0.88 (s, 3H) 1.24 (m, J=15.81, 6.99 Hz, 2H) 1.56 (m, 3H) 3.96 (d, J=6.99 Hz, 2H) 7.16 (m, 1H) 7.26 (t, J=7.35 Hz, 1H) 7.53 (t, J=7.72 Hz, 1H) 7.64 (d, J=7.72 Hz, 1H) 7.80 (d, J=5.15 Hz, 1H) 15.96 (s, 1H).

### EXAMPLE 137

### 4-(3-butenyl)-6-(1,1-dioxido-4H-1,2,4-benzothiadiazin-3-yl)-7-hydroxythieno[3,2-b]pyridin-5(4H)-one

### EXAMPLE 137A

### 1-but-3-enyl-2H-thieno[3,2-d][1,3]oxazine-2,4(1H)-dione

The title compound was prepared according to the procedure of Example 1B substituting the product of Example 110A for the product of Example 1A and substituting 4-bromo-but-1-ene for n-butyl bromide (0.09 g, 56%).

### EXAMPLE 137B

### 4-(3-butenyl)-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 137A for the product of Example 1B (0.062 g, 38%). MS (ESI−) m/z 399.9 (M−H)⁻. The sodium salt of the title compound was prepared according to the procedure of Example 1D. ¹H NMR (300 MHz, DMSO-d₆) δ 2.34 (q, J=7.23 Hz, 2H) 4.04 (m, 2H) 5.05 (m, 2H) 5.87 (m, 1H) 7.18 (m, 2H) 7.25 (t, J=7.17 Hz, 1H) 7.53 (m, 1H) 7.64 (d, J=6.62 Hz, 1H) 7.79 (d, J=5.52 Hz, 1H) 15.93 (s, 1H).

### EXAMPLE 138

### 7-benzyl-5-(1,1-dioxido-4H-1,2,4-benzothiadiazin-3-yl)-4-hydroxy-1-phenyl-1.7-dihydro-6H-pyrazolo[3,4-b]pyridin-6-one

### EXAMPLE 138A

### ethyl 5-(benzylamino)-1-phenyl-1H-pyrazole-4-carboxylate

The title compound was prepared according to the procedure of Example 1B substituting ethyl 5-amino-1-phenyl-4-pyrazole-carboxylate for the product of Example 1A and substituting benzyl bromide for n-butyl bromide (0.990 g, 83%). MS (ESI−) m/z 320(M−H)⁻.

### EXAMPLE 138B

### ethyl 5-[benzyl(3-ethoxy-3-oxopropanoyl)amino]-1-phenyl-1H-pyrazole-4-carboxylate

The title compound was prepared (51% yield) from the product of Example 138A and ethyl malonyl chloride according to the procedure of Rowley, and co-workers as described in J. Med. Chem., 1993, 36, 3386-3396. MS (ESI−) m/z 434 (M−H)⁺.

### EXAMPLE 138C

### methyl 7-benzyl-4-hydroxy-6-oxo-1-phenyl-6,7-dihydro-1H-pyrazolo[3,4-b]pyridine-5-carboxylate

The title compound was prepared from the product of Example 138B and sodium methoxide according to the procedure of Rowley, and co-workers as described in J. Med. Chem., 1993, 36, 3386-3396. MS (ESI−) m/z 374 (M−H)⁻.

### EXAMPLE 138D

### N-[2-(aminosulfonyl)phenyl]-7-benzyl-4-hydroxy-6-oxo-1-phenyl-6,7-dihydro-1H-pyrazolo[3,4-b]pyridine-5-carboxamide

The title compound was prepared according to the procedure of Example 84C substituting the product of Example 138C for the product of Example 84B and 2-aminobenzenesulfonamide for 2-amino-5-bromobenzenesulfonamide (0.014 g, 61%). MS (ESI+) m/z 516 (M+H)⁺.

### EXAMPLE 138E

### 7-benzyl-5-(1,1-dioxido-4H-1,2,4-benzothiadiazin-3-yl)-4-hydroxy-1-phenyl-1,7-dihydro-6H-pyrazolo[3,4-b]pyridin-6-one

The title compound was prepared according to the procedure of Example 84D substituting the product of Example 138D for the product of Example 84C (0.061 g, 84%). MS (ESI−) m/z 496 (M−H)⁻. The sodium salt of the title compound was prepared according to the procedure of Example 1D. ¹H NMR (300 MHz, DMSO-d₆) δ 4.92 (s, 2H), 6.53 (m, 2H), 7.21 (m, 9H), 7.43 (t, J=7.35 Hz, 1H), 7.54 (td, J=7.81, 1.65 Hz, 1H), 7.64 (dd, J=7.91, 1.29 Hz, 1H), 7.88 (s, 1H), 16.05 (s, 1H).

### EXAMPLE 139

### 4-benzyl-6-(1,1-dioxido-4H-1,2,4-benzothiadiazin-3-yl)-2,7-dihydroxy[1,3]thiazolo[4,5-b]pyridin-5(4H)-one

### EXAMPLE 139A

### methyl 4-(benzylamino)-2-(methylthio)-1,3-thiazole-5-carboxylate

The title compound was prepared according to the procedure of Example 1B substituting 4-amino-2-methylthio-5-thiazolecarboxylic acid methyl ester for the product of Example 1A and substituting benzyl bromide for n-butyl bromide (0.411 g, 57%). MS (ESI+) m/z 295 (M+H)⁺.

### EXAMPLE 139B

### methyl 4-[benzyl(3-ethoxy-3-oxopropanoyl)amino]-2-(methylthio)-1,3-thiazole-5-carboxylate

The title compound was prepared according to the procedure of Example 138B substituting the product of Example 139A for the product of Example 138A (0.147 g, 30%). MS (ESI+) m/z 409 (M+H)⁺.

### EXAMPLE 139C

### ethyl 4-benzyl-7-hydroxy-2-(methylthio)-5-oxo-4,5-dihydro[1,3]thiazolo[4,5-b]pyridine-6-carboxylate

The title compound was prepared according to the procedure of Example 138C substituting the product of Example 139B for the product of Example 138B (0.111 g, 82%). MS (ESI−) m/z 375 (M−H)⁻.

### EXAMPLE 139D

### N-[2-(aminosulfonyl)phenyl]-4-benzyl-7-hydroxy-2-(methylthio)-5-oxo-4,5-dihydro[1,3]thiazolo[4,5-b]pyridine-6-carboxamide

The title compound was prepared according to the procedure of Example 84C substituting the product of Example 139C for the product of Example 84B and 2-aminobenzenesulfonamide for 2-amino-5-bromobenzenesulfonamide (0.114 g, 75%). MS (ESI−) m/z 501 (M−H)⁻.

### EXAMPLE 139E

### 4-benzyl-6-(1,1-dioxido-4H-1,2,4-benzothiadiazin-3-yl)-2,7-dihydroxy[1,3]thiazolo[4,5-b]pyridin-5(4H)-one

The title compound was prepared according to the procedure of Example 84D substituting the product of Example 139D for the product of Example 84C (0.108 g, 60%). MS (ESI−) m/z 453 (M−H)⁻. ¹H NMR (300 MHz, DMSO-d₆) δ 5.37 (s, 2H), 7.29 (m, 5H), 7.44 (t, J=7.72 Hz, 1H), 7.50 (d, J=7.72 Hz, 1H), 7.67 (td, J=7.72, 1.47 Hz, 1H), 7.82 (d, J=7.72 Hz, 1H), 14.01 (s, 1H), 14.32 (s, 1H). The sodium salt of the title compound was prepared according to the procedure of Example 1D. ¹H NMR (300 MHz, DMSO-d₆) δ 5.13 (s, 2H), 7.04 (d, J=8.09 Hz, 1H), 7.20 (m, 6H), 7.45 (t, J=7.35 Hz, 1H), 7.56 (d, J=7.72 Hz, 1H), 17.25 (s, 1H).

### EXAMPLE 140

### 4-[(2-chloro-1,3-thiazol-5-yl)methyl]-6-(1,1-dioxido-4H-1,2,4-benzothiadiazin-3-yl)-7-hydroxythieno[3,2-b]pyridin-5(4H)-one

### EXAMPLE 140A

### 1-[(2-chloro-1,3-thiazol-5-yl)methyl]-2H-thieno[3,2-d][1,3]oxazine-2,4(1H)-dione

The title compound was prepared according to the procedure of Example 1B substituting the product of Example 104A for the product of Example 1A and substituting 2-chloro-5-bromomethylthiazole for n-butyl bromide (0.341 g, 75%). MS (DCI/NH₃) m/z 301 (M+H)⁺. ¹H NMR (300 MHz, DMSO-d₆) δ 5.35 (s, 2H), 7.60 (d, J=5.15 Hz, 1H), 7.89 (s, 1H), 8.38 (d, J=5.52 Hz, 1H),

### EXAMPLE 140B

### 4-[(2-chloro-1,3-thiazol-5-yl)methyl]-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 140A for the product of Example 1B (0.134 g, 40%). MS (ESI−) m/z 477 (M−H)⁻. ¹H NMR (300 MHz, DMSO-d₆) δ 5.64 (s, 2H), 7.55 (t, J=7.17 Hz, 1H), 7.67 (d, J=7.72 Hz, 1H), 7.78 (t, J=7.17 Hz, 1H), 7.86 (d, J=5.52 Hz, 1H), 7.93 (d, J=7.72 Hz, 1H), 7.95 (s, 1H), 8.43 (d, J=5.52 Hz, 1H), 14.10 (s, 1H). The sodium salt of the title compound was prepared according to the procedure of Example 1D.

### EXAMPLE 141

### 6-(1,1-dioxido-4H-1,2,4-benzothiadiazin-3-yl)-7-hydroxy-4-[(5-methyl-3-pyridinyl)methyl]thieno[3,2-b]pyridin-5(4H)-one

### EXAMPLE 141A

### 1-[(5-methylpyridin-3-yl)methyl]-2H-thieno[3,2-d][1,3]oxazine-2,4(1H)-dione

The title compound was prepared according to the procedure of Example 1B substituting the product of Example 110A for the product of Example 1A and substituting 3-methyl-5-chloromethylpyridine for n-butyl bromide (0.255 g, 38%). MS (DCI/NH₃) m/z 275 (M+H)⁺. ¹H NMR (300 MHz, DMSO-d₆) δ 2.27 (s, 3H), 5.21 (s, 2H), 7.31 (d, J=5.52 Hz, 1H), 7.63 (s, 1H), 8.29 (d, J=5.15 Hz, 1H), 8.34 (d, J=1.47 Hz, 1H), 8.47 (d, J=1.84 Hz, 1H).

### EXAMPLE 141B

### 6-(1,1-dioxido-4H-1,2,4-benzothiadiazin-3-yl)-7-hydroxy-4-[(5-methyl-3-pyridinyl)methyl]thieno[3,2-b]pyridin-5(4H)-one

The title compound was prepared according to the procedure of Example 1D substituting the product of Example 141A for the product of Example 1B (0.175 g, 43%). MS (ESI−) m/z 451 (M−H)⁻. ¹H NMR (300 MHz, DMSO-d₆) δ 2.25 (s, 3H), 5.54 (s, 2H), 7.53 (m, 3H), 7.64 (d, J=7.72 Hz, 1H), 7.75 (td, J=7.72, 1.47 Hz, 1H), 7.92 (d, J=7.35 Hz, 1H), 8.34 (d, J=5.15 Hz, 1H), 8.34 (s, 1H), 8.45 (d, J=1.47 Hz, 1H), 14.30 (s, 1H). The sodium salt of the title compound was prepared according to the procedure of Example 1D.

### EXAMPLE 142

### 6-(1,1-dioxido-4H-1,2,4-benzothiadiazin-3-yl)-7-hydroxy-4-[(2-methyl-1,3-thiazol-5-yl)methyl]thieno[3,2-b]pyridin-5(4H)-one

### EXAMPLE 142A

### 1-[(2-methyl-1,3-thiazol-5-yl)methyl]-2H-thieno[3,2-d][1,3]oxazine-2,4(1H)-dione

The title compound was prepared according to the procedure of Example 1B substituting the product of Example 110A for the product of Example 1A and substituting 2-methyl-5-chloromethylthiazole for n-butyl bromide (0.308 g, 55%). MS (DCI/NH₃) m/z 281 (M+H)⁺. ¹H NMR (300 MHz, DMSO-d₆) δ 2.59 (s, 3H), 5.34 (s, 2H), 7.58 (d, J=5.52 Hz, 1H), 7.81 (s, 1H), 8.37 (d, J=5.52 Hz, 1H).

### EXAMPLE 142B

### 6-(1,1-dioxido-4H-1,2,4-benzothiadiazin-3-yl)-7-hydroxy-4-[(2-methyl-1,3-thiazol-5-yl)methyl]thieno[3,2-b]pyridin-5(4H)-one

The title compound was prepared according to the procedure of Example 1D substituting the product of Example 142A for the product of Example 1B (0.151 g, 40%). MS (DCI/NH₃) m/z 459 (M+H)⁺. ¹H NMR (300 MHz, DMSO-d₆) δ 2.56 (s, 3H), 5.65 (s, 2 H), 7.56 (t, J=7.17 Hz, 1H), 7.68 (d, J=7.72 Hz, 1H), 7.79 (m, 3H), 7.93 (d, J=7.72 Hz, 1H), 8.42 (d, J=5.52 Hz, 1H), 14.18 (s, 1H). The sodium salt of the title compound was prepared according to the procedure of Example 1D.

### EXAMPLE 143

### 4-[(5-chloro-2-thienyl)methyl]-6-(1,1-dioxido-4H-1,2,4-benzothiadiazin-3-yl)-7-hydroxythieno[3,2-b]pyridin-5(4H)-one

### EXAMPLE 143A

### 1-[(5-chlorothien-2-yl)methyl]-2H-thieno[3,2-d][1,3]oxazine-2,4(1H)-dione

The title compound was prepared according to the procedure of Example 1B substituting the product of Example 110A for the product of Example 1A and substituting 2-chloro-5-chloromethylthiophene for n-butyl bromide (0.601 g, 100%).

### EXAMPLE 143B 4-[(5-chloro-2-thienyl)methyl]-6-(1,1-dioxido-4H-1,2,4-benzothiadiazin-3-yl)-7-hydroxythieno[3,2-b]pyridin-5(4H)-one

The title compound was prepared according to the procedure of Example 1D substituting the product of Example 143A for the product of Example 1B (0.115 g, 12%). MS (APCI) m/z 478 (M+H)⁺. ¹H NMR (300 MHz, DMSO-D d₆) δ 5.59 (s, 2H), 6.99 (d, J=3.68 Hz, 1H), 7.23 (d, J=4.04 Hz, 1H), 7.56 (t, J=7.72 Hz, 1H), 7.68 (d, J=7.72 Hz, 1H), 7.78 (m, 1H), 7.80 (d, J=5.88 Hz, 1H), 7.93 (d, J=8.09 Hz, 1H), 8.41 (d, J=5.52 Hz, 1H), 14.18 (s, 1H). The sodium salt of the title compound was prepared according to the procedure of Example 1D.

### EXAMPLE 144

### 6-(1,1-dioxido-4H-1,2,4-benzothiadiazin-3-yl)-7-hydroxy-4-[(2-methyl-1,3-thiazol-4-yl)methyl]thieno[3,2-b]pyridin-5(4H)-one

### EXAMPLE 144A

### 1-[(2-methyl-1,3-thiazol-4-yl)methyl]-2H-thieno[3,2-d][1,3]oxazine-2,4(1H)-dione

The title compound was prepared according to the procedure of Example 1B substituting the product of Example 110A for the product of Example 1A and substituting 2-methyl-4-chloromethylthiazole hydrochloride for n-butyl bromide (0.200 g, 36%).

### EXAMPLE 144B

### 6-(1,1-dioxido-4H-1,2,4-benzothiadiazin-3-yl)-7-hydroxy-4-[(2-methyl-1,3-thiazol-4-yl)methyl]thieno[3,2-b]pyridin-5(4H)-one

The title compound was prepared according to the procedure of Example 1D substituting the product of Example 144A for the product of Example 1B (0.127 g, 38%). MS (ESI+) m/z 459 (M+H)⁺. ¹H NMR (300 MHz, DMSO-d₆) δ 2.60 (s, 3H), 5.55 (s, 2H), 7.54 (t, J=6.99 Hz, 1H), 7.54 (d, J=5.52 Hz, 1H), 7.65 (d, J=7.72 Hz, 1H), 7.76 (m, 1H), 7.92 (d, J=6.62 Hz, 1H), 8.34 (d, J=5.51 Hz, 1H), 14.30 (s, 1H),

### EXAMPLE 145

### 4-benzyl-6-(1,1-dioxido-4H-1,2,4-benzothiadiazin-3-yl)-7-hydroxy-2-(methylsulfanyl) [1,3]thiazolo[4,5-b]pyridin-5(4H)-one

### EXAMPLE 145A

### 4-benzyl-2-(methylthio)-5H-[1,3]thiazolo[4,5-d][1,3]oxazine-5,7(4H)-dione

The title compound was prepared according to the procedure of Example 3B substituting the product of Example 139A for the product of Example 3A (0.048 g, 92%). MS (DCI/NH₃) m/z 324 (M+NH₄)⁺.

### EXAMPLE 145B

### 4-benzyl-6-(1,1-dioxido-4H-1,2,4-benzothiadiazin-3-yl)-7-hydroxy-2-(methylsulfanyl)[1,3]thiazolo[4,5-b]pyridin-5(4H)-one

The title compound was prepared according to the procedure of Example 1D substituting the product of Example 145A for the product of Example 1B (0.037 g, 51%). MS (ESI) m/z 485 (M+H)⁺. The sodium salt of the title compound was prepared according to the procedure of Example 1D. ¹H NMR (300 MHz, DMSO-d₆) δ 2.73 (s, 3H), 5.31 (s, 2H), 7.25 (m, 7H), 7.53 (td, J=7.72, 1.47 Hz, 1H), 7.64 (dd, J=7.91, 1.29 Hz, 1H), 15.52 (s, 1H).

### EXAMPLE 146

### 4-benzyl-6-(1,1-dioxido-4H-1,2,4-benzothiadiazin-3-yl)-7-hydroxy-2-(methylsulfonyl) [1,3]thiazolo[4,5-b]pyridin-5(4H)-one

The title compound was prepared as a white solid from the product of Example 145B and 3-chloroperoxybenzoic acid according to the procedure of Leysen, and co-workers described in J. Heterocyclic Chem., 1984, 21, 401-406. MS (ESI) m/z 515 (M−H)⁻; ¹H NMR (300 MHz, DMSO-d₆) δ 3.59 (s, 3H), 5.51 (s, 2H), 7.32 (m, 5H), 7.51 (m, 2H), 7.69 (m, 1H), 7.85 (d, J=7.72 Hz, 1H), 13.95 (s, 1H).

### EXAMPLE 147

### 2-amino-4-benzyl-6-(1,1-dioxido-4H-1,2,4-benzothiadiazin-3-yl)-7-hydroxy[1,3]thiazolo[4,5-b]pyridin-5(4H)-one

The product of Example 146 (0.011 g, 0.02 mmol) was reacted with ammonia (0.5 M in dioxane, 1.3 mL, 0.64 mmol) in a pressure tube at 70° C. for 17 hours. The reaction was cooled and the resulting precipitate was collected by filtration and dried to give the title compound as a white solid (0.009 g, 100%). MS (ESI) m/z 452 (M−H)⁻; ¹H NMR (300 MHz, DMSO-d₆) δ 5.43 (s, 2H), 6.91 (s, 1H), 7.07 (s, 1H), 7.30 (m, 4H), 7.52 (dd, J=24.27, 8.82 Hz, 2H), 7.69 (t, J=7.54 Hz, 1H), 7.85 (d, J=8.82 Hz, 1H), 9.03 (br s, 1H), 14.57 (brs, 1H).

### EXAMPLE 148

### 4-benzyl-6-(1,1-dioxido-4H-1,2,4-benzothiadiazin-3-yl)-7-hydroxy[1,3]thiazolo[4,5-b]pyridin-5(4H)-one

The product of Example 147 (0.0085 g, 0.019 mmol) was reacted with tert-butyl nitrite (5 mL, 0.037 mmol) in DMF (0.3 mL) at 60° C. for 1 hour. The reaction was cooled, and the crude mixture was purified by column chromatography with silica gel eluting with hexane and ethyl acetate (1:1) to give the title compound as a yellow solid (0.0045 g, 54%). MS (ESI) m/z 437 (M−H)⁻; ¹H NMR (300 MHz, DMSO-d₆) δ 5.53 (s, 2H), 7.25 (m, 1H), 7.31 (m, 4H), 7.43 (m, 2H), 7.66 (m, 1H), 7.80 (d, J=8.46 Hz, 1H), 9.48 (s, 1H), 14.56 (br s, 1H).

### 759163 Example 149

### 3-(1,1-dioxido-4H-1,2,4-benzothiadiazin-3-yl)-4-hydroxy-1-(2-phenylpropyl)-1,8-naphthyridin-2(1H)-one

### EXAMPLE 149A

### ethyl 2-[(2-phenylpropyl)amino]nicotinate

The title compound was prepared according to the procedure of Example 3A substituting (+)-beta-methylphenethylamine for 2-ethyl-butylamine (0.44 g, 58%). MS (DCI+) m/z 285 (M+H)−; ¹H NMR (300 MHz, DMSO-D₆) δ 1.25 (m, 6H), 3.07 (m, 1H), 3.64 (m, 3H), 4.22 (q, J=6.99 Hz, 1H), 6.61 (dd, J=7.72, 4.78 Hz, 1H), 7.21 (m, 1H), 7.30 (m, 4H), 7.87 (t, J=5.52 Hz, 1H), 8.05 (m, 1H), 8.29 (m, 1H).

### EXAMPLE 149B

### 1-(2-phenylpropyl)-2H-pyrido[2,3-d [1,3]oxazine-2,4(1H)-dione

The title compound was prepared according to the procedure of Example 3B substituting the product of Example 149A for the product of Example 3A (0.44 g, 99%). MS (DCI+j m/z 283 (M+H)−; ¹H NMR (300 MHz, DMSO-D₆) δ 1.26 (d, J=6.99 Hz, 3H), 3.37 (m, 1H), 4.21 (dd, J=13.24, 6.25 Hz, 1H), 4.36 (m, 1H), 7.21 (m, 1H), 7.29 (m, 4H), 7.38 (dd, J=7.72, 4.78 Hz, 1H), 8.39 (dd, J=7.72, 1.84 Hz, 1H), 8.77 (dd, J=4.78, 1.84 Hz, 1H).

### EXAMPLE 149C

### 3-(1,1-dioxido-4H-1,2,4-benzothiadiazin-3-yl)-4-hydroxy-1-(2-phenylpropyl)-1,8-naphthyridin-2(1H)-one

The title compound was prepared according to the procedure of Example 1D substituting the product of Example 149B for the product of Example 1B (0.045 g, 62%). MS (ESI−) m/z 459 (M−H)⁻; ¹H NMR (300 MHz, DMSO-D₆) δ 1.23 (d, J=7.35 Hz, 3H), 3.47 (m, 1H), 4.59 (dd, J=12.50, 6.62 Hz, 1H), 4.75 (m, 1H), 7.16 (m, 1H), 7.28 (m, 4H), 7.45 (dd, J=7.91, 4.60 Hz, 1H), 7.56 (t, J=7.54 Hz, 1H), 7.69 (m, 1H), 7.78 (m, 1H), 7.93 (d, J=8.09 Hz, 1H), 8.53 (dd, J=8.09, 1.84 Hz, 1H), 8.80 (dd, J=4.60, 1.65 Hz, 1H), 14.13 (brs, 1H). The sodium salt of the title compound was prepared according to the procedure of Example 1d. ¹H NMR (300 MHz, DMSO-D₆) δ 1.12 (d, J=6.99 Hz, 3H), 3.42 (m, 1H), 4.30 (dd, J=12.50, 5.52 Hz, 1H), 4.67 (dd, J=12.32, 9.74 Hz, 1H), 7.12 (dd, J=7.72, 4.78 Hz, 1H), 7.18 (m, 1H), 7.30 (m, 6H), 7.56 (m, 1H), 7.67 (d, J=7.72 Hz, 1H), 8.36 (dd, J=7.54, 2.02 Hz, 1H), 8.50 (dd, J=4.78, 1.84 Hz, 1H), 15.92 (s, 1H).

### EXAMPLE 150

### 8-benzyl-6-(1,1-dioxido-4H-1,2,4-benzothiadiazin-3-yl)-5-hydroxy-2-(methylsulfanyl)pyrido[2,3-d]pyrimidin-7(8H)-one

### EXAMPLE 150A

### ethyl 4-(benzylamino)-2-(methylthio)pyrimidine-5-carboxylate

The title compound was prepared according to the procedure of Example 108A substituting benzyl amine for 1-amino-2-ethyl-butane (0.97 g, 92%). MS (DCI/NH₃) m/z 304 (M+H)+¹H NMR (300 MHz, DMSO-d₆) δ 1.32 (q, J=7.48 Hz, 3H) 2.41 (s, 3H) 4.30 (q, J=7.11 Hz, 2H) 4.73 (d, J=5.88 Hz, 2H) 7.30 (m, 5H) 8.58 (s, 1H) 8.89 (t, J=5.70 Hz, 1H)

### EXAMPLE 150B

### 4-(benzylamino)-2-(methylthio)pyrimidine-5-carboxylic Acid

The title compound was prepared according to the procedure of Example 108B substituting the product of Example 150A for the product of Example 108A. (0.185 g, 78%).

### EXAMPLE 150C

### 1-benzyl-7-(methylthio)-2H-pyrimido[4,5-d][1,31]oxazine-2,4(1H)-dione

The title compound was prepared according to the procedure of Example 108C substituting the product of Example 150B for the product of Example 108B (0.145 g, 72%). MS (DCI/NH₃) m/z 302 (M+H)⁺

### EXAMPLE 150D

### 8-benzyl-6-(1,1-dioxido-4H-1,2,4-benzothiadiazin-3-yl)-5-hydroxy-2-(methylsulfanyl)pyrido[2,3-d]pyrimidin-7(8H)-one

The title compound was prepared according to the procedure of Example 1D substituting the product of Example 150C for the product of Example 1B (0.042 g, 18%). MS (ESI−) m/z 478 (M−H)⁻. The sodium salt of the title compound was prepared according to the procedure of Example 1D. ¹H NMR (300 MHz, DMSO-d₆) δ 2.48 (s, 3H) 5.41 (s, 2H) 7.26 (m, 7H) 7.57 (m, 1H) 7.67 (dd, J=7.54, 0.92 Hz, 1H) 8.91 (s, 1H) 15.42 (s, 1H).

### EXAMPLE 151

### 8-benzyl-6-(1,1-dioxido-4H-1,2,4-benzothiadiazin-3-yl)-5-hydroxypyrido[2,3-d]pyrimidin-7(8H)-one

The title compound was prepared according to the procedure of Example 109 substituting the product of Example 151D for the product of Example 108D (0.019 g, 58%). MS (ESI−) m/z 432 (M−H)⁻; ¹H NMR (300 MHz, DMSO-d₆) δ 5.44 (s, 2H) 7.20 (m, 1H) 7.30 (m, 7H) 7.57 (m, 1H) 7.68 (d, J=8.09 Hz, 1H) 8.94 (s, 1H) 9.12 (s, 1H) 15.32 (s, 1H).

### EXAMPLE 152

### 3-(1,1-dioxido-4H-1,2,4-benzothiadiazin-3-yl)-4-hydroxy-1-(3-hydroxybutyl)-2-(1H)-quinolinone

A solution of the product of Example 73 (0.12 g, 0.30 mmol) in tetrahydrofuran (6 mL) was treated with 3.0 M methyl magnesium bromide (0.11 mL, 0.33 mmol) at −50° C., then stirred at room temperature for 1 hour. The solution was diluted with 1N HCl and water then filtered. The resulting solid was triturated with dichloromethane and filtered. The filtrate was concentrated to give the title compound (0.050 g, 40%). MS (DCI/NH₃) m/z 415 (M+H)⁺; ¹H NMR (300 MHz, DMSO-d₆) δ 1.14 (d, J=6.25 Hz, 3H) 1.75 (dd, J=9.19, 5.52 Hz, 2H) 3.78 (m, 1H) 4.57 (in, 2H) 7.54 (m, 2H) 7.77 (m, 2H) 7.94 (d, J=7.35 Hz, 1H) 8.58 (dd, J=7.91, 2.02 Hz, 1H) 8.90 (dd, J=4.78, 1.84 Hz, 1H) 14.12 (s, 1H).

### EXAMPLE 153

### 1-benzyl-3-(1,1-dioxido-4H-1,2,4-benzothiadiazin-3-yl)-4-hydroxythieno[3,4-b]pyridin-2(1H)-one

### EXAMPLE 153A

### 4H-thieno[3,4-d][1,3]oxazine-2,4(1H)-dione

The title compound was prepared according to procedure of Example 119A substituting ethyl 3-aminothiophene-5-carboxylate hydrochloride for methyl 3-amino-5-phenylthiophene-carboxylate (0.86 g, 50%). MS (DCI/NH₃) m/z 187 (M+NH₄)⁺; ¹H NMR (300 MHz, DMSO-d₆) δ 6.90 (d, J=9.93 Hz, 1H) 8.65 (d, J=9.93 Hz, 1H) 11.57 (brs, 1H).

### EXAMPLE 153B

### 1-benzyl-4H-thieno[3,4-d][1,3]oxazine-2,4(1H)-dione

The title compound was prepared according to the procedure of Example 1B substituting the product of Example 153A for the product of Example 1A and substituting benzyl bromide for n-butyl bromide (0.33 g, 91%).

### EXAMPLE 153C

### 1-benzyl-3-(1,1-dioxido-4H-1,2,4-benzothiadiazin-3-yl)-4-hydroxythieno[3,4-b]pyridin-2(1H)-one

The title compound was prepared according to the procedure of Example 1D substituting the product of Example 153B for the product of Example 1B (0.028 g, 5%). MS (ESI−) m/z 436 (M−H)⁻; ¹H NMR (300 MHz, DMSO-d₆) δ 5.13 (s, 2H) 6.68 (d, J=3.31 Hz, 1H) 7.21 (m, 2H) 7.28 (m, 5H) 7.54 (m, 1H) 7.64 (m, 1H) 7.99 (d, J=3.31 Hz, 1H) 15.83 (s, 1H).

### EXAMPLE 154

### 4-[(5-bromo-2-thienyl)methyl]-6-(111-dioxido-4H-1,2,4-benzothiadiazin-3-yl)-7-hydroxythieno[3,2-b]pyridin-5(4H)-one

### EXAMPLE 154A

### 1-[(5-bromothien-2-yl)methyl]-2H-thieno[3,2-d][1,3]oxazine-2,4(1H)-dione

The title compound was prepared according to the procedure of Example 1B substituting the product of Example 110A for the product of Example 1A and substituting 2-bromo-5-bromomethyl-thiophene for n-butyl bromide (0.25 g, 82%).

### EXAMPLE 154B

### 4-[(5-bromo-2-thienyl)methyl]-6-(11-dioxido-4H-1,2,4-benzothiadiazin-3-yl)-7-hydroxythieno[3,2-b]pyridin-5(4H)-one

The title compound was prepared according to the procedure of Example 1D substituting the product of Example 154A for the product of Example 1B (0.219 g, 58%). MS (ESI−) m/z 521 (M−H)⁻. The sodium salt of the title compound was prepared according to the procedure of Example 1D. ¹H NMR (300 MHz, DMSO-d₆) δ 5.28 (s, 2H) 7.02 (d, J=3.68 Hz, 1H) 7.09 (d, J=3.68 Hz, 1H) 7.20 (d, J=8.09 Hz, 1H) 7.27 (m, 1H) 7.37 (d, J=5.15 Hz, 1H) 7.54 (ddd, J=8.55, 7.26, 1.47 Hz, 1H) 7.66 (dd, J=7.72, 1.47 Hz, 1H) 7.81 (d, J=5.15 Hz, 1H) 15.80 (s, 1H).

### EXAMPLE 155

### 1-butyl-3-(1,1-dioxido-4H-1,2,4-benzothiadiazin-3-yl)-2(1H)-pyridinone

### EXAMPLE 155A

### 1-butyl-2-oxo-1,2-dihydropyridine-3-carboxylic acid

To a solution of 2-hydroxy-nicotinic acid (0.50 g, 3.59 mmol) and potassium hydroxide (0.40 g, 7.13 mmol) in 4:1 methanol: water (6 mL) at room temperature, was added 1-iodobutane (0.74 mL, 6.42 mmol). This solution was heated at 60° C. for 30 minutes, then cooled to room temperature and diluted with water and 1N HCl. The resulting solid was filtered and dried to give the title compound (0.27 g, 39%). MS (DCI/NH₃) m/z 196 (M+H)⁺; ¹H NMR (300 MHz, DMSO-d₆) δ 0.91 (m, 3H) 1.30 (m, 2H) 1.69 (m, 2H) 4.10 (m, 2H) 6.73 (m, 1H) 8.27 (dd, J=6.62, 1.84 Hz, 1H) 8.38 (dd, J=7.35, 2.21 Hz, 1H) 14.68 (s, 1H).

### EXAMPLE 155B

### N-[2-(aminosulfonyl)phenyl]-1-butyl-2-oxo-1,2-dihydropyridine-3-carboxamide

A solution of the product of Example 155A and 2-aminobenzene sulfonamide (0.24 g, 1.39 mmol) in tetrahydrofuran (8 mL) at room temperature and treated with TBTU (O-(1H-benzotriazol-1-yl)-N,N,N′,N′-tetramethyluronium tetrafluoroborate) and triethylamine (0.58 mL, 4.15 mmol). After 18 hours, the mixture was poured into water, extracted with ethyl acetate, dried over sodium sulfate, filtered and the filtrate evaporated under vacuum and purified by preparative HPLC on a Waters Symmetry C8 column (40 mm×100 mm, 7 um particle size) using a gradient of 10% to 100% acetonitrile: 0.1% aqueous TFA over 12 min (15 min run time) at a flow rate of 70 mL/min to yield the title compound.

### EXAMPLE 155C

### 1-butyl-3-(1,1-dioxido-4H-1,2,4-benzothiadiazin-3-yl)-2(1H)-pyridinone

The title compound was prepared according to the procedure of Example 84D and purified by preparative HPLC on a Waters Symmetry C8 column (40 mm X 100 mm, 7 um particle size) using a gradient of 10% to 100% acetonitrile:0.1% aqueous TFA over 12 min (15 min run time) at a flow rate of 70 mL/min. MS DCI/NH₃) m/z 332 (M+H)⁺. The sodium salt of the title compound was prepared according to the procedure of Example 1D. ¹H NMR (300 MHz, DMSO-d₆) δ 0.93 (t, J=7.35 Hz, 3H) 1.34 (td, J=14.89, 7.35 Hz, 2H) 1.73 (ddd, J=14.89, 7.72, 7.54 Hz, 2H) 4.13 (m, 2H) 6.73 (dd, J=7.35, 6.62 Hz, 1H) 7.50 (m, 1H) 7.59 (d, J=7.35 Hz, 1H) 7.71 (m, 1H) 7.85 (dd, J=7.91, 1.29 Hz, 1H) 8.30 (dd, J=6.43, 2.02 Hz, 1H) 8.62 (dd, J=7.54, 2.02 Hz, 1H) 13.76 (s, 1H).

### EXAMPLE 156

### 3-(1,1-dioxido-4H-1,2,4-benzothiadiazin-3-yl)-1,8-naphthyridine-2,4-diol

The product of Example 73 (0.12 g, 0.30 mmol) in tetrahydrofuran (6 mL) was reacted with 3.0 M methyl magnesium bromide (0.11 mL, 0.33 mmol) at −50° C., and then stirred at room temperature for 1 hour. The solution was diluted with 1N HCl and filtered. The resulting solid was triturated with dichloromethane and filtered to yield the product. MS (DCI/NH₃) m/z 343 (M+H)⁺; ¹H NMR (300 MHz, DMSO-d₆) δ 7.46 (dd, J=7.91, 4.60 Hz, 1H) 7.57 (m, 1H) 7.64 (d, J=7.72 Hz, 1H) 7.79 (ddd, J=8.36, 7.26, 1.29 Hz, 1H) 7.94 (d, J=7.35 Hz, 1H) 8.49 (dd, J=8.09, 1.84 Hz, 1H) 8.80 (dd, J=4.60, 1.65 Hz, 1H) 12.92 (s, 1H) 14.28 (s, 1H).

### EXAMPLE 157

### 1-(benzyloxy)-3-(1,1-dioxido-4H-1,2,4-benzothiadiazin-3-yl)-4-hydroxy-1,8-naphthyridin-2(1H)-one

### EXAMPLE 157A

### ethyl 2-[(benzyloxy)amino]nicotinate

2-Chloro-nicotinic acid ethyl ester (4.55 g, 24.6 mmol), O-benzylhydroxyamine hydrochloride (7.85 g, 49.2 mmol) and N,N-diisopropylethylamine (6.36 g, 49.2 mmol) in 10 mL 1,4-dioxane were reacted in a sealed tube at 120° C. for 48 hours. The reaction mix was partitioned between ethyl acetate and 5% aqueous sodium bicarbonate. The aqueous layer was re-extracted with ethyl acetate (2×50 mL). The organic layers were combined and dried over sodium sulfate, filtered, and concentrated. The residue was purified by column chromatography on silica gel eluting with hexane and ethyl acetate (9:1) to provide the title compound (3.5 g, 53%). MS (DCI) m/z 273 (M+H)⁺.

### EXAMPLE 157B

### ethyl 2-[(benzyloxy)(3-ethoxy-3-oxopropanoyl)amino]nicotinate

A solution of the product of Example 157A (1.2 g, 4.4 mmol) and triethylamine (0.49 g, 4.8 mmol) in dichloromethane (25 mL) was treated dropwise with ethyl chloromalonate (0.73 g, 4.8 mmol), stirred for 2 hr and partitioned between ethyl acetate and water and the layers were separated. The ethyl acetate layer was washed with brine, dried (Na₂SO₄), and concentrated. The residue was purified by column chromatography on silica gel eluting with hexane and ethyl acetate (3:1) to provide the title compound (1.1 g, 65%). MS (DCI) m/z 387 (M+H)⁺.

### EXAMPLE 157C

### ethyl 1-(benzyloxy)-4-hydroxy-2-oxo-1,2-dihydro-1,8-naphthyridine-3-carboxylate

A solution of the product of Example 157B (0.386 g, 1.0 mmol) in ethanol (5 mL) was treated with 21% sodium ethoxide in ethanol (0.324 g, 1.0 mmol), stirred for 30 minutes and partitioned between ethyl acetate and 5% aqueous HCl and the layers were separated. The ethyl acetate layer was washed with brine, dried (Na₂SO₄), and concentrated to provide the title compound (0.28 g, 82%). MS (DCI) m/z 341(M+H)⁺.

### EXAMPLE 157D

### N-[2-(aminosulfonyl)phenyl]-1-(benzyloxy)-4-hydroxy-2-oxo-1,2-dihydro-1,8-naphthyridine-3-carboxamide

The title compound was prepared according to the procedure of Example 84C substituting the product of Example 157C for the product of Example 84B and substituting 2-aminosulfonamide for the product of Example 84A (340 mg, 89% yield). MS (DCI) m/z 467 (M+H)⁺.

### EXAMPLE 157E

### 1-(benzyloxy)-3-(1,1-dioxido-4H-1,2,4-benzothiadiazin-3-yl)-4-hydroxy-1,8-naphthyridin-2(1H)-one

The title compound was prepared according to the procedure of Example 84D substituting the product of Example 157D for the product of Example 84C (0.082 g, 87%). MS (ESI−) m/z 447 (M−H)⁻. The sodium salt of the title compound was prepared according to the procedure of Example 1D. ¹H NMR (300 MHz, DMSO-d₆) δ 5.12 (s, 2H) 7.22 (dd, J=7.72, 4.78 Hz, 1H) 7.30 (m, 2H) 7.44 (m, 3H) 7.57 (m, 1H) 7.70 (m, 3H) 8.41 (dd, J=7.72, 1.84 Hz, 1H) 8.61 (dd, J=4.78, 1.84 Hz, 1H) 15.70 (s, 1H).

### EXAMPLE 158

### 3-(1,1-dioxido-4H-1,2,4-benzothiadiazin-3-yl)-4-hydroxy-1-isobutoxy-1,8-naphthyridin-2(1H)-one

### EXAMPLE 158A

### ethyl 2-(isobutoxyamino)nicotinate

The title compound was prepared according to the procedure of Example 157A substituting O-isobutylhydroxylamine hydrochloride for O-benzylhydroxyamine hydrochloride (0.372 g, 34%). MS (DCI) m/z 239 (M+H)⁺.

### EXAMPLE 158B

### ethyl 2-[(3-ethoxy-3-oxopropanoyl)(isobutoxy)amino]nicotinate

The title compound was prepared according to the procedure of Example 157B substituting the product of Example 158A for the product of Example 157A (0.230 g, 42%). MS (DCI) m/z 353 (M+H)⁺.

### EXAMPLE 158C

### ethyl 4-hydroxy-1-isobutoxy-2-oxo-1,2-dihydro-1,8-naphthyridine-3-carboxylate

The title compound was prepared according to the procedure of Example 157C substituting the product of Example 158B for the product of 157B (0.200 g, 99%). MS (DCI) m/z 307 (M+H)⁺.

### EXAMPLE 158D

### N-[2-(aminosulfonyl)phenyl]-4-hydroxy-1-isobutoxy-2-oxo-1,2-dihydro-1,8-naphthyridine-3-carboxamide

The title compound was prepared according to the procedure of Example 84C substituting the product of Example 158C for the product of Example 84B and substituting 2-aminosulfonamide for the product of Example 84A (0.225 g, 86%). MS (DCI) m/z 433 (M+H)⁺.

### EXAMPLE 158E

### 3-(1,1-dioxido-4H-1,2,4-benzothiadiazin-3-yl)-4-hydroxy-1-isobutoxy-1,8-naphthyridin-2(1H)-one

The title compound was prepared according to the procedure of Example 84D substituting the product of Example 158D for the product of Example 84C (0.200 g, 93%). MS (ESI−) m/z 413 (M−H)⁻. The sodium salt of the title compound was prepared according to the procedure of Example 1d. ¹H NMR (300 MHz, DMSO-d₆) d 1.05 (d, J=6.62 Hz, 6H) 2.08 (m, 1H) 3.88 (d, J=6.62 Hz, 2H) 7.18 (dd, J=7.72, 4.78 Hz, 1H) 7.29 (m, 2H) 7.55 (m, 1H) 7.67 (d, J=7.72 Hz, 1H) 8.37 (dd, J=7.72, 1.84 Hz, 1H) 8.55 (dd, J=4.78, 1.84 Hz, 1H) 15.72 (s, 1H).

### EXAMPLE 159

### 1-butyl-3-(1,1-dioxido-4H-1,2,4-benzothiadiazin-3-yl)-4-hydroxy-1,5-naphthyridin-2(1H)-one

### EXAMPLE 159A

### 2H-pyrido[3,2-d][1,3]oxazine-2,4(1H)-dione

The title compound was prepared as a minor bi-product (0.50 g, 4%) from 2,3-pyridinecarboxylic anhydride (11.4 g, 76 mmol) and trimethylsilyl azide (11.0 mL, 80 mmol) according to the procedure of Le Count, D. J. and co-workers described in Synthesis, 1982, 972-973. ¹H NMR (300 MHz, DMSO-d₆) δ 7.56 (dd, J=8.46, 1.47 Hz, 1H) 7.71 (dd, J=8.46, 4.41 Hz, 1H) 8.51 (dd, J=4.41, 1.47 Hz, 1H) 11.78 (s, 1H).

### EXAMPLE 159B

### 1-butyl-2H-pyrido[3,2-d][1,3]oxazine-2,4(1H)-dione

The title compound was prepared according to the procedure of Example 1B substituting the product of Example 159A for the product of Example 1A (0.12 g, 35%). ¹H NMR (300 MHz, DMSO-d₆) δ 0.92 (t, J=7.35 Hz, 3H) 1.40 (m, J=15.26, 7.17 Hz, 2H) 1.60 (m, 2H) 3.98 (m, 2H) 7.81 (dd, J=8.82, 4.41 Hz, 1H) 7.97 (dd, J=8.64, 1.29 Hz, 1H) 8.55 (dd, J=4.23, 1.29 Hz, 1H).

### EXAMPLE 159C

### 1-butyl-3-(1,1-dioxido-4H-1,2,4-benzothiadiazin-3-yl)-4-hydroxy-1,5-naphthyridin-2(1H)-one

The title compound was prepared according to the procedure of Example 1D substituting the product of Example 159B for the product of Example 1B (0.053 g, 25%). MS (ESI−) m/z 397 (M−H)⁻. The sodium salt of the title compound was prepared according to the procedure of Example 1D. ¹H NMR (300 MHz, DMSO-d₆) δ 0.94 (t, J=7.17 Hz, 3H) 1.39 (m, 2H) 1.54 (m, 2H) 4.07 (t, J=7.72 Hz, 2H) 7.28 (m, 2H) 7.56 (m, 2H) 7.68 (dd, J=7.91, 1.29 Hz, 1H) 7.77 (d, J=8.46 Hz, 1H) 8.39 (d, J=4.04 Hz, 1H) 16.15 (s, 1H).

### EXAMPLE 160

### 1-benzyl-3-(1,1-dioxido-4H-1,2,4-benzothiadiazin-3-yl)-4-hydroxy-1,5-naphthyridin-2(1H)-one

### EXAMPLE 160A

### 1-benzyl-2H-pyrido[3,2-d][1,3]oxazine-2,4(1H)-dione

The title compound was prepared according to the procedure of Example 1B substituting the product of Example 159A for the product of Example 1A and substituting benzyl bromide for n-butyl bromide (0.92 g, 60%). ¹H NMR (300 MHz, DMSO-d₆) δ 5.28 (s, 2H) 7.33 (m, 3H) 7.43 (m, 2H) 7.70 (m, 2H) 8.54 (dd, J=3.86, 1.65 Hz, 1H).

### EXAMPLE 160B

### ethyl 1-benzyl-4-hydroxy-2-oxo-1,2-dihydro-1,5-naphthyridine-3-carboxylate

The title compound was prepared according to the procedure of Example 89A substituting the product of Example 160A for the product of Example 1B (0.110 g, 23%). MS (DCI) m/z 325 (M+H)⁺.

### EXAMPLE 160C

### N-[2-(aminosulfonyl)phenyl]-1-benzyl-4-hydroxy-2-oxo-1,2-dihydro-1,5-naphthyridine-3-carboxamide

The title compound was prepared according to the procedure of Example 89B substituting the product of Example 160B for the product of Example 89A and 2-aminobenzenesulfonamide for 2-amino-4-chlorobenzenesulfonamide (0.12 g, 86%). MS (ESI−) m/z 449 (M−H)⁻.

### EXAMPLE 160D

### 1-benzyl-3-(1,1-dioxido-4H-1,2,4-benzothiadiazin-3-yl)-4-hydroxy-1,5-naphthyridin-2(1H)-one

The title compound was prepared according to the procedure of Example 84C substituting the product of Example 160C for the product of Example 84B (0.120 g, 99%). MS (ESI−) m/z 431 (M−H)⁻. The sodium salt of the title compound was prepared according to the procedure of Example 1D. ¹H NMR (300 MHz, DMSO-d₆) δ 5.39 (s, 2H) 7.25 (m, 7H) 7.40 (dd, J=8.46, 4.41 Hz, 1H) 7.57 (m, 2H) 7.68 (d, J=8.09 Hz, 1H) 8.35 (d, J=4.04 Hz, 1H) 16.11 (s, 1H).

### EXAMPLE 161

### 1-benzyl-4-chloro-3-(1,1-dioxido-4H-1,2,4-benzothiadiazin-3-yl)-1,8-naphthyridin-2(1H)-one

The title compound was prepared from the product of Example 15B and phosphoryl chloride according to the procedure of Stadlbauer, W. and co-workers described in Journal of Heterocyclic Chemistry, 35, 1998, 627-636 (2.07 g, 88%). MS (ESI−) m/z 449 (M−H)⁻; ¹H NMR (300 MHz, DMSO-d₆) δ 5.68 (s, 2H) 7.29 (m, 6H) 7.57 (m, 2H) 7.75 (m, 1H) 7.92 (dd, J=7.91, 1.29 Hz, 1H) 8.56 (dd, J=8.09, 1.47 Hz, 1H) 8.87 (dd, J=4.60, 1.65 Hz, 1H) 12.73 (s, 1H).

### EXAMPLE 162

### 3-(1,1-dioxido-4H-1,2,4-benzothiadiazin-3-yl)-4-hydroxy-1-{[(1E)-phenylmethylene]amino}-2(1H)-quinolinone

### EXAMPLE 162A

### 2-[(2E)-2-benzylidenehydrazino]benzoic acid

The title compound was prepared from 2-hydrazinobenzoic acid hydrochloride (1.89 g, 10.0 mmol) and benzaldehyde (1.06 g, 10.0 mmol) according to the procedure of Fischer, E. and co-workers described in Chem. Ber., 35, 1902, 2318 (2.4 g, quantitative yield). MS (DCI) m/z 241 (M+H)⁺.

### EXAMPLE 162B

### 1-{[(1E)-phenylmethylenel amino 1-2H-3,1-benzoxazine-2,4(1H)-dione

A solution of the product of Example 162A (1.2 g, 5.0 mmol) and potassium hydroxide (0.336 g, 6.0 mmol) in 15 ml of water at 0° C. was treated dropwise with 20% phosgene in toluene (3.5 ml, 6.5 mmol), stirred for 1 hour, treated with 1M NaOH to reach a pH of 10 and extracted 3×30 mL with ethyl acetate. The ethyl acetate extracts were combined, washed with brine, dried (Na₂SO₄), and concentrated to provide the title compound (0.32 g, 24%). MS (DCI) m/z 267 (M+H)⁺.

### EXAMPLE 162C

### 3-(1,1-dioxido-4H-1,2,4-benzothiadiazin-3-yl)-4-hydroxy-1-}[(1E)-phenylmethylene]amino 1-2(1H)-quinolinone

The title compound was prepared according to the procedure of Example 1D substituting the product of Example 162B for the product of Example 1B (0.110 g, 49%). MS (ESI−) m/z 443 (M−H)⁻. The sodium salt of the title compound was prepared according to the procedure of Example 1D. ¹H NMR (300 MHz, DMSO-d₆) δ 7.16 (m, 1H) 7.30 (m, 2H) 7.54 (m, 6H) 7.67 (dd, J=8.09, 1.47 Hz, 1H) 7.99 (m, 2H) 8.13 (dd, J=7.91, 1.29 Hz, 1H) 9.04 (s, 1H) 16.09 (s, 1H).

### EXAMPLE 163

### 1-amino-3-(1,1-dioxido-4H-1,2,4-benzothiadiazin-3-yl)-4-hydroxy-2(1H)-quinolinone

A solution of the product of Example 162C (0.075 g, 0.17 mmol) in 10% aqueous potassium hydroxide (5 mL) was refluxed for 2 hours, cooled, treated with 12 M HCl to pH 3 which produced a precipitate. The solid was collected by filtration, washed repeatedly with water and dried to constant mass to give the desired product (0.050 g, 83%). MS (ESI−) m/z 355 (M−H)⁻. The sodium salt of the title compound was prepared according to the procedure of Example 1D. ¹H NMR (300 MHz, DMSO-d₆) δ 5.31 (s, 2H) 7.05 (t, J=8.09 Hz, 1H) 7.27 (m, 2H) 7.53 (m, 2H) 7.67 (m, 2H) 8.07 (dd, J=8.09, 1.47 Hz, 1H) 16.38 (s, 1H).

### EXAMPLE 164

### 3-(1,1-dioxido-4H-1,2,4-benzothiadiazin-3-yl)-4-hydroxy-1-(2-phenylethyl)-1,8-naphthyridin-2(1H)-one

### EXAMPLE 164A

### ethyl 2-[(2-phenylethyl)amino]nicotinate

The title compound was prepared according to the procedure of Example 3A substituting phenethylamine for 2-ethyl-butylamine (1.98 g, 73%). MS (DCI) m/z 271 (M+H)⁺.

### EXAMPLE 164B

### 1-(2-phenylethyl)-2H-pyrido[2,3-d][1,3]oxazine-2,4(1H)-dione

The title compound was prepared according to the procedure of Example 3B substituting the product of 164A for the product of Example 3A (0.53 g, 99%). MS (DCI) m/z 269 (M+H)⁺.

### EXAMPLE 164C

### 3-(1,1-dioxido-4H-1,2,4-benzothiadiazin-3-yl)-4-hydroxy-1-(2-phenylethyl)-1,8-naphthyridin-2(1H)-one

The title compound was prepared according to the procedure of Example 1D substituting the product of Example 164B for the product of Example 1B (0.132 g, 59%). MS (ESI−) m/z 445 (M−H)⁻. The sodium salt of the title compound was prepared according to the procedure of Example 1D. ¹H NMR (300 MHz, DMSO-d₆) δ 2.87 (m, 2H) 4.47 (m, 2 H) 7.16 (dd, J=7.72, 4.78 Hz, 1H) 7.29 (m, 7H) 7.57 (m, 1H) 7.67 (d, J=7.72 Hz, 1H) 8.40 (dd, J=7.72, 1.84 Hz, 1H) 8.57 (dd, J=4.78, 1.84 Hz, 1H) 15.90 (s, 1H).

### EXAMPLE 165

### 1-butyl-4-chloro-3-(1,1-dioxido-4H-1,2,4-benzothiadiazin-3-yl)-1,8-naphthyridin-2(1H)-one

The title compound was prepared according to the procedure of Example 161 substituting the product of Example 1D for the product of Example 15B.

### EXAMPLE 166

### 4-amino-1-butyl-3-(1,1-dioxido-4H-1,2,4-benzothiadiazin-3-yl)-1,8-naphthyridin-2(1H)-one

A solution of the product of Example 165 (0.10 g, 0.24 mmol) and ammonia (2 ml of a 2 M solution in methanol, 4.0 mmol) was stirred in a sealed tube at 100° C. for 2 hours, allowed to cool to room temperature. The resulting solid collected by filtration and washed with methanol (2 ml) to give the title compound as a brown solid (0.019 g, 20%). MS (ESI−) m/z 396 (M−H)⁻; ¹H NMR (300 MHz, DMSO-d₆) δ 0.94 (t, J=7.35 Hz, 3H), 1.38 (m, 2H), 1.66 (m, 2H), 4.44 (t, J=7.35 Hz, 2H), 7.48 (m, 2H), 7.55 (d, J=8.09 Hz, 1H), 7.70 (t, J=8.46 Hz, 1H), 7.84 (dd, J=7.72, 1.10 Hz, 1H), 8.77 (d, J=8.09 Hz, 1H), 8.82 (dd, J=4.78, 1.47 Hz, 1H), 9.84 (brs, 1H).

### EXAMPLE 167

### 1-butyl-3-(1,1-dioxido-4H-1,2,4-benzothiadiazin-3-yl)-4-(methylamino)-1,8-naphthyridin-2(1H)-one

The title compound was prepared according to the procedure of Example 166 substituting methylamine (2 M solution in methanol) for ammonia (2 M solution in methanol) as a brown solid (0.023 g, 23%). MS (ESI−) m/z 410 (M−H)⁻; ¹H NMR (300 MHz, DMSO-d₆) δ 0.91 (t, J=7.17 Hz, 3H), 1.34 (m, 2H), 1.60 (m, 2H), 2.95 (d, J=5.15 Hz, 3H), 4.31 (m, J=7.36 Hz, 2H), 7.36 (dd, J=8.09, 4.78 Hz, 1H), 7.40 (d, J=8.46 Hz, 1H), 7.49 (t, J=8.09 Hz, 1H), 7.71 (m, 2H), 7.85 (dd, J=7.91, 1.29 Hz, 1H), 8.56 (dd, J=8.27, 1.29 Hz, 1H), 8.69 (dd, J=4.60, 1.29 Hz, 1H), 12.44 (brs, 1H).

### EXAMPLE 168

### 1-butyl-4-(dimethylamino)-3-(1,1-dioxido-4H-1,2,4-benzothiadiazin-3-yl)-1,8-naphthyridin-2(1H)-one

The title compound was prepared according to the procedure of Example 166 substituting dimethylamine (2 M solution in methanol) for ammonia (2 M solution in methanol) as a brown solid (0.015 g, 15%). MS (ESI−) m/z 424 (M−H)⁻; ¹H NMR (300 MHz, DMSO-d₆) δ 0.93 (t, J=7.35 Hz, 3H), 1.36 (m, 2H), 1.63 (m, 2H), 2.99 (s, 6H), 4.36 (t, J=7.72 Hz, 2H), 7.38 (m, 2H), 7.51 (m, 1H), 7.73 (m, 1H), 7.88 (dd, J=8.09, 1.47 Hz, 1H), 8.36 (dd, J=8.09, 1.47 Hz, 1H), 8.71 (dd, J=4.78, 1.84 Hz, 1H), 12.45 (s, 1H).

### EXAMPLE 169

### 1-butyl-3-(1,1-dioxido-4H-1,2,4-benzothiadiazin-3-yl)-4-hydrazino-1,8-naphthyridin-2(1H)-one

The title compound was prepared according to the procedure of Example 166 substituting hydrazine for ammonia (2 M solution in methanol) as a brown solid (0.026 g, 26%). MS (ESI−) m/z 411 (M−H)⁻; ¹H NMR (300 MHz, DMSO-d₆) δ 0.94 (t, J=7.35 Hz, 3H), 1.39 (m, 2H), 1.64 (m, 2H), 3.35 (brs, 3H), 4.41 (t, J=7.72 Hz, 2H), 7.04 (t, J=7.54 Hz, 1H), 7.42 (dd, J=7.72, 4.78 Hz, 1H), 7.57 (m, 1H), 7.83 (dd, J=7.91, 1.65 Hz, 1H), 8.49 (dd, J=7.72, 1.84 Hz, 1H), 8.64 (d, J=8.46 Hz, 1H), 8.68 (dd, J=4.78, 1.84 Hz, 1H), 9.65 (s, 1H).

### EXAMPLE 170

### 4-azido-1-butyl-3-(1,1-dioxido-4H-1,2,4-benzothiadiazin-3-yl)-1.8-naphthyridin-2(1H)-one

A solution of the product of Example 165 (0.1 g, 0.24 mmol) and sodium azide (0.037 g, 0.571 mmol) in dimethylformamide (2.5 ml) was stirred at 80° C. for 1.5 hours, allowed to cool to room temperature and concentrated under reduced pressure. The crude residue was purified by a C8 HPLC column eluting with 20% to 80% acetonitrile in water with 1% trifluoroacetic acid to give the title compound (0.025 g, 26% after column purification). MS (ESI−) m/z 422 (M−H)⁻; ¹H NMR (300 MHz, DMSO-d₆) δ 0.94 (t, J=7.35 Hz, 3H), 1.38 (m, 2H), 1.67 (m, 2H), 4.42 (t, J=7.54 Hz, 2H), 7.41 (d, J=7.72 Hz, 1H), 7.46 (dd, J=7.91, 4.60 Hz, 1H), 7.56 (m, 1H), 7.76 (m, 1H), 7.91 (dd, J=8.09, 1.10 Hz, 1H), 8.41 (dd, J=8.09, 1.84 Hz, 1H), 8.84 (dd, J=4.41, 1.84 Hz, 1H), 12.74 (s, 1H).

### EXAMPLE 171

### 1-butyl-3-(1,1-dioxido-4H-1,2,4-benzothiadiazin-3-yl)-4-[(2-hydroxyethyl)amino]-1,8-naphthyridin-2(1H)-one

The title compound was prepared according to the procedure of Example 166 substituting ethanolamine (0.25 g, 4.0 mmol) and anhydrous methanol (2 ml) for ammonia (2 M solution in methanol) (0.02 g, 19%). MS (ESI−) m/z 440 (M−H)⁻; ¹H NMR (300 MHz, DMSO-d₆) δ 0.92 (t, J=7.35 Hz, 3H), 1.35 (m, 2H), 1.61 (m, 2H), 2.71 (m, 1H), 3.40 (m, 1H), 3.47 (m, 2H), 3.57 (m, 2H), 4.32 (t, J=7.36 Hz, 2H), 7.35 (m, 1H), 7.39 (d, J=6.99 Hz, 1H), 7.44 (t, J=7.72 Hz, 1H), 7.51 (brs, 1H), 7.67 (m, 1H), 7.81 (dd, J=7.91, 1.29 Hz, 1H), 8.66 (dd, J=8.09, 1.47 Hz, 1H), 8.69 (dd, J=4.78, 1.47 Hz, 1H).

### EXAMPLE 172

### 3-(1,1-dioxido-4H-1,2,4-benzothiadiazin-3-yl)-4-hydroxy-1-propoxyquinolin-2(1H)-one

### EXAMPLE 172A

### ethyl 2-(hydroxyamino)benzoate

The title compound is prepared from ethyl-2-nitrobenzoate according to the procedure of Entwistle and Gilkerson described in Tetrahedron, 34, 1978, 213-215.

### EXAMPLE 172B

### ethyl 2-(propoxyamino)benzoate

The title compound is prepared according to the procedure of Example 1B substituting the product of Example 172A for the product of Example 1A and substituting npropyl bromide for n-butyl bromide.

### EXAMPLE 172C

### ethyl 2-[(3-ethoxy-3-oxopropanoyl)(propoxy)amino]benzoate

The title compound is prepared according to the procedure of Example 157B substituting the product of Example 172B for the product of Example 157A.

### EXAMPLE 172D

### ethyl 4-hydroxy-2-oxo-1-propoxy-1,2-dihydroquinoline-3-carboxylate

The title compound is prepared according to the procedure of Example 157C substituting the product of Example 172C for the product of Example 157B.

### EXAMPLE 172E

### N-[2-(aminosulfonyl)phenyl]-4-hydroxy-2-oxo-1-propoxy-1,2-dihydroquinoline-3-carboxamide

The title compound is prepared according to the procedure of Example 84C substituting the product of Example 172D for the product of Example 84B and substituting 2-aminosulfonamide for the product of Example 84A.

### EXAMPLE 172F

### 3-(1,1-dioxido-4H-1,2,4-benzothiadiazin-3-yl)-4-hydroxy-1-propoxyquinolin-2(1H)-one

The title compound is prepared according to the procedure of Example 84D substituting the product of Example 172E for the product of Example 84C. The sodium salt of the title compound is prepared according to the procedure of Example 1D.

### EXAMPLE 173

### 7-benzyl-5-(1,1-dioxido-4H-1,2,4-benzothiadiazin-3-yl)-4-hydroxy-3-(hydroxymethyl)-7,7a-dihydrothieno[2,3-b]pyridin-6(3aH)-one

### EXAMPLE 173A

### methyl 2-amino-4-(hydroxymethyl)thiophene-3-carboxylate

A solution of methyl cyanoacetate (1.18 mL, 13.28 mmol) and sodium sulfide nonahydrate (3.20 g, 13.28 mmol) in methanol (25 mL) at 0° C. was treated with 1-acetoxy-3-chloroacetone (2.0 g, 13.28 mmol). The cold bath was removed and triethylamine (1.86 mL, 13.28 mmol) was added dropwise. The solution was stirred at room temperature for 20 hours then diluted with water and extracted into ethyl acetate. The organic layer was dried over sodium sulfate, filtered, and the solvent removed under vacuum to provide the titled compound (1.25 g, 51%). MS (DCI/NH₃) M/Z 188 (M+H)⁺; ¹H NMR (300 MHz, DMSO-d₆) δ 3.68 (s, 3H) 4.45 (dd, J=5.52, 1.47 Hz, 2H) 4.88 (t, J=5.70 Hz, 1H) 6.12 (s, 1H) 7.28 (s, 2H)

### EXAMPLE 173B

### methyl 2-amino-4-({[tert-butyl(dimethyl)silyl]oxy)methyl)thiophene-3-carboxylate

A solution of the product of Example 173A (1.25g, 6.70 mmol) and N,N-diisopropylethylamine (0.71 mL, 7.35 mmol) in dichloromethane at 0° C. was treated with t-butyldimethylsilyl trifluoromethanesulfonate (0.85 mL, 6.70 mmol). After stirring at 0° C. for 1 hour, the solution was poured into water, extracted into dichloromethane, and dried over sodium sulfate. The solvent was removed under vacuum to provide the titled compound (0.87 g, 78%). MS (DCI/NH₃) m/z 302 (M+H)⁺; ¹H NMR (300 MHz, DMSO-d₆) δ 0.00 (m, 6H) 0.84 (s, 9H) 3.62 (s, 3H) 4.59 (d, J=1.47 Hz, 2H) 6.03 (m, 1H) 7.22 (s, 2H).

### EXAMPLE 173C

### methyl 2-(benzylamino)-4-(f [tert-butyl(dimethyl)silyl]oxy}methyl)thiophene-3-carboxylate

A solution of the product of Example 173B (0.36 g, 1.20 mmol) and potassium carbonate (0.185 g, 1.30 mmol) in acetonitrile (5 mL) was treated with benzyl bromide (0.16 mL, 1.25 mmol) at 45° C. for 24 hours. The solution was poured into water and extracted into ethyl acetate (2×). The combined organic layers were concentrated and purified by flash chromatography eluting with dichloromethane to provide the titled compound (0.17 g, 36%). MS (DCI/NH₃) m/z 392 (M+H)⁺; ¹H NMR (300 MHz, DMSO-d₆) δ 0.00 (m, 6H) 0.84 (s, 9H) 3.67 (m, 3H) 4.38 (d, J=5.88 Hz, 2H) 4.62 (d, J=1.47 Hz, 2H) 6.12 (s, 1H) 7.28 (m, 5H) 8.16 (t, J=6.07 Hz, 1H).

### EXAMPLE 173D

### 1-benzyl-5-({[tert-butyl(dimethyl)silyl]oxy}methyl)-2H-thieno[2,3-d][1,3]oxazine-2,4(1H)-dione

The title compound was prepared according to the procedure of Example 3B substituting the product of Example 173C for the product of Example 3A (0.015 g, 83%). MS (DCI/NH₃) m/z 404 (M+H)+

### EXAMPLE 173E

### 7-benzyl-5-(1,1-dioxido-4H-1,2,4-benzothiadiazin-3-yl)-4-hydroxy-3-(hydroxymethyl)-7,7A-dihydrothieno[2,3-b]pyridin-6(3AH)-one

The title compound was prepared according to the procedure of Example 1D substituting the product of Example 173D for the product of Example 1B. (0.013 g, 8%). MS (DCI/NH₃) m/z 468 (M+H)⁺; ¹H NMR (300 MHz, DMSO-d₆) δ 4.78 (s, 2H) 5.42 (s, 2H) 7.13 (s, 1H) 7.32 (m, 5H) 7.53 (t, J=7.17 Hz, 1H) 7.64 (d, J=9.93 Hz, 1H) 7.75 (m, 1H) 7.91 (d, J=6.99 Hz, 1H).

### EXAMPLE 174

### 1-benzyl-3-(6-chloro-1,1-dioxido-4H-thieno[3,2-e][1,2,4]thiadiazin-3-yl)-4-hydroxy-1,8-naphthyridin-2(1H)-one

### EXAMPLE 174A

### 3-amino-5-chlorothiophene-2-sulfonamide

The title compound is prepared according to the procedure of Hansen, J. and coworkers as described in J. of Medicinal Chemistry 2002, 45, 4171-4187.

### EXAMPLE 174B

### N-[2-(aminosulfonyl)-5-chlorothien-3-yl]-1-benzyl-4-hydroxy-2-oxo-1,2-dihydro-1,8-naphthyridine-3-carboxamide

The title compound is prepared according to the procedure of Example 84C substituting the product of Example 174A for the product of Example 84A and substituting 3-amino-5-chlorothiophene-2-sulfonamide for 2-amino-5-bromobenzenesulfonamide.

### EXAMPLE 174C

### 1-benzyl-3-(6-chloro-1,1-dioxido-4H-thieno[3,2-e][1,2,4]thiadiazin-3-yl)-4-hydroxy-1,8-naphthyridin-2(1H)-one

The title compound is prepared according to the procedure of Example 84D substituting the product of Example 174B for the product of Example 84C.

### EXAMPLE 175

### 1-benzyl-3-(6-chloro-1,1-dioxido-4H-thieno[3,2-e][1,2,4]thiadiazin-3-yl)-4-hydroxyguinolin-2(1H)-one

### EXAMPLE 175A

### N-[2-(aminosulfonyl)-5-chlorothien-3-yl]-1-benzyl-4-hydroxy-2-oxo-1,2-dihydroguinoline-3-carboxamide

The title compound is prepared according to the procedure of Example 84C substituting the product of Example 174A for the product of Example 84A and substituting the product of Example 99A for the product of example Example 84B.

### EXAMPLE 175B

### 1-benzyl-3-(6-chloro-11-dioxido-4H-thieno[3,2-el [1,2,4]thiadiazin-3-yl)-4-hydroxyquinolin-2(1H)-one

The title compound is prepared according to the procedure of Example 84D substituting the product of Example 175A for the product of Example 84C.

### EXAMPLE 176

### 3-[5-(aminomethyl)-1,1-dioxido-4H-1,2,4-benzothiadiazin-3-yl]-1-benzyl-4-hydroxy-1,8-naphthyridin-2(1H)-one

The product of Example 97 (91.6 mg, 0.2002 mmol) and Raney-nickel (0.94 g) in tetrahydrofuran (92 mL) and triethylamine (4.5 mL) was hydrogenated at 60 psi H₂ pressure at 500 for 2 days, with additional Raney-nickel (0.94 g) being added after 24 hrs. The reaction was cooled to room temperature, filtered, and concentrated by rotary evaporation to a greenish-yellow solid. The residue was purified by preparative HPLC on a Waters Symmetry C8 column (40 mm×100 mm, 7 um particle size) using a gradient of 10% to 100% acetonitrile:0.1% aqueous TFA over 12 min (15 min run time) at a flow rate of 70 mL/min to give the title compound as a white solid (11 mg, 12%). MS (ESI−)⁻ m/z 460 (M−H)⁻; ¹H NMR (300 MHz, DMSO-d₆) δ 4.31 (s, 2H) 5.64 (s, 2H) 7.28 (m, 6H) 7.49 (m, 1H) 7.74 (d, J=7.35 Hz, 1H) 7.85 (d, J=7.72 Hz, 1H) 8.48 (m, 3H) 8.68 (m, 1H). The sodium salt of the title compound was prepared according to the procedure of Example 1D. ¹H NMR (300 MHz, DMSO-d₆) δ 4.16 (s, 2H) 5.54 (s, 2H) 7.24 (m, 7H) 7.65 (d, J=7.72 Hz, 2H) 8.43 (dd, J=7.54, 1.65 Hz, 1H) 8.50 (dd, J=4.41, 1.84 Hz, 1H).

### EXAMPLE 177

### 8-benzyl-3-chloro-6-(1,1-dioxido-4H-1,2,4-benzothiadiazin-3-yl)-5-hydroxypyrido[2,3-c]pyridazin-7(8H)-one

### EXAMPLE 177A

### 3-(benzylamino)-6-chloropyridazine-4-carboxylic Acid

2,5-Dichloro-pyridizine-3-carboxylate (0.40 g, 2.07 mmol) in toluene (8 mL) was reacted with triethylamine (0.72 m]L, 5.20 mmol) and benzyl amine (0.23 mL, 2.07 mmol) at 90° C. for 8 hours. The solution was partitioned between water and ethyl acetate. The organic layer was dried over sodium sulfate, filtered, and concentrated to yield the title compound (0.257 g, 47%). MS (DCI/NH₃) m/z 264 (M+H⁺)⁺.

### EXAMPLE 177B

### 8-benzyl-3-chloro-5H-pyridazino[3,4-d][1,3]oxazine-5,7(8H)-dione

The title compound is prepared according to the procedure of Example 108C substituting the product of Example 177A for the product of Example 108B.

### EXAMPLE 177C

### 8-benzyl-3-chloro-6-(1,1-dioxido-4H-1,2,4-benzothiadiazin-3-yl)-5-hydroxypyrido[2,3-c]pyridazin-7(8H)-one

The title compound is prepared according to the procedure of Example 1D substituting the product of Example 177B for the product of Example 1B.

### EXAMPLE 178

### 8-benzyl-6-(1,1-dioxido-4H-1,2,4-benzothiadiazin-3-yl)-5-hydroxy-3-(methylthio)pyrido[2,3-c]pyridazin-7(8H)-one

The product of Example 177 is reacted with methanethiol in toluene at elevated temperatures the reaction was concentrated give the title compound.

### EXAMPLE 179

### 8-benzyl-6-(1,1-dioxido-4H-1,2,4-benzothiadiazin-3-yl)-5-hydroxypyrido[2,3-c]pyridazin-7(8H)-one

The title compound is produced by the procedure of Example 109 substituting the product of Example 178 for the product of Example 108D.

### EXAMPLE 180

### 1-benzyl-3-(1,1-dioxido-4H-1,2,4-benzothiadiazin-3-yl)-4-hydroxy-1,6-naphthyridin-2(1H)-one

### EXAMPLE 180A

### methyl 4-(benzylamino)nicotinate

The title compound is prepared from 3-carbomethoxy-4-chloropyridine and benzylamine according to the procedure of Winn, et. al. as described in J. Med. Chem., 36, 1993, 2676-2688.

### EXAMPLE 180B

### 1-benzyl-2H-pyrido[4,3-d]]1,3]oxazine-2,4(1H)-dione

The title compound is prepared according to the procedure of Example 3B substituting the product of Example 180A for the product of Example 3A.

### EXAMPLE 180C

### 1-benzyl-3-(1,1-dioxido-4H-1,2,4-benzothiadiazin-3-yl)-4-hydroxy-1,6-naphthyridin-2(1H)-one

The title compound is prepared according to the procedure of Example 1D substituting the product of Example 180B for the product of Example 1B.

### EXAMPLE 181

### 1-benzyl-3-(1,1-dioxido-4H-1,2,4-benzothiadiazin-3-yl)-4-hydroxy-1,7-naphthyridin-2(1H)-one

### EXAMPLE 181A

### 2H-pyrido[3,4-d][1,3]oxazine-2,4(1H)-dione

The title compound is prepared according to the procedure of Example 110A from 3-aminoisonicotinic acid.

### EXAMPLE 181B

### 1-benzyl-2H-pyrido[3,4-d][1,3]oxazine-2,4(1H)-dione

The title compound is prepared according to the procedure of Example 1B substituting the product of Example 181A for the product of Example 1A, substituting DMF for DMA, and substituting benzyl bromide for n-butyl bromide, respectively.

### EXAMPLE 181C

### 1-benzyl-3-(1,1-dioxido-4H-1,2,4-benzothiadiazin-3-yl)-4-hydroxy-1,7-naphthyridin-2(1H)-one

The title compound is prepared according to the procedure of Example 1D substituting the product of Example 181C for the product of Example 1B.

### EXAMPLE 182

### 1-(benzylamino)-3-(1,1-dioxido-4H-1,2,4-benzothiadiazin-3-yl)-4-hydroxyquinolin-2(1H)-one

A slurry of the product of Example 162 (0.133 g, 0.3 mmol) and 10% palladium on carbon (0.02 g, catalytic amount) in THF (25 mL) was hydrogenated under 1 atmosphere of hydrogen for 4 hours, filtered through Celite and the filtrate was concentrated. The residue was slurried in 1 mL DMSO/5 mL MeOH for 15 minutes and the solid was collected by filtration and dried under vacuum to give the title compound (0.08 g, 60%). MS (ESI−) m/z 445 (M−H)⁻. The sodium salt of the title compound was prepared according to the procedure of Example 1D. ¹H NMR (300 MHz, DMSO-d₆) δ 3.93 (s, 2H) 6.09 (t, J=6.99 Hz, 1H) 7.09 (t, J=7.35 Hz, 1H) 7.35 (m, 5H) 7.54 (m, 4H) 7.69 (t, J=8.82 Hz, 2H) 8.10 (dd, J=7.91, 1.29 Hz, 1H) 16.28 (s, 1H).

The following additional compounds of the present invention, can be prepared by one skilled in the art using known synthetic methodology or by using synthetic methodology described in the Schemes and Examples contained herein. The additional compounds encompassed by the following tables can be described by taking one core from Table 1, one R¹ substituent from Table 2 (wherein X₁ represents the Core Ring Structure), and when needed Y₁ and/or Y₂ substituent from Table 3.

TABLE 1
Examples of Core Ring Structures

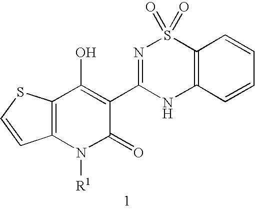

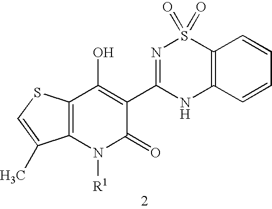

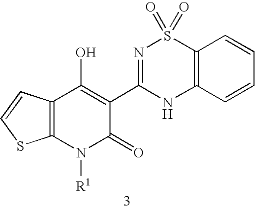

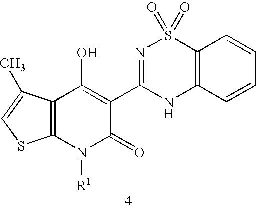

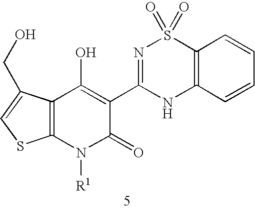

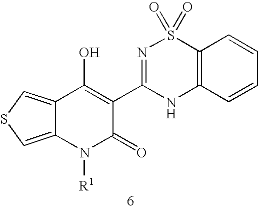

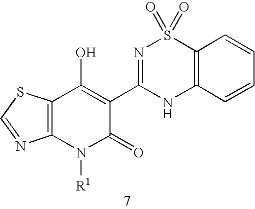

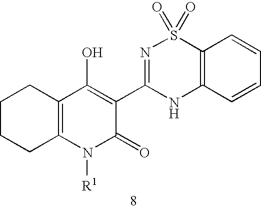

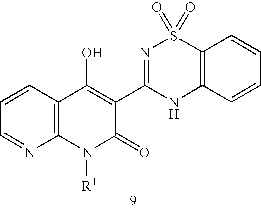

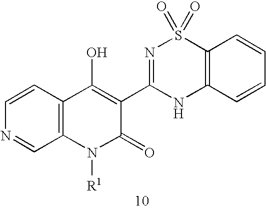

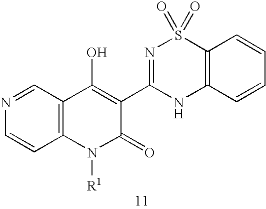

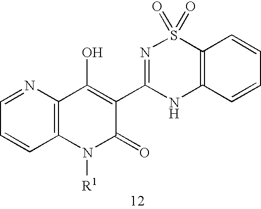

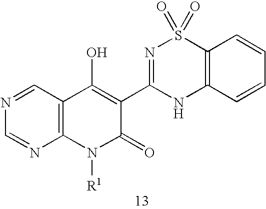

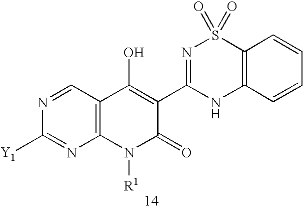

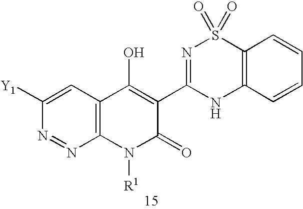

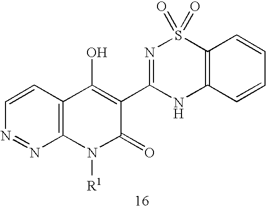

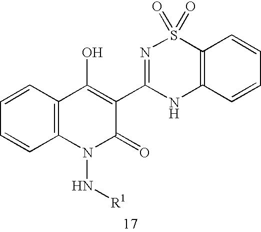

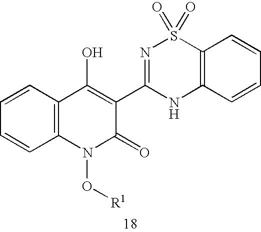

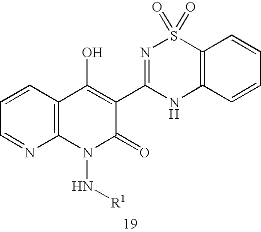

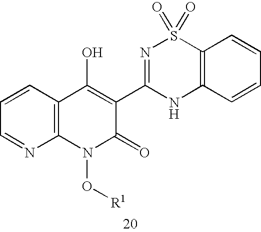

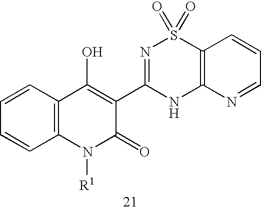

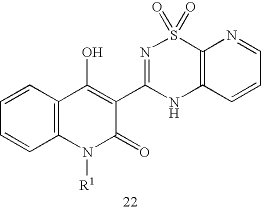

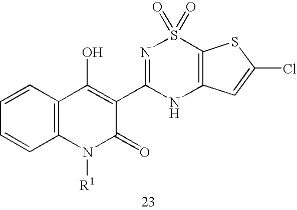

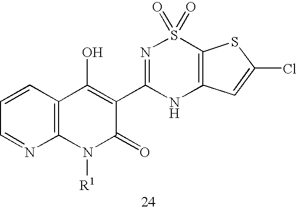

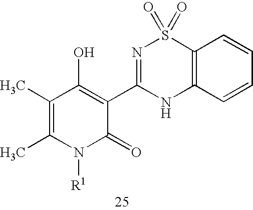

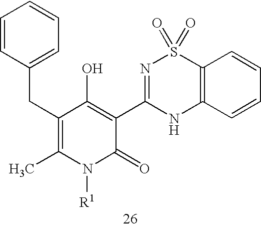

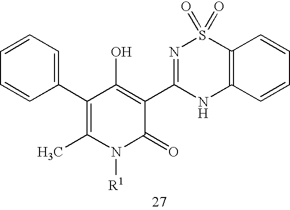

28

TABLE 2
Examples of R¹ Substituents
X₁—H
1
X₁—CH₃
2

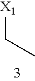

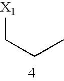

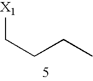

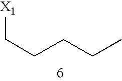

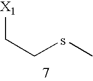

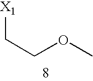

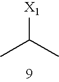

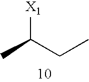

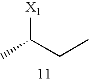

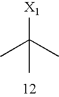

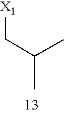

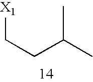

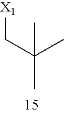

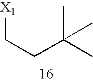

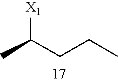

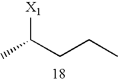

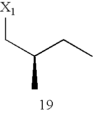

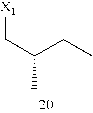

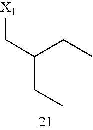

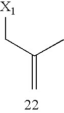

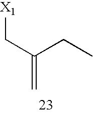

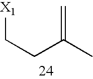

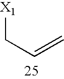

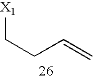

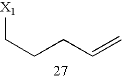

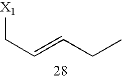

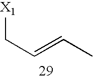

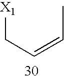

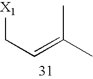

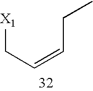

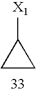

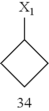

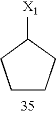

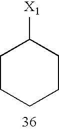

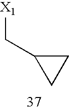

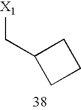

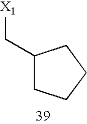

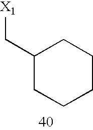

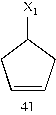

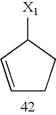

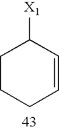

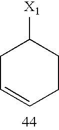

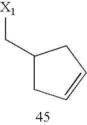

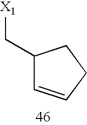

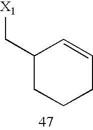

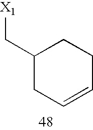

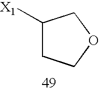

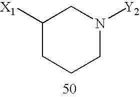

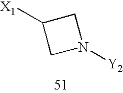

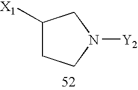

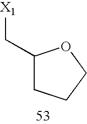

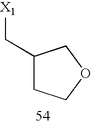

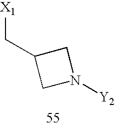

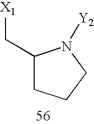

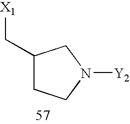

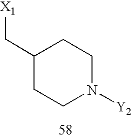

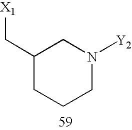

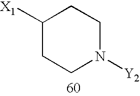

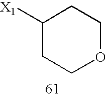

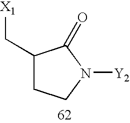

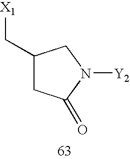

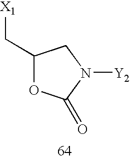

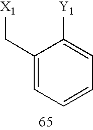

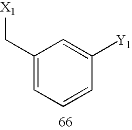

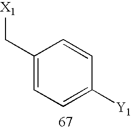

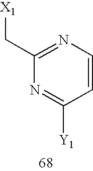

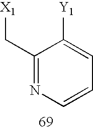

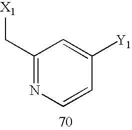

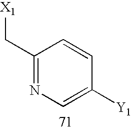

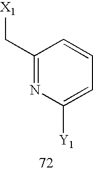

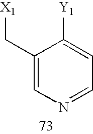

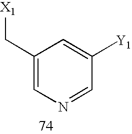

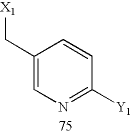

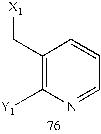

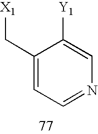

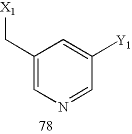

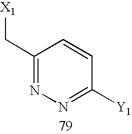

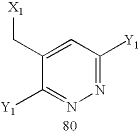

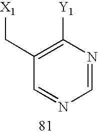

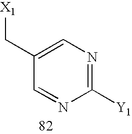

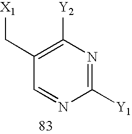

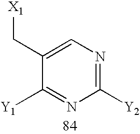

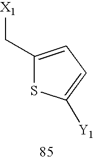

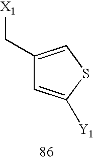

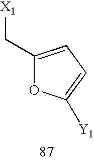

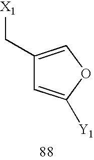

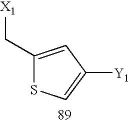

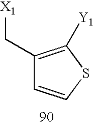

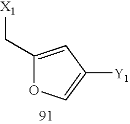

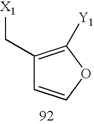

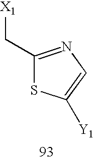

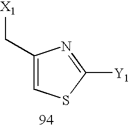

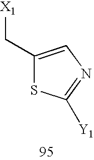

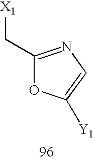

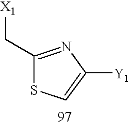

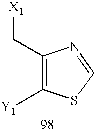

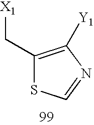

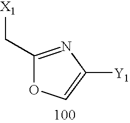

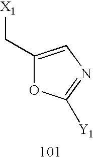

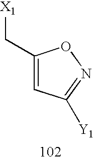

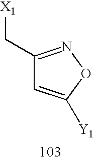

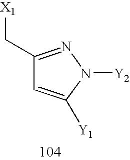

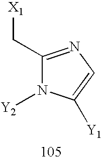

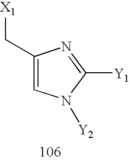

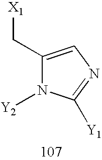

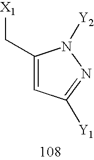

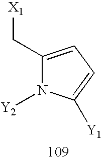

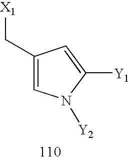

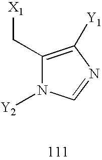

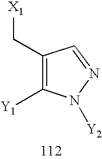

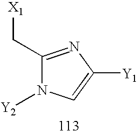

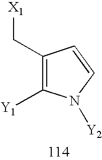

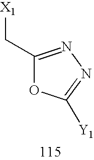

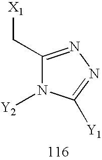

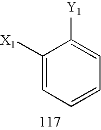

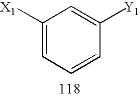

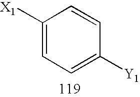

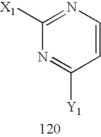

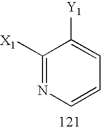

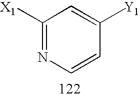

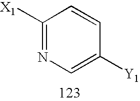

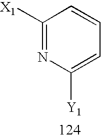

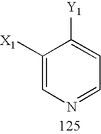

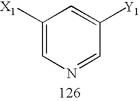

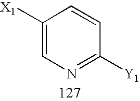

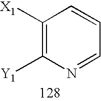

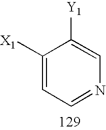

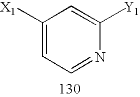

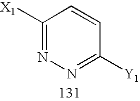

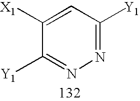

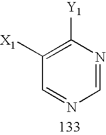

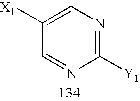

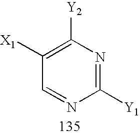

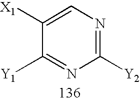

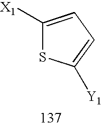

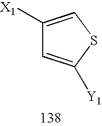

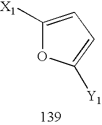

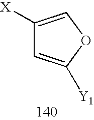

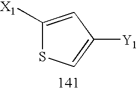

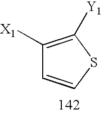

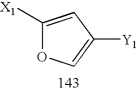

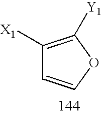

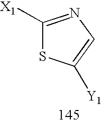

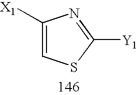

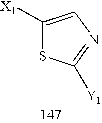

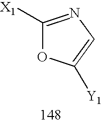

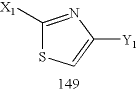

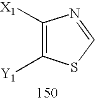

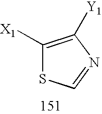

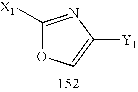

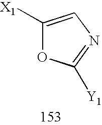

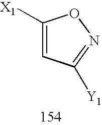

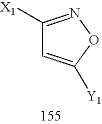

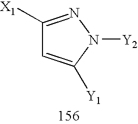

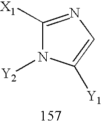

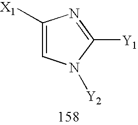

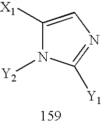

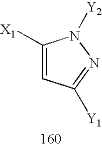

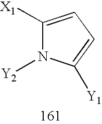

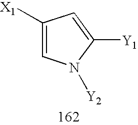

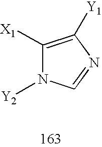

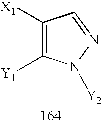

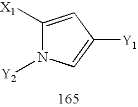

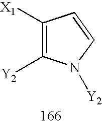

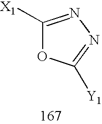

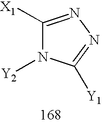

TABLE 3
Substituents of Y₁ and Y₂

| | | | |
|---|---|---|---|
| H | CH₃ | —CH₂CH₃ | —CH(CH₃)₂ |
| —F | —Cl | —Br | NO₂ |
| —CN | —OCH₃ | —NHCH₃ | —N(CH₃)₂ |

Y₂

| | | | |
|---|---|---|---|
| H | CH₃ | —CH₂CH₃ | —CH(CH₃)₂ |
| —COCH₃ | —CO₂CH₃ | | |

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of formula (I) 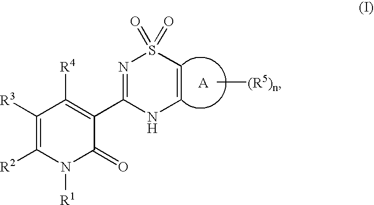
or a therapeutically acceptable salt thereof, wherein
n is 0, 1, 2, 3, or 4;
A is a five- or six-membered ring selected from the group consisting of aryl, cycloalkyl, cycloalkenyl, heteroaryl and heterocycle; wherein the five- or six-membered ring is optionally fused to a second five, or six-membered ring selected from the group consisting of aryl, cycloalkyl, heteroaryl and heterocycle;
R¹ is selected from the group consisting of hydrogen, alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, aryl, arylalkenyl, arylalkyl, arylsulfanylalkyl, arylsulfonylalkyl, carboxyalkyl, cyanoalkyl, cycloalkenyl, cycloalkenylalkyl, cycloalkyl, cycloalkylalkenyl, cycloalkylalkyl, formylalkyl, haloalkoxyalkyl, haloalkyl, heteroaryl, heteroarylalkenyl, heteroarylalkyl, heteroarylsulfonylalkyl, heterocycle, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— and RₖO—;
R² and R³ are independently selected from the group consisting of hydrogen, alkenyl, alkoxyalkyl, alkoxycarbonyl, alkyl, alkylcarbonyl, aryl, arylalkyl, arylcarbonyl, heteroaryl, heteroarylalkyl, heteroarylcarbonyl, heterocyclecarbonyl, cyano, halo and RₐR_{b}NC(O)—; or
R² and R³, together with the carbon atoms to which they are attached form a ring selected from the group consisting of aryl, cycloalkyl, heteroaryl and heterocycle;
R⁴ is selected from the group consisting of alkoxy, arylalkoxy, aryloxy, halo, hydroxy, RₐR_{b}N—, N₃—, R_{c}S—;
each R⁵ is independently selected from the group consisting of alkenyl, alkoxy, alkoxyalkyl, alkoxycarbonyl, alkyl, alkylcarbonyl, alkylsulfonyl, alkynyl, aryl, arylalkyl, arylcarbonyl, aryloxy, aryloxyalkyl, arylalkoxy, arylsulfonyl, halo, haloalkyl, heteroaryl, heteroarylalkyl, heteroarylcarbonyl, heterocycle, heterocyclealkyl, heterocyclecarbonyl, hydroxy, hydoxyalkyl, cycloalkyl, cyano, nitro, RₐR_{b}N—, RₐR_{b}Nalkyl and RₐR_{b}NC(O)—;
Rₐ and R_{b} are independently selected from the group consisting of hydrogen, alkenyl, alkoxyalkyl, alkoxycarbonyl, alkoxycarbonylalkyl, alkyl, alkylcarbonyl, alkylcarbonylalkyl, alkylsulfonyl, alkylsulfonylalkyl, aryl, arylalkenyl, arylalkyl, arylcarbonyl, arylsulfonyl, arylsulfonylalkyl, cyanoalkyl, cycloalkenyl, cycloalkenylalkyl, cycloalkyl, cycloalkylalkyl, cycloalkylalkenyl, formylalkyl, haloalkyl, heteroaryl, heteroarylalkenyl, heteroarylalkyl, heteroarylcarbonyl, heteroarylsulfonylalkyl, heterocycle, heterocyclealkenyl, heterocyclealkyl, heterocyclecarbonyl, heteroarylsulfonyl, hydroxyalkyl, nitroalkyl, R_{c}R_{d}N-, R_{c}R_{d}Nalkyl-, R_{c}R_{d}NalkylC(O)—, R_{c}R_{d}NC(O)Oalkyl-, R_{c}R_{d}NC(O)N(Rₑ)alkyl-; wherein the aryl, the aryl part of the arylalkenyl, the aryl part of the arylalkyl, the aryl part of the arylsulfanylalkyl, the aryl part of the arylsulfonylalkyl, and the aryl part of the arylsulfonyl can be substituted with 0, 1, 2, 3 or 4 substituents independently selected from the group consisting of alkoxy, alkyl, alkylcarbonyl, cyano, halo, hydroxy, nitro, R_{c}OC(O)N(Rₑ)- and R_{c}R_{d}NC(O)—; wherein the heteroaryl, the heteroaryl part of the heteroarylalkyl, the heteroaryl part of the heteroarylsulfonylalkyl, the heteroaryl part of the heteroarylalkenyl, and the heteroaryl part of the heteroarylsulfonyl can be substituted with 0, 1, 2, 3 or 4 substituents independently selected from the group consisting of alkoxy, alkyl, alkylcarbonyl, cyano, halo, hydroxy, nitro, R_{c}OC(O)N(Rₑ)- and R_{c}R_{d}NC(O)—; the heterocycle, the heterocycle part of the heterocyclealkyl, the heterocyclealkenyl can be substituted with 0, 1, 2, 3 or 4 substituents independently selected from the group consisting of alkoxy, alkyl, alkylcarbonyl, cyano, halo, hydroxy, nitro, R_{c}OC(O)N(Rₑ)- and R_{c}R_{d}NC(O)—;
R_{c}, R_{d}, and Rₑ are independently selected from the group consisting of hydrogen, alkenyl, alkyl and cycloalkyl;
R_{f} and R_{g} are independently selected from the group consisting of hydrogen, alkyl, alkenyl, aryl, arylalkyl, cycloalkyl, cycloalkylalkyl, cycloalkenyl, heterocycle, heterocyclealkyl, heteroaryl, and heteroarylalkyl; or
R_{f} and R_{g} together with the carbon atom to which they are attached form a ring selected from the group consisting of cycloalkyl, cycloalkenyl and heterocycle;
Rₖ is selected from the group consisting of alkenyl, alkoxyalkyl, alkoxycarbonyl, alkoxycarbonylalkyl, alkyl, alkylcarbonylalkyl, alkylsulfanylalkyl, alkylsulfonylalkyl, aryl, arylalkyl, arylsulfanylalkyl, carboxyalkyl, cyanoalkyl, cycloalkenyl, cycloalkenylalkyl, cycloalkyl, cycloalkylalkyl, formylalkyl, haloalkoxyalkyl, haloalkyl, heteroaryl, heteroarylalkyl, heterocycle, heterocyclealkyl, hydroxyalkyl, nitroalkyl, R_{c}R_{d}Nalkyl-, R_{c}R_{d}NC(O)— and R_{c}R_{d}NC(O)alkyl;
provided that when R² and R³, together with the carbon atoms to which they are attached, form a phenyl ring, and R¹ is alkyl, alkenyl, arylalkyl, aryl or heteroaryl, and R⁴ is alkoxy, arylalkoxy, aryloxy, hydroxy or R_{c}S—, then A is other than phenyl.

2. The compound of claim 1 wherein
A is phenyl; and
R² and R³, together with the carbon atoms to which they are attached form a heteroaryl ring; or
A is a five- or six-membered heteroaryl ring; and
R² and R³, together with the carbon atoms to which they are attached form an aryl ring; or
A is a five- or six-membered heteroaryl ring; and
R² and R³, together with the carbon atoms to which they are attached form a heteroaryl ring;
R¹ is alkenyl, alkoxyalkyl, alkoxycarbonylalkyl, alkyl, alkylcarbonylalkyl, alkylsulfanylalkyl, alkylsulfinylalkyl, alkylsulfonylalkyl, alkynyl, arylalkenyl, arylalkyl, arylsulfanylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—;
R⁴ is hydroxy, RₐR_{b}N— or R_{c}S—; and wherein n, R⁵, Rₐ, R_{b}, R_{c}, Rₑ, R_{d}, R_{f}, R_{g}, Rₖ are as defined therein.

3. The compound of claim 2 wherein
A is phenyl; and
R² and R³, together with the carbon atoms to which they are attached form a heteroaryl ring; or
A is a five- or six-membered heteroaryl ring; and
R² and R³, together with the carbon atoms to which they are attached form an aryl ring; or
A is a five- or six-membered heteroaryl ring; and
R² and R³, together with the carbon atoms to which they are attached form a heteroaryl ring;
R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—;
R⁴ is hydroxy, RₐR_{b}N— or R_{c}S—;
and wherein n, R⁵, Rₐ, R_{b}, R_{c}, Rₑ, R_{d}, R_{f}, R_{g}, Rₖ are as defined therein.

4. The compound of claim 3 wherein
A is phenyl; and
R² and R³, together with the carbon atoms to which they are attached form a heteroaryl ring; or
A is a five- or six-membered heteroaryl ring; and
R² and R³, together with the carbon atoms to which they are attached form an aryl ring; or
A is a five- or six-membered heteroaryl ring; and
R² and R³, together with the carbon atoms to which they are attached form a heteroaryl ring;
R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—;
R⁴ is hydroxy or R_{b}NH—, wherein R_{b} is hydrogen, alky, alkylcarbonyl, or alkylsulfonyl, or HS—;
and wherein n, R⁵, Rₐ, R_{c}, Rₑ, R_{d}, R_{f}, R_{g}, Rₖ are as defined therein.

5. The compound of claim 3 wherein
A is phenyl; and
R² and R³, together with the carbon atoms to which they are attached form a heteroaryl ring; or
A is a five- or six-membered heteroaryl ring; and
R² and R³, together with the carbon atoms to which they are attached form an aryl ring; or
A is a five- or six-membered heteroaryl ring; and
R² and R³, together with the carbon atoms to which they are attached form a heteroaryl ring;
R¹ is alkenyl, alkoxyalkyl, alkoxycarbonylalkyl, alkyl, alkylcarbonylalkyl, alkylsulfonylalkyl, alkynyl, arylalkenyl, arylalkyl, arylsulfonylalkyl, carboxyalkyl, cyanoalkyl, cycloalkenylalkyl, cycloalkylalkenyl, cycloalkylalkyl, formylalkyl, haloalkoxyalkyl, haloalkyl, heteroarylalkenyl, heteroarylalkyl, heteroarylsulfonylalkyl, heterocyclealkenyl, heterocyclealkyl, hydroxyalkyl, nitroalkyl, RₐR_{b}N—, RₐR_{b}Nalkyl-, RₐR_{b}NC(O)alkyl-, RₐR_{b}NC(O)Oalkyl-, RₐR_{b}NC(O)NR_{c}alkyl-, R_{f}R_{g}C═N— or RₖO—;
R⁴ is hydroxy, R_{b}NH—, wherein R_{b} is hydrogen, alkyl,
and wherein n, R⁵, Rₐ, R_{c}, Rₑ, R_{d}, R_{f}, R_{g}, Rₖ are as defined therein.

6. The compound of claim 1 wherein
A is phenyl; and
R² and R³, together with the carbon atoms to which they are attached form an aryl ring;
R¹ is RₐR_{b}N—, R_{f}R_{g}C═N— or RₖO—;
R⁴ is halo, alkoxy, aryloxy, hydroxy, RₐR_{b}N— or R_{c}S—;
and wherein n, R⁵, Rₐ, R_{b}, Rₑ, Rₑ, R_{d}, R_{f}, R_{g}, Rₖ are as defined therein.

7. The compound of claim 6 wherein
A is phenyl; and
R² and R³, together with the carbon atoms to which they are attached form an aryl ring;
R¹ is RₐR_{b}N—, R_{f}R_{g}C═N— or RₖO—;
R⁴ is hydroxy, RₐR_{b}N— or R_{c}S—;
and wherein n, R⁵, Rₐ, R_{b}, R_{c}, Rₑ, R_{d}, R_{f}, R_{g}, Rₖ are as defined therein.

8. The compound of claim 6 wherein
A is phenyl; and
R² and R³, together with the carbon atoms to which they are attached form an aryl ring;
R¹ is RₐR_{b}N—, R_{f}R_{g}C═N— or RₖO—;
R⁴ is hydroxy, R_{b}NH—, wherein R_{b} is hydrogen, alkyl, alkylcarbonyl, or alkylsulfonyl, or HS—;
and wherein n, R⁵, Rₐ, Rₑ, Rₑ, R_{d}, R_{f}, R_{g}, Rₖ are as defined therein.

9. The compound of claim 6 wherein
A is phenyl; and
R² and R³, together with the carbon atoms to which they are attached form an aryl ring;
R¹ is RₐR_{b}N—, R_{f}R_{g}C═N— or RₖO—;
R⁴ is hydroxy or R_{b}NH—, wherein R_{b} is selected from the group consisting of hydrogen, alkyl,
and wherein n, R⁵, Rₐ, R_{c}, Rₑ, R_{d}, R_{f}, R_{g}, Rₖ are as defined therein.

10. The compound according to claim 1, selected from the group consisting of
1-butyl-3-(1,1-dioxido-4H-1,2,4-benzothiadiazin-3-yl)-4-hydroxy-1,8-naphthyridin-2(1H)-one;
1-[(5-chloro-2-thienyl)methyl]-3-(1,1-dioxido-4H-1,2,4-benzothiadiazin-3-yl)-4-hydroxy-1,8-naphthyridin-2(1H)-one; 3-(1,1-dioxido-4H-1,2,4-benzothiadiazin-3-yl)-1-(2-ethylbutyl)-4-hydroxy-1,8-naphthyridin-2(1H)-one;
1-[(5-bromo-2-thienyl)methyl]-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-(3-methylbenzyl)-1,8-naphthyridin-2(1H)-one;
3-(1,1-dioxido-4H-1,2,4-benzothiadiazin-3-yl)-4-hydroxy-1-(3-nitrobenzyl)-1,8-naphthyridin-2(1H)-one;
3-(1,1-dioxido-4H-1,2,4-benzothiadiazin-3-yl)-4-hydroxy-1-(3-thienylmethyl)-1,8-naphthyridin-2(1H)-one;
1-(3-chlorobenzyl)-3-(1,1-dioxido-4H-1,2,4-benzothiadiazin-3-yl)-4-hydroxy-1,8-naphthyridin-2(1H)-one;
1-(3-bromobenzyl)-3-(1,1-dioxido-4H-1,2,4-benzothiadiazin-3-yl)-4-hydroxy-1,8-naphthyridin-2(1H)-one;
1-[(2-chloro-1,3-thiazol-5-yl)methyl]-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-1-(3-fluorobenzyl)-4-hydroxy-1,8-naphthyridin-2(1H)-one;
3-(1,1-dioxido-4H-1,2,4-benzothiadiazin-3-yl)-4-hydroxy-1-(3-methylbutyl)-1,8-naphthyridin-2(1H)-one;
1-(cyclobutylmethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[(5-methyl-2-thienyl)methyl]-1,8-naphthyridin-2(1H)-one;
1-benzyl-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[(5-methyl-3-pyridinyl)methyl]-1,8-naphthyridin-2(1H)-one;
1-[(2-chloro-4-pyridinyl)methyl]-3-(1,1-dioxido-4H-1,2,4-benzothiadiazin-3-yl)-4-hydroxy-1,8-naphthyridin-2(1H)-one; 1-[(5-bromo-3-pyridinyl)methyl]-3-(1,1-dioxido-4H-1,2,4-benzothiadiazin-3-yl)-4-hydroxy-1,8-naphthyridin-2(1H)-one; 1-(cyclohexylmethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[(2S)-2-methylbutyl]-1,8-naphthyridin-2(1H)-one;
3-(1,1-dioxido-4H-1,2,4-benzothiadiazin-3-yl)-4-hydroxy-1-(4-methylbenzyl)-1,8-naphthyridin-2(1H)-one;
3-(1,1-dioxido-4H-1,2,4-benzothiadiazin-3-yl)-4-hydroxy-1-[(5-nitro-2-furyl)methyl]-1,8-naphthyridin-2(1H)-one;
1-(1-benzothien-2-ylmethyl)-3-(1,1-dioxido-4H-1,2,4-benzothiadiazin-3-yl)-4-hydroxy-1,8-naphthyridin-2(1H)-one; 3-(1,1-dioxido-4H-1,2,4-benzothiadiazin-3-yl)-4-hydroxy-1-(3-methoxybenzyl)-1,8-naphthynrdin-2(1H)-one;
3-(1,1-dioxido-4H-1,2,4-benzothiadiazin-3-yl)-4-hydroxy-1-(3-iodobenzyl)-1,8-naphthyridin-2(1H)-one; 1-[(3,5-dimethyl-4-isoxazolyl)methyl]-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[2-(3-thienyl)ethyl]-1,8-naphthyridin-2(1H)-one;
3-(1,1-dioxido-4H-1,2,4-benzothiadiazin-3-yl)-4-hydroxy-1-(4-pyridinylmethyl)-1,8-naphthyridin-2(1H)-one;
1-(4-bromobenzyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-neopentyl-1,8-naphthyridin-2(1H)-one;
1-{[(1S,2R,5S)-6,6-dimethylbicyclo[3.1.1]hept-2-yl]methyl}-3-(1,1-dioxido-4H-1,2,4-benzothiadiazin-3-yl)-4-hydroxy-1,8-naphthyridin-2(1H)-one;
3-{[3-(1,1-dioxido-4H-1,2,4-benzothiadiazin-3-yl)-4-hydroxy-2-oxo-1,8-naphthyridin-1 (2H)-yl]methyl}benzonitrile;
3-(1,1-dioxido-4H-1,2,4-benzothiadiazin-3-yl)-4-hydroxy-1-(3-pyridinylmethyl)-1,8-naphthyridin-2(1H)-one;
1-(1-adamantylmethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[3-(trifluoromethyl)benzyl]-1,8-naphthyridin-2(1H)-one;
3-(1,1-dioxido-4H-1,2,4-benzothiadiazin-3-yl)-4-hydroxy-1-[(2-methyl-1,3-thiazol-5-yl)methyl]-1,8-naphthyridin-2(1H)-one;
1-(2-cyclohexylethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-(4-methoxybenzyl)-1,8-naphthyridin-2(1H)-one;
3-(1,1-dioxido-4H-1,2,4-benzothiadiazin-3-yl)-4-hydroxy-1-(2-methylbenzyl)-1,8-naphthyridin-2(1H)-one;
1-(cyclopropylmethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-(1,3-thiazol-4-ylmethyl)-1,8-naphthyridin-2(1H)-one;
3-(1,1-dioxido-4H-1,2,4-benzothiadiazin-3-yl)-4-hydroxy-1-[(5-phenyl-2-thienyl)methyl]-1,8-naphthyridin-2(1H)-one;
3-(1,1-dioxido-4H-1,2,4-benzothiadiazin-3-yl)-4-hydroxy-1-(4-methyl-3-pentenyl)-1,8-naphthyridin-2(1H)-one;
4-{[3-(1,1-dioxido-4H-1,2,4-benzothiadiazin-3-yl)-4-hydroxy-2-oxo-1,8-naphthyridin-1 (2H)-yl]methyl}benzonitrile;
1-[2-(1-cyclohexen-1-yl)ethyl]-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[(2-methyl-1,3-thiazol-4-yl)methyl]-1,8-naphthyridin-2(1H)-one;
2-{[3-(1,1-dioxido-4H-1,2,4-benzothiadiazin-3-yl)-4-hydroxy-2-oxo-1,8-naphthyridin-1 (2H)-yl]methyl}benzonitrile;
3-(11,1-dioxido-4H-1,2,4-benzothiadiazin-3-yl)-4-hydroxy-1-[(5-methyl-3-isoxazolyl)methyl]-1,8-naphthyridin-2(1H)-one;
3-(1,1-dioxido-4H-1,2,4-benzothiadiazin-3-yl)-4-hydroxy-1-(1-naphthylmethyl)-1,8-naphthyridin-2(1H)-one;
3-(1,1-dioxido-4H-1,2,4-benzothiadiazin-3-yl)-4-hydroxy-1-(2-pyridinylmethyl)-1,8-naphthyridin-2(1H)-one;
1-(4-tert-butylbenzyl)-3-(1,1-dioxido-4H-1,2,4-benzothiadiazin-3-yl)-4-hydroxy-1,8-naphthyridin-2(1H)-one;
ethyl [3-(1,1-dioxido-4H-1,2,4-benzothiadiazin-3-yl)-4-hydroxy-2-oxo-1,8-naphthyridin-1 (2H)-yl]acetate;
[3-(1,1-dioxido-4H-1,2,4-benzothiadiazin-3-yl)-4-hydroxy-2-oxo-1,8-naphthyridin-1(2H)-yl]acetic acid;
3-(1,1-dioxido-4H-1,2,4-benzothiadiazin-3-yl)-4-hydroxy-1-(3-phenoxybenzyl)-1,8-naphthyridin-2(1H)-one;
1-allyl-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-(2-naphthylmethyl)-1,8-naphthyridin-2(1H)-one;
3-(1,1-dioxido-4H-1,2,4-benzothiadiazin-3-yl)-4-hydroxy-1-[(1R)-1-phenylethyl]-1,8-naphthyridin-2(1H)-one;
1-[(5-tert-butyl-2-thienyl)methyl]-3-(1,1-dioxido-4H-1,2,4-benzothiadiazin-3-yl)-4-hydroxy-1,8-naphthyridin-2(1H)-one;
1-(1,1 ′-biphenyl-4-ylmethyl)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[2-(1H-indol-3-yl)ethyl]-1,8-naphthyridin-2(1H)-one;
3-(1,1-dioxido-4H-1,2,4-benzothiadiazin-3-yl)-1-[(6-ethoxy-2-pyridinyl)methyl]-4-hydroxy-1,8-naphthyridin-2(1H)-one;
1-benzyl-3-(1,1-dioxido-4H-1,2,4-benzothiadiazin-3-yl)-4-hydroxy-7-methyl-1,8-naphthyridin-2(1H)-one;
3-(1,1-dioxido-4H-1,2,4-benzothiadiazin-3-yl)-4-hydroxy-1-[(6-methyl-2-pyridinyl)methyl]-1,8-naphthyridin-2(1H)-one;
3-(1,1-dioxido-4H-1,2,4-benzothiadiazin-3-yl)-1-(1-ethylpropyl)-4-hydroxy-1,8-naphthyridin-2(1H)-one;
3-(1,1-dioxido-4H-1,2,4-benzothiadiazin-3-yl)-4-hydroxy-1-[(1S)-1-phenylethyl]-1,8-naphthyridin-2(1H)-one;
2-{2-[3-(1,1-dioxido-4H-1,2,4-benzothiadiazin-3-yl)-4-hydroxy-2-oxo-1,8-naphthyridin-1 (2H)-yl]ethyl}-1H-isoindole-1,3(2H)-dione;
3-(1,1-dioxido-4H-1,2,4-benzothiadiazin-3-yl)-4-hydroxy-1-(3-hydroxypropyl)-1,8-naphthyridin-2(1H)-one;
1-cyclopentyl-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-1-[2-(1,3-dioxolan-2-yl)ethyl]-4-hydroxy-1,8-naphthyridin-2(1H)-one;
1-(2,3-dihydroxypropyl)-3-(1,1-dioxido-4H-1,2,4-benzothiadiazin-3-yl)-4-hydroxy-1,8-naphthyridin-2(1H)-one;
1-cycloheptyl-3-(1,1-dioxido-4H-1,2,4-benzothiadiazin-3-yl)-4-hydroxy-1,8-naphthyridin-2(1H)-one;
1-(3-anilinopropyl)-3-(1,1-dioxido-4H-1,2,4-benzothiadiazin-3-yl)-4-hydroxy-1,8-naphthyridin-2(1H)-one;
3-[3-(1,1-dioxido-4H-1,2,4-benzothiadiazin-3-yl)-4-hydroxy-2-oxo-1,8-naphthyridin-1(2H)-yl]propanal;
methyl 4-{[3-(1,1-dioxido-4H-1,2,4-benzothiadiazin-3-yl)-4-hydroxy-2-oxo-1,8-naphthyridin-1 (2H)-yl]methyl} benzoate;
ethyl 5-{[3-(1,1-dioxido-4H-1,2,4-benzothiadiazin-3-yl)-4-hydroxy-2-oxo-1,8-naphthyridin-1(2H)-yl]methyl}-2-furoate;
1-[3-(dimethylamino)propyl]-3-(1,1-dioxido-4H-1,2,4-benzothiadiazin-3-yl)-4-hydroxy-1,8-naphthyridin-2(1H)-one;
1-{3-[[2-(dimethylamino)ethyl](methyl)amino]propyl}-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[3-(4-methyl-1-piperazinyl)propyl]-1,8-naphthyridin-2(1H)-one;
1-(2-aminoethyl)-3-(1,1-dioxido-4H-1,2,4-benzothiadiazin-3-yl)-4-hydroxy-1,8-naphthyridin-2(1H)-one;
1-[3-(diethylamino)propyl]-3-(1,1-dioxido-4H-1,2,4-benzothiadiazin-3-yl)-4-hydroxy-1,8-naphthyridin-2(1H)-one;
1-cyclohexyl-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-[3-(4-morpholinyl)propyl]-1,8-naphthyridin-2(1H)-one;
5-{[3-(1,1-dioxido-4H-1,2,4-benzothiadiazin-3-yl)-4-hydroxy-2-oxo-1,8-naphthyridin-1 (2H)-yl]methyl}-2-furoic acid;
1-benzyl-3-(7-bromo-1,1-dioxido-4H-1,2,4-benzothiadiazin-3-yl)-4-hydroxy-1,8-naphthyridin-2(1H)-one;
1-benzyl-3-(1,1-dioxido-7-phenyl-4H-1,2,4-benzothiadiazin-3-yl)-4-hydroxy-1,8-naphthyridin-2(1H)-one;
1-benzyl-3-(7-cyclohexyl-1,1-dioxido-4H-1,2,4-benzothiadiazin-3-yl)-4-hydroxy-1,8-naphthyridin-2(1H)-one;
1-benzyl-3-(7-tert-butyl-1,1-dioxido-4H-1,2,4-benzothiadiazin-3-yl)-4-hydroxy-1,8-naphthyridin-2(1H)-one;
1-benzyl-4-hydroxy-3-(7-methyl-1,1-dioxido-4H-1,2,4-benzothiadiazin-3-yl)-1,8-naphthyridin-2(1H)-one;
1-butyl-3-(6-chloro-1,1-dioxido-4H-1,2,4-benzothiadiazin-3-yl)-4-hydroxy-1,8-naphthyridin-2(1H)-one;
1-benzyl-3-(8-bromo-5-methyl-1,1-dioxido-4H-1,2,4-benzothiadiazin-3-yl)-4-hydroxy-1,8-naphthyridin-2(1H)-one;
1-benzyl-3-(8-fluoro-5-methyl-1,1-dioxido-4H-1,2,4-benzothiadiazin-3-yl)-4-hydroxy-1,8-naphthyridin-2(1H)-one;
1-benzyl-4-hydroxy-3-(5-isopropyl-1,1-dioxido-4H-1,2,4-benzothiadiazin-3-yl)-1,8-naphthyridin-2(1H)-one;
1-benzyl-4-hydroxy-3-(5-methyl-1,1-dioxido-4H-1,2,4-benzothiadiazin-3-yl)-1,8-naphthyridin-2(1H)-one;
1-benzyl-3-(5-bromo-1,1-dioxido-4H-1,2,4-benzothiadiazin-3-yl)-4-hydroxy-1,8-naphthyridin-2(1H)-one;
1-benzyl-3-(1,1-dioxido-5-propyl-4H-1,2,4-benzothiadiazin-3-yl)-4-hydroxy-1,8-naphthyridin-2(1H)-one;
1-benzyl-3-(5-ethyl-1,1-dioxido-4H-1,2,4-benzothiadiazin-3-yl)-4-hydroxy-1,8-naphthyridin-2(1H)-one;
3-(1-benzyl-4-hydroxy-2-oxo-1,2-dihydro-1,8-naphthyridin-3-yl)-4H-1,2,4-benzothiadiazine-5-carbonitrile 1,1-dioxide;
1-butyl-3-(1,1-dioxido-4H-pyrido[3,2-e][1,2,4]thiadiazin-3-yl)-4-hydroxy-2(1H)-quinolinone;
1-benzyl-3-(1,1-dioxido-4H-pyrido[3,2-e][1,2,4]thiadiazin-3-yl)-4-hydroxy-2(1H)-quinolinone;
1-benzyl-3-(1,1-dioxido-4H-pyrido[3,2-e][1,2,4]thiadiazin-3-yl)-4-hydroxy-1,8-naphthyridin-2(1H)-one;
5-chloro-3-(1,1-dioxido-4H-pyrido[3,2-e][1,2,4]thiadiazin-3-yl)-4-hydroxy-1-(3-methylbutyl)-2(1H)-quinolinone;
1-benzyl-3-(1,1-dioxido-4H-pyrido[3,2-e][1,2,4]thiadiazin-3-yl)-4-hydroxy-5-methyl-2(1H)-quinolinone;
3-(1,1-dioxido-4H-pyrido[3,2-e][1,2,4]thiadiazin-3-yl)-4-hydroxy-1-[(2-methyl-1,3-thiazol-5-yl)methyl]-2(1H)-quinolinone;
1-benzyl-4-hydroxy-3-(7-methyl-1,1-dioxido-4H-pyrido[2,3-e][1,2,4]thiadiazin-3-yl)-2(1H)-quinolinone;
1-butyl-4-hydroxy-3-(7-methyl-1,1-dioxido-4H-pyrido[2,3-e][1,2,4]thiadiazin-3-yl)-1,8-naphthyridin-2(1H)-one;
3-(1,1-dioxido-4H-1,2,4-benzothiadiazin-3-yl)-4-hydroxy-1-(2-thienylmethyl)-1,8-naphthyridin-2(1H)-one;
1-(benzyloxy)-3-(1,1-dioxido-4H-1,2,4-benzothiadiazin-3-yl)-4-hydroxy-1,8-naphthyridin-2(1H)-one;
6-(11,1-dioxido-4H-1,2,4-benzothiadiazin-3-yl)-8-(2-ethylbutyl)-5-hydroxy-2-(methylsulfanyl)pyrido[2,3-d]pyrimidin-7(8H)-one;
6-(11,1-dioxido-4H-1,2,4-benzothiadiazin-3-yl)-8-(2-ethylbutyl)-5-hydroxypyrido[2,3-d]pyrimidin-7(8H)-one;
4-benzyl-6-(1,1-dioxido-4H-1,2,4-benzothiadiazin-3-yl)-7-hydroxythieno[3,2-b]pyridin-5(4H)-one;
4-butyl-6-(1,1-dioxido-4H-1,2,4-benzothiadiazin-3-yl)-7-hydroxythieno[3,2-b]pyridin-5(4H)-one;
6-(11,1-didxido-4H-1,2,4-benzothiadiazin-3-yl)-7-hydroxy-4-(4-pyridinylmethyl)thieno[3,2-b]pyridin-5(4H)-one;
1-(3-bromobenzyl)-3-(1,1-dioxido-4H-1,2,4-benzothiadiazin-3-yl)-4-hydroxy-5,6,7,8-tetrahydro-2(1H)-quinolinone;
5-(1,1-dioxido-4H-1,2,4-benzothiadiazin-3-yl)-4-hydroxy-7-(4-pyridinylmethyl)thieno[2,3-b]pyridin-6(7H)-one;
6-(1,1-dioxido-4H-1,2,4-benzothiadiazin-3-yl)-7-hydroxy-4-(3-pyridinylmethyl)thieno[3,2-b]pyridin-5(4H)-one;
7-benzyl-5-(1,1-dioxido-4H-1,2,4-benzothiadiazin-3-yl)-4-hydroxythieno[2,3-b]pyridin-6(7H)-one;
4-(cyclopropylmethyl)-6-(1,1-dioxido-4H-1,2,4-benzothiadiazin-3-yl)-7-hydroxythieno[3,2-b]pyridin-5(4H)-one;
5-(1,1-dioxido-4H-1,2,4-benzothiadiazin-3-yl)-4-hydroxy-7-(3-methylbutyl)thieno[2,3-b]pyridin-6(7H)-one;
4-benzyl-6-(1,1-dioxido-4H-1,2,4-benzothiadiazin-3-yl)-7-hydroxy-2-phenylthieno[3,2-b]pyridin-5(4H)-one;
4-benzyl-6-(1,1-dioxido-4H-1,2,4-benzothiadiazin-3-yl)-7-hydroxy-3-methylthieno[3,2-b]pyridin-5(4H)-one;
1-benzyl-3-(1,1-dioxido-4H-1,2,4-benzothiadiazin-3-yl)-4-hydroxy-5,6,7,8-tetrahydro-2(1H)-quinolinone;
1-benzyl-3-(1,1-dioxido-4H-1,2,4-benzothiadiazin-3-yl)-4-hydroxy-2(1H)-pyridinone;
1-benzyl-3-(1,1-dioxido-4H-1,2,4-benzothiadiazin-3-yl)-4-hydroxy-5,6-dimethyl-2(1H)-pyridinone;
7-benzyl-5-(1,1-dioxido-4H-1,2,4-benzothiadiazin-3-yl)-4-hydroxy-3-methylthieno[2,3-b]pyridin-6(7H)-one;
6-(1,1-dioxido-4H-1,2,4-benzothiadiazin-3-yl)-7-hydroxy-4-(3-methylbutyl)thieno[3,2-b]pyridin-5(4H)-one;
6-(1,1-dioxido-4H-1,2,4-benzothiadiazin-3-yl)-4-(2-ethylbutyl)-7-hydroxythieno[3,2-b]pyridin-5(4H)-one;
1-benzyl-3-(1,1-dioxido-4H-1,2,4-benzothiadiazin-3-yl)-4-hydroxy-6-methyl-5-phenyl-2(1H)-pyridinone;
3-(1,1-dioxido-4H-1,2,4-benzothiadiazin-3-yl)-4-hydroxy-5,6-dimethyl-1-(3-methylbutyl)-2(1H)-pyridinone;
3-(1,1-dioxido-4H-1,2,4-benzothiadiazin-3-yl)-1-(2-ethylbutyl)-4-hydroxy-5,6-dimethyl-2(1H)-pyridinone;
1-benzyl-3-(1,1-dioxido-4H-1,2,4-benzothiadiazin-3-yl)-4-hydroxy-6-phenyl-2(1H)-pyridinone;
5-(1,1-dioxido-4H-1,2,4-benzothiadiazin-3-yl)-4-hydroxy-7-(3-methyl-2-butenyl)thieno[2,3-b]pyridin-6(7H)-one;
1,5-dibenzyl-3-(1,1-dioxido-4H-1,2,4-benzothiadiazin-3-yl)-4-hydroxy-6-methyl-2(1H)-pyridinone;
3-(1,1-dioxido-4H-1,2,4-benzothiadiazin-3-yl)-1-(2-ethylbutyl)-4-hydroxy-6-methyl-5-phenyl-2(1H)-pyridinone;
6-(1,1-dioxido-4H-1,2,4-benzothiadiazin-3-yl)-7-hydroxy-4-pentylthieno[3,2-b]pyridin-5(4H)-one;
5-(1,1-dioxido-4H-1,2,4-benzothiadiazin-3-yl)-4-hydroxy-3-methyl-7-(3-methylbutyl)thieno[2,3-b]pyridin-6(7H)-one;
6-(1,1-dioxido-4H-1,2,4-benzothiadiazin-3-yl)-7-hydroxy-4-(4-methylpentyl)thieno[3,2-b]pyridin-5(4H)-one;
4-(3-butenyl)-6-(1,1-dioxido-4H-1,2,4-benzothiadiazin-3-yl)-7-hydroxythieno[3,2-b]pyridin-5(4H)-one;
7-benzyl-5-(1,1-dioxido-4H-1,2,4-benzothiadiazin-3-yl)-4-hydroxy-1-phenyl-1,7-dihydro-6H-pyrazolo[3,4-b]pyridin-6-one;
4-benzyl-6-(1,1-dioxido-4H-1,2,4-benzothiadiazin-3-yl)-2,7-dihydroxy[1,3]thiazolo[4,5-b]pyridin-5(4H)-one;
4-[(2-chloro-1,3-thiazol-5-yl)methyl]-6-(1,1-dioxido-4H-1,2,4-benzothiadiazin-3-yl)-7-hydroxythieno[3,2-b]pyridin-5(4H)-one;
6-(1,1-dioxido-4H-1,2,4-benzothiadiazin-3-yl)-7-hydroxy-4-[(5-methyl-3-pyridinyl)methyl]thieno[3,2-b]pyridin-5(4H)-one;
6-(1,1-dioxido-4H-1,2,4-benzothiadiazin-3-yl)-7-hydroxy-4-[(2-methyl-1,3-thiazol-5-yl)methyl]thieno[3,2-b]pyridin-5(4H)-one;
4-[(5-chloro-2-thienyl)methyl]-6-(1,1-dioxido-4H-1,2,4-benzothiadiazin-3-yl)-7-hydroxythieno[3,2-b]pyridin-5(4H)-one;
6-(1,1-dioxido-4H-1,2,4-benzothiadiazin-3-yl)-7-hydroxy-4-[(2-methyl-1,3-thiazol-4-yl)methyl]thieno[3,2-b]pyridin-5(4H)-one;
4-benzyl-6-(1,1-dioxido-4H-1,2,4-benzothiadiazin-3-yl)-7-hydroxy-2-(methylsulfanyl)[1,3]thiazolo[4,5-b]pyridin-5(4H)-one;
4-benzyl-6-(1,1-dioxido-4H-1,2,4-benzothiadiazin-3-yl)-7-hydroxy-2-(methylsulfonyl)[1,3]thiazolo[4,5-b]pyridin-5(4H)-one;
2-amino-4-benzyl-6-(1,1-dioxido-4H-1,2,4-benzothiadiazin-3-yl)-7-hydroxy[1,3]thiazolo[4,5-b]pyridin-5(4H)-one;
4-benzyl-6-(1,1-dioxido-4H-1,2,4-benzothiadiazin-3-yl)-7-hydroxy[1,3]thiazolo[4,5-b]pyridin-5(4H)-one;
3-(1,1-dioxido-4H-1,2,4-benzothiadiazin-3-yl)-4-hydroxy-1-(2-phenylpropyl)-1,8-naphthyridin-2(1H)-one;
8-benzyl-6-(1,1-dioxido-4H-1,2,4-benzothiadiazin-3-yl)-5-hydroxy-2-(methylsulfanyl)pyrido[2,3-d]pyrimidin-7(8H)-one;
8-benzyl-6-(1,1-dioxido-4H-1,2,4-benzothiadiazin-3-yl)-5-hydroxypyrido[2,3-d]pyrimidin-7(8H)-one;
3-(11,1-dioxido-4H-1,2,4-benzothiadiazin-3-yl)-4-hydroxy-1-(3-hydroxybutyl)-2(1H)-quinolinone;
1-benzyl-3-(1,1-dioxido-4H-1,2,4-benzothiadiazin-3-yl)-4-hydroxythieno[3,4-b]pyridin-2(1H)-one;
4-[(5-bromo-2-thienyl)methyl]-6-(1,1-dioxido-4H-1,2,4-benzothiadiazin-3-yl)-7-hydroxythieno[3,2-b]pyridin-5(4H)-one;
1-butyl-3-(1,1-dioxido-4H-1,2,4-benzothiadiazin-3-yl)-2(1H)-pyridinone;
3-(1,1-dioxido-4H-1,2,4-benzothiadiazin-3-yl)-1,8-naphthyridine-2,4-diol;
1-(benzyloxy)-3-(1,1-dioxido-4H-1,2,4-benzothiadiazin-3-yl)-4-hydroxy-1,8-naphthyridin-2(1H)-one;
3-(1,1-dioxido-4H-1,2,4-benzothiadiazin-3-yl)-4-hydroxy-1-isobutoxy-1,8-naphthyridin-2(1H)-one;
1-butyl-3-(1,1-dioxido-4H-1,2,4-benzothiadiazin-3-yl)-4-hydroxy-1,5-naphthyridin-2(1H)-one;
1-benzyl-3-(1,1-dioxido-4H-1,2,4-benzothiadiazin-3-yl)-4-hydroxy-1,5-naphthyridin-2(1H)-one;
1-benzyl-4-chloro-3-(1,1-dioxido-4H-1,2,4-benzothiadiazin-3-yl)-1,8-naphthyridin-2(1H)-one;
3-(1,1-dioxido-4H-1,2,4-benzothiadiazin-3-yl)-4-hydroxy-1-{[(1E)-phenylmethylene]amino}-2(1H)-quinolinone;
1-amino-3-(1,1-dioxido-4H-1,2,4-benzothiadiazin-3-yl)-4-hydroxy-2(1H)-quinolinone;
3-(1,1-dioxido-4H-1,2,4-benzothiadiazin-3-yl)-4-hydroxy-1-(2-phenylethyl)-1,8-naphthyridin-2(1H)-one;
1-butyl-4-chloro-3-(1,1-dioxido-4H-1,2,4-benzothiadiazin-3-yl)-1,8-naphthyridin-2(1H)-one;
4-amino-1-butyl-3-(1,1-dioxido-4H-1,2,4-benzothiadiazin-3-yl)-1,8-naphthyridin-2(1H)-one;
1-butyl-3-(1,1-dioxido-4H-1,2,4-benzothiadiazin-3-yl)-4-(methylamino)-1,8-naphthyridin-2(1H)-one;
1-butyl-4-(dimethylamino)-3-(1,1-dioxido-4H-1,2,4-benzothiadiazin-3-yl)-1,8-naphthyridin-2(1H)-one;
1-butyl-3-(1,1-dioxido-4H-1,2,4-benzothiadiazin-3-yl)-4-hydrazino-1,8-naphthyridin-2(1H)-one;
4-azido-1-butyl-3-(1,1-dioxido-4H-1,2,4-benzothiadiazin-3-yl)-1,8-naphthyridin-2(1H)-one;
1-butyl-3-(1,1-dioxido-4H-1,2,4-benzothiadiazin-3-yl)-4-[(2-hydroxyethyl)amino]-1,8-naphthyridin-2(1H)-one;
3-(1,1-dioxido-4H-1,2,4-benzothiadiazin-3-yl)-4-hydroxy-1-propoxyquinolin-2(1H)-one;
7-benzyl-5-(1,1-dioxido-4H-1,2,4-benzothiadiazin-3-yl)-4-hydroxy-3-(hydroxymethyl)-7,7a-dihydrothieno[2,3-b]pyridin-6(3aH)-one;
1-benzyl-3-(6-chloro-1,1-dioxido-4H-thieno[3,2-e][1,2,4]thiadiazin-3-yl)-4-hydroxy-1,8-naphthyridin-2(1H)-one;
1-benzyl-3-(6-chloro-1,1-dioxido-4H-thieno[3,2-e][1,2,4]thiadiazin-3-yl)-4-hydroxyquinolin-2(1H)-one;
3-[5-(aminomethyl)-1,1-dioxido-4H-1,2,4-benzothiadiazin-3-yl]-1-benzyl-4-hydroxy-1,8-naphthyridin-2(1H)-one;
8-benzyl-3-chloro-6-(1,1-dioxido-4H-1,2,4-benzothiadiazin-3-yl)-5-hydroxypyrido[2,3-c]pyridazin-7(8H)-one;
8-benzyl-6-(1,1-dioxido-4H-1,2,4-benzothiadiazin-3-yl)-5-hydroxy-3-(methylthio)pyrido[2,3-c]pynridazin-7(8H)-one;
8-benzyl-6-(1,1-dioxido-4H-1,2,4-benzothiadiazin-3-yl)-5-hydroxypyrido[2,3-c]pyridazin-7(8H)-one;
1-benzyl-3-(1,1-dioxido-4H-1,2,4-benzothiadiazin-3-yl)-4-hydroxy-1,6-naphthyridin-2(1H)-one;
1-benzyl-3-(1,1-dioxido-4H-1,2,4-benzothiadiazin-3-yl)-4-hydroxy-1,7-naphthyridin-2(1H)-one; and
1-(benzylamino)-3-(1,1-dioxido-4H-1,2,4-benzothiadiazin-3-yl)-4-hydroxyquinolin-2(1H)-one,
or pharmaceutically acceptable salts therof.

11. A pharmaceutical composition comprising a compound of claim 1 or a pharmaceutically acceptable salt thereof, in combination with a pharmaceutically acceptable carrier.

12. A method of treating or preventing infection which comprises administering to a patient in need of such treatment a therapeuctially effective amount of a compound of claim 1 or a pharmaceutically acceptable salt thereof.

13. The method of claim 12 wherein the infection is hepatitis C virus.

14. A method of inhibiting HCV polymerase comprises administering to a patient in need of such treatment a therapeuctially effective amount of a compound of claim 1 or a pharmaceutically acceptable salt thereof.


SEQUENCE LISTING (1 sequence)

SEQ ID NO: 1 - RNA, 171 nt, Hepatitis C Virus
gggcgaauugggcccucuagaugcaugcucgagcggccgccagugugauggauaucugcagaauucgcccuugguggcuccaucuuagcccuagucacggcuagcugugaaagguccgugagccgcuugacugcagagagugcugauacuggccucucugcagaucaaguc